(19) United States
(12) Patent Application Publication
(10) Pub. No.: US 2011/0097395 A1
(43) Pub. Date: Apr. 28, 2011

(54) Oral Pharmaceutical Compositions of Buprenorphine and Method of Use

(76) Inventors: Najib Babul, Blue Bell, PA (US); Ashish Kumar Rehni, Patiala (IN)

(21) Appl. No.: 12/988,209

(22) PCT Filed: Mar. 9, 2009
(86) PCT No.: PCT/US09/01502

§ 371 Date: Oct. 15, 2010

Related U.S. Application Data
(60) Provisional application No. 61/064,505, filed on Mar. 8, 2008.

(51) Int. Cl. A61K 31/485; A61K 9/00; A61K 9/48; A61K 9/14; A61P 25/36; A61P 25/30; A61P 13/00; A61P 31/22

(52) U.S. Cl. 424/451; 514/282; 424/400; 424/490

(57) ABSTRACT

The present invention is directed to oral pharmaceutical compositions of buprenorphine and it pharmaceutically acceptable salts and the use thereof.

This application claims the benefit of U.S. Provisional Application No. 61/064,505, filed Mar. 8, 2008, which is herein incorporated by reference in its entirety.

### FIELD OF THE INVENTION

The present invention is directed to oral pharmaceutical compositions of buprenorphine and it pharmaceutically acceptable salts and the use thereof.

### BACKGROUND TO THE INVENTION

Currently, medical practitioners may choose from several well-accepted classes of pharmaceutical agents in their attempts to alleviate and prevent pain. Nonlimiting examples of agents used include nonsteroidal anti-inflammatory agents (NSAIDs), e.g., aspirin, ibuprofen, ketoprofen, diclofenac; opioids, e.g., morphine, hydromorphone, hydrocodone, oxycodone, tramadol, and codeine; cyclooxygenase-2 (COX-2) selective NSAIDs, e.g., celecoxib, valdecoxib, etoricoxib, lumiracoxib, and rofecoxib; acetaminophen; tricyclic antidepressants, e.g., amitriptyline, desipramine, nortriptyline; non-tricyclic antidepressants, e.g., doxepin, duloxetine, paroxetine, venlafaxine; antiepileptics, e.g., gabapentin, pregabalin, carbamazepine, oxcarbazepine, lamotrigine; voltage sensitive N-type calcium channel blockers, e.g., ziconotide and alpha adrenergic agonists, e.g., clonidine.

Of the many challenges that occur when pharmacologically treating any disease or pathological condition, including pain, alleviating the symptoms without causing counterproductive side effects is often the greatest. This challenge presents itself when medical practitioners use medicinal agents to treat pain. Although the aforementioned pharmacological classes are frequently effective for the treatment of certain types of pain, the chronic and acute use of these analgesic agents produces a number of significant, undesirable side effects.

Morphine is extensively utilized for the management of severe pain due to its global availability, extensive clinical experience, significant pharmacokinetic and pharmacodynamic data, low cost and the availability of various extended release formulations (Babul et. al, J Clin Pharmacol, 1998; 38:74-81). However, the incidence and severity of side effects limits the use of morphine in some patients (Hagen and Babul, Cancer 1997; 79:1428-37). In patients with renal impairment, morphine's principal metabolites, morphine-3-glucuronide and morphine-6-glucuronide can accumulate. Morphine-3-glucuronide accumulation has been implicated in hyperalgesia, respiratory stimulation, and behavioral excitatory properties through nonopioid receptor mechanisms. Morphine-6-glucuronide accumulation has been implicated in increasing levels of nausea and sedation in patients with renal impairment (Babul and Darke, Clin Pharm Ther, 1993; 54:286-92). Similarly, the principal metabolite of hydromorphone, hydromorphone-3-glucuronide can accumulate in patients with renal impairment and has been found to be more neurotoxic than morphine-3-glucuronide (Babul and Darke, Pain, 1992; 51:260-61; Hagen et al., J Clin Pharmacol, 1995; 35:38-45; Babul et al., J Pain Symptom Manage, 1995; 10:184-86; Wright et al., Life Sci, 1998; 63:401-11; Wright et al., Life Sci, 2001; 69:409-20.).

An important goal of analgesic therapy is to achieve continuous relief of pain. Regular administration of an analgesic is generally required to ensure that the next dose is given before the effects of the previous dose have worn off. (Principles of Analgesic Use in the Treatment of Acute Pain and Cancer Pain, Fifth Ed., American, Pain Society (2003); Evidence Based Report of the U.S. Agency for Healthcare Research and Quality (AHRQ) on the Management of Cancer Pain, Report No. 35, AHRQ Publication No. 02-E002, October 2001; Can et al. J Nat Cancer Inst Monograph 2004; 32:23-31; Agency for Health Care Policy and Research Clinical Practice Guidelines for Cancer Pain Management, Guideline No. 9, AHCPR Publication No. 94-0592, March 1994; Agency for Health Care Policy and Research Clinical Practice Guideline for Acute Pain Management, Guideline No. 1, AHCPR Publication No. 92-0032, February, 1992; Guideline for the Management of Cancer Pain in Adults, American Pain Society, 2005; Guideline for the Management of Pain in Osteoarthritis, Rheumatoid Arthritis, and Juvenile Chronic Arthritis, 2^{nd} Ed., American Pain Society, 2002).

Conventional (so called “immediate-release”, “rapid release” or “short acting”) opioid analgesics have been demonstrated to provide short-lived plasma levels, thereby requiring dosing every 4-6 hours in chronic pain. In contrast, extended release oral opioids are designed to maintain effective plasma levels throughout a 12 or 24-hour dosing interval. Use of extended release opioids can result in fewer interruptions in sleep, reduced dependence on caregivers, improved compliance, enhanced quality of life outcomes, and increased control over the management of their pain. In addition, such formulations can provide more constant plasma concentrations and clinical effects, less frequent peak to trough fluctuations and fewer side effects, compared with short acting opioids (Babul et al. Journal of Pain and Symptom Management 2004; 28:59-71; Matsumoto et al., Pain Medicine 2005; 6:357-66; Dhaliwal et al., Journal of Pain Symptom Management 1995; 10:612-23; Hays et al., Cancer 1994; 74:1808-16; Arkinstall et al., Pain 1995; 62:169-78; Hagen et al., Journal of Clinical Pharmacology 1995; 35:38-45; Peloso et al., Journal of Rheumatology 2000; 27:764-71).

The introduction of extended release morphine (MS Contin™) revolutionized the management of cancer pain. MS Contin™ gained wide-spread acceptance due to its global availability, significant pharmacokinetic and pharmacodynamic data, and the convenience of a extended-release formulation. However, the incidence and severity of side effects limits the use of morphine in some patients (Hagen and Babul, Cancer 1997; 79:1428-37).

Clinicians treating cancer pain with opioids have reported significant variability among patients in efficacy and side effects with available opioid analgesics. Patients with poor analgesic efficacy or safety outcomes on one opioid frequently tolerate another opioid well. This clinical observation led to the development of oxycodone ER (OxyContin™). Due to the limitations associated with extended release morphine noted above and the “stigma” associated with its use (i.e., association with addiction, advanced cancer, dying and death), extended release oxycodone gained rapid acceptance by patients with chronic non-cancer pain. However, its widespread use for the treatment of chronic non-malignant pain was also associated with its diversion into the non-medical supply for use both by addicts and recreational drug users

Among the many side effects of opioids are nausea, vomiting, constipation, sedation, fatigue, pruritus, blurred vision, urinary retention, respiratory depression, convulsions, mood changes and alterations of the endocrine and autonomic nervous systems. Many of these side effects are sufficiently bothersome as to require: i) use of additional medications to treat the iatrogenic symptoms; ii) more intensive patient management; iii) use of lower doses that leave patients in continued pain; or iv) in other cases, complete discontinuation of analgesic therapy. Opioids can also produce potentially fatal respiratory depression at high doses. (Evidence Based Report of the U.S. Agency for Healthcare Research and Quality (AHRQ) on the Management of Cancer Pain, Report No. 35, AHRQ Publication No. 02-E002, October 2001; Carr et al. J Nat Cancer Inst Monograph 2004; 32:23-31; Agency for Health Care Policy and Research Clinical Practice Guidelines for Cancer Pain Management, Guideline No. 9, AHCPR Publication No. 94-0592, March 1994; Guideline for the Management of Cancer Pain in Adults, American Pain Society, 2005).

The Evidence Based Report of the U.S. Agency for Healthcare Research and Quality (AHRQ) on the Management of Cancer Pain (Report No. 35, AHRQ Publication No. 02-E002, October 2001; Can et al. J Nat Cancer Inst Monograph 2004; 32:23-31) supports the three tier approach. “The first tier, for mild to moderate pain, consists of NSAIDs and acetaminophen with or without adjuvant medications. As pain escalates or persists, treatment progresses to the second tier, in which a weak opioid, such as codeine or hydrocodone, is added to the NSAID with or without an adjuvant drug.” “If pain still persists, treatment progresses to the third tier: substitution of the “weak” opioid for a “strong” opioid (i.e., one more readily titrated to doses with greater analgesic efficacy). The latter category includes morphine, hydromorphone, methadone, fentanyl, and levorphanol, all full opioid agonists at the morphine or mu receptor.” The AHRQ report further notes that “if pain relief is not achieved at the maximum recommended dose of a particular NSAID or opioid, it should be discontinued and another drug from the same class tried before abandoning that class. Case series indicate that on an individual basis, other drugs from the same class may prove more effective or be better tolerated.”

The Agency for Health Care Policy and Research Clinical Practice Guidelines for Cancer Pain Management (Guideline No. 9, AHCPR Publication No. 94-0592, March 1994) states: “It is usually advisable to observe the patient's response to several different opioids, sequentially, before switching routes of administration or trying an anesthetic, neurosurgical, or other invasive approach to relieve persistent pain. For example, patients who experience dose-limiting sedation, nausea, or mental clouding on oral morphine should be switched to an equianalgesic dose of hydromorphone or fentanyl. The dose of the second opioid should then be adjusted. Sequential analgesic trials should be based on regular assessments of pain, with continuous attention to antineoplastic and noninvasive nonpharmacologic therapies”.

According to the recently issued American Pain Society Guideline for the Management of Cancer Pain in Adults (2005, pp. 59-60), “For patients who experience inadequate pain relief, or an unacceptable level of side effects from a specific opioid that limits dose escalation, pain control can be achieved through opioid rotations. For example, patients who experience dose-limiting sedation, nausea, or mental clouding from oral morphine can be switched to an equianalgesic dose of hydromorphone or fentanyl. Dosage of the second opioid should then be adjusted so that relief is achieved with an acceptable level of side effects. It is usually advisable to observe a patient's response to several different opioids, administered sequentially, before switching routes of administration or trying an anesthetic, neurosurgical, or other invasive approach to relieve persistent cancer pain”.

A number of oral immediate release oral formulations of opioid analgesics have been described in the art, including codeine, hydrocodone, hydromorphone, isomethadone, levorphanol, meperidine, methadone, morphine, oxycodone, oxymorphone, pentazocine, propoxyphene, tapentadol, tramadol, or their pharmaceutically acceptable salts.

A number of oral extended release formulations of opioid analgesics have been developed or commercialized, including morphine, hydromorphone, oxycodone, hydrocodone and oxymorphone.

No oral extended release formulations of opioid analgesics have been developed or commercialized, i

Buprenorphine or [5α,7α(S)]-17-(Cyclopropylmethyl)-α-(1,1-dimethylethyl)-4,5-epoxy-18,19-dihydro-3-hydroxy-6-methoxy-α-methyl-6,14-ethenomorphinan-7-methanol or it pharmaceutically acceptable salts have been used for the treatment of a variety of medical conditions, including pain and opioid addiction disorders. It is a semi-synthetic opioid first derived in 1966 from thebaine, an alkaloid from the poppy Papaver somniferum.

Buprenorphine is a partial agonist at the mu (μ)-opioid receptor. To our knowledge, no oral extended release formulations of buprenorphine or other partial mu (μ)-opioid opioid analgesics have been developed or commercialized.

Buprenorphine has been commercially available as a parenteral formulation for intravenous and intramuscular use. According to the FDA's Orange Book, parenteral buprenorphine was approved prior to Jan. 1, 1982. Buprenorphine has been widely reported to have very poor oral bioavailability and is believed to be ineffective when given orally. For this reason, pharmaceutical companies have made elaborate efforts to develop alternative non-invasive methods of delivering buprenorphine into systemic circulation. Foremost among these methods is the sublingual delivery of buprenorphine for the treatment of pain. This approach has provided only modest efficacy and has been a commercially failure in a number of countries.

On Oct. 8, 2002, two new sublingual formulations of buprenorphine were approved by the FDA in the USA for the “treatment of opioid dependence” but not for pain. One formulation contains buprenorphine alone (Subutex™) and the other contains buprenorphine in combination with naloxone (Suboxone™). According to the FDA approved U.S. prescribing information, “Suboxone™ and Subutex™ tablets should be placed under the tongue until they are dissolved. For doses requiring the use of more than two tablets, patients are advised to either place all the tablets at once or alternatively (if they cannot fit in more than two tablets comfortably) place two tablets at a time under the tongue. Either way, the patients should continue to hold the tablets under the tongue until they dissolve; swallowing the tablets reduces the bioavailability of the drug. To ensure consistency in bioavailability, patients should follow the same manner of dosing with continued use of the product.” (accessed on Jan. 22, 2008; http://www.fda.gov/cder/foi/label/2002/20732lbl.pdf.)

According to the manufacturer's patient instructions approved by the FDA on how to take Suboxone™ or Subutex™, “(i) Put the tablets under your tongue and let them melt. This will take 2 to 10 minutes. Do not chew or swallow the tablets. The medicine will not work this way and you may get withdrawal symptoms; (ii) If your doctor tells you to take more than 1 tablet, you will be told to: (a) take all tablets at the same time together under your tongue, or take 2 tablets, put them under your tongue. After they melt, put the next tablet or tablets under your tongue right away; (b) hold the tablets under your tongue until they melt completely. The medicine will not work if swallowed and you may get withdrawal symptoms. (c) Do not change the way you are told to take your medicine or you may get too little or too much medicine.” (Accessed Jan. 22, 2008, http://www.fda.gov/cder/foi/label/2002/207321 ppi.pdf).

Major disadvantages with sublingual administration of buprenorphine include but are not limited to: (i) highly variable pharmacokinetics and pharmacodynamics; (ii) variability of patient's ability to adhere to the instructions about oral retention of drug; (iii) the development of a depot of buprenorphine on in the oral tissue; (iv) an unpleasant taste and after-taste; (v) a sensation of “gagging”; (vi) durability of a robust effect over the course of 24 hours; and (vii) increased risk of drug abuse through tampering of the dosage form and subsequent intravenous, intranasal and inhalational use.

It is widely recognized that the dose of opioids required to treat pain is highly variable. For example, some patients can be managed with a few mg of morphine while other patients require grams of morphine per day (Babul and Hagen, Proceedings of the American Society for Clinical Pharmacology and Therapeutics, Mar. 24-27, 2002, Atlanta, Ga.). For this reason, patients receiving opioids are titrated to clinical effect by increasing the dose. An increase in dose should provide a dose proportional increase in bioavailability. Unfortunately, sublingual buprenorphine fails to provide dose proportional bioavailability at particularly high doses, thereby limiting its clinical utility. For example, a 16 mg sublingual dose of buprenorphine provides only 70% dose normalized bioavailability of a 4 mg sublingual dose.

Another limitation with the sublingual route is the high peak concentration of buprenorphine. Both peak concentrations of opioids and the rate of rise in concentrations have variously been implicated in the causation of various opioid side effects such as nausea, drowsiness, dizziness and (acute) cognitive impairment. Orally administered buprenorphine can be formulated to provide significantly lower peak concentrations than sublingual buprenorphine. Such an effect is also desirable in opioid dependent individuals to minimize the “rush” or euphoric effect of high peak concentrations.

Oral formulations of buprenorphine, with their differential opioid receptor binding and clinical effects have the potential to be a drug of choice in opioid rotation regimens in patients with suboptimal efficacy and/or safety on other orally available opioids (e.g., oxycodone, morphine, oxymorphone, hydrocodone), thereby providing an effective strategic intervention for such patients (Hagen and Babul, Cancer 1997; 79:1428-37).

Other efforts to provide alternative non-invasive methods of delivering buprenorphine into systemic circulation have included intranasal (see, for example, Linhardt et al, Int J Pharm, 2000; 205 (1-2):159-63) and transdermal administration (see, for example, U.S. Pat. Nos. 7,270,830, 6,344,212, 6,004,969, 5,968,547, 5,240,711, and 5,069,909).

Two transdermal formulations of buprenorphine have been commercialized in some countries, including the U.K., for the treatment of pain. One formulation is designed for application to the skin about twice a week (Transtec™) and another formulation is designed for administration once-a-week (BuTrans™). Major disadvantages with transdermal administration of buprenorphine include but are not limited to: (i) cost of goods; (ii) wide inter- and intra-individual variability if rate and extent of absorption; (iii) delay with onset of clinically meaningful therapeutic effect; (iv) poor skin adhesion over sweaty and hairy skin and in tropical climatic zones; (v) cutaneous adverse reactions to buprenorphine and/or its adhesives and other excipients; (vi) reduce flexibility in dose titration in relation to changing clinical status; (vii) the potential for variable absorption in the presence of pronounced fever; (viii) formation of a skin depot of buprenorphine, despite removal of the patch; and (ix) poor adhesion during vigorous physical activity, bathing or water sports.

Other important disadvantages with transdermal administration of buprenorphine include but are not limited to: (i) a delayed and highly variable time to minimum effective plasma concentrations; (ii) a delayed and highly variable time to maximum plasma concentrations; (iii) a delayed time to steady state concentration; (iv) a wide peak to trough plasma concentration fluctuation; (v) a wide variability in peak concentration (Cₘₐₓ); (vi) a wide variability in extent of absorption (AUC_{0-τ}); and (vii) inconsistent delivery over the dosing interval.

Clinical studies of transdermal buprenorphine have shown local skin reactions in approximately one-third of patients. It is believed that a majority of these reactions are due to the patch material or adhesive, since such reactions have been seen in both active and placebo groups in clinical trials. Pruritus and erythema are the most common local reactions and they are usually of mile to moderate intensity.

The development of reliable transdermal formulations is challenging as such formulations must provide consistent delivery of drug over a prolonged period of time when applied under variable conditions (e.g., skin sites of varying adiposities, differing degrees of sweating, changes in temperature with physical activity and fever, and presence of moisture when bathing). Additionally, such formulations need to provide reliable skin adhesion or adhesivity while being painless to remove at the end of the dosing interval.

At the completion of treatment with a transdermal dosage form of buprenorphine, a considerable amount of residual buprenorphine remains in the dosage form. This can readily be extracted and diverted into the illicit drug market.

Addicts and recreational drug also abuse of transdermal opioids through tampering and extraction of drug for subsequent oral ingestion, snorting, inhalation or intravenous injection. Such tampering has been known to include extraction of the active substance from the transdermal reservoir or matrix by needle aspiration, oral ingestion of the active substance from the transdermal system, and solvent extraction of the active substance from the transdermal system.

Transdermal buprenorphine dosage forms are not yet available in the USA; therefore it is difficult to fully predict their likely patterns of abuse. In contrast, transdermal forms of the opioid fentanyl have been widely available in the USA for a considerable period of time. Transdermal fentanyl has been an important addition to the therapeutic armamentarium for the treatment of pain. However, it continues to be abused and misused for its addiction potential (Liapas et al., J Psychopharmacol, 2004; Tharp et al., Am J Forensic Med Pathol, 2004; Jost et al., Dtsch Med Wochenschr, 2004; Milligan et al., J Pain, 2001; Kuhlman et al., J Anal Toxicol, 2003; Arvanitis and Satonik, et al., Am J Emerg Med, 2002; Marquardt et al., Ann Pharmacother, 1995; Poklis, J Toxicol Clin Toxicol, 1995; De Sio et al., Anesthesiology, 1993; Lilleng et al., J Forensic Sci, 2004; Kuhlman et al, J Anal Toxicol, 2003; Reeves and Ginifer, Med J Aust, 2003; Pizon and Brooks, Vet Hum Toxicol, 2004; Nevin, Emerg Med Serv, 2004; Barrueto, Vet Hum Toxicol, 2004; Marquardt and Tharratt, Toxicol Clin Toxicol, 1994).

The abuse of fentanyl patch has involved a variety of methods including steeping the patch in hot water (“fentanyl tea bag”); inhalation of patch contents; solvent extraction, followed by intravenous use; needle aspiration, followed by intravenous use; mechanical extraction, followed by intravenous use; solvent extraction, followed by oral, mL; nasal and inhalation use; mechanical extraction, followed by oral, mL; nasal and inhalation use; transdermal application of the contents of the tampered patch; combustion of the patch, followed by inhalation; and a combination of the above methods.

Underscoring its concerns about drug abuse, recently, the U.S. Food and Drug Administration made a precedential decision rejecting the approval of a bioequivalent (“A/B generic”) patch of transdermal fentanyl, solely of the basis of the amount of fentanyl in the patch, rather than the amount of fentanyl absorbed into systemic circulation compared to the innovator (Duragesic™).

On Jul. 8, 2005, the FDA posted a Safety Alert and on Jul. 15, 2005, they issued a Public Health Advisory about the deaths resulting from use of the transdermal fentanyl patch. At that time, there were 120 deaths reported and the FDA was investigating the deaths to determine if they were due to inappropriate use or defective patches. According to the FDA, the deaths spanned the time period beginning in 1990 when the drug was introduced and stemmed from legitimate use rather than abuse of the medication. FDA noted that it was “investigating reports of death and other serious adverse events related to narcotic overdose in patients using the fentanyl transdermal patch for pain control.” FDA further noted that it “recently conducted a review of fatalities reported to the voluntary adverse event reporting system that were possibly due to unintentional overdose from the fentanyl transdermal patch. In many cases, establishing whether the overdose was unintentional was difficult, because the information provided in the report was incomplete and patients who were being treated with the fentanyl patch often had underlying diseases or conditions that could have contributed to their deaths (such as cancer). Factors identified as possibly related to unintentional overdose included: use of high doses of the fentanyl patch and/or multiple patches (sometimes in combination with other drugs), possible medication errors, accidental exposure (e.g., coming in contact with a discarded patch), application of a heat source to the patch possibly resulting in increased fentanyl absorption, injection or ingestion of the patch contents, and suspected transdermal patch malfunction (e.g., leaking patches). In addition, several patients reported poor adhesion of the patches to the skin.”

In 2005, the Los Angeles Times reported that the Los Angeles County Coroner's office has investigated more than 230 deaths involving fentanyl (FDA Slow to Sound Alarm on Pain Drug Fentanyl's overdose rate and side effects show holes in monitoring medicine on the market, Nov. 25, 2005, Page A1). Of those 230-plus cases, 127 were classified as “accidental deaths,” suggesting that the victims had inadvertently received too much of the drug through the pain patch. According to an report in the LA Times, 115 additional fentanyl deaths were reported from the state of Florida in the year 2004, as well as an alarming rise in emergency-room cases involving the drug. According to the Federal Substance Abuse and Mental Health Services Administration (SAMHSA), emergency-room admissions involving fentanyl rose from 28 in 1998 to 1506 in 2004.

According to an FDA report introduced at a judicial proceeding, between April 2003 and June 2004, a total of 2.5 million Duragesic™ patches were recalled due to customer reports of leakage. There have been numerous additional recalls of transdermal fentanyl patches by multiple manufacturers.

Thus there is a need for optimized alternative opioid formulations for the treatment of acute and chronic pain. There is also a need for optimized opioid formulations for the treatment of cancer pain and neuropathic pain. There is also a need for alternative opioid formulations for the treatment of opioid and buprenorphine-responsive conditions other than pain.

There is a need for optimized formulations of non-transdermal opioids that provide reduced pharmacokinetic and pharmacodynamic variability, a rapid onset of effect, a sustained duration of effect, consistent effects over time, improved safety and tolerability. There is also a need for optimized pharmaceutical formulations of oral opioids that deliver the drug into systemic circulation more efficiently and with improved patient and prescriber acceptance.

There is a need for oral (i.e., orally ingested, as opposed to lingual, sublingual or buccal) formulations of buprenorphine that are therapeutically effective for the treatment of various medical conditions, including but not limited to pain and opioid addiction disorders.

There is a need for oral formulations of buprenorphine that are therapeutically efficient and that can provide immediate release and/or sustained release of the buprenorphine.

The applicant has demonstrated that oral administration of buprenorphine can produce robust, dose dependent analgesia.

A variety of opioid analgesics have demonstrated their efficacy in acute pain, chronic cancer and non-cancer pain and in pain states.

The oral route of administration (i.e., oral ingestion) is the most widely used and most widely preferred method of drug administration. It is simple, reliable and readily accessible. Under most conditions of use, particularly outside the hospital setting, it is the recommended method of drug administration. Even in settings of skilled nursing care, where there are technical and human resources to initiate and manage parenteral therapy, the goal is to rapidly transition patients from parenteral medications to oral medications. Some generally cited exceptions to the use of the oral route include: (i) drugs with poor oral bioavailability; (ii) drugs requiring a rapid onset of effect; (iii) where venous access already exists (e.g., in the peri-operative or intensive care setting); (iii) where the oral route provides unreliable or inconsistent clinical effects. Oral administration of buprenorphine in immediate release form and in sustained release form has either not been practiced or has been dismissed as unreliable or clinically unacceptable for some of the reasons noted above.

Contrary to this view, the applicant asserts that orally administered buprenorphine can provide acceptable pharmacokinetics, pharmacodynamics, clinical efficacy and safety. Administration of buprenorphine by the oral route provides significantly greater flexibility in dosage form design, clinical utility and patient acceptability. In the USA, buprenorphine is classified as a Schedule III drug. According to the DEA and WHO, Schedule III drugs have lower potential for abuse than the drugs in Schedules I and II (e.g., fentanyl, codeine, hydrocodone, hydromorphone, methadone, meperidine, morphine, oxycodone, and oxymorphone). In addition, since sustained release drugs are the standard of care for the management of many chronic conditions and sustained release opioids are the standard of care for the management of chronic pain, an orally effective buprenorphine, with its reduced abuse potential compared with Schedule II opioids, has the potential to provide fewer interruptions in sleep, reduced dependence on caregivers, improved compliance, enhanced quality of life outcomes, and increased control over the management of their pain. In addition, such formulations can provide more constant plasma concentrations and clinical effects, less frequent peak to trough fluctuations and fewer side effects, compared with sublingual buprenorphine. Similarly, for the treatment of opioid addiction disorders (where sublingual buprenorphine is approved), oral sustained release buprenorphine can provide similar attributes as seen in patients with pain and has the potential to become the standard of care. When compared with sublingual administration, oral immediate and sustained release buprenorphine may be associated with reduced peak to trough fluctuation in concentrations and clinical effects, such as drug craving. Furthermore, in many cases, such dosage forms have a reduced potential for abuse and diversion than sublingual formulations of buprenorphine which are designed to rapidly dissolve in the oral cavity, thereby reducing subsequent intravenous, intranasal and inhalational abuse.

To the applicants knowledge, no examples or working prototype formulations of any oral buprenorphine have been described in the prior art. Buprenorphine is a challenging drug to develop orally due to it's high intrinsic clearance and the potential for substantial pharmacokinetic variability.

To the applicant's knowledge, there are no data demonstrating the efficacy of any oral pharmaceutical compositions of buprenorphine for acute pain, chronic pain, cancer pain and neuropathic pain.

To the applicant's knowledge, there are no data demonstrating the efficacy of any oral immediate release pharmaceutical compositions of buprenorphine for the treatment of pain, opioid dependence, addiction disorders and buprenorphine or opioid responsive medical conditions.

To the applicant's knowledge, there are no data demonstrating the efficacy of any oral sustained release pharmaceutical compositions of buprenorphine for the treatment of pain, opioid dependence, addiction disorders and buprenorphine or opioid responsive medical conditions.

There is a need for new pharmaceutical compositions and methods for the treatment of pain, opioid dependence, addiction disorders and buprenorphine or opioid responsive medical conditions.

There is a need for new oral opioid pharmaceutical compositions and methods for the treatment of pain, opioid dependence, addiction disorders and buprenorphine or opioid responsive medical conditions have high efficacy, good tolerability, are an alternative to the transdermal and sublingual/buccal routes, and provide consistent pharmacokinetics and pharmacodynamics.

### DESCRIPTION OF THE INVENTION

The present invention is directed at oral pharmaceutical compositions of buprenorphine and their use for the treatment of buprenorphine responsive medical conditions.

The present invention is directed at oral pharmaceutical composition for the treatment of pain, opioid dependence, addiction disorders and other buprenorphine and opioid responsive medical conditions comprising a therapeutically effective amount of buprenorphine or a pharmaceutically acceptable salt of buprenorphine, or a mixture thereof, said dosage form not intended to provide significant oro-mucosal, lingual, sublingual or buccal absorption, said dosage form not intended for oro-mucosal, lingual, sublingual or buccal administration.

The present invention relates to oral buprenorphine pharmaceutical compositions and methods for the treatment of pain.

The present invention relates to oral buprenorphine pharmaceutical compositions and methods for the treatment of addiction disorders.

The present invention relates to oral buprenorphine pharmaceutical compositions and methods for the treatment of conditions other than pain and addiction disorders amenable to treatment with buprenorphine or opioid analgesics.

It is an object of certain preferred embodiments of the present invention to provide oral extended release pharmaceutical compositions of buprenorphine that have greater bioavailability (AUC) than oral immediate release formulations.

It is an object of certain preferred embodiments of the present invention to provide oral immediate release and oral extended release pharmaceutical compositions of buprenorphine that provide a greater plasma AUC ratio of norbuprenorphine (an active metabolite of buprenorphine) to buprenorphine than is attained by sublingually administered dosage forms.

It is an object of certain preferred embodiments of the present invention to provide oral immediate release and oral extended release pharmaceutical compositions of buprenorphine that provide a greater plasma AUC ratio of norbuprenorphine to buprenorphine than is attained by transdermal dosage forms.

It is an object of certain preferred embodiments of the present invention to provide oral immediate release and oral extended release pharmaceutical compositions of buprenorphine that provide a greater plasma AUC ratio of norbuprenorphine to buprenorphine than is attained by intranasally administered dosage forms.

It is an object of certain preferred embodiments of the present invention to substantially improve the efficiency and quality of pain management in human patients experiencing moderate or severe pain.

It is an object of certain preferred embodiments of the present invention to substantially improve the efficiency and quality of pain management in human patients experiencing mild to moderate pain.

It is an object of certain preferred embodiments of the present invention to substantially improve the efficiency and quality of pain management in human patients experiencing moderate or severe pain.

It is an object of certain preferred embodiments of the present invention to provide bioavailable oral buprenorphine formulations suitable for up to once-daily administration which substantially improve the efficiency and quality of pain management.

It is an object of some embodiments of the invention to provide oral pharmaceutical compositions of buprenorphine and methods for the treatment of pain, addiction disorders and other buprenorphine responsive disorders, wherein the dosage form is administered at a prespecified dosing regimen. In some embodiments, said dosing regimen associated with reduced side effects, improved tolerability, improved efficiency of therapeutic response, reduced breakthrough symptoms (e.g., breakthrough pain) and reduced treatment discontinuation due to side effects.

It is an object of certain preferred embodiments of the present invention to substantially improve the efficiency and quality of addiction disorder management, particularly opioid addiction disorders and poly-substance abuse disorders in human patients.

It is an object of certain preferred embodiments of the present invention to treat pain and addiction disorders in patients who have a high risk of opioid abuse, continued opioid abuse and relapse from an illicit opioid free state.

It is an object of certain preferred embodiments of the present invention to treat pain and addiction disorders in patients who have a suboptimal efficacy or safety response to sublingual buprenorphine.

It is an object of certain preferred embodiments of the present invention to treat pain and addiction disorders in patients who have a suboptimal efficacy or safety response to transdermal buprenorphine.

It is an object of certain preferred embodiments of the present invention to treat pain and addiction disorders in patients who require a prolonged duration of effect that is provided by sublingual buprenorphine.

It is an object of certain preferred embodiments of the present invention to treat addiction disorders in patients who have a suboptimal efficacy or safety response to methadone.

It is an object of certain preferred embodiments of the present invention to treat pain in patients who have a suboptimal efficacy or safety response with other orally approved opioids (e.g., opioids described for oral administration in the FDA's Orange Book. Goodman & Gilman's The Pharmacological Basis of Therapeutics (Brunton, Lazo and Parker, eds, 11th ed., McGraw Hill (2005); Principles of Analgesic Use in the Treatment of Acute Pain and Cancer Pain, Fifth Ed., American, Pain Society (2003); Evidence Based Report of the U.S. Agency for Healthcare Research and Quality (AHRQ) on the Management of Cancer Pain, Report No. 35, AHRQ Publication No. 02-E002, October 2001; Can et al. J Nat Cancer Inst Monograph 2004; 32:23-31; Agency for Health Care Policy and Research Clinical Practice Guidelines for Cancer Pain Management, Guideline No. 9, AHCPR Publication No. 94-0592, March 1994; Agency for Health Care Policy and Research Clinical Practice Guideline for Acute Pain Management, Guideline No. 1, AHCPR Publication No. 92-0032, February, 1992; Guideline for the Management of Cancer Pain in Adults, American Pain Society, 2005; Guideline for the Management of Pain in Osteoarthritis, Rheumatoid Arthritis, and Juvenile Chronic Arthritis, 2^{nd} Ed., American Pain Society, 2002), e.g., morphine, codeine, oxycodone, oxymorphone, hydromorphone, methadone, hydrocodone).

It is an object of certain preferred embodiments of the present invention to treat pain in patients who have a suboptimal efficacy or safety response with other orally approved extended release opioids (e.g., MS Contin™, Kadian™, Avinza™, Ultram™ ER, Opana™ ER, Palladone™, Jurnista™)

There is a need for oral formulations of buprenorphine that are therapeutically effective for the treatment of various medical conditions, including but not limited to pain, cough, dyspnea and opioid addiction disorders.

To the applicant's knowledge, there are no data demonstrating the efficacy of any oral pharmaceutical compositions of buprenorphine for the treatment of any medical disorder including cancer pain, neuropathic pain, cough, dyspnea, restless leg syndrome, fibromyalgia, acute herpes zoster, visceral pain and breakthrough pain.

To the applicant's knowledge, there are no data demonstrating the efficacy of any oral extended release pharmaceutical compositions of buprenorphine for the treatment of any pain state, opioid dependence, addiction disorders and other buprenorphine responsive medical conditions.

In some embodiments, the present invention is directed at oral pharmaceutical composition for the treatment of pain, opioid dependence, addiction disorders, cough, dyspnea, restless leg syndrome and other buprenorphine responsive medical conditions comprising a therapeutically effective amount of buprenorphine or a pharmaceutically acceptable salt of buprenorphine, or a mixture thereof.

The present invention relates to oral buprenorphine pharmaceutical compositions and methods for the treatment of pain, including acute pain, chronic pain, cancer pain, neuropathic pain, cough, dyspnea, fibromyalgia, acute herpes zoster, visceral pain and breakthrough pain.

The present invention relates to oral buprenorphine pharmaceutical compositions and methods for the treatment of acute and chronic cough, including iatrogenic cough, post-infectious cough, cough secondary to asthma, COPD, lung cancer, gastroesophageal reflux disease, respiratory bacterial and viral infections, and upper airway cough syndrome.

The present invention relates to oral buprenorphine pharmaceutical compositions and methods for the treatment of urinary incontinence.

The present invention relates to oral buprenorphine pharmaceutical compositions and methods for the treatment of restless leg syndrome.

The present invention also relates to oral buprenorphine pharmaceutical compositions and methods for the treatment of addiction disorders.

The present invention relates to oral buprenorphine pharmaceutical compositions and methods for the treatment of conditions other than pain and addiction disorders amenable to treatment with buprenorphine.

It is an object of certain preferred embodiments of the present invention to substantially improve the efficiency and quality of pain management in human patients experiencing pain which is unresponsive or suboptimally responsive to opioids classified as Schedule II (C-II) opioids under the United States Controlled Substance Act and regulations (as amended).

It is an object of some preferred embodiments to provide an oral pharmaceutical composition comprising a therapeutically effective amount of buprenorphine which produces less moderate to severe sedation or drowsiness than after an equal amount or dose of commercially available immediate release sublingual formulation of buprenorphine listed in FDA's Orange Book.

It is an object of some preferred embodiments to provide an oral pharmaceutical composition comprising a therapeutically effective amount of buprenorphine which produces less moderate to severe nausea than after an equal amount or dose of commercially available immediate release sublingual formulation of buprenorphine listed in FDA's Orange Book.

It is an object of some preferred embodiments to provide an oral pharmaceutical composition comprising a therapeutically effective amount of buprenorphine which produces less dizziness than after an equal amount or dose of commercially available immediate release sublingual formulation of buprenorphine listed in FDA's Orange Book.

It is an object of some preferred embodiments to provide an oral pharmaceutical composition comprising a therapeutically effective amount of buprenorphine which produces less dry mouth than after an equal amount or dose of commercially available immediate release sublingual formulation of buprenorphine listed in FDA's Orange Book.

It is an object of some preferred embodiments to provide an oral pharmaceutical composition comprising a therapeutically effective amount of buprenorphine which has less abuse potential than an equal amount or dose of commercially available immediate release sublingual formulation of buprenorphine listed in FDA's Orange Book (e.g., produces lower abuse scores for “drug effects”, “drug liking”, “coasting”, “take again”, as defined herein).

It is an object of some preferred embodiments to provide an oral pharmaceutical composition comprising a therapeutically effective amount of buprenorphine which produces less neurologic, cognitive, motor and psychomotor impairment than an equal amount or dose of commercially available immediate release sublingual formulation of buprenorphine listed in FDA's Orange Book (e.g., produces lower impairment scores for “critical tracking task”, “stop signal task” and “Tower of London” (TOL), as defined herein).

It is an object of certain preferred embodiments of the present invention to provide bioavailable oral buprenorphine formulations which provide a substantially increased duration of effect as compared to immediate release buprenorphine formulations.

It is an object of certain preferred embodiments of the present invention to provide bioavailable formulations for oral administration suitable for up to every 1, 2, 4, 6, 8, 12, 24, 36, 48 hour and 72 hour administration.

It is an object of certain preferred embodiments of the present invention to provide bioavailable formulations of buprenorphine for oral administration suitable for up to every 1, 2, 4, 6, 8, 12, 24, 36, 48 hour and 72 hour administration which provide an early onset and sustained duration of therapeutic effect.

It is an object of certain preferred embodiments of the present invention to provide bioavailable formulations of buprenorphine for oral administration suitable for up to every 1, 2, 4, 6, 8, 12, 24, 36, 48 hour and 72 hour administration which provide an early onset and sustained duration of therapeutic effect, without the need for a loading dose.

It is an object of certain preferred embodiments of the present invention to provide oral buprenorphine formulations.

It is an object of certain preferred embodiments of the present invention to provide oral buprenorphine formulations which provide a therapeutic effect for up to about 0.5, 1, 2, 4, 6, 8, 12, 24, 36, 48 hour and 72 hours.

It is an object of certain preferred embodiments of the invention to provide an oral buprenorphine formulation which provides a sustained duration of therapeutic effect.

It is an object of certain preferred embodiments of the invention to provide an oral buprenorphine formulation which provides an early onset and sustained duration of therapeutic effect.

It is an object of certain preferred embodiments of the invention to provide a method and formulations of oral buprenorphine for the treatment of pain.

It is an object of certain preferred embodiments of the invention to provide a method and formulations of oral buprenorphine for the treatment of opioid addiction disorders.

It is an object of certain preferred embodiments of the invention to provide a method and formulations of oral buprenorphine for the treatment of opioid dependence.

It is an object of certain preferred embodiments of the invention to provide a method and formulations of oral buprenorphine intended as therapeutic substitutes for unprescribed, illicit, or medically unsanctioned pharmaceutical grade or street grade opioids.

It is an object of certain preferred embodiments of the invention to provide a method and formulations of oral buprenorphine intended as part of an opioid substitution or opioid maintenance therapy in patients with an opioid addiction disorder or a polysubstance abuse disorder.

It is an object of certain preferred embodiments of the invention to provide a method and formulations of oral buprenorphine for the treatment of conditions other than pain and addiction disorders.

It is an object of certain preferred embodiments of the invention to provide a method and formulations of oral buprenorphine, said formulations and methods not having a propensity of substantial drug accumulation.

It is an object of certain preferred embodiments of the invention to provide a method and formulations of oral buprenorphine, said formulations having a reduced potential for drug abuse and drug diversion.

It is an object of certain preferred embodiments of the invention to provide a method and formulations of oral buprenorphine for the treatment of cough, dyspnea, restless leg syndrome, fibromyalgia, acute herpes zoster, visceral pain, breakthrough pain, opioid dependence and urinary incontinence.

In some preferred embodiments one or more or all of the specifications, embodiments and claims of the invention applicable to a human subject are also applicable to other mammals.

It is an object of certain preferred embodiments of the invention to provide a method and formulations of oral buprenorphine, said formulations having a reduced intrasubject pharmacokinetic variability.

It is an object of certain preferred embodiments of the invention to provide a method and formulations of oral buprenorphine, said formulations having a reduced intersubject pharmacokinetic variability.

It is an object of certain preferred embodiments of the invention to provide a method and formulations of oral buprenorphine, said formulations having a reduced peak to trough fluctuation.

It is an object of certain preferred embodiments of the invention to provide a method and formulations of oral buprenorphine, said formulations having a shorter time to therapeutic concentrations or having a shorter time to steady-state.

It is an object of certain preferred embodiments of the invention to provide a method and formulations of oral buprenorphine for the treatment of pain, said formulations suitable for use in acute pain, including acute postsurgical pain.

It is an object of certain preferred embodiments of the invention to provide a method and formulations of oral buprenorphine for the treatment of pain, said formulations suitable for use in chronic pain.

It is an object of certain preferred embodiments of the invention to provide a method and formulations of oral buprenorphine for the treatment of pain, said formulations suitable for use in cancer pain.

It is an object of certain preferred embodiments of the invention to provide a method and formulations of oral buprenorphine for the treatment of pain, said formulations suitable for use in neuropathic pain.

It is an object of certain preferred embodiments of the invention to provide a method and formulations of oral buprenorphine for the treatment of pain, said formulations suitable for use in somatic, visceral and idiopathic pain.

It is an object of certain preferred embodiments of the invention to provide a method and formulations of oral buprenorphine for the treatment of pain, said formulations suitable for use in breakthrough pain or various etiologies, including cancer, chronic pain and neuropathic pain.

In some preferred embodiments, the invention comprises pharmaceutical composition comprising a therapeutically effective amount of buprenorphine or a pharmaceutically acceptable salt of buprenorphine or a mixture thereof wherein all the specifications of the invention applicable to the treatment of pain and addiction disorders are also applicable to the treatment of medical conditions other than pain and addiction disorders.

In some preferred embodiments, the invention provides oral buprenorphine formulations which when evaluated versus other buprenorphine dosage forms in fasted healthy subjects provide a relative mean Cₘₐₓ whose 90% confidence interval is outside the 80.00% to 125.00, under single-dose fasted test conditions in healthy subjects.

In some preferred embodiments, the invention provides oral buprenorphine formulations which when evaluated versus other buprenorphine dosage forms in fasted healthy subjects provide a relative mean AUC_{0-τ} whose 90% confidence interval is outside the 80.00% to 125.00, under single-dose fasted test conditions in healthy subjects.

In some preferred embodiments, the invention provides oral buprenorphine formulations which when evaluated versus other buprenorphine dosage forms in fasted healthy subjects provide a relative mean AUC_{0-inf} whose 90% confidence interval is outside the 80.00% to 125.00, under single-dose fasted test conditions in healthy subjects.

Specifically excluded from this invention are buprenorphine dosage forms which are administered by the lingual, sublingual, oro-mucosal, transmucosal and buccal routes. Lingual, sublingual, oro-mucosal, transmucosal and buccal routes are intended to provide absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach). Such formulations and their method of administration are well known in the art and include lozenges, transmucosal films, buccal products, mucoretentive products, orally disintegrating tablets, fast dissolving tablets, fast dispersing tablets, fast disintegrating dosage forms, provided they are administered for absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach).

Some or all of the above objects and others are achieved by embodiments of the present invention, which is directed in part to a dosage form of oral buprenorphine.

In some preferred embodiments, the dosage form of oral buprenorphine releases substantially more buprenorphine into systemic circulation during the first half of the intended dosing frequency than during the second half of the intended dosing frequency.

In some preferred embodiments, the dosage form of oral buprenorphine releases at least as much buprenorphine into systemic circulation during the first one-third of the intended dosing frequency as during the remainder of the intended dosing frequency.

In some preferred embodiments, the dosage form of oral buprenorphine releases substantially more buprenorphine into systemic circulation during the first one-third of the intended dosing frequency than during the remainder of the intended dosing frequency.

In some preferred embodiments, the invention comprises an oral pharmaceutical composition for the prevention or treatment of pain comprising a therapeutically effective amount of buprenorphine or a pharmaceutically acceptable salt of buprenorphine or a mixture thereof.

It is an object of certain preferred embodiments of the invention to provide a method and formulations of oral buprenorphine, said formulations not having a propensity for toxicity.

It is an object of certain embodiments of the present invention to provide oral buprenorphine formulations in immediate release form.

It is an object of certain embodiments of the present invention to provide oral buprenorphine formulations in controlled release form.

It is an object of certain embodiments of the present invention to provide oral buprenorphine formulations with both immediate release and controlled release forms.

It is an object of certain embodiments of the present invention to provide oral buprenorphine formulations in pulsatile release form.

It is an object of certain embodiments of the present invention to provide oral buprenorphine wherein the buprenorphine is dispersed within a matrix.

In certain preferred embodiments the oral dosage form of the present invention comprises a matrix which includes a sustained release material and buprenorphine or a pharmaceutically acceptable salt thereof. In certain preferred embodiments, the matrix is compressed into a tablet and may be optionally overcoated with a coating that in addition to the sustained release material of the matrix may control the release of the buprenorphine or pharmaceutically acceptable salt thereof from the formulation, such that blood levels of active ingredient are maintained within the therapeutic range over an extended period of time. In certain alternate embodiments, the matrix is encapsulated.

In certain preferred embodiments, the sustained release oral dosage form of the present invention comprises a plurality of pharmaceutically acceptable sustained release matrices comprising buprenorphine, the dosage form maintaining the blood plasma levels of buprenorphine within the therapeutic range over an extended period of time when administered to patients.

In certain preferred embodiments the sustained release oral dosage form of the present invention is an osmotic dosage form which comprises a single layer or bilayer core comprising buprenorphine; an expandable polymer; a semipermeable membrane surrounding the core; and a passageway disposed in the semipermeable membrane for sustained release of the buprenorphine or pharmaceutically acceptable salt thereof, such that blood levels of active ingredient are maintained within the therapeutic range over an extended period of time when administered to patients.

Other oral osmotic delivery systems may be used for the oral administration of buprenorphine.

In some preferred embodiments of the invention, the oral buprenorphine is interdispersed and are not isolated from each other in two distinct layers.

In some preferred embodiments of the invention, the oral buprenorphine is in the form of multiparticulates.

In some preferred embodiments of the invention, the oral buprenorphine is dispersed in a matrix

In some preferred embodiments of the invention, the oral buprenorphine is in the form of multiparticulates can be dispersed in a matrix or contained in a capsule.

In some preferred embodiments of the invention, the oral buprenorphine is in the form of multiparticulates can be dispersed in a matrix and compressed into a tablet.

In some preferred embodiments of the invention, the oral buprenorphine is in a matrix that is in the form of pellets.

In some preferred embodiments of the invention, the oral buprenorphine is in coated beads.

In some preferred embodiments, the dosage form of the invention comprises a compressed tablet, compressed capsule or uncompressed capsule. In other embodiments, the dosage form comprises a liquid fill capsule.

In some preferred embodiments, the oral dosage in immediate or sustained release form provides therapeutic effects that persist despite the low or undectable buprenorphine concentrations.

In some preferred embodiments, the dosage form of the invention comprises an oral formulation (e.g., tablet or capsule) which is coated to prevent substantial direct contact of buprenorphine with oral cavity (e.g. tongue, oral mucosa), oropharyngeal mucosal surface, esophagus or stomach.

In some preferred embodiments, the dosage form of the invention comprises an oral formulation which is coated with a film or polymer. In some preferred embodiments, the dosage form of the invention comprises buprenorphine in an enteric coating.

In some preferred embodiments, the dosage form provides an oral pharmaceutical composition comprising a therapeutically effective amount of buprenorphine; said therapeutically effective amount in a reservoir comprising: (i) buprenorphine or a pharmaceutically acceptable salt of buprenorphine, or a mixture thereof; (ii) a membrane layer, said membrane being substantially permeable to buprenorphine; wherein the dosage form substantially releases the buprenorphine from the dosage form to render said dosage form suitable for extended release to a human patient.

In some preferred embodiments, the dosage form provides an oral pharmaceutical composition comprising a plurality of pharmaceutically acceptable beads coated with a therapeutically effective amount of buprenorphine or a pharmaceutically acceptable salt of buprenorphine, or a mixture thereof; and overcoated with controlled release material to render said dosage form suitable for extended release oral administration to a human patient.

In some preferred embodiments, the dosage form provides an oral pharmaceutical composition comprising (i) a drug layer comprising a therapeutically effective amount of buprenorphine; and (ii) a displacement layer comprising an osmopolymer; and (b) a semipermeable wall surrounding the bilayer core having a passageway disposed therein for the release of said buprenorphine or a pharmaceutically acceptable salt thereof; said dosage form suitable for extended release oral administration to a human patient.

In some preferred embodiments, the oral dosage from is a controlled release material suitable for extended release in a human patient of the dosage form comprises a matrix. In some preferred embodiments, the said matrix is a plurality of multiparticulate matrices. In some preferred embodiments, the multiparticulates are compressed into a tablet. In some preferred embodiments, the multiparticulates are disposed in a pharmaceutically acceptable capsule.

In some preferred embodiments, the controlled release material of the oral dosage form of the invention is selected from the group consisting of hydrophobic polymers, hydrophilic polymers, gums, protein derived materials, waxes, shellac, oils, fats and mixtures thereof.

In some preferred embodiments, the controlled release material of the oral dosage form of the invention is selected from the group consisting of hydrogenated Type I or Type II vegetable oils, polyoxyethylene stearates and distearates, glycerol monostearate, and non-polymeric, non-water soluble liquids carbohydrate-based substances or poorly water soluble, high melting point (mp=40 to 100° C.) waxes and mixtures thereof.

In some preferred embodiments, the oral dosage form comprises a plurality of pharmaceutically acceptable beads coated with drug and overcoated with controlled release material.

In some preferred embodiments, the oral dosage form comprises (i) a drug layer; and (ii) a displacement layer comprising an osmopolymer; and (b) a semipermeable wall surrounding the bilayer core having a passageway disposed therein for the release of said drug.

In some preferred embodiments, the oral dosage form comprises a compressed tablet, compressed capsule or uncompressed capsule. In some preferred embodiments, the oral dosage form comprises a liquid fill capsule.

In some preferred embodiments, the in vivo pharmacokinetic parameters of the specifications, embodiments and claims are derived or determined under fed conditions. In other preferred embodiments, the in vivo pharmacokinetic parameters are derived or determined under fasted conditions.

In some preferred embodiments, the present invention specifically excludes immediate release dosage forms.

In some preferred embodiments, the present invention specifically excludes dosage forms devoid or material to render the dosage form as controlled release.

Some or all of the above objects and others are achieved by embodiments of the present invention, which is directed in part to a dosage form of oral buprenorphine.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of chronic pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of neuropathic pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of cancer pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain, excluding acute pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain, excluding acute postsurgical pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain which is unresponsive to other oral formulations of opioid analgesics, particularly full or pure mu opioid agonists.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain which is unresponsive to other oral formulations of pure or full mu-opioid receptor agonists.

In some preferred embodiments, the dosage form provides an oral pharmaceutical composition comprising a therapeutically effective amount of buprenorphine or a pharmaceutically acceptable salt of buprenorphine or a mixture thereof; said dosage form intended for the treatment of pain which is unresponsive to sublingual buprenorphine.

In some preferred embodiments, the dosage form provides an oral pharmaceutical composition comprising a therapeutically effective amount of buprenorphine or a pharmaceutically acceptable salt of buprenorphine or a mixture thereof; said dosage form intended for the treatment of pain which is unresponsive to transdermal buprenorphine.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain in patients with an addiction disorder or at significant risk of an addiction disorder.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of addiction disorders.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of opioid addiction disorders.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of addiction disorders unresponsive to methadone.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for treatment unresponsive to sublingual, lingual, or buccal buprenorphine.

In some preferred embodiments, the dosage form provides an oral extended release pharmaceutical composition comprising a therapeutically effective amount of buprenorphine or a pharmaceutically acceptable salt of buprenorphine or a mixture thereof; said dosage form intended solely for treatment unresponsive to oral immediate release buprenorphine.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment unresponsive to buprenorphine dosage forms intended to be significantly absorbed through the oral cavity oral mucosa (e.g., lozenges, orally disintegrating tablets, fast dissolving tablets, effervescent tablets, buccal dosage forms, sublingual dosage forms, lingual dosage forms or muco-retentive dosage forms),

The invention is also directed to kits of the dosage forms, including kits for titration disclosed herein.

In another aspect, the invention relates to a method for prevention or treatment of pain, cough, dyspnea, opioid addiction disorders, restless leg syndrome, fibromyalgia, acute herpes zoster, visceral pain, breakthrough pain, opioid dependence and urinary incontinence, comprising oral administration of a dosage form containing buprenorphine or a pharmaceutically acceptable salt of buprenorphine or a mixture thereof.

In another aspect, the invention relates to a method for prevention or treatment of buprenorphine responsive or opioid responsive medical conditions, comprising oral administration of a dosage form containing buprenorphine or a pharmaceutically acceptable salt of buprenorphine or a mixture thereof.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said dosing regimen associated with reduced side effects, improved tolerability, improved efficiency of therapeutic response, reduced breakthrough symptoms (e.g., breakthrough pain) and reduced treatment discontinuation due to side effects.

In some preferred embodiments, the dosage form provides an oral pharmaceutical composition comprising a therapeutically effective amount of buprenorphine; optionally, an oral controlled release material to render said dosage form suitable for extended release in a human patient; said dosage form administered at a prespecified dosing regimen; said dosing regimen associated with reduced side effects, improved tolerability, improved efficiency of therapeutic response, reduced breakthrough symptoms (e.g., breakthrough pain) and reduced treatment discontinuation due to side effects.

In some preferred embodiments, the dosage form provides an oral pharmaceutical composition comprising a therapeutically effective amount of buprenorphine; said dosage form suitable for oral immediate release in a human patient; said dosage form administered at a prespecified dosing regimen; said dosing regimen associated with reduced side effects, improved tolerability, improved efficiency of therapeutic response, reduced breakthrough symptoms (e.g., breakthrough pain) and reduced treatment discontinuation due to side effects.

In some preferred embodiments, the dosage form provides an oral pharmaceutical composition comprising a therapeutically effective amount of buprenorphine; optionally; said dosage form intended to treat pediatric patients; said dosage form administered at a prespecified dosing regimen; said dosing regimen as provided herein, except that the dose or total daily dose (as applicable) is multiplied by the ratio obtained from the child's weight in kilograms divided by 70 kilograms.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose of about 10 mg to about 20 mg for about 4 to about 10 days, then about 22 mg to about 40 mg for about 4 to about 10 days, and then about 50 mg to 600 mg for at least 1 day and optionally thereafter; or a dose of about 5 mg to about 20 mg for about 2 to about 7 days, then about 22 mg to about 60 mg for at least 1 day and optionally thereafter; or a dose of about 2 mg to about 20 mg for about 1 to about 7 days, then about 5 mg to about 40 mg for at least 1 day and optionally thereafter; or a dose of about 10 mg to about 20 mg for about 2 to about 7 days, then about 22 mg to about 40 mg for about 2 to about 7 days, and then about 50 mg to 600 mg for at least 1 day and optionally thereafter; or a dose of about 10 mg to about 15 mg for about 4 to about 10 days, then about 18 mg to about 40 mg for about 4 to about 10 days, and then about 50 mg to 600 mg for at least 1 day and optionally thereafter; or a dose of about 10 mg to about 15 mg for about 2 to about 7 days, then about 18 mg to about 40 mg for about 2 to about 7 days, and then about 50 mg to 600 mg for at least 1 day and optionally thereafter; or a dose of about 5 mg to about 8 mg for about 4 to about 10 days, then about 10 mg to about 20 mg for about 4 to about 10 days, and then about 25 mg to 600 mg for at least 1 day and optionally thereafter; or a dose of about 5 mg to about 8 mg for about 2 to about 7 days, then about 10 mg to about 20 mg for about 2 to about 7 days, and then about 25 mg to 600 mg for at least 1 day and optionally thereafter; or a dose of about 5 mg to about 8 mg for about 2 to about 7 days, then about 15 mg to about 20 mg for about 2 to about 7 days, and then about 25 mg to 600 mg for at least 1 day and optionally thereafter; or a dose of about 5 mg to about 10 mg for about 2 to about 7 days, then about 12 mg to about 20 mg for about 2 to about 7 days, and then about 25 mg to 600 mg for at least 1 day and optionally thereafter; or a dose of about 5 mg to about 10 mg for about 4 to about 10 days, then about 12 mg to about 20 mg for about 4 to about 10 days, and then about 25 mg to 600 mg for at least 1 day and optionally thereafter; or a dose of about 5 mg to about 10 mg for about 4 to about 10 days, then about 15 mg to about 20 mg for about 4 to about 10 days, and then about 25 mg to 600 mg for at least 1 day and optionally thereafter; or a dose of about 5 mg for about 4 to about 10 days, then about 10 mg for about 4 to about 10 days, and then about 15 mg to 600 mg for at least 1 day and optionally thereafter; or a dose of about 10 mg for about 4 to about 10 days, then about 15 mg for about 4 to about 10 days, and then about 20 mg to 600 mg for at least 1 day and optionally thereafter; or a dose of about 10 mg for about 4 to about 10 days, then about 20 mg for about 4 to about 10 days, and then about 25 mg to 600 mg for at least 1 day and optionally thereafter; or a dose of about 5 mg to about 10 mg for about 2 to about 7 days, then about 12 mg to about 20 mg for about 2 to about 7 days, and then about 25 mg to 600 mg for at least 1 day and optionally thereafter; or a dose of about 5 mg to about 10 mg for about 2 to about 7 days, then about 15 mg to about 20 mg for about 2 to about 7 days, and then about 25 mg to 600 mg for at least 1 day and optionally thereafter; or a dose of about 5 mg for about 2 to about 7 days, then about 10 mg for about 2 to about 7 days, and then about 15 mg to 600 mg for at least 1 day and optionally thereafter; or a dose of about 10 mg for about 2 to about 7 days, then about 15 mg for about 2 to about 7 days, and then about 20 mg to 600 mg for at least 1 day and optionally thereafter; or a dose of about 10 mg for about 2 to about 7 days, then about 20 mg for about 2 to about 7 days, and then about 25 mg to 600 mg for at least 1 day and optionally thereafter; or a dose of about 5 mg for about 4 to about 10 days, and then about 10 mg to 600 mg for at least 1 day and optionally thereafter; or a dose of about 10 mg for about 4 to about 10 days, and then about 15 mg to 600 mg for at least 1 day and optionally thereafter; or a dose of about 15 mg for about 4 to about 10 days, and then about 20 mg to 600 mg for at least 1 day and optionally thereafter; or a dose of about 20 mg for about 4 to about 10 days, and then about 25 mg to 600 mg for at least 1 day and optionally thereafter; or a dose of about 5 mg for about 2 to about 7 days, and then about 10 mg to 600 mg for at least 1 day and optionally thereafter; or a dose of about 10 mg for about 2 to about 7 days, and then about 15 mg to 600 mg for at least 1 day and optionally thereafter; or a dose of about 15 mg for about 2 to about 7 days, and then about 20 mg to 600 mg for at least 1 day and optionally thereafter; or a dose of about 20 mg for about 2 to about 7 days, and then about 25 mg to 600 mg for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said regimen comprising administering a dose of about 1 mg to about 5 mg for about 4 to about 10 days, then about 6 mg to about 10 mg for about 4 to about 10 days, and then about 12 mg to 200 for at least 1 day and optionally thereafter; or a dose of about 5 mg to about 20 mg for about 2 to about 7 days, then about 22 mg to about 60 mg for at least 1 day and optionally thereafter; or a dose of about 2 mg to about 20 mg for about 1 to about 7 days, then about 5 mg to about 40 mg for at least 1 day and optionally thereafter; or a dose of about 2 mg to about 8 mg for about 2 to about 7 days, then about 10 mg to about 15 mg for about 2 to about 7 days, and then about 16 mg to 200 for at least 1 day and optionally thereafter; or a dose of about 2 mg to about 5 mg for about 2 to about 7 days, then about 6 mg to about 10 mg for about 2 to about 7 days, and then about 12 mg to 200 for at least 1 day and optionally thereafter; or about 5 mg to about 8 mg for about 4 to about 10 days, then about 10 mg to about 20 mg for about 4 to about 10 days, and then about 25 mg to 200 for at least 1 day and optionally thereafter; or a dose of about 5 mg to about 8 mg for about 2 to about 7 days, then about 10 mg to about 20 mg for about 2 to about 7 days, and then about 25 mg to 200 for at least 1 day and optionally thereafter; or a dose of about 5 mg to about 8 mg for about 2 to about 7 days, then about 15 mg to about 20 mg for about 2 to about 7 days, and then about 25 mg to 200 for at least 1 day and optionally thereafter; or a dose of about 5 mg to about 10 mg for about 2 to about 7 days, then about 12 mg to about 20 mg for about 2 to about 7 days, and then about 25 mg to 200 for at least 1 day and optionally thereafter; or a dose of about 5 mg to about 10 mg for about 4 to about 10 days, then about 12 mg to about 20 mg for about 4 to about 10 days, and then about 25 mg to 200 for at least 1 day and optionally thereafter; or a dose of about 5 mg to about 10 mg for about 4 to about 10 days, then about 15 mg to about 20 mg for about 4 to about 10 days, and then about 25 mg to 200 for at least 1 day and optionally thereafter; or a dose of about 5 mg for about 4 to about 10 days, then about 10 mg for about 4 to about 10 days, and then about 15 mg to 200 for at least 1 day and optionally thereafter; or a dose of about 10 mg for about 4 to about 10 days, then about 15 mg for about 4 to about 10 days, and then about 20 mg to 200 for at least 1 day and optionally thereafter; or a dose of about 10 mg for about 4 to about 10 days, then about 20 mg for about 4 to about 10 days, and then about 25 mg to 200 for at least 1 day and optionally thereafter; or a dose of about 5 mg to about 10 mg for about 2 to about 7 days, then about 12 mg to about 20 mg for about 2 to about 7 days, and then about 25 mg to 200 for at least 1 day and optionally thereafter; or a dose of about 5 mg to about 10 mg for about 2 to about 7 days, then about 15 mg to about 20 mg for about 2 to about 7 days, and then about 25 mg to 200 for at least 1 day and optionally thereafter; or a dose of about 5 mg for about 2 to about 7 days, then about 10 mg for about 2 to about 7 days, and then about 15 mg to 200 for at least 1 day and optionally thereafter; or a dose of about 10 mg for about 2 to about 7 days, then about 15 mg for about 2 to about 7 days, and then about 20 mg to 200 for at least 1 day and optionally thereafter; or a dose of about 10 mg for about 2 to about 7 days, then about 20 mg for about 2 to about 7 days, and then about 25 mg to 200 for at least 1 day and optionally thereafter; or a dose of about 5 mg for about 4 to about 10 days, and then about 10 mg to 200 for at least 1 day and optionally thereafter; or a dose of about 10 mg for about 4 to about 10 days, and then about 15 mg to 200 for at least 1 day and optionally thereafter; or a dose of about 15 mg for about 4 to about 10 days, and then about 20 mg to 200 for at least 1 day and optionally thereafter; or a dose of about 20 mg for about 4 to about 10 days, and then about 25 mg to 200 for at least 1 day and optionally thereafter; or a dose of about 5 mg for about 2 to about 7 days, and then about 10 mg to 200 for at least 1 day and optionally thereafter; or a dose of about 10 mg for about 2 to about 7 days, and then about 15 mg to 200 for at least 1 day and optionally thereafter; or a dose of about 15 mg for about 2 to about 7 days, and then about 20 mg to 200 for at least 1 day and optionally thereafter; or a dose of about 20 mg for about 2 to about 7 days, and then about 25 mg to 200 for at least 1 day and optionally thereafter.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said regimen comprising administering a dose of about 1 mg to about 5 mg for about 2 to about 4 days, then about 6 mg to about 10 mg for about 2 to about 4 days, and then about 12 mg to 200 for at least 1 day and optionally thereafter; or a dose of about 2 mg to about 8 mg for about 1 to about 4 days, then about 10 mg to about 15 mg for about 1 to about 4 days, and then about 16 mg to 200 for at least 1 day and optionally thereafter; or a dose of about 2 mg to about 5 mg for about 1 to about 4 days, then about 6 mg to about 10 mg for about 1 to about 4 days, and then about 12 mg to 200 for at least 1 day and optionally thereafter; or about 5 mg to about 8 mg for about 2 to about 4 days, then about 10 mg to about 20 mg for about 2 to about 4 days, and then about 25 mg to 200 for at least 1 day and optionally thereafter; or a dose of about 5 mg to about 8 mg for about 1 to about 4 days, then about 10 mg to about 20 mg for about 1 to about 4 days, and then about 25 mg to 200 for at least 1 day and optionally thereafter; or a dose of about 5 mg to about 8 mg for about 1 to about 4 days, then about 15 mg to about 20 mg for about 1 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 2 to about 4 days, then about 12 mg to about 20 mg for about 2 to about 4 days, and then about 25 mg to 200 for at least 1 day and optionally thereafter; or a dose of about 5 mg to about 10 mg for about 2 to about 4 days, then about 15 mg to about 20 mg for about 2 to about 4 days, and then about 25 mg to 200 for at least 1 day and optionally thereafter; or a dose of about 5 mg for about 2 to about 4 days, then about 10 mg for about 2 to about 4 days, and then about 15 mg to 200 for at least 1 day and optionally thereafter; or a dose of about 10 mg for about 2 to about 4 days, then about 15 mg for about 2 to about 4 days, and then about 20 mg to 200 for at least 1 day and optionally thereafter; or a dose of about 10 mg for about 2 to about 4 days, then about 20 mg for about 2 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 1 to about 4 days, then about 15 mg to about 20 mg for about 1 to about 4 days, and then about 25 mg to 200 for at least 1 day and optionally thereafter; or a dose of about 5 mg for about 1 to about 4 days, then about 10 mg for about 1 to about 4 days, and then about 15 mg to 200 for at least 1 day and optionally thereafter; or a dose of about 10 mg for about 1 to about 4 days, then about 15 mg for about 1 to about 4 days, and then about 20 mg to 200 for at least 1 day and optionally thereafter; or a dose of about 10 mg for about 1 to about 4 days, then about 20 mg for about 1 to about 4 days, and then about 25 mg to 200 for at least 1 day and optionally thereafter; or a dose of about 5 mg for about 2 to about 4 days, and then about 10 mg to 200 for at least 1 day and optionally thereafter; or a dose of about 10 mg for about 2 to about 4 days, and then about 15 mg to 200 for at least 1 day and optionally thereafter; or a dose of about 15 mg for about 2 to about 4 days, and then about 20 mg to 200 for at least 1 day and optionally thereafter; or a dose of about 20 mg for about 2 to about 4 days, and then about 25 mg to 200 for at least 1 day and optionally thereafter; or a dose of about 5 mg for about 1 to about 4 days, and then about 10 mg to 200 for at least 1 day and optionally thereafter; or a dose of about 10 mg for about 1 to about 4 days, and then about 15 mg to 200 for at least 1 day and optionally thereafter; or a dose of about 15 mg for about 1 to about 4 days, and then about 20 mg to 200 for at least 1 day and optionally thereafter; or a dose of about 20 mg for about 1 to about 4 days, and then about 25 mg to 200 for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a total daily dose of a dose of about 10 mg to about 20 mg for about 4 to about 10 days, then about 22 mg to about 40 mg for about 4 to about 10 days, and then about 50 mg to 600 mg for at least 1 day and optionally thereafter; or a dose of about 5 mg to about 20 mg for about 2 to about 7 days, then about 22 mg to about 60 mg for at least 1 day and optionally thereafter; or a dose of about 10 mg to about 20 mg for about 2 to about 7 days, then about 22 mg to about 40 mg for about 2 to about 7 days, and then about 50 mg to 600 mg for at least 1 day and optionally thereafter; or a dose of about 10 mg to about 15 mg for about 4 to about 10 days, then about 18 mg to about 40 mg for about 4 to about 10 days, and then about 50 mg to 600 mg for at least 1 day and optionally thereafter; or a dose of about 10 mg to about 15 mg for about 2 to about 7 days, then about 18 mg to about 40 mg for about 2 to about 7 days, and then about 50 mg to 600 mg for at least 1 day and optionally thereafter; or about 5 mg to about 8 mg for about 4 to about 10 days, then about 10 mg to about 20 mg for about 4 to about 10 days, and then about 25 mg to 600 mg for at least 1 day and optionally thereafter; or a dose of about 5 mg to about 8 mg for about 2 to about 7 days, then about 10 mg to about 20 mg for about 2 to about 7 days, and then about 25 mg to 600 mg for at least 1 day and optionally thereafter; or a dose of about 5 mg to about 8 mg for about 2 to about 7 days, then about 15 mg to about 20 mg for about 2 to about 7 days, and then about 25 mg to 600 mg for at least 1 day and optionally thereafter; or a dose of about 5 mg to about 10 mg for about 2 to about 7 days, then about 12 mg to about 20 mg for about 2 to about 7 days, and then about 25 mg to 600 mg for at least 1 day and optionally thereafter; or a dose of about mg to about 10 mg for about 4 to about 10 days, then about 12 mg to about 20 mg for about 4 to about 10 days, and then about 25 mg to 600 mg for at least 1 day and optionally thereafter; or a dose of about 5 mg to about 10 mg for about 4 to about 10 days, then about 15 mg to about 20 mg for about 4 to about 10 days, and then about 25 mg to 600 mg for at least 1 day and optionally thereafter; or a dose of about 5 mg for about 4 to about 10 days, then about 10 mg for about 4 to about 10 days, and then about 15 mg to 600 mg for at least 1 day and optionally thereafter; or a dose of about 10 mg for about 4 to about 10 days, then about 15 mg for about 4 to about 10 days, and then about 20 mg to 600 mg for at least 1 day and optionally thereafter; or a dose of about 10 mg for about 4 to about 10 days, then about 20 mg for about 4 to about 10 days, and then about 25 mg to 600 mg for at least 1 day and optionally thereafter; or a dose of about 5 mg to about 10 mg for about 2 to about 7 days, then about 12 mg to about 20 mg for about 2 to about 7 days, and then about 25 mg to 600 mg for at least 1 day and optionally thereafter; or a dose of about 5 mg to about 10 mg for about 2 to about 7 days, then about 15 mg to about 20 mg for about 2 to about 7 days, and then about 25 mg to 600 mg for at least 1 day and optionally thereafter; or a dose of about 5 mg for about 2 to about 7 days, then about 10 mg for about 2 to about 7 days, and then about 15 mg to 600 mg for at least 1 day and optionally thereafter; or a dose of about 10 mg for about 2 to about 7 days, then about 15 mg for about 2 to about 7 days, and then about 20 mg to 600 mg for at least 1 day and optionally thereafter; or a dose of about 10 mg for about 2 to about 7 days, then about 20 mg for about 2 to about 7 days, and then about 25 mg to 600 mg for at least 1 day and optionally thereafter; or a dose of about 5 mg for about 4 to about 10 days, and then about 10 mg to 600 mg for at least 1 day and optionally thereafter; or a dose of about 10 mg for about 4 to about 10 days, and then about 15 mg to 600 mg for at least 1 day and optionally thereafter; or a dose of about 15 mg for about 4 to about 10 days, and then about 20 mg to 600 mg for at least 1 day and optionally thereafter; or a dose of about 20 mg for about 4 to about 10 days, and then about 25 mg to 600 mg for at least 1 day and optionally thereafter; or a dose of about 5 mg for about 2 to about 7 days, and then about 10 mg to 600 mg for at least 1 day and optionally thereafter; or a dose of about 10 mg for about 2 to about 7 days, and then about 15 mg to 600 mg for at least 1 day and optionally thereafter; or a dose of about 15 mg for about 2 to about 7 days, and then about 20 mg to 600 mg for at least 1 day and optionally thereafter; or a dose of about 20 mg for about 2 to about 7 days, and then about 25 mg to 600 mg for at least 1 day and optionally thereafter; said total daily dose administered about six times-a-day (Q4H or Q4H PRN) to once-a-day (QD or QD PRN). In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said regimen comprising administering a total daily dose of about 1 mg to about 5 mg for about 4 to about 10 days, then about 6 mg to about 10 mg for about 4 to about 10 days, and then about 12 mg to 200 for at least 1 day and optionally thereafter; or a dose of about 5 mg to about 20 mg for about 2 to about 7 days, then about 22 mg to about 60 mg for at least 1 day and optionally thereafter; or a dose of about 2 mg to about 8 mg for about 2 to about 7 days, then about 10 mg to about 15 mg for about 2 to about 7 days, and then about 16 mg to 200 for at least 1 day and optionally thereafter; or a dose of about 2 mg to about 5 mg for about 2 to about 7 days, then about 6 mg to about 10 mg for about 2 to about 7 days, and then about 12 mg to 200 for at least 1 day and optionally thereafter; or about 5 mg to about 8 mg for about 4 to about 10 days, then about 10 mg to about 20 mg for about 4 to about 10 days, and then about 25 mg to 200 for at least 1 day and optionally thereafter; or a dose of about 5 mg to about 8 mg for about 2 to about 7 days, then about 10 mg to about 20 mg for about 2 to about 7 days, and then about 25 mg to 200 for at least 1 day and optionally thereafter; or a dose of about 5 mg to about 8 mg for about 2 to about 7 days, then about 15 mg to about 20 mg for about 2 to about 7 days, and then about 25 mg to 200 for at least 1 day and optionally thereafter; or a dose of about 5 mg to about 10 mg for about 2 to about 7 days, then about 12 mg to about 20 mg for about 2 to about 7 days, and then about 25 mg to 200 for at least 1 day and optionally thereafter; or a dose of about 5 mg to about 10 mg for about 4 to about 10 days, then about 12 mg to about 20 mg for about 4 to about 10 days, and then about 25 mg to 200 for at least 1 day and optionally thereafter; or a dose of about 5 mg to about 10 mg for about 4 to about 10 days, then about 15 mg to about 20 mg for about 4 to about 10 days, and then about 25 mg to 200 for at least 1 day and optionally thereafter; or a dose of about 5 mg for about 4 to about 10 days, then about 10 mg for about 4 to about 10 days, and then about 15 mg to 200 for at least 1 day and optionally thereafter; or a dose of about 10 mg for about 4 to about 10 days, then about 15 mg for about 4 to about 10 days, and then about 20 mg to 200 for at least 1 day and optionally thereafter; or a dose of about 10 mg for about 4 to about 10 days, then about 20 mg for about 4 to about 10 days, and then about 25 mg to 200 for at least 1 day and optionally thereafter; or a dose of about 5 mg to about 10 mg for about 2 to about 7 days, then about 12 mg to about 20 mg for about 2 to about 7 days, and then about 25 mg to 200 for at least 1 day and optionally thereafter; or a dose of about 5 mg to about 10 mg for about 2 to about 7 days, then about 15 mg to about 20 mg for about 2 to about 7 days, and then about 25 mg to 200 for at least 1 day and optionally thereafter; or a dose of about 5 mg for about 2 to about 7 days, then about 10 mg for about 2 to about 7 days, and then about 15 mg to 200 for at least 1 day and optionally thereafter; or a dose of about 10 mg for about 2 to about 7 days, then about 15 mg for about 2 to about 7 days, and then about 20 mg to 200 for at least 1 day and optionally thereafter; or a dose of about 10 mg for about 2 to about 7 days, then about 20 mg for about 2 to about 7 days, and then about 25 mg to 200 for at least 1 day and optionally thereafter; or a dose of about 5 mg for about 4 to about 10 days, and then about 10 mg to 200 for at least 1 day and optionally thereafter; or a dose of about 10 mg for about 4 to about 10 days, and then about 15 mg to 200 for at least 1 day and optionally thereafter; or a dose of about 15 mg for about 4 to about 10 days, and then about 20 mg to 200 for at least 1 day and optionally thereafter; or a dose of about 20 mg for about 4 to about 10 days, and then about 25 mg to 200 for at least 1 day and optionally thereafter; or a dose of about 5 mg for about 2 to about 7 days, and then about 10 mg to 200 for at least 1 day and optionally thereafter; or a dose of about 10 mg for about 2 to about 7 days, and then about 15 mg to 200 for at least 1 day and optionally thereafter; or a dose of about 15 mg for about 2 to about 7 days, and then about 20 mg to 200 for at least 1 day and optionally thereafter; or a dose of about 20 mg for about 2 to about 7 days, and then about 25 mg to 200 for at least 1 day and optionally thereafter; said total daily dose administered about six times-a-day (Q4H or Q4H PRN) to once-a-day (QD or QD PRN).

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said regimen comprising administering a total daily dose of about 1 mg to about 5 mg for about 1 to about 4 days, then about 6 mg to about 10 mg for about 1 to about 4 days, and then about 12 mg to 200 for at least 1 day and optionally thereafter; or a dose of about 2 mg to about 8 mg for about 1 to about 4 days, then about 10 mg to about 15 mg for about 1 to about 4 days, and then about 16 mg to 200 for at least 1 day and optionally thereafter; or a dose of about 2 mg to about 5 mg for about 1 to about 4 days, then about 6 mg to about 10 mg for about 1 to about 4 days, and then about 12 mg to 200 for at least 1 day and optionally thereafter; or about 5 mg to about 8 mg for about 1 to about 4 days, then about 10 mg to about 20 mg for about 1 to about 4 days, and then about 25 mg to 200 for at least 1 day and optionally thereafter; or a dose of about 5 mg to about 8 mg for about 1 to about 4 days, then about 10 mg to about 20 mg for about 1 to about 4 days, and then about 25 mg to 200 for at least 1 day and optionally thereafter; or a dose of about 5 mg to about 8 mg for about 1 to about 4 days, then about 15 mg to about 20 mg for about 1 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 1 to about 4 days, then about 15 mg to about 20 mg for about 1 to about 4 days, and then about 25 mg to 200 for at least 1 day and optionally thereafter; or a dose of about 5 mg for about 1 to about 4 days, then about 10 mg for about 1 to about 4 days, and then about 15 mg to 200 for at least 1 day and optionally thereafter; or a dose of about 10 mg for about 1 to about 4 days, then about 15 mg for about 1 to about 4 days, and then about 20 mg to 200 for at least 1 day and optionally thereafter; or a dose of about 10 mg for about 1 to about 4 days, then about 20 mg for about 1 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 1 to about 4 days, then about 15 mg to about 20 mg for about 1 to about 4 days, and then about 25 mg to 200 for at least 1 day and optionally thereafter; or a dose of about 5 mg for about 1 to about 4 days, then about 10 mg for about 1 to about 4 days, and then about 15 mg to 200 for at least 1 day and optionally thereafter; or a dose of about 10 mg for about 1 to about 4 days, then about 15 mg for about 1 to about 4 days, and then about 20 mg to 200 for at least 1 day and optionally thereafter; or a dose of about 10 mg for about 1 to about 4 days, then about 20 mg for about 1 to about 4 days, and then about 25 mg to 200 for at least 1 day and optionally thereafter; or a dose of about 5 mg for about 1 to about 4 days, and then about 10 mg to 200 for at least 1 day and optionally thereafter; or a dose of about 10 mg for about 1 to about 4 days, and then about 15 mg to 200 for at least 1 day and optionally thereafter; or a dose of about 15 mg for about 1 to about 4 days, and then about 20 mg to 200 for at least 1 day and optionally thereafter; or a dose of about 20 mg for about 1 to about 4 days, and then about 25 mg to 200 for at least 1 day and optionally thereafter; or a dose of about 5 mg for about 1 to about 4 days, and then about 10 mg to 200 for at least 1 day and optionally thereafter; or a dose of about 10 mg for about 1 to about 4 days, and then about 15 mg to 200 for at least 1 day and optionally thereafter; or a dose of about 15 mg for about 1 to about 4 days, and then about 20 mg to 200 for at least 1 day and optionally thereafter; or a dose of about 20 mg for about 1 to about 4 days, and then about 25 mg to 200 for at least 1 day and optionally thereafter; said total daily dose administered about six times-a-day (Q4H or Q4H PRN) to once-a-day (QD or QD PRN).

In some preferred embodiments, the dosage form provides an oral pharmaceutical composition comprising a therapeutically effective amount of buprenorphine; optionally; said dosage form intended to treat pediatric patients; said dosage form administered at a prespecified dosing regimen; said dosing regimen providing a mean buprenorphine and norbuprenorphine area under the plasma concentration time curve (AUC) as provided herein, except that the AUC is multiplied by the ratio obtained from the child's weight in kilograms divided by 70 kilograms.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release in a human patient; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter.

In a preferred embodiment, the dosage form containing buprenorphine or a pharmaceutically acceptable salt of buprenorphine or a mixture thereof is an extended release form. Oral, extended release buprenorphine has several distinct advantages over oral immediate release opioids and over sublingual, lingual and buccal dosage forms, including fewer interruptions in sleep, reduced dependence on caregivers, improved compliance, enhanced quality of life outcomes, and increased control over the management of their malady (e.g., pain). In addition, such formulations can provide more constant plasma concentrations and clinical effects, less frequent peak to trough fluctuations and fewer side effects.

In some preferred embodiments, the dosage form provides an oral pharmaceutical composition comprising a therapeutically effective amount of buprenorphine or a pharmaceutically acceptable salt of buprenorphine or a mixture thereof; optionally, a controlled release material to render said dosage form suitable for up to every 24 hour (once-a-day) administration to a human patient; said dosage form providing at least 10% of the steady state concentration of buprenorphine and norbuprenorphine after administration of one dose at its intended dosing frequency. In other preferred embodiments, the dosage form provides at least about 15%, or at least about 20%, or at least about 30%, or at least about 40%, or at least about 50%, or at least about 60%, or at least about 70%, or at least about 75%, or at least about 80%, or at least about 85%, or at least about 90% of the steady state therapeutic concentration of buprenorphine after administration of one dose or two doses at their intended dosing frequency.

In some preferred embodiments, the invention provides a method of providing relief in a human patient suffering from pain comprising a therapeutically effective amount of oral buprenorphine or a pharmaceutically acceptable salt of buprenorphine or a mixture thereof.

In some preferred embodiments, the invention provides a method of providing relief in a human patient suffering from cough, dyspnea, opioid addiction disorders, restless leg syndrome, fibromyalgia, acute herpes zoster, visceral pain, breakthrough pain, opioid dependence and urinary incontinence comprising a therapeutically effective amount of oral buprenorphine or a pharmaceutically acceptable salt of buprenorphine or a mixture thereof.

In some preferred embodiments, the invention provides a method of providing relief in a human patient suffering from a buprenorphine responsive medical condition comprising a therapeutically effective amount of oral buprenorphine or a pharmaceutically acceptable salt of buprenorphine or a mixture thereof.

In some preferred embodiments, the invention comprises an oral pharmaceutical composition comprising a therapeutically effective amount of buprenorphine and controlled release material to render said dosage form suitable for three times a day administration (or about every eight hours administration).

In some preferred embodiments, the oral pharmaceutical composition provides a therapeutic effect for about 1, 2, 3, 4, 5, or 6 hours.

In some preferred embodiments, the oral pharmaceutical composition is used on a time contingent basis.

In some preferred embodiments, the oral pharmaceutical composition is used on a pain contingent basis.

In some preferred embodiments, the QID or Q6H oral pharmaceutical composition provides a therapeutic effect for about 6 hours.

In some preferred embodiments, the TID or Q8H oral pharmaceutical composition provides a therapeutic effect for about 8 hours.

In some preferred embodiments, the Q4H or Q4H PRN oral pharmaceutical composition provides a Cₘₐₓ of buprenorphine and norbuprenorphine at about 1 to about 4 hours.

In some preferred embodiments, the Q4H or Q4H PRN oral pharmaceutical composition provides a Cₘᵢₙ of buprenorphine and norbuprenorphine at about 3 to 6 hours.

In some preferred embodiments, the Q4H or Q4H PRN oral pharmaceutical composition provides a mean buprenorphine AUC_{0-inf} after first administration or AUC₀₋₄ at steady state of about 50 pg·hr/mL to about 20,000 pg·hr/mL.

In some preferred embodiments, the Q4H or Q4H PRN oral pharmaceutical composition provides a mean of buprenorphine and norbuprenorphine C₄/Cmax ratio of 0.05 to about 1.25.

In some preferred embodiments, the Q4H or Q4H PRN oral pharmaceutical composition provides a buprenorphine and norbuprenorphine percent fluctuation of less than 400%.

In some preferred embodiments, the Q4H or Q4H PRN oral pharmaceutical composition provides of buprenorphine and norbuprenorphine W₅₀ of 0.5 to about 3.5 hours.

In some preferred embodiments, the Q4H or Q4H PRN oral pharmaceutical composition provides a HVD of buprenorphine and norbuprenorphine of 0.75 to about 3.75 hours.

In some preferred embodiments, the Q4H or Q4H PRN oral pharmaceutical composition provides an AI of buprenorphine and norbuprenorphine of not more that 4.0.

In some preferred embodiments, the Q6H or Q6H PRN oral pharmaceutical composition provides a Cₘₐₓ of buprenorphine and norbuprenorphine at about 1 to about 6 hours.

In some preferred embodiments, the Q6H or Q6H PRN oral pharmaceutical composition provides a Cₘᵢₙ of buprenorphine and norbuprenorphine at about 3 to 8 hours.

In some preferred embodiments, the Q6H or Q6H PRN oral pharmaceutical composition provides a mean buprenorphine AUC_{0-inf} after first administration or AUC₀₋₄ at steady state of about 50 pg·hr/mL to about 20,000 pg·hr/mL.

In some preferred embodiments, the Q6H or Q6H PRN oral pharmaceutical composition provides a mean of buprenorphine and norbuprenorphine C₄/Cmax ratio of 0.05 to about 1.25.

In some preferred embodiments, the Q6H or Q6H PRN oral pharmaceutical composition provides an buprenorphine and norbuprenorphine percent fluctuation of less than 400%.

In some preferred embodiments, the Q6H or Q6H PRN oral pharmaceutical composition provides of buprenorphine and norbuprenorphine W₅₀ of 1.0 to about 5 hours.

In some preferred embodiments, the Q6H or Q6H PRN oral pharmaceutical composition provides a HVD of buprenorphine and norbuprenorphine of 1.25 to about 5.5 hours.

In some preferred embodiments, the Q6H or Q6H PRN oral pharmaceutical composition provides an AI of buprenorphine and norbuprenorphine of not more that 4.0.

In some preferred embodiments, the TID or Q8H oral pharmaceutical composition provides a Cₘₐₓ of buprenorphine and norbuprenorphine at about 1 to about 6 hours.

In some preferred embodiments, the TID or Q8H oral pharmaceutical composition provides a Cₘᵢₙ of buprenorphine and norbuprenorphine at about 6 to 10 hours.

In some preferred embodiments, the TID or Q8H oral pharmaceutical composition provides a mean buprenorphine AUC_{0-inf} after first administration or AUC₀₋₈ at steady state of about 125 pg·hr/mL to about 150,000 pg·hr/mL, or about 125 pg·hr/mL to about 100,000 pg·hr/mL, or about 125 pg·hr/mL to about 75,000 pg·hr/mL, or about 125 pg·hr/mL to about 50,000 pg·hr/mL, or about 125 pg·hr/mL to about 30,000 pg·hr/mL, or about 125 pg·hr/mL to about 20,000 pg·hr/mL, or

In some preferred embodiments, the TID or Q8H oral pharmaceutical composition provides a mean of buprenorphine and norbuprenorphine C₈/Cmax ratio of 0.05 to about 1.25.

In some preferred embodiments, the TID or Q8H oral pharmaceutical composition provides an buprenorphine and norbuprenorphine percent fluctuation of less than 400%.

In some preferred embodiments, the TID or Q8H oral pharmaceutical composition provides of buprenorphine and norbuprenorphine W₅₀ of 1.5 to about 6.5 hours.

In some preferred embodiments, the TID or Q8H oral pharmaceutical composition provides a HVD of buprenorphine and norbuprenorphine of 2 to about 7 hours.

In some preferred embodiments, the TID or Q8H oral pharmaceutical composition provides an AI of buprenorphine and norbuprenorphine of not more than 4.0.

In some preferred embodiments, the invention comprises an oral pharmaceutical composition comprising a therapeutically effective amount of buprenorphine; said dosage from providing a Cₘₐₓ of buprenorphine and norbuprenorphine from about 200 pg/mL to about 40,000 pg/mL. In other preferred embodiments, the dosage form provides a Cₘₐₓ of buprenorphine and norbuprenorphine of about 400 pg/mL to about 40,000 pg/mL, or about 600 pg/mL to about 40,000 pg/mL or about 800 pg/mL to about 40,000 pg/mL, or about 1000 pg/mL to about 40,000 pg/mL or about 2000 pg/mL to about 40,000 pg/mL, or about 3000 pg/mL to about 40,000 pg/mL or about 4000 pg/mL to about 40,000 pg/mL or about 5000 pg/mL to about 40,000 pg/mL, or about 6000 pg/mL to about 40,000 pg/mL or about 7000 pg/mL to about 40,000 pg/mL, or about 8000 pg/mL to about 40,000 pg/mL or about 200 pg/mL to about 35,000 pg/mL or about 200 pg/mL to about 30,000 pg/mL, or about 200 pg/mL to about 25,000 pg/mL or about 200 pg/mL to about 20,000 pg/mL, or about 200 pg/mL to about 15,000 pg/mL or about 200 pg/mL to about 10,000 pg/mL or about 200 pg/mL to about 8,000 pg/mL, or about 200 pg/mL to about 7,000 pg/mL or about 200 pg/mL to about 6,000 pg/mL, or about 200 pg/mL to about 5,000 pg/mL.

In some preferred embodiments, the invention comprises an oral pharmaceutical composition comprising a therapeutically effective amount of buprenorphine; said dosage from providing a Cₘₐₓ of buprenorphine and norbuprenorphine from about 20 pg/mL to about 6000 pg/mL. In other preferred embodiments, the dosage form provides a Cₘₐₓ of buprenorphine and norbuprenorphine of about 40 pg/mL to about 6000 pg/mL, or about 50 pg/mL to about 6000 pg/mL, or about 75 pg/mL to about 6000 pg/mL, or about 100 pg/mL to about 6000 pg/mL, or about 125 pg/mL to about 6000 pg/mL, or about 150 pg/mL to about 6000 pg/mL, or about 175 pg/mL to about 6000 pg/mL, or about 200 pg/mL to about 6000 pg/mL, or about 225 pg/mL to about 6000 pg/mL, or about 250 pg/mL to about 6000 pg/mL, or about 300 pg/mL to about 6000 pg/mL, or about 350 pg/mL to about 6000 pg/mL, or about 400 pg/mL to about 6000 pg/mL, or about 450 pg/mL to about 6000 pg/mL, or about 500 pg/mL to about 6000 pg/mL, or about 600 pg/mL to about 6000 pg/mL, or about 700 pg/mL to about 6000 pg/mL, or about 800 pg/mL to about 6000 pg/mL, or about 1000 pg/mL to about 6000 pg/mL, or about 1200 pg/mL to about 6000 pg/mL, or about 50 pg/mL to about 5000 pg/mL, or about 100 pg/mL to about 5000 pg/mL, or about 50 pg/mL to about 4000 pg/mL, or about 100 pg/mL to about 4000 pg/mL, or about 200 pg/mL to about 4000 pg/mL, or about 50 pg/mL to about 3000 pg/mL, or about 100 pg/mL to about 3000 pg/mL, or about 50 pg/mL to about 2000 pg/mL, or about 100 pg/mL to about 2000 pg/mL, or about 200 pg/mL to about 2000 pg/mL, or about 50 pg/mL to about 1500 pg/mL, or about 100 pg/mL to about 1500 pg/mL, or about 50 pg/mL to about 1000 pg/mL, or about 100 pg/mL to about 1000 pg/mL, or about 50 pg/mL to about 800 pg/mL, or about 100 pg/mL to about 800 pg/mL, or about 20 pg/mL to about 5000 pg/mL, or about 20 pg/mL to about 4500 pg/mL, or about 20 pg/mL to about 4000 pg/mL, or about 20 pg/mL to about 3500 pg/mL, or about 20 pg/mL to about 3000 pg/mL, or about 20 pg/mL to about 2500 pg/mL, or about 20 pg/mL to about 2000 pg/mL, or about 20 pg/mL to about 1800 pg/mL, or about 20 pg/mL to about 1600 pg/mL, or about 20 pg/mL to about 1500 pg/mL, or about 20 pg/mL to about 1400 pg/mL, or about 20 pg/mL to about 1200 pg/mL, or about 20 pg/mL to about 1100 pg/mL, or about 20 pg/mL to about 1000 pg/mL, or about 20 pg/mL to about 900 pg/mL, or about 20 pg/mL to about 800 pg/mL, or about 20 pg/mL to about 700 pg/mL, or about 20 pg/mL to about 600 pg/mL, or about 20 pg/mL to about 500 pg/mL, or about 100 pg/mL to about 2000 pg/mL, or about 100 pg/mL to about 1800 pg/mL, or about 100 pg/mL to about 1600 pg/mL, or about 100 pg/mL to about 1500 pg/mL, or about 100 pg/mL to about 1400 pg/mL, or about 100 pg/mL to about 1200 pg/mL, or about 100 pg/mL to about 1100 pg/mL, or about 100 pg/mL to about 100 pg/mL, or about 1000 pg/mL to about 900 pg/mL, or about 100 pg/mL to about 800 pg/mL, or about 100 pg/mL to about 700 pg/mL, or about 100 pg/mL to about 600 pg/mL, or about 100 pg/mL to about 500 pg/mL, or about 200 pg/mL to about 1600 pg/mL, or about 200 pg/mL to about 1500 pg/mL, or about 200 pg/mL to about 1400 pg/mL, or about 200 pg/mL to about 1200 pg/mL, or about 200 pg/mL to about 1100 pg/mL, or about 200 pg/mL to about 1000 pg/mL, or about 200 pg/mL to about 900 pg/mL, or about 200 pg/mL to about 800 pg/mL, or about 200 pg/mL to about 700 pg/mL, or about 200 pg/mL to about 600 pg/mL, or about 200 pg/mL to about 500 pg/mL. In some preferred embodiments, the aforementioned Cₘₐₓ being achieved with oral pharmaceutical compositions comprising a controlled release material to render said dosage form suitable for extended release.

In some preferred embodiments, the invention comprises an oral pharmaceutical composition comprising a therapeutically effective amount of buprenorphine; said dosage from providing a Cₘₐₓ of buprenorphine and norbuprenorphine occurring from a mean of about 0.25 to about 30 hours. In other preferred embodiments, the dosage form provides a Cₘₐₓ of buprenorphine and norbuprenorphine occurring from a mean of about 0.5 to about 30 hours, or from a mean of about 1 to about 30 hours, or about 1 to about 26 hours, or about 1 to about 24 hours, or about 1 to about 20 hours, or about 1 to about 18 hours, or about 1 to about 16 hours, or about 1 to about 14 hours, or about 1 to about 12 hours, or about 1 to about 10 hours, or about 1 to about 8 hours, or about 1 to about 6 hours, or about 1 to about 4 hours, or about 1 to about 3 hours, or about 2 to about 30 hours, or about 4 to about 30 hours, or about 4 to about 24 hours, or about 6 to about 24 hours, or about 8 to about 24 hours, or about 10 to about 20 hours, or about 12 to about 24 hours, or about 18 to about 24 hours, or about 2 to about 12 hours, or about 3 to about 12 hours, or about 3 to about 8 hours, or about 4 to about 10 hours, or about 4 to about 12 hours, or about 4 to about 9 hours, or about 5 to about 8 hours. In some preferred embodiments, the aforementioned time to Cₘₐₓ being achieved with oral pharmaceutical compositions comprising a controlled release material to render said dosage form suitable for extended release.

In some preferred embodiments, the invention comprises an oral pharmaceutical composition comprising a therapeutically effective amount of buprenorphine; a controlled release material to render said dosage form suitable for extended release in a human patient.

In some preferred embodiments, the invention comprises an oral pharmaceutical composition comprising a therapeutically effective amount of buprenorphine; said dosage from providing a Cₘᵢₙ of buprenorphine and norbuprenorphine of about 0 pg/mL to about 20,000 pg/mL or 1 pg/mL to about 20,000 pg/mL. In other preferred embodiments, the dosage form provides a Cₘᵢₙ of buprenorphine and norbuprenorphine of less than about 20,000 pg/mL, or less than about 18,000 pg/mL, or less than about 16,000 pg/mL, or less than about 15,000 pg/mL, or less than about 14,000 pg/mL, or less than about 12,000 pg/mL, or less than about 10,000 pg/mL, or less than about 9,000 pg/mL, or less than about 8,000 pg/mL, or less than about 7,000 pg/mL, or less than about 6,000 pg/mL, or less than about 5,000 pg/mL, or less than about 4,000 pg/mL, or less than about 3,000 pg/mL, or less than about 2,000 pg/mL, or less than about 1,000 pg/mL,

In some preferred embodiments, the invention comprises an oral pharmaceutical composition comprising a therapeutically effective amount of buprenorphine; said dosage from providing a Cₘᵢₙ of buprenorphine and norbuprenorphine of about 0 pg/mL to about 3000 pg/mL or 1 pg/mL to about 3000 pg/mL. In other preferred embodiments, the dosage form provides a Cₘᵢₙ of buprenorphine and norbuprenorphine of less than about 2500 pg/mL, or less than about 2000 pg/mL, or less than about 1800 pg/mL, or less than about 1600 pg/mL, or less than about 1800 pg/mL, or less than about 1500 pg/mL, or less than about 1400 pg/mL, or less than about 1200 pg/mL, or less than about 1100 pg/mL, or less than about 1000 pg/mL, or less than about 900 pg/mL, or less than about 800 pg/mL, or less than about 700 pg/mL, or less than about 600 pg/mL, or less than about 500 pg/mL, or less than about 400 pg/mL, or less than about 300 pg/mL, or less than about 250 pg/mL, or less than about 200 pg/mL, or less than about 175 pg/mL, or less than about 150 pg/mL, or less than about 120 pg/mL, or less than about 100 pg/mL, or less than about 90 pg/mL, or less than about 80 pg/mL, or less than about 70 pg/mL, or less than about 60 pg/mL, or less than about 50 pg/mL, or less than about 40 pg/mL, or less than about 30 pg/mL, or less than about 20 pg/mL, or less than about 15 pg/mL, or less than about 10 pg/mL, or less than about 5 pg/mL, or less than about 2 pg/mL, or up to about 3000 pg/mL. In some preferred embodiments, the aforementioned Cₘᵢₙ being achieved with oral pharmaceutical compositions comprising a controlled release material to render said dosage form suitable for extended release.

In some preferred embodiments, the invention comprises an oral pharmaceutical composition comprising a therapeutically effective amount of buprenorphine; said dosage from providing a Cₘᵢₙ of buprenorphine and norbuprenorphine measured from a mean of about 0.5 to about 30 hours. In other preferred embodiments, the dosage form provides a Cₘᵢₙ of buprenorphine and norbuprenorphine measured from a mean of about 0.5 to about 28 hours, or about 1 to about 28 hours, or about 1 to 24 hours, or about 1 to about 20 hours, or about 1 to about 18 hours, or about 1 to about 16 hours, or about 1 to about 12 hours, or about 1 to 10 hours, or about 1 to about 8 hours, or about 1 to about 6 hours, or about 1 to about 4 hours, about 2 to about 24 hours, or about 3 to 24 hours, or about 4 to about 24 hours, or about 6 to about 24 hours, or about 8 to about 24 hours, about 2 to about 12 hours, or about 3 to 10 hours, or about 3 to about 8 hours, or about 4 to about 8 hours, or about 6 to about 10 hours. In some preferred embodiments, the aforementioned Cₘᵢₙ being achieved with oral pharmaceutical compositions comprising a controlled release material to render said dosage form suitable for extended release.

In some preferred embodiments, the invention comprises an oral pharmaceutical composition comprising a therapeutically effective amount of buprenorphine; said dosage form providing a systemic exposure as assessed by the mean of buprenorphine and norbuprenorphine area under the plasma concentration time curves to 24 hours post-dose (AUC₀₋₂₄) of about 500 pg·hr/mL to about 500,000 pg·hr/mL. In other preferred embodiments, the dosage form provides an AUC₀₋₂₄ of buprenorphine and norbuprenorphine of about 50 pg·hr/mL to about 475000 pg·hr/mL, or about 500 pg·hr/mL to about 450000 pg·hr/mL, or about 500 pg·hr/mL to about 425000 pg·hr/mL, or about 500 pg·hr/mL to about 400000 pg·hr/mL, or about 500 pg·hr/mL to about 375000 pg·hr/mL, or about 500 pg·hr/mL to about 350000 pg·hr/mL, or about 500 pg·hr/mL to about 325000 pg·hr/mL, or about 500 pg·hr/mL to about 300000 pg·hr/mL, or about 500 pg·hr/mL to about 275000 pg·hr/mL, or about 500 pg·hr/mL to about 250000 pg·hr/mL, or about 500 pg·hr/mL to about 225000 pg·hr/mL, or about 500 pg·hr/mL to about 200000 pg·hr/mL, or about 500 pg·hr/mL to about 175000 pg·hr/mL, or about 500 pg·hr/mL to about 150000 pg·hr/mL, or about 500 pg·hr/mL to about 125000 pg·hr/mL, or about 500 pg·hr/mL to about 100000 pg·hr/mL, or about 500 pg·hr/mL to about 75000 pg·hr/mL, or about 500 pg·hr/mL to about 50000 pg·hr/mL, or about 1000 pg·hr/mL to about 500000 pg·hr/mL, or about 1500 pg·hr/mL to about 500000 pg·hr/mL, or about 2000 pg·hr/mL to about 500000 pg·hr/mL, or about 3500 pg·hr/mL to about 500000 pg·hr/mL, or about 4000 pg·hr/mL to about 500000 pg·hr/mL, or about 5000 pg·hr/mL to about 500000 pg·hr/mL, or about 6000 pg·hr/mL to about 500000 pg·hr/mL, or about 8000 pg·hr/mL to about 500000 pg·hr/mL, or about 9000 pg·hr/mL to about 500000 pg·hr/mL, or about 10000 pg·hr/mL to about 500000 pg·hr/mL, or about 1000 pg·hr/mL to about 300000 pg·hr/mL, or about 3000 pg·hr/mL to about 300000 pg·hr/mL.

In some preferred embodiments, the invention comprises an oral pharmaceutical composition comprising a therapeutically effective amount of buprenorphine; said dosage form providing a systemic exposure as assessed by the mean of buprenorphine and norbuprenorphine area under the plasma concentration time curves to infinity (AUC_{0-inf}) of about 500 pg·hr/mL to about 500,000 pg·hr/mL. In other preferred embodiments, the dosage form provides an AUC_{0-inf} of buprenorphine and norbuprenorphine of about 50 pg·hr/mL to about 475000 pg·hr/mL, or about 500 pg·hr/mL to about 450000 pg·hr/mL, or about 500 pg·hr/mL to about 425000 pg·hr/mL, or about 500 pg·hr/mL to about 400000 pg·hr/mL, or about 500 pg·hr/mL to about 375000 pg·hr/mL, or about 500 pg·hr/mL to about 350000 pg·hr/mL, or about 500 pg·hr/mL to about 325000 pg·hr/mL, or about 500 pg·hr/mL to about 300000 pg·hr/mL, or about 500 pg·hr/mL to about 275000 pg·hr/mL, or about 500 pg·hr/mL to about 250000 pg·hr/mL, or about 500 pg·hr/mL to about 225000 pg·hr/mL, or about 500 pg·hr/mL to about 200000 pg·hr/mL, or about 500 pg·hr/mL to about 175000 pg·hr/mL, or about 500 pg·hr/mL to about 150000 pg·hr/mL, or about 500 pg·hr/mL to about 125000 pg·hr/mL, or about 500 pg·hr/mL to about 100000 pg·hr/mL, or about 500 pg·hr/mL to about 75000 pg·hr/mL, or about 500 pg·hr/mL to about 50000 pg·hr/mL, or about 1000 pg·hr/mL to about 500000 pg·hr/mL, or about 1500 pg·hr/mL to about 500000 pg·hr/mL, or about 2000 pg·hr/mL to about 500000 pg·hr/mL, or about 3500 pg·hr/mL to about 500000 pg·hr/mL, or about 4000 pg·hr/mL to about 500000 pg·hr/mL, or about 5000 pg·hr/mL to about 500000 pg·hr/mL, or about 6000 pg·hr/mL to about 500000 pg·hr/mL, or about 8000 pg·hr/mL to about 500000 pg·hr/mL, or about 9000 pg·hr/mL to about 500000 pg·hr/mL, or about 10000 pg·hr/mL to about 500000 pg·hr/mL, or about 1000 pg·hr/mL to about 300000 pg·hr/mL, or about 3000 pg·hr/mL to about 300000 pg·hr/mL.

In some preferred embodiments, the invention comprises an oral pharmaceutical composition comprising a therapeutically effective amount of buprenorphine; said dosage form providing a systemic exposure as assessed by the mean of buprenorphine and norbuprenorphine area under the plasma concentration time curves to 24 hours post-dose (AUC₀₋₂₄) of about 50 pg·hr/mL to about 80000 pg·hr/mL. In other preferred embodiments, the dosage form provides an AUC₀₋₂₄ of buprenorphine and norbuprenorphine of about 50 pg·hr/mL to about 70000 pg·hr/mL, or about 50 pg·hr/mL to about 60000 pg·hr/mL, or about 50 pg·hr/mL to about 50000 pg·hr/mL, or about 50 pg·hr/mL to about 40000 pg·hr/mL, or about 50 pg·hr/mL to about 30000 pg·hr/mL, or about 50 pg·hr/mL to about 25000 pg·hr/mL, or about 50 pg·hr/mL to about 20000 pg·hr/mL, or about 50 pg·hr/mL to about 18000 pg·hr/mL, or about 50 pg·hr/mL to about 15000 pg·hr/mL, or about 50 pg·hr/mL to about 14000 pg·hr/mL, or about 50 pg·hr/mL to about 12000 pg·hr/mL, or about 50 pg·hr/mL to about 11000 pg·hr/mL, or about 50 pg·hr/mL to about 10000 pg·hr/mL, or about 50 pg·hr/mL to about 9000 pg·hr/mL, 50 pg·hr/mL to about 8500 pg·hr/mL, or about 50 pg·hr/mL to about 7000 pg·hr/mL, or about 50 pg·hr/mL to about 5000 pg·hr/mL, or about 50 pg·hr/mL to about 3500 pg·hr/mL, or about 50 pg·hr/mL to about 2500 pg·hr/mL, 50 pg·hr/mL to about 1700 pg·hr/mL, or about 50 pg·hr/mL to about 1200 pg·hr/mL, or about 100 pg·hr/mL to about 40000 pg·hr/mL, or about 500 pg·hr/mL to about 20000 pg·hr/mL, or about 500 pg·hr/mL to about 10000 pg·hr/mL, or about 1000 pg·hr/mL to about 20000 pg·hr/mL, or about 1000 pg·hr/mL to about 10000 pg·hr/mL, or about 1000 pg·hr/mL to about 8000 pg·hr/mL, 2000 pg·hr/mL to about 20000 pg·hr/mL, or about 2000 pg·hr/mL to about 15000 pg·hr/mL, or about 2000 pg·hr/mL to about 10000 pg·hr/mL, or about 2000 pg·hr/mL to about 8000 pg·hr/mL, or about 3000 pg·hr/mL to about 40000 pg·hr/mL, or about 3000 pg·hr/mL to about 30000 pg·hr/mL, or about 3000 pg·hr/mL to about 20000 pg·hr/mL, or about 3000 pg·hr/mL to about 15000 pg·hr/mL, or about 4000 pg·hr/mL to about 40000 pg·hr/mL, or about 4000 pg·hr/mL to about 30000 pg·hr/mL, or about 4000 pg·hr/mL to about 20000 pg·hr/mL, or about 5000 pg·hr/mL to about 30000 pg·hr/mL, or about 5000 pg·hr/mL to about 20000 pg·hr/mL, or about 5000 pg·hr/mL to about 15000 pg·hr/mL, or about 6000 pg·hr/mL to about 80000 pg·hr/mL, or about 6000 pg·hr/mL to about 40000 pg·hr/mL, or about 6000 pg·hr/mL to about 30000 pg·hr/mL, or about 100 pg·hr/mL to about 80000 pg·hr/mL, about 200 pg·hr/mL to about 80000 pg·hr/mL, or about 300 pg·hr/mL to about 80000 pg·hr/mL, or about 500 pg·hr/mL to about 80000 pg·hr/mL, or about 700 pg·hr/mL to about 80000 pg·hr/mL, or about 800 pg·hr/mL to about 80000 pg·hr/mL, or about 1000 pg·hr/mL to about 80000 pg·hr/mL, or about 1200 pg·hr/mL to about 80000 pg·hr/mL, or about 1500 pg·hr/mL to about 80000 pg·hr/mL, or about 2000 pg·hr/mL to about 80000 pg·hr/mL, or about 2500 pg·hr/mL to about 80000 pg·hr/mL, or about 3000 pg·hr/mL to about 80000 pg·hr/mL, or about 3500 pg·hr/mL to about 80000 pg·hr/mL, or about 4000 pg·hr/mL to about 80000 pg·hr/mL, or about 5000 pg·hr/mL to about 80000 pg·hr/mL, 6000 pg·hr/mL to about 80000 pg·hr/mL, or about 8000 pg·hr/mL to about 80000 pg·hr/mL, or about 10000 pg·hr/mL to about 80000 pg·hr/mL, or about 12000 pg·hr/mL to about 80000 pg·hr/mL, or about 15000 pg·hr/mL to about 80000 pg·hr/mL, or about 100 pg·hr/mL to about 40000 pg·hr/mL, about 200 pg·hr/mL to about 40000 pg·hr/mL, or about 300 pg·hr/mL to about 40000 pg·hr/mL, or about 500 pg·hr/mL to about 40000 pg·hr/mL, or about 700 pg·hr/mL to about 40000 pg·hr/mL, or about 800 pg·hr/mL to about 40000 pg·hr/mL, or about 1000 pg·hr/mL to about 40000 pg·hr/mL, or about 1200 pg·hr/mL to about 40000 pg·hr/mL, or about 1500 pg·hr/mL to about 40000 pg·hr/mL, or about 2000 pg·hr/mL to about 40000 pg·hr/mL, or about 2500 pg·hr/mL to about 40000 pg·hr/mL, or about 3000 pg·hr/mL to about 40000 pg·hr/mL, or about 3500 pg·hr/mL to about 40000 pg·hr/mL, or about 4000 pg·hr/mL to about 40000 pg·hr/mL, or about 5000 pg·hr/mL to about 40000 pg·hr/mL, 6000 pg·hr/mL to about 40000 pg·hr/mL, or about 8000 pg·hr/mL to about 40000 pg·hr/mL, or about 10000 pg·hr/mL to about 40000 pg·hr/mL, or about 12000 pg·hr/mL to about 40000 pg·hr/mL, or about 15000 pg·hr/mL to about 40000 pg·hr/mL. In some preferred embodiments, the aforementioned AUC₀₋₂₄ being achieved with oral pharmaceutical compositions comprising a controlled release material to render said dosage form suitable for extended release.

In some preferred embodiments, the invention comprises an oral pharmaceutical composition comprising a therapeutically effective amount of buprenorphine; said dosage form providing a systemic exposure as assessed by the mean buprenorphine and norbuprenorphine area under the plasma concentration time curve to infinity (AUC_{0-inf}) of about 50 pg·hr/mL to about 80000 pg·hr/mL. In other preferred embodiments, the dosage form provides an AUC_{0-inf} of buprenorphine and norbuprenorphine of about 50 pg·hr/mL to about 70000 pg·hr/mL, or about 50 pg·hr/mL to about 60000 pg·hr/mL, or about 50 pg·hr/mL to about 50000 pg·hr/mL, or about 50 pg·hr/mL to about 40000 pg·hr/mL, or about 50 pg·hr/mL to about 30000 pg·hr/mL, or about 50 pg·hr/mL to about 25000 pg·hr/mL, or about 50 pg·hr/mL to about 20000 pg·hr/mL, or about 50 pg·hr/mL to about 18000 pg·hr/mL, or about 50 pg·hr/mL to about 15000 pg·hr/mL, or about 50 pg·hr/mL to about 14000 pg·hr/mL, or about 50 pg·hr/mL to about 12000 pg·hr/mL, or about 50 pg·hr/mL to about 11000 pg·hr/mL, or about 50 pg·hr/mL to about 10000 pg·hr/mL, or about 50 pg·hr/mL to about 9000 pg·hr/mL, 50 pg·hr/mL to about 8500 pg·hr/mL, or about 50 pg·hr/mL to about 7000 pg·hr/mL, or about 50 pg·hr/mL to about 5000 pg·hr/mL, or about 50 pg·hr/mL to about 3500 pg·hr/mL, or about 50 pg·hr/mL to about 2500 pg·hr/mL, 50 pg·hr/mL to about 1700 pg·hr/mL, or about 50 pg·hr/mL to about 1200 pg·hr/mL, or about 100 pg·hr/mL to about 40000 pg·hr/mL, or about 500 pg·hr/mL to about 20000 pg·hr/mL, or about 500 pg·hr/mL to about 10000 pg·hr/mL, or about 1000 pg·hr/mL to about 20000 pg·hr/mL, or about 1000 pg·hr/mL to about 10000 pg·hr/mL, or about 1000 pg·hr/mL to about 8000 pg·hr/mL, 2000 pg·hr/mL to about 20000 pg·hr/mL, or about 2000 pg·hr/mL to about 15000 pg·hr/mL, or about 2000 pg·hr/mL to about 10000 pg·hr/mL, or about 2000 pg·hr/mL to about 8000 pg·hr/mL, or about 3000 pg·hr/mL to about 40000 pg·hr/mL, or about 3000 pg·hr/mL to about 30000 pg·hr/mL, or about 3000 pg·hr/mL to about 20000 pg·hr/mL, or about 3000 pg·hr/mL to about 15000 pg·hr/mL, or about 4000 pg·hr/mL to about 40000 pg·hr/mL, or about 4000 pg·hr/mL to about 30000 pg·hr/mL, or about 4000 pg·hr/mL to about 20000 pg·hr/mL, or about 5000 pg·hr/mL to about 30000 pg·hr/mL, or about 5000 pg·hr/mL to about 20000 pg·hr/mL, or about 5000 pg·hr/mL to about 15000 pg·hr/mL, or about 6000 pg·hr/mL to about 80000 pg·hr/mL, or about 6000 pg·hr/mL to about 40000 pg·hr/mL, or about 6000 pg·hr/mL to about 30000 pg·hr/mL, or about 100 pg·hr/mL to about 80000 pg·hr/mL, about 200 pg·hr/mL to about 80000 pg·hr/mL, or about 300 pg·hr/mL to about 80000 pg·hr/mL, or about 500 pg·hr/mL to about 80000 pg·hr/mL, or about 700 pg·hr/mL to about 80000 pg·hr/mL, or about 800 pg·hr/mL to about 80000 pg·hr/mL, or about 1000 pg·hr/mL to about 80000 pg·hr/mL, or about 1200 pg·hr/mL to about 80000 pg·hr/mL, or about 1500 pg·hr/mL to about 80000 pg·hr/mL, or about 2000 pg·hr/mL to about 80000 pg·hr/mL, or about 2500 pg·hr/mL to about 80000 pg·hr/mL, or about 3000 pg·hr/mL to about 80000 pg·hr/mL, or about 3500 pg·hr/mL to about 80000 pg·hr/mL, or about 4000 pg·hr/mL to about 80000 pg·hr/mL, or about 5000 pg·hr/mL to about 80000 pg·hr/mL, 6000 pg·hr/mL to about 80000 pg·hr/mL, or about 8000 pg·hr/mL to about 80000 pg·hr/mL, or about 10000 pg·hr/mL to about 80000 pg·hr/mL, or about 12000 pg·hr/mL to about 80000 pg·hr/mL, or about 15000 pg·hr/mL to about 80000 pg·hr/mL, or about 100 pg·hr/mL to about 40000 pg·hr/mL, about 200 pg·hr/mL to about 40000 pg·hr/mL, or about 300 pg·hr/mL to about 40000 pg·hr/mL, or about 500 pg·hr/mL to about 40000 pg·hr/mL, or about 700 pg·hr/mL to about 40000 pg·hr/mL, or about 800 pg·hr/mL to about 40000 pg·hr/mL, or about 1000 pg·hr/mL to about 40000 pg·hr/mL, or about 1200 pg·hr/mL to about 40000 pg·hr/mL, or about 1500 pg·hr/mL to about 40000 pg·hr/mL, or about 2000 pg·hr/mL to about 40000 pg·hr/mL, or about 2500 pg·hr/mL to about 40000 pg·hr/mL, or about 3000 pg·hr/mL to about 40000 pg·hr/mL, or about 3500 pg·hr/mL to about 40000 pg·hr/mL, or about 4000 pg·hr/mL to about 40000 pg·hr/mL, or about 5000 pg·hr/mL to about 40000 pg·hr/mL, 6000 pg·hr/mL to about 40000 pg·hr/mL, or about 8000 pg·hr/mL to about 40000 pg·hr/mL, or about 10000 pg·hr/mL to about 40000 pg·hr/mL, or about 12000 pg·hr/mL to about 40000 pg·hr/mL, or about 15000 pg·hr/mL to about 40000 pg·hr/mL. In some preferred embodiments, the aforementioned AUC_{0-inf} being achieved with oral pharmaceutical compositions comprising a controlled release material to render said dosage form suitable for extended release.

In some preferred embodiments, the dosage form provides an oral pharmaceutical composition comprising a therapeutically effective amount of buprenorphine; said dosage form providing a systemic exposure as assessed by the mean buprenorphine and norbuprenorphine area under the plasma concentration time curve (AUC₀₋₂₄) of up to 80000 pg·hr/mL, or up to 70000 pg·hr/mL, or up to 60000 pg·hr/mL, or up to 50000 pg·hr/mL, or up to 40000 pg·hr/mL, or up to 38000 pg·hr/mL, or up to 35000 pg·hr/mL, or up to 33000 pg·hr/mL, or up to 31000 pg·hr/mL, or up to 30000 pg·hr/mL, or up to 29000 pg·hr/mL, or up to 28000 pg·hr/mL, or up to 26000 pg·hr/mL, or up to 25000 pg·hr/mL, up to 23000 pg·hr/mL, or up to 22000 pg·hr/mL, or up to 21000 pg·hr/mL, or up to 20000 pg·hr/mL, or up to 18000 pg·hr/mL, or up to 17000 pg·hr/mL, or up to 15000 pg·hr/mL, or up to 14000 pg·hr/mL, or up to 12000 pg·hr/mL, or up to 10000 pg·hr/mL.

In some preferred embodiments, the dosage form provides an oral pharmaceutical composition comprising a therapeutically effective amount of buprenorphine; said dosage form providing at least 80% of the steady state therapeutic concentration of buprenorphine and norbuprenorphine after administration of ≦three doses at their intended dosing frequency.

In some preferred embodiments, the dosage form provides an oral pharmaceutical composition comprising a therapeutically effective amount of buprenorphine; said dosage form after administration to a human patient providing a Cₘᵢₙ/Cₘₐₓ ratio of buprenorphine and norbuprenorphine of 0.1 to about 1.25. In other preferred embodiments, the dosage form provides a Cₘᵢₙ/Cₘₐₓ ratio of buprenorphine and norbuprenorphine of about 0.1 to about 1.15, about 0.1 to about 1.19, about 0.1 to about 1, about 0.1 to about 0.9, about 0.1 to about 0.85, or about 0.1 to about 0.8, or about 0.1 to about 0.7, or about 0.1 to about 0.6, or about 0.1 to about 0.5, or about 0.1 to about 0.4, or about 0.1 to about 0.3, or about 0.2 to about 1.0, or about 0.25 to about 1.25, or about 0.25 to about 1.25, or about 0.4 to about 1.25, or about 0.5 to about 1.25, or about 0.65 to about 1.25, or about 0.75 to about 1.25, or about 0.2 to about 0.9, or about 0.3 to about 0.9, or about 0.3 to about 0.8, or about 0.4 to about 0.8, or about 0.4 to about 0.7, or about 0.4 to about 0.6.

In some preferred embodiments, the dosage form provides an oral pharmaceutical composition comprising a therapeutically effective amount of buprenorphine; said dosage form after administration to a human patient providing a percent fluctuation of buprenorphine and norbuprenorphine of less than 400%. In other preferred embodiments, the dosage form provides a percent fluctuation of buprenorphine and norbuprenorphine of less than 350%, or less than 300%, or less than 250%, or less than 200%, or less than 150%, or less than 100%, or less than 75%, or less than 50%, or less than 25%.

In some preferred embodiments, the dosage form provides an oral pharmaceutical composition comprising a therapeutically effective amount of buprenorphine; said dosage form after administration to a human patient providing a W₅₀ of buprenorphine and norbuprenorphine of about 1 to about 6 hours for each 6 hour time period of intended dosing frequency and intended duration of action. In other preferred embodiments, the dosage form provides a W₅₀ of buprenorphine and norbuprenorphine for each 6 hour time period of intended dosing frequency and intended duration of action of about 1 to about 5 hours, or about 1 to about 4 hours, or about 1 to about 3 hours, or about 1 to about 2 hours, or 2 to about 6 hours, or about 3 to about 6 hours, or about 4 to about 6 hours, or about 2 to about 4 hours.

In some preferred embodiments, the dosage form provides an oral pharmaceutical composition comprising a therapeutically effective amount of buprenorphine; said dosage form after administration to a human patient providing a HVD of buprenorphine and norbuprenorphine of about 1.5 to about 6 hours for each 6 hour time period of intended dosing frequency and intended duration of action. In other preferred embodiments, the dosage form provides a HVD of buprenorphine and norbuprenorphine for each 6 hour time period of intended dosing frequency and intended duration of action of about 1.5 to about 5 hours, or about 1.5 to about 4 hours, or about 1.5 to about 3 hours, or about 1.5 to about 2 hours, or 2 to about 6 hours, or about 3 to about 6 hours, or about 4 to about 6 hours, or about 2 to about 4 hours.

In some preferred embodiments, the dosage form provides an oral pharmaceutical composition comprising a therapeutically effective amount of buprenorphine; said dosage form after administration to a human patient providing an AI of buprenorphine and norbuprenorphine of not more than 4.0. In other preferred embodiments, the dosage form provides an AI of buprenorphine and norbuprenorphine of not more than about 3.5, or not more than about 3.0, or not more than about 2.5, or not more than about 2, or not more than about 1.75, or not more than about 1.5, or not more than about 1.25, or not more than about 1, or not more than about 0.75, or not more than about 0.5, or not more than about 0.25.

In some preferred embodiments, the in vivo pharmacokinetic parameters of the specifications, embodiments and claims are achieved with oral pharmaceutical compositions comprising a controlled release material to render said dosage form suitable for extended release.

In some preferred embodiments, the specifications and claims are achieved with oral pharmaceutical compositions comprising a controlled release material to render said dosage form suitable for extended release.

In some preferred embodiments, the dosage form provides an oral pharmaceutical composition comprising a therapeutically effective amount of buprenorphine; and a controlled release material with gastroretentive properties to render said dosage form suitable for extended release oral administration to a human patient.

In some preferred embodiments, the dosage form provides an oral pharmaceutical composition comprising a therapeutically effective amount of buprenorphine; and a controlled release material with osmotic release to render said dosage form suitable for extended release oral administration to a human patient.

In some preferred embodiments, the dosage form provides an oral pharmaceutical composition comprising a therapeutically effective amount of buprenorphine; and a controlled release material with zero-order or pseudo-zero-order release to render said dosage form suitable for extended release oral administration to a human patient.

In some preferred embodiments, the dosage form provides an oral pharmaceutical composition comprising a therapeutically effective amount of buprenorphine and a controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a percent fluctuation of buprenorphine and norbuprenorphine of less than 400%. In other preferred embodiments, the dosage form provides a percent fluctuation of buprenorphine and norbuprenorphine of less than about 375%, or less than about 350%, or less than about 325%, or less than about 300%, or less than about 275%, or less than about 250%, or less than about 225%, or less than about 200%, or less than about 175%, or less than about 150%, or less than about 125%, or less than about 100%, or less than about 75%, or less than about 50%, or less than about 25%. In some preferred embodiments, the aforementioned percent fluctuation being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient, providing an AI of buprenorphine and norbuprenorphine of not more than 4.0. In other preferred embodiments, the dosage form provides an AI of buprenorphine and norbuprenorphine of not more than about 3.75, or not more than about 3.5, or not more than about 3.25, or not more than about 3, or not more than about 2.75, or not more than about 2.5, or not more than about 2, or not more than about 1.5, not more than about 1.25, or not more than about 1, or not more than about 0.75. In some preferred embodiments, the aforementioned AI ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cₘₐₓ of about 20 pg/mL to about 2000 pg/mL. In other preferred embodiments, the dosage form provides a mean buprenorphine and norbuprenorphine Cₘₐₓ of about 20 pg/mL to 1800 pg/mL, or about 20 pg/mL to 1600 pg/mL, or about 20 pg/mL to 1500 pg/mL, or about 20 pg/mL to 1400 pg/mL, or about 20 pg/mL to 1200 pg/mL, or about 20 pg/mL to 1100 pg/mL, or about 20 pg/mL to 1000 pg/mL, or about 20 pg/mL to 900 pg/mL, or about 20 pg/mL to 800 pg/mL, or about 20 pg/mL to 700 pg/mL, or about 20 pg/mL to 600 pg/mL, or about 20 pg/mL to 500 pg/mL, or about 20 pg/mL to 400 pg/mL, or about 20 pg/mL to 300 pg/mL, or about 20 pg/mL to 200 pg/mL, or about 50 pg/mL to 1800 pg/mL, or about 50 pg/mL to 1600 pg/mL, or about 50 pg/mL to 1500 pg/mL, or about 50 pg/mL to 1400 pg/mL, or about 50 pg/mL to 1500 pg/mL, or about 50 pg/mL to 1100 pg/mL, or about 50 pg/mL to 1000 pg/mL, or about 50 pg/mL to 900 pg/mL, or about 50 pg/mL to 800 pg/mL, or about 50 pg/mL to 700 pg/mL, or about 50 pg/mL to 600 pg/mL, or about 50 pg/mL to 500 pg/mL, or about 50 pg/mL to 400 pg/mL, or about 50 pg/mL to 300 pg/mL, or about 100 pg/mL to 1800 pg/mL, or about 100 pg/mL to 1600 pg/mL, or about 100 pg/mL to 11000 pg/mL, or about 100 pg/mL to 1400 pg/mL, or about 100 pg/mL to 1200 pg/mL, or about 100 pg/mL to 1100 pg/mL, or about 100 pg/mL to 1000 pg/mL, or about 100 pg/mL to 900 pg/mL, or about 100 pg/mL to 800 pg/mL, or about 100 pg/mL to 700 pg/mL, or about 100 pg/mL to 600 pg/mL, or about 100 pg/mL to 1000 pg/mL, or about 100 pg/mL to 400 pg/mL, or about 100 pg/mL to 300 pg/mL, or about 100 pg/mL to 1000 pg/mL, or about 200 pg/mL to 1600 pg/mL, or about 200 pg/mL to 1600 pg/mL, or about 200 pg/mL to 12000 pg/mL, or about 200 pg/mL to 1400 pg/mL, or about 200 pg/mL to 12000 pg/mL, or about 200 pg/mL to 1200 pg/mL, or about 200 pg/mL to 2000 pg/mL, or about 200 pg/mL to 900 pg/mL, or about 200 pg/mL to 800 pg/mL, or about 200 pg/mL to 700 pg/mL, or about 200 pg/mL to 600 pg/mL, or about 200 pg/mL to 2000 pg/mL, or about 200 pg/mL to 400 pg/mL, or about 200 pg/mL to 300 pg/mL, or about 200 pg/mL to 2000 pg/mL. In some preferred embodiments, the aforementioned Cₘₐₓ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cₘₐₓ of less than about 2000 pg/mL. In other preferred embodiments, the dosage form provides a mean buprenorphine and norbuprenorphine Cₘₐₓ of less than about 1800 pg/mL, or less than about 1700 pg/mL, or less than about 1600 pg/mL, or less than about 1500 pg/mL, or less than about 1400 pg/mL, or less than about 1200 pg/mL, or less than about 100 pg/mL, or less than about 900 pg/mL, or less than about 800 pg/mL, or less than about 700 pg/mL, or less than about 600 pg/mL, or less than about 500 pg/mL, or less than about 400 pg/mL, or less than about 300 pg/mL, or less than about 200 pg/mL, or less than about 100 pg/mL, or less than about 50 pg/mL. In some preferred embodiments, the aforementioned Cₘₐₓ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cₘᵢₙ of about 1 pg/mL to about 1000 pg/mL. In other preferred embodiments, the dosage form provides a mean buprenorphine and norbuprenorphine Cₘᵢₙ of about 1 pg/mL to 950 pg/mL, or about 1 pg/mL to 900 pg/mL, or about 1 pg/mL to 850 pg/mL, or about 1 pg/mL to 800 pg/mL, or about 1 pg/mL to 750 pg/mL, or about 1 pg/mL to 700 pg/mL, or about 1 pg/mL to 650 pg/mL, or about 1 pg/mL to 600 pg/mL, or about 1 pg/mL to 550 pg/mL, or about 1 pg/mL to 500 pg/mL, or about 1 pg/mL to 450 pg/mL, or about 1 pg/mL to 400 pg/mL, or about 1 pg/mL to 350 pg/mL, or about 1 pg/mL to 300 pg/mL, or about 1 pg/mL to 250 pg/mL, or about 1 pg/mL to 200 pg/mL, or about 1 pg/mL to 150 pg/mL, or about 1 pg/mL to 100 pg/mL, or about 1 pg/mL to 90 pg/mL, or about 1 pg/mL to 80 pg/mL, or about 1 pg/mL to 70 pg/mL, or about 1 pg/mL to 60 pg/mL, or about 1 pg/mL to 50 pg/mL, or about 1 pg/mL to 40 pg/mL, or about 1 pg/mL to 30 pg/mL, or about 1 pg/mL to 20 pg/mL, or about 1 pg/mL to 10 pg/mL, or about 5 pg/mL to 1000 pg/mL, or about 5 pg/mL to 800 pg/mL, or about 5 pg/mL to 600 pg/mL, or about 5 pg/mL to 500 pg/mL, or about 10 pg/mL to 1000 pg/mL, or about 10 pg/mL to 500 pg/mL, or about 10 pg/mL to 100 pg/mL, or about 20 pg/mL to 1000 pg/mL, or about 20 pg/mL to 500 pg/mL, or about 20 pg/mL to 200 pg/mL, or about 50 pg/mL to 1000 pg/mL, or about 50 pg/mL to 500 pg/mL, or about 50 pg/mL to 300 pg/mL, or about 100 pg/mL to 1000 pg/mL, or about 100 pg/mL to 500 pg/mL, or greater than 1 pg/mL, or greater than 5 pg/mL, or greater than 10 pg/mL, or greater than 20 pg/mL, or greater than 30 pg/mL, or greater than 40 pg/mL, or greater than 50 pg/mL, or greater than 70 pg/mL, or greater than 100 pg/mL, or greater than 200 pg/mL, or greater than 300 pg/mL. In some preferred embodiments, the aforementioned Cₘᵢₙ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cₘᵢₙ of less than about 1000 pg/mL. In other preferred embodiments, the dosage form provides a mean buprenorphine and norbuprenorphine Cₘᵢₙ of less than about 900 pg/mL, or less than about 800 pg/mL, or less than about 700 pg/mL, or less than about 600 pg/mL, or less than about 500 pg/mL, or less than about 400 pg/mL, or less than about 300 pg/mL, or less than about 200 pg/mL, or less than about 100 pg/mL, or less than about 90 pg/mL, or less than about 80 pg/mL, or less than about 70 pg/mL, or less than about 60 pg/mL, or less than about 50 pg/mL, or less than about 40 pg/mL, or less than about 30 pg/mL, or less than about 20 pg/mL, or less than about 10 pg/mL, or less than about 5 pg/mL, or less than about 1 pg/mL. In some preferred embodiments, the aforementioned Cₘᵢₙ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cₘᵢₙ of greater than about 600 pg/mL, or greater than about 500 pg/mL, or greater than about 400 pg/mL, or greater than about 300 pg/mL, or greater than about 200 pg/mL, or greater than about 100 pg/mL, or greater than about 90 pg/mL, or greater than about 80 pg/mL, or greater than about 70 pg/mL, or greater than about 60 pg/mL, or greater than about 50 pg/mL, or greater than about 40 pg/mL, or greater than about 30 pg/mL, or greater than about 20 pg/mL, or greater than about 10 pg/mL, or greater than about 5 pg/mL, or greater than about 1 pg/mL.

In some preferred embodiments, the dosage form provides an oral pharmaceutical composition comprising a therapeutically effective amount of buprenorphine and, optionally, controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a greater than about 60%, 65%, 70%, 75%, 80%, 85%, 90%, 95%, or 100% of the total absorbed dose into systemic circulation (as measured by bioavailability) during the first half of the intended dosing frequency.

In some preferred embodiments, the dosage form provides an oral pharmaceutical composition comprising a therapeutically effective amount of buprenorphine and, optionally, controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a greater than about 50%, 55%, 60%, 65%, 70%, 75%, 80%, 85%, 90%, 95%, or 100% of the total absorbed dose into systemic circulation (as measured by bioavailability) during the first one-third of the intended dosing frequency.

In some preferred embodiments, the dosage form provides an oral pharmaceutical composition comprising a therapeutically effective amount of buprenorphine and, optionally, controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a greater than about 50%, 55%, 60%, 65%, 70%, 75%, 80%, 85%, 90%, 95%, or 100% of the total absorbed dose into systemic circulation (as measured by bioavailability) during the first one-quarter of the intended dosing frequency.

In some preferred embodiments, in order to attain the specifications and claims of the invention, it is necessary or critical to incorporate a controlled release material in the dosage form.

In some preferred embodiments, the aforementioned embodiments which provide a greater amount of the total absorbed dose into systemic circulation (as measured by bioavailability) during the first half, first one-third or first one quarter of the intended dosing frequency result in reduced frequency or duration of buprenorphine related side effects.

In some preferred embodiments, the dosage form comprises a therapeutically effective amount of oral buprenorphine or a pharmaceutically acceptable salt of buprenorphine, or a mixture thereof, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the in vivo specifications, including pharmacokinetic specifications achieved with oral pharmaceutical compositions which are devoid of a controlled release material to render said dosage form extended release.

In some preferred embodiments, the dosage form comprises a therapeutically effective amount of oral buprenorphine or a pharmaceutically acceptable salt of buprenorphine, or a mixture thereof, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the in vitro specifications, including dissolution rate specifications achieved with oral pharmaceutical compositions which are devoid of a controlled release material to render said dosage form extended release.

Pharmacokinetic parameters in the specifications (e.g., AUC, Cmax) referring to “buprenorphine and norbuprenorphine” mean buprenorphine alone and norbuprenorphine alone and not a summation of the two.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the in-vitro release rate is substantially independent of pH in that a difference, at any given time, between an amount of buprenorphine released at one pH and an amount released at any other pH, when measured in-vitro using the USP Basket or Paddle Method of USP Drug Release test of U.S. Pharmacopeia (2003) at 100 rpm in 900 ml aqueous buffer, is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine Cₘₐₓ is substantially independent of food intake in that a difference, at any given time, between the Cₘₐₓ of buprenorphine administered in fasted state and the Cₘₐₓ of buprenorphine and norbuprenorphine administered in fed state (using a standardized meal) is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine Cₘₐₓ is substantially independent of food intake in that a difference, at any given time, between the Cₘₐₓ of buprenorphine and norbuprenorphine administered in fasted state and the Cₘₐₓ of buprenorphine administered after a standardized high fat meal is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine Cₘₐₓ is substantially independent of alcohol intake in that a difference, at any given time, between the Cₘₐₓ of buprenorphine and norbuprenorphine administered with about 30 to about 24 mL of a 40% ethanol solution and the Cₘₐₓ of buprenorphine administered without concurrent alcohol (i.e., in an alcohol free state) is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the AUC₀₋₁₂, AUC₀₋₂₄ and AUC_{0-inf} after single-dose administration are substantially independent of food intake in that a difference, at any given time, between the said AUC of buprenorphine and norbuprenorphine when the dosage form administered in fasted state and the said AUC of buprenorphine and norbuprenorphine when the dosage form is administered in fed state (using a standardized meal) is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine AUC₀₋₁₂, AUC₀₋₂₄ and AUC_{0-inf} is substantially independent of food intake in that a difference, at any given time, between the said AUC when administered in fasted state and the AUC when administered after a standardized high fat meal is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine AUC₀₋₁, AUC₀₋₂, and AUC₀₋₄ is substantially independent of alcohol intake in that a difference, at any given time, between the said AUC when administered with about 30 to about 24 mL of a 40% ethanol solution and the said AUC when administered without concurrent alcohol (i.e., in an alcohol free state) is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine and, optionally, controlled release material to render said dosage form suitable for three times-a-day (Q8H or Q8H PRN) administration to a human patient, said dosage form after administration to a human patient providing a Cₘₐₓ of buprenorphine and norbuprenorphine at 0.75 to about 7 hours; and said dosage form providing a therapeutic effect for at least about 8 hours. In other preferred embodiments, the dosage form provides a Cₘₐₓ of buprenorphine and norbuprenorphine at about 0.75 to about 6.5 hours or about 0.75 to about 6 hours, or about 0.75 to about 5 hours, or about 0.75 to about 4 hours, or about 0.75 to about 3.5 hours, or about 0.75 to about 3 hours, or 0.75 to about 2.5 hours, or about 0.75 to about 2 hours, or about 0.75 to about 1.5 hours, or about 1 to about 7 hours, or about 1.5 to about 7 hours, or about 2 to about 7 hours, or about 2.5 to about 7 hours, or 3 to about 7 hours, or about 3.5 to about 7 hours, or about 4 to about 7 hours, or about 4.5 to about 7 hours, or about 5 to about 6 hours, or about 5.5 to about 7 hours, or about 2 to about 6, or about 2.5 to about 5.5 hours, or about 3 to about 5 hours, or about 3 to about 6. In some preferred embodiments, the aforementioned Cₘₐₓ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and, optionally, controlled release material to render said dosage form suitable for three times-a-day (Q8H or Q8H PRN) administration to a human patient, said dosage form after administration to a human patient providing a Cₘᵢₙ of buprenorphine and norbuprenorphine at about 6 to about 10 hours; and said dosage form providing a therapeutic effect for at least about 8 hours. In other preferred embodiments, the dosage form provides a Cₘᵢₙ of buprenorphine and norbuprenorphine at about 6 to about 9 hours, or about 6 to about 8.5 hours, or about 6 to about 8 hours, or about 6 to about 7.5 hours, or about 6 to about 7 hours, or about 6.5 to about 10 hours, or about 7 to about 10 hours, or about 8 to about 10 hours, or about 8 hours. In some preferred embodiments, the aforementioned Cₘᵢₙ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and, optionally, controlled release material to render said dosage form suitable for three times-a-day (Q8H or Q8H PRN) administration to a human patient, said dosage form after administration to a human patient providing a mean buprenorphine and norbuprenorphine AUC_{0-inf} after first administration or AUC₀₋₈ at steady state of about 125 pg·hr/mL to about 40000 pg·hr/mL; and said dosage form providing a therapeutic effect for at least about 8 hours. In other preferred embodiments, the dosage form provides a mean buprenorphine and norbuprenorphine AUC_{0-inf} after first administration or AUC₀₋₈ at steady state of about 125 pg·hr/mL to about 35000 pg·hr/mL, or about 125 pg·hr/mL to about 30000 pg·hr/mL, or about 125 pg·hr/mL to about 28000 pg·hr/mL, or about 125 pg·hr/mL to about 25000 pg·hr/mL, or about 125 pg·hr/mL to about 22000 pg·hr/mL, or about 125 pg·hr/mL to about 20000 pg·hr/mL, or about 125 pg·hr/mL to about 18000 pg·hr/mL, or about 125 pg·hr/mL to about 15000 pg·hr/mL, or about 125 pg·hr/mL to about 12500 pg·hr/mL, or about 125 pg·hr/mL to about 10000 pg·hr/mL, or about 125 pg·hr/mL to about 8000 pg·hr/mL, or about 125 pg·hr/mL to about 7500 pg·hr/mL, or about 125 pg·hr/mL to about 7000 pg·hr/mL, or about 125 pg·hr/mL to about 6000 pg·hr/mL, or about 125 pg·hr/mL to about 5500 pg·hr/mL, or about 125 pg·hr/mL to about 5000 pg·hr/mL, or about 125 pg·hr/mL to about 4000 pg·hr/mL, or about 125 pg·hr/mL to about 3500 pg·hr/mL, or about 125 pg·hr/mL to about 3000 pg·hr/mL, or about 125 pg·hr/mL to about 25000 pg·hr/mL, or about 125 pg·hr/mL to about 2000 pg·hr/mL, or about 125 pg·hr/mL to about 1800 pg·hr/mL, or about 125 pg·hr/mL to about 1500 pg·hr/mL, or about 125 pg·hr/mL to about 1000 pg·hr/mL, or about 125 pg·hr/mL to about 800 pg·hr/mL, or about 125 pg·hr/mL to about 700 pg·hr/mL, or about 125 pg·hr/mL to about 6000 pg·hr/mL, or about 125 pg·hr/mL to about 500 pg·hr/mL, or about 125 pg·hr/mL to about 400 pg·hr/mL, or about 175 pg·hr/mL to about 40000 pg·hr/mL, or about 200 pg·hr/mL to about 40000 pg·hr/mL, or about 300 pg·hr/mL to about 40000 pg·hr/mL, or about 400 pg·hr/mL to about 40000 pg·hr/mL, or about 500 pg·hr/mL to about 40000 pg·hr/mL, or about 600 pg·hr/mL to about 40000 pg·hr/mL, or about 700 pg·hr/mL to about 40000 pg·hr/mL, or about 800 pg·hr/mL to about 40000 pg·hr/mL, or about 1000 pg·hr/mL to about 40000 pg·hr/mL, or about 1200 pg·hr/mL to about 40000 pg·hr/mL, or about 1500 pg·hr/mL to about 40000 pg·hr/mL, or about 2000 pg·hr/mL to about 40000 pg·hr/mL, or about 3000 pg·hr/mL to about 4000 pg·hr/mL, or about 4000 pg·hr/mL to about 40000 pg·hr/mL, or about 5000 pg·hr/mL to about 40000 pg·hr/mL, or about 6000 pg·hr/mL to about 40000 pg·hr/mL, or about 8000 pg·hr/mL to about 40000 pg·hr/mL, or about 10000 pg·hr/mL to about 40000 pg·hr/mL, or about 300 pg·hr/mL to about 30000 pg·hr/mL, or about 300 pg·hr/mL to about 20000 pg·hr/mL, or about 300 pg·hr/mL to about 15000 pg·hr/mL, or about 300 pg·hr/mL to about 10000 pg·hr/mL, or about 300 pg·hr/mL to about 8000 pg·hr/mL, or about 300 pg·hr/mL to about 6000 pg·hr/mL, or about 300 pg·hr/mL to about 5000 pg·hr/mL, or about 300 pg·hr/mL to about 4000 pg·hr/mL, or about 300 pg·hr/mL to about 3000 pg·hr/mL, or about 300 pg·hr/mL to about 2500 pg·hr/mL, or about 300 pg·hr/mL to about 2000 pg·hr/mL, or about 300 pg·hr/mL to about 1500 pg·hr/mL, or about 300 pg·hr/mL to about 1000 pg·hr/mL, or about 600 pg·hr/mL to about 30000 pg·hr/mL, or about 600 pg·hr/mL to about 20000 pg·hr/mL, or about 600 pg·hr/mL to about 15000 pg·hr/mL, or about 600 pg·hr/mL to about 10000 pg·hr/mL, or about 600 pg·hr/mL to about 8000 pg·hr/mL, or about 600 pg·hr/mL to about 6000 pg·hr/mL, or about 600 pg·hr/mL to about 5000 pg·hr/mL, or about 600 pg·hr/mL to about 4000 pg·hr/mL, or about 600 pg·hr/mL to about 3000 pg·hr/mL, or about 600 pg·hr/mL to about 2500 pg·hr/mL, or about 600 pg·hr/mL to about 2000 pg·hr/mL, or about 600 pg·hr/mL to about 1500 pg·hr/mL, or about 600 pg·hr/mL to about 1000 pg·hr/mL, or about 1000 pg·hr/mL to about 30000 pg·hr/mL, or about 1000 pg·hr/mL to about 20000 pg·hr/mL, or about 1000 pg·hr/mL to about 15000 pg·hr/mL, or about 1000 pg·hr/mL to about 10000 pg·hr/mL, or about 1000 pg·hr/mL to about 8000 pg·hr/mL, or about 1000 pg·hr/mL to about 10000 pg·hr/mL, or about 1000 pg·hr/mL to about 5000 pg·hr/mL, or about 1000 pg·hr/mL to about 4000 pg·hr/mL, or about 1000 pg·hr/mL to about 3000 pg·hr/mL, or about 1000 pg·hr/mL to about 2500 pg·hr/mL, or about 1000 pg·hr/mL to about 2000 pg·hr/mL, or about 1000 pg·hr/mL to about 1500 pg·hr/mL, or about 2000 pg·hr/mL to about 30000 pg·hr/mL, or about 2000 pg·hr/mL to about 20000 pg·hr/mL, or about 2000 pg·hr/mL to about 15000 pg·hr/mL, or about 2000 pg·hr/mL to about 20000 pg·hr/mL, or about 2000 pg·hr/mL to about 8000 pg·hr/mL, or about 2000 pg·hr/mL to about 20000 pg·hr/mL, or about 2000 pg·hr/mL to about 5000 pg·hr/mL, or about 2000 pg·hr/mL to about 4000 pg·hr/mL, or about 2000 pg·hr/mL to about 3000 pg·hr/mL, or about 2000 pg·hr/mL to about 2500 pg·hr/mL, or about 2000 pg·hr/mL to about 2000 pg·hr/mL, or about 2000 pg·hr/mL to about 1500 pg·hr/mL, or about 4000 pg·hr/mL to about 30000 pg·hr/mL, or about 4000 pg·hr/mL to about 20000 pg·hr/mL, or about 6000 pg·hr/mL to about 15000 pg·hr/mL, or about 6000 pg·hr/mL to about 15000 pg·hr/mL, or about 100 pg·hr/mL to about 4000 pg·hr/mL, or about 100 pg·hr/mL to about 3000 pg·hr/mL. In some preferred embodiments, the aforementioned AUC being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for three times-a-day (Q8H or Q8H PRN) administration to a human patient, said dosage form after administration to a human patient providing a C₈/Cₘₐₓ ratio of buprenorphine and norbuprenorphine 0.1 to about 1.25; and said dosage form providing a therapeutic effect for at least about 8 hours. In other preferred embodiments, the dosage form provides a C₈/Cₘₐₓ ratio of buprenorphine and norbuprenorphine of about 0.1 to about 1, or about 0.1 to about 0.8, or about 0.1 to about 0.75, or about 0.1 to about 0.6, or 0.1 to about 0.5, or about 0.1 to about 0.4, or about 0.1 to about 0.35, or about 0.25 to about 0.95, or about 0.4 to about 0.95, or about 0.5 to about 0.95, or about 0.65 to about 0.95, or about 0.75 to about 0.95, or about 0.3 to about 0.8, or about 0.4 to about 0.75, or about 0.5 to about 0.75. In some preferred embodiments, the aforementioned C₈/Cₘₐₓ ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for three times-a-day (Q8H or Q8H PRN) administration to a human patient, said dosage form after administration to a human patient, providing a W₅₀ of buprenorphine and norbuprenorphine of 1 to about 7 hours; and said dosage form providing a therapeutic effect for at least about 8 hours. In other preferred embodiments, the dosage form provides a W₅₀ of buprenorphine and norbuprenorphine of about 1 to about 6 hours, or about 1 to about 5 hours, or about 1 to about 5.5 hours, or about 1 to about 5 hours, or 1 to about 4.5 hours, or about 1 to about 4 hours, or about 1 to about 3.5 hours, or about 1 to about 3 hours, or about 1 to about 2.5 hours, or about 1 to about 2 hours, or about 1.5 to about 7 hours, or about 2 to about 6 hours, or 2 to about 5.5 hours, or about 2 to about 5 hours, or about 2 to about 4.5 hours, or about 2 to about 4 hours, or about 2 to about 3.5 hours, or about 2.5 to about 6.5 hours, or about 2.5 to about 6 hours, or about 2.5 to about 5 hours, or about 2.5 to about 4.5 hours, or 3 to about 6.5 hours, or about 3 to about 6 hours, or about 3 to about 5 hours, or about 3 to about 6.5 hours. In some preferred embodiments, the aforementioned W₅₀ ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for three times-a-day (Q8H or Q8H PRN) administration to a human patient, said dosage form after administration to a human patient, providing a HVD of buprenorphine and norbuprenorphine of 1.5 to about 7 hours; and said dosage form providing a therapeutic effect for at least about 8 hours. In other preferred embodiments, the dosage form provides a HVD of buprenorphine and norbuprenorphine of about 1.5 to about 6 hours, or about 1.5 to about 5 hours, or about 1.5 to about 5.5 hours, or about 1.5 to about 5 hours, or 1.5 to about 4.5 hours, or about 1.5 to about 4 hours, or about 1.5 to about 3.5 hours, or about 1.5 to about 3 hours, or about 1.5 to about 2.5 hours, or about 1.5 to about 2 hours, or about 1.5 to about 7 hours, or about 2 to about 6 hours, or 2 to about 5.5 hours, or about 2 to about 5 hours, or about 2 to about 4.5 hours, or about 2 to about 4 hours, or about 2 to about 3.5 hours, or about 2.5 to about 6.5 hours, or about 2.5 to about 6 hours, or about 2.5 to about 5 hours, or about 2.5 to about 4.5 hours, or 3 to about 6.5 hours, or about 3 to about 6 hours, or about 3 to about 5 hours, or about 3 to about 6.5 hours. In some preferred embodiments, the aforementioned HVD ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine, and, optionally, controlled release material to render said dosage form suitable for three times-a-day (Q8H or Q8H PRN) administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 10% to about 90% at 4 hours, from about 15% to about 95% at 6 hours, and greater than about 65% at 8 hours. In other preferred embodiments, the dosage form provides said an in-vitro release rate of from 0% to about 40% at 1 hour, from about 5% to about 60% at 2 hours, from about 10% to about 70% at 4 hours, from about 15% to about 90% at 6 hours, and greater than about 50% at 8 hours.

In some preferred embodiments, the dosage form provides an oral pharmaceutical composition comprising a therapeutically effective amount of buprenorphine, and, optionally, controlled release material to render said dosage form suitable for three times-a-day (Q8H or Q8H PRN) administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 80% at 6 hours, and greater than about 65% at 8 hours; said in-vitro release rate being substantially independent of pH in that a difference, at any given time, between an amount of buprenorphine released at one pH and an amount released at any other pH, when measured in-vitro using the USP Basket or Paddle Method of USP Drug Release test of U.S. Pharmacopeia (2003) at 100 rpm in 900 ml aqueous buffer, is no greater than 30%. In other preferred embodiments, said pH independent in-vitro release rate is from 0% to about 47.5% at 1 hour, from about 10% to about 65% at 2 hours, from about 10% to about 90% at 4 hours, from about 15% to about 95% at 6 hours, and greater than about 65% at 8 hours or from 0% to about 40% at 1 hour, from about 5% to about 60% at 2 hours, from about 10% to about 70% at 4 hours, from about 15% to about 90% at 6 hours, and greater than about 50% at 8 hours.

In some preferred embodiments, the dosage form provides an oral pharmaceutical composition comprising a therapeutically effective amount of buprenorphine and controlled release material to render said dosage form suitable for twice-a-day administration to a human patient, said dosage form after administration to a human patient providing a Cₘₐₓ of buprenorphine and norbuprenorphine at 2 to about 10 hours; and said dosage form providing a therapeutic effect for at least about 12 hours. In other preferred embodiments, the dosage form provides a Cₘₐₓ of buprenorphine and norbuprenorphine at about 2 to about 8 hour or about 2 to about 6 hours, or about 2 to about 5 hours, or about 2 to about 7 hours, or about 2 to about 4.5 hours, or about 2 to about 4 hours, or 2 to about 3.5 hours, or about 2 to about 3 hours, or about 3 to about 10 hours, or about 3.5 to about 10 hours, or about 4 to about 10 hours, or about 4.5 to about 10 hours, or about 5 to about 10 hours, or 5 to about 10 hours, or about 6 to about 10 hours, or about 3 to about 8 hours, or about 3 to about 7 hours, or about 3 to about 6 hours, or about 4 to about 8 hours, or about 4 to about 6. In some preferred embodiments, the aforementioned Cₘₐₓ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for twice-a-day administration to a human patient, said dosage form after administration to a human patient providing a Cₘᵢₙ of buprenorphine and norbuprenorphine at about 10 to about 14 hours; and said dosage form providing a therapeutic effect for at least about 12 hours. In other preferred embodiments, the dosage form provides a Cₘᵢₙ of buprenorphine and norbuprenorphine at about 10 to about 13 hours, or about 10 to about 12.5 hours, or about 10 to about 12 hours, or about 10 to about 11.5 hours, or about 10 to about 11 hours, or about 10.5 to about 14 hours, or about 11 to about 14 hours, or about 12 to about 14 hours. In some preferred embodiments, the aforementioned Cₘᵢₙ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for twice-a-day administration to a human patient, said dosage form after administration to a human patient providing a mean buprenorphine and norbuprenorphine AUC_{0-inf} after first administration or AUC₀₋₁₂ at steady state of about 125 pg·hr/mL to about 250000 pg·hr/mL; and said dosage form providing a therapeutic effect for at least about 12 hours. In other preferred embodiments, the dosage form provides a mean buprenorphine and norbuprenorphine AUC_{0-inf} after first administration or AUC₀₋₁₂ at steady state of about 125 pg·hr/mL to about 200000 pg·hr/mL, or about 125 pg·hr/mL to about 150000 pg·hr/mL, or about 125 pg·hr/mL to about 100000 pg·hr/mL, or about 125 pg·hr/mL to about 50000 pg·hr/mL, or about 125 pg·hr/mL to about 22000 pg·hr/mL, or about 125 pg·hr/mL to about 20000 pg·hr/mL, or about 125 pg·hr/mL to about 18000 pg·hr/mL, or about 125 pg·hr/mL to about 15000 pg·hr/mL, or about 125 pg·hr/mL to about 12500 pg·hr/mL, or about 125 pg·hr/mL to about 10000 pg·hr/mL, or about 125 pg·hr/mL to about 8000 pg·hr/mL, or about 125 pg·hr/mL to about 7500 pg·hr/mL, or about 125 pg·hr/mL to about 7000 pg·hr/mL, or about 125 pg·hr/mL to about 6000 pg·hr/mL, or about 125 pg·hr/mL to about 5500 pg·hr/mL, or about 125 pg·hr/mL to about 5000 pg·hr/mL, or about 125 pg·hr/mL to about 4000 pg·hr/mL, or about 125 pg·hr/mL to about 3500 pg·hr/mL, or about 125 pg·hr/mL to about 3000 pg·hr/mL, or about 125 pg·hr/mL to about 25000 pg·hr/mL, or about 125 pg·hr/mL to about 2000 pg·hr/mL, or about 125 pg·hr/mL to about 1800 pg·hr/mL, or about 125 pg·hr/mL to about 1500 pg·hr/mL, or about 125 pg·hr/mL to about 1000 pg·hr/mL, or about 125 pg·hr/mL to about 800 pg·hr/mL, or about 125 pg·hr/mL to about 700 pg·hr/mL, or about 125 pg·hr/mL to about 6000 pg·hr/mL, or about 125 pg·hr/mL to about 500 pg·hr/mL, or about 125 pg·hr/mL to about 400 pg·hr/mL, or about 175 pg·hr/mL to about 40000 pg·hr/mL, or about 200 pg·hr/mL to about 40000 pg·hr/mL, or about 300 pg·hr/mL to about 40000 pg·hr/mL, or about 400 pg·hr/mL to about 40000 pg·hr/mL, or about 500 pg·hr/mL to about 40000 pg·hr/mL, or about 600 pg·hr/mL to about 40000 pg·hr/mL, or about 700 pg·hr/mL to about 40000 pg·hr/mL, or about 800 pg·hr/mL to about 40000 pg·hr/mL, or about 1000 pg·hr/mL to about 40000 pg·hr/mL, or about 1200 pg·hr/mL to about 40000 pg·hr/mL, or about 1500 pg·hr/mL to about 40000 pg·hr/mL, or about 2000 pg·hr/mL to about 40000 pg·hr/mL, or about 3000 pg·hr/mL to about 4000 pg·hr/mL, or about 4000 pg·hr/mL to about 40000 pg·hr/mL, or about 5000 pg·hr/mL to about 40000 pg·hr/mL, or about 6000 pg·hr/mL to about 40000 pg·hr/mL, or about 8000 pg·hr/mL to about 40000 pg·hr/mL, or about 10000 pg·hr/mL to about 40000 pg·hr/mL, or about 300 pg·hr/mL to about 30000 pg·hr/mL, or about 300 pg·hr/mL to about 20000 pg·hr/mL, or about 300 pg·hr/mL to about 15000 pg·hr/mL, or about 300 pg·hr/mL to about 10000 pg·hr/mL, or about 300 pg·hr/mL to about 8000 pg·hr/mL, or about 300 pg·hr/mL to about 6000 pg·hr/mL, or about 300 pg·hr/mL to about 5000 pg·hr/mL, or about 300 pg·hr/mL to about 4000 pg·hr/mL, or about 300 pg·hr/mL to about 3000 pg·hr/mL, or about 300 pg·hr/mL to about 2500 pg·hr/mL, or about 300 pg·hr/mL to about 2000 pg·hr/mL, or about 300 pg·hr/mL to about 1500 pg·hr/mL, or about 300 pg·hr/mL to about 1000 pg·hr/mL, or about 600 pg·hr/mL to about 30000 pg·hr/mL, or about 600 pg·hr/mL to about 20000 pg·hr/mL, or about 600 pg·hr/mL to about 15000 pg·hr/mL, or about 600 pg·hr/mL to about 10000 pg·hr/mL, or about 600 pg·hr/mL to about 8000 pg·hr/mL, or about 600 pg·hr/mL to about 6000 pg·hr/mL, or about 600 pg·hr/mL to about 5000 pg·hr/mL, or about 600 pg·hr/mL to about 4000 pg·hr/mL, or about 600 pg·hr/mL to about 3000 pg·hr/mL, or about 600 pg·hr/mL to about 2500 pg·hr/mL, or about 600 pg·hr/mL to about 2000 pg·hr/mL, or about 600 pg·hr/mL to about 1500 pg·hr/mL, or about 600 pg·hr/mL to about 1000 pg·hr/mL, or about 1000 pg·hr/mL to about 30000 pg·hr/mL, or about 1000 pg·hr/mL to about 20000 pg·hr/mL, or about 1000 pg·hr/mL to about 15000 pg·hr/mL, or about 1000 pg·hr/mL to about 10000 pg·hr/mL, or about 1000 pg·hr/mL to about 8000 pg·hr/mL, or about 1000 pg·hr/mL to about 10000 pg·hr/mL, or about 1000 pg·hr/mL to about 5000 pg·hr/mL, or about 1000 pg·hr/mL to about 4000 pg·hr/mL, or about 1000 pg·hr/mL to about 3000 pg·hr/mL, or about 1000 pg·hr/mL to about 2500 pg·hr/mL, or about 1000 pg·hr/mL to about 2000 pg·hr/mL, or about 1000 pg·hr/mL to about 1500 pg·hr/mL, or about 2000 pg·hr/mL to about 30000 pg·hr/mL, or about 2000 pg·hr/mL to about 20000 pg·hr/mL, or about 2000 pg·hr/mL to about 15000 pg·hr/mL, or about 2000 pg·hr/mL to about 20000 pg·hr/mL, or about 2000 pg·hr/mL to about 8000 pg·hr/mL, or about 2000 pg·hr/mL to about 20000 pg·hr/mL, or about 2000 pg·hr/mL to about 5000 pg·hr/mL, or about 2000 pg·hr/mL to about 4000 pg·hr/mL, or about 2000 pg·hr/mL to about 3000 pg·hr/mL, or about 2000 pg·hr/mL to about 2500 pg·hr/mL, or about 2000 pg·hr/mL to about 2000 pg·hr/mL, or about 2000 pg·hr/mL to about 1500 pg·hr/mL, or about 4000 pg·hr/mL to about 30000 pg·hr/mL, or about 4000 pg·hr/mL to about 20000 pg·hr/mL, or about 6000 pg·hr/mL to about 15000 pg·hr/mL, or about 6000 pg·hr/mL to about 15000 pg·hr/mL, or about 100 pg·hr/mL to about 4000 pg·hr/mL, or about 100 pg·hr/mL to about 3000 pg·hr/mL. In some preferred embodiments, the aforementioned AUC being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for twice-a-day administration to a human patient, said dosage form after administration to a human patient providing a C₁₂/Cₘₐₓ ratio of buprenorphine and norbuprenorphine 0.1 to about 1.25; and said dosage form providing a therapeutic effect for at least about 12 hours. In other preferred embodiments, the dosage form provides a C₁₂/Cₘₐₓ ratio of buprenorphine and norbuprenorphine of about 0.1 to about 1, or about 0.1 to about 0.8, or about 0.1 to about 0.75, or about 0.1 to about 0.6, or 0.1 to about 0.5, or about 0.1 to about 0.4, or about 0.1 to about 0.35, or about 0.25 to about 0.95, or about 0.4 to about 0.95, or about 0.5 to about 0.95, or about 0.65 to about 0.95, or about 0.75 to about 0.95, or about 0.3 to about 0.8, or about 0.4 to about 0.75, or about 0.5 to about 0.75. In some preferred embodiments, the aforementioned C₁₂/Cₘₐₓ ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and, optionally, controlled release material to render said dosage form suitable for twice-a-day administration to a human patient, said dosage form after administration to a human patient, providing a W₅₀ of buprenorphine and norbuprenorphine of 2 to about 11 hours; and said dosage form providing a therapeutic effect for at least about 12 hours. In other preferred embodiments, the dosage form provides a W₅₀ of buprenorphine and norbuprenorphine of about 2 to about 10 hours, or about 2 to about 9 hours, or about 2 to about 9 hours, or about 2 to about 8 hours, or 2 to about 7 hours, or about 2 to about 6 hours, or about 2 to about 5 hours, or about 2 to about 4 hours, or about 3 to about 10 hours, or about 4 to about 10 hours, or about 5 to about 10 hours, or about 6 to about 10 hours, or 7 to about 10 hours, or about 3 to about 8 hours, or about 4 to about 8 hours, or about 4 to about 7 hours, or about 3 to about 6 hours.

In some preferred embodiments, the dosage form provides an oral pharmaceutical composition comprising a therapeutically effective amount of buprenorphine and, optionally, controlled release material to render said dosage form suitable for twice-a-day administration to a human patient, said dosage form after administration to a human patient, providing a HVD of buprenorphine and norbuprenorphine of 1.5 to about 10 hours; and said dosage form providing a therapeutic effect for at least about 12 hours. In other preferred embodiments, the dosage form provides an HVD of buprenorphine and norbuprenorphine of about 1.5 to about 9 hours, or about 1.5 to 8 hours, or about 1.5 to about 7 hours, or about 1.5 to 6 hours, or about 1.5 to about 5 hours, or about 1.5 to about 4 hours, or about 2 to about 10 hours, or about 3 to 10 hours, or about 4 to about 10 hours, or about 5 to 10 hours, or about 6 to about 10 hours, or about 8 to 10 hours, about 3 to about 8 hours, or about 4 to 8 hours, or about 5 to about 7 hours, or about 3 to 6 hours, or about 3 to about 8 hours, or about 5 to about 8 hours.

In some preferred embodiments, the dosage form provides an oral pharmaceutical composition comprising a therapeutically effective amount of buprenorphine, and, optionally, controlled release material to render said dosage form suitable for twice-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours. In other preferred embodiments, the dosage form provides said an in-vitro release rate of from 0% to about 40% at 1 hour, from about 5% to about 55% at 2 hours, from about 10% to about 60% at 4 hours, from about 15% to about 70% at 6 hours, from about 25% to about 80% at 9 hours, and greater than about 50% at 12 hours

In some preferred embodiments, the dosage form provides an oral pharmaceutical composition comprising a therapeutically effective amount of buprenorphine, and, optionally, controlled release material to render said dosage form suitable for twice-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours.

In some preferred embodiments, the dosage form provides an oral pharmaceutical composition comprising a therapeutically effective amount of buprenorphine, and, optionally, controlled release material to render said dosage form suitable for twice-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours; said in-vitro release rate being substantially independent of pH in that a difference, at any given time, between an amount of buprenorphine released at one pH and an amount released at any other pH, when measured in-vitro using the USP Basket or Paddle Method of USP Drug Release test of U.S. Pharmacopeia (2003) at 100 rpm in 900 ml aqueous buffer, is no greater than 30%.

In some preferred embodiments, the dosage form provides an oral pharmaceutical composition comprising a therapeutically effective amount of buprenorphine and, optionally, controlled release material to render said dosage form suitable for once-a-day administration to a human patient, said dosage form after administration to a human patient providing a Cₘₐₓ of buprenorphine and norbuprenorphine at about 3 to about 20 hours; and said dosage form providing a therapeutic effect for at least about 24 hours. In some preferred embodiments, the dosage form provides a Cₘₐₓ at about 3 to about 18 hours, or about 3 to about 15 hours, or about 3 to about 12 hours, or at about 3 to about 10 hours, or at about 3 to about 8 hours, or at about 3 to about 7 hours, or at about 3 to about 7 hours, or about 4 to about 20 hours, or about 5 to about 20 hours, or about 6 to about 20 hours, or at about 8 to about 20 hours, or at about 10 to about 20 hours, or at about 12 to about 20 hours, or at about 14 to about 20 hours, or about 18 to about 20 hours, or about 4 to about 18 hours, or about 4 to about 16 hours, or at about 4 to about 12 hours, or at about 4 to about 8 hours, or at about 4 to about 10 hours, or at about 3 to about 6 hours.

In some preferred embodiments, the dosage form provides an oral pharmaceutical composition comprising a therapeutically effective amount of buprenorphine and, optionally, controlled release material to render said dosage form suitable for once-a-day administration to a human patient, said dosage form after administration to a human patient providing a Cₘᵢₙ of buprenorphine and norbuprenorphine at about 20 to about 28 hours; and said dosage form providing a therapeutic effect for at least about 24 hours. In some preferred embodiments, the dosage form provides a Cₘᵢₙ at about 20 to about 26 hours, or about 20 to about 27 hours, or about 20 to about 25 hours, or about 20 to about 24 hours, or about 20 to about 23 hours, or about 21 to about 28 hours, or about 22 to about 28 hours, or about 23 to about 28 hours, or about 23.5 to about 28 hours, or about 22 to 26 hours.

In some preferred embodiments, the dosage form provides an oral pharmaceutical composition comprising a therapeutically effective amount of buprenorphine; said dosage form providing a therapeutic effect longer than would be expected based on the prevailing plasma concentrations. For example, under normal circumstances, many drugs provide duration of effect that is at least partly correlated with or dependent on the prevailing plasma concentrations of drug. In some preferred embodiments of the invention, the dosage form provides persistent therapeutic effects despite short lived, low or negligible prevailing plasma concentrations.

In some preferred embodiments of the invention, the dosage form provides sustained therapeutic effects of up to about 4, or about 6, or about 8, or about 12, or about 18 or about 20 or about 24 hours despite being administered in immediate release form.

In some preferred embodiments, the dosage form provides an oral pharmaceutical composition comprising a therapeutically effective amount of buprenorphine; said dosage from providing a Cmax of buprenorphine and norbuprenorphine from about 0.25 hours to about 30 hours.

In some preferred embodiments, the dosage form provides an oral pharmaceutical composition comprising a therapeutically effective amount of buprenorphine; said dosage from providing a Cₘᵢₙ of buprenorphine and norbuprenorphine from about 1 hour to about 30 hours.

In some preferred embodiments, the dosage form provides an oral pharmaceutical composition comprising a therapeutically effective amount of buprenorphine and controlled release material to render said dosage form suitable for once-a-day administration to a human patient, said dosage form after administration to a human patient providing a buprenorphine and norbuprenorphine mean AUC_{0-inf} after first administration or AUC₀₋₂₄ at steady state of about 2250 pg·hr/mL to about 500000 pg·hr/mL; and said dosage form providing a therapeutic effect for at least about 24 hours. In other preferred embodiments, the dosage form provides a mean buprenorphine and norbuprenorphine AUC_{0-inf} after first administration or AUC₀₋₂₄ at steady state of about 250 pg·hr/mL to about 350000 pg·hr/mL, or about 250 pg·hr/mL to about 250000 pg·hr/mL, or about 250 pg·hr/mL to about 150000 pg·hr/mL, or about 250 pg·hr/mL to about 100000 pg·hr/mL, or about 250 pg·hr/mL to about 50000 pg·hr/mL, or about 250 pg·hr/mL to about 25000 pg·hr/mL, or about 250 pg·hr/mL to about 20000 pg·hr/mL, or about 250 pg·hr/mL to about 18000 pg·hr/mL, or about 250 pg·hr/mL to about 15000 pg·hr/mL, or about 250 pg·hr/mL to about 14000 pg·hr/mL, or about 250 pg·hr/mL to about 12000 pg·hr/mL, or about 250 pg·hr/mL to about 11000 pg·hr/mL, or about 250 pg·hr/mL to about 10000 pg·hr/mL, or about 250 pg·hr/mL to about 9000 pg·hr/mL, 250 pg·hr/mL to about 8500 pg·hr/mL, or about 250 pg·hr/mL to about 7000 pg·hr/mL, or about 250 pg·hr/mL to about 5000 pg·hr/mL, or about 250 pg·hr/mL to about 4000 pg·hr/mL, or about 250 pg·hr/mL to about 3000 pg·hr/mL, or about 500 pg·hr/mL to about 70000 pg·hr/mL, or about 500 pg·hr/mL to about 60000 pg·hr/mL, or about 500 pg·hr/mL to about 50000 pg·hr/mL, or about 500 pg·hr/mL to about 40000 pg·hr/mL, or about 500 pg·hr/mL to about 30000 pg·hr/mL, or about 500 pg·hr/mL to about 25000 pg·hr/mL, or about 500 pg·hr/mL to about 20000 pg·hr/mL, or about 500 pg·hr/mL to about 18000 pg·hr/mL, or about 500 pg·hr/mL to about 15000 pg·hr/mL, or about 500 pg·hr/mL to about 14000 pg·hr/mL, or about 500 pg·hr/mL to about 12000 pg·hr/mL, or about 500 pg·hr/mL to about 11000 pg·hr/mL, or about 500 pg·hr/mL to about 10000 pg·hr/mL, or about 500 pg·hr/mL to about 9000 pg·hr/mL, 500 pg·hr/mL to about 8500 pg·hr/mL, or about 500 pg·hr/mL to about 7000 pg·hr/mL, or about 500 pg·hr/mL to about 5000 pg·hr/mL, or about 500 pg·hr/mL to about 4000 pg·hr/mL, or about 500 pg·hr/mL to about 3000 pg·hr/mL, or about 1000 pg·hr/mL to about 70000 pg·hr/mL, or about 1000 pg·hr/mL to about 60000 pg·hr/mL, or about 1000 pg·hr/mL to about 50000 pg·hr/mL, or about 1000 pg·hr/mL to about 40000 pg·hr/mL, or about 1000 pg·hr/mL to about 30000 pg·hr/mL, or about 1000 pg·hr/mL to about 25000 pg·hr/mL, or about 1000 pg·hr/mL to about 20000 pg·hr/mL, or about 1000 pg·hr/mL to about 18000 pg·hr/mL, or about 1000 pg·hr/mL to about 15000 pg·hr/mL, or about 1000 pg·hr/mL to about 14000 pg·hr/mL, or about 1000 pg·hr/mL to about 12000 pg·hr/mL, or about 1000 pg·hr/mL to about 11000 pg·hr/mL, or about 1000 pg·hr/mL to about 10000 pg·hr/mL, or about 1000 pg·hr/mL to about 9000 pg·hr/mL, 1000 pg·hr/mL to about 81000 pg·hr/mL, or about 1000 pg·hr/mL to about 7000 pg·hr/mL, or about 1000 pg·hr/mL to about 5000 pg·hr/mL, or about 1000 pg·hr/mL to about 4000 pg·hr/mL, or about 1000 pg·hr/mL to about 3000 pg·hr/mL, or about 2000 pg·hr/mL to about 70000 pg·hr/mL, or about 2000 pg·hr/mL to about 60000 pg·hr/mL, or about 2000 pg·hr/mL to about 50000 pg·hr/mL, or about 2000 pg·hr/mL to about 40000 pg·hr/mL, or about 2000 pg·hr/mL to about 30000 pg·hr/mL, or about 2000 pg·hr/mL to about 25000 pg·hr/mL, or about 2000 pg·hr/mL to about 20000 pg·hr/mL, or about 2000 pg·hr/mL to about 18000 pg·hr/mL, or about 2000 pg·hr/mL to about 15000 pg·hr/mL, or about 2000 pg·hr/mL to about 14000 pg·hr/mL, or about 2000 pg·hr/mL to about 12000 pg·hr/mL, or about 2000 pg·hr/mL to about 12000 pg·hr/mL, or about 2000 pg·hr/mL to about 10000 pg·hr/mL, or about 2000 pg·hr/mL to about 9000 pg·hr/mL, 2000 pg·hr/mL to about 82000 pg·hr/mL, or about 2000 pg·hr/mL to about 7000 pg·hr/mL, or about 2000 pg·hr/mL to about 5000 pg·hr/mL, or about 2000 pg·hr/mL to about 4000 pg·hr/mL, or about 2000 pg·hr/mL to about 3000 pg·hr/mL, or about 1500 pg·hr/mL to about 8000 pg·hr/mL, or about 1500 pg·hr/mL to about 6000 pg·hr/mL, 3000 pg·hr/mL to about 10000 pg·hr/mL, or about 3000 pg·hr/mL to about 9000 pg·hr/mL, or about 1200 pg·hr/mL to about 12000 pg·hr/mL, or about 4000 pg·hr/mL to about 9000 pg·hr/mL, or about 4000 pg·hr/mL to about 12000 pg·hr/mL. In some preferred embodiments, the aforementioned AUC_{0-inf} being achieved with oral pharmaceutical compositions comprising a controlled release material to render said dosage form suitable for extended release.

In some preferred embodiments, the dosage form provides an oral pharmaceutical composition comprising a therapeutically effective amount of buprenorphine and controlled release material to render said dosage form suitable for once-a-day administration to a human patient, said dosage form after administration to a human patient providing a buprenorphine and norbuprenorphine mean AUC_{0-inf} after first administration or AUC₀₋₂₄ at steady state of not more than about 80000 pg·hr/mL; and said dosage form providing a therapeutic effect for at least about 24 hours. In other preferred embodiments, the dosage form provides a mean buprenorphine and norbuprenorphine AUC_{0-inf} after first administration or AUC₀₋₂₄ at steady state of not more than about 70000 pg·hr/mL, or not more than about 60000 pg·hr/mL, or not more than about 50000 pg·hr/mL, or not more than about 40000 pg·hr/mL, or not more than about 30000 pg·hr/mL, or not more than about 25000 pg·hr/mL, or not more than about 20000 pg·hr/mL, or not more than about 18000 pg·hr/mL, or not more than about 15000 pg·hr/mL, or not more than about 14000 pg·hr/mL, or not more than about 12000 pg·hr/mL, or not more than about 11000 pg·hr/mL, or not more than about 10000 pg·hr/mL, or not more than about 9000 pg·hr/mL, or not more than about 8500 pg·hr/mL, or not more than about 7000 pg·hr/mL, or not more than about 5000 pg·hr/mL, or not more than about 4000 pg·hr/mL, or not more than about 3000 pg·hr/mL, or not more than about 2500 pg·hr/mL, or not more than about 2000 pg·hr/mL, or not more than about 1500 pg·hr/mL. In some preferred embodiments, the aforementioned AUC_{0-inf} being achieved with oral pharmaceutical compositions comprising a controlled release material to render said dosage form suitable for extended release.

In some preferred embodiments, the dosage form provides an oral pharmaceutical composition comprising a therapeutically effective amount of buprenorphine and controlled release material to render said dosage form suitable for once-a-day administration to a human patient, said dosage form after administration to a human patient providing a C₁₆/C₂₄ ratio of buprenorphine and norbuprenorphine 0.1 to about 1.25; and said dosage form providing a therapeutic effect for at least about 24 hours. In other preferred embodiments, the dosage form provides a C₁₆/C₂₄ ratio of buprenorphine and norbuprenorphine of about 0.1 to about 1, or about 0.1 to about 0.8, or about 0.1 to about 0.75, or about 0.1 to about 0.6, or 0.1 to about 0.5, or about 0.1 to about 0.4, or about 0.1 to about 0.35, or about 0.25 to about 0.95, or about 0.4 to about 0.95, or about 0.5 to about 0.95, or about 0.65 to about 0.95, or about 0.75 to about 0.95, or about 0.3 to about 0.8, or about 0.4 to about 0.75, or about 0.5 to about 0.75. In some preferred embodiments, the aforementioned C₁₆/C₂₄ ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and, optionally, controlled release material to render said dosage form suitable for once-a-day administration to a human patient, said dosage form after administration to a human patient, providing a W₅₀ of buprenorphine and norbuprenorphine of 4 to about 22 hours; and said dosage form providing a therapeutic effect for at least about 24 hours. In other preferred embodiments, the dosage form provides a W₅₀ of buprenorphine and norbuprenorphine of about 4 to about 20 hours, or about 4 to about 19 hours, or about 4 to about 18 hours, or 4 to about 16 hours, or 4 to about 14 hours, or about 4 to about 12 hours, or about 4 to about 10 hours, or about 4 to about 8 hours, or about 6 to about 20 hours, or about 8 to about 20 hours, or about 10 to about 20 hours, or about 12 to about 20 hours, or 14 to about 20 hours, or about 6 to about 16 hours, or about 8 to about 16 hours, or about 8 to about 14 hours, or about 6 to about 12 hours.

In some preferred embodiments, the dosage form provides an oral pharmaceutical composition comprising a therapeutically effective amount of buprenorphine and, optionally, controlled release material to render said dosage form suitable for once-a-day administration to a human patient, said dosage form after administration to a human patient, providing a HVD of buprenorphine and norbuprenorphine of 3 to about 20 hours; and said dosage form providing a therapeutic effect for at least about 24 hours. In other preferred embodiments, the dosage form provides an HVD of buprenorphine and norbuprenorphine of about 3 to about 18 hours, or about 3 to 16 hours, or about 3 to about 14 hours, or about 3 to 12 hours, or about 3 to about 10 hours, or about 3 to about 8 hours, or about 4 to about 20 hours, or about 6 to 20 hours, or about 8 to about 20 hours, or about 10 to 20 hours, or about 12 to about 20 hours, or about 16 to 20 hours, about 6 to about 16 hours, or about 8 to 16 hours, or about 10 to about 14 hours, or about 6 to 12 hours, or about 6 to about 16 hours, or about 10 to about 16 hours.

In some preferred embodiments, the dosage form provides an oral pharmaceutical composition comprising a therapeutically effective amount of buprenorphine, and, optionally, controlled release material to render said dosage form suitable for once-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours.

In some preferred embodiments, the dosage form provides an oral pharmaceutical composition comprising a therapeutically effective amount of buprenorphine, and, optionally, controlled release material to render said dosage form suitable for once-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours; said in-vitro release rate being substantially independent of pH in that a difference, at any given time, between an amount of buprenorphine released at one pH and an amount released at any other pH, when measured in-vitro using the USP Basket or Paddle Method of USP Drug Release test of U.S. Pharmacopeia (2003) at 100 rpm in 900 ml aqueous buffer, is no greater than 30%.

In some preferred embodiments, the oral dosage form of the invention provides an in-vitro release of from about 2% to about 50% by weight of the buprenorphine or a pharmaceutically acceptable salt of buprenorphine from the dosage form at one hour when measured by the USP Basket Method at 100 rpm in 700 ml of Simulated Gastric Fluid (SGF) at 37° C. In other preferred embodiments, under the same dissolution conditions, said dosage form provides an in-vitro release rate by weight of the buprenorphine or a pharmaceutically acceptable salt of buprenorphine from the dosage form at one hour from about 2% to about 45%, or from about 2% to about 60%, or from about 5% to about 40%, or from about 5% to about 60%, or from about 10% to about 70%, or from about 10% to about 80%, or from about 15% to about 90%, or from about 60 to about 100%, or from about 80 to about 100%, or greater than about 1%, or greater than about 5%, or greater than about 15%, or greater than about 40%, or greater than about 60%, or greater than about 80%, or greater than about 90%, or greater than about 95%.

In some preferred embodiments, the oral dosage form of the invention provides at least about 5%, or 10%, or 15%, or 20%, or 30% or 40%, or 50% or 60%, or 70%, or 80%, or 100% lower variability in buprenorphine Cₘₐₓ (as defined by the coefficient of variation or C.V.) than after the sublingual dosage form of buprenorphine, each given according to its intended route and method of administration.

In some preferred embodiments, the oral dosage form of the invention provides at least about 5%, or 10%, or 15%, or 20%, or 30% or 40%, or 50% or 60%, or 70%, or 80%, or 100% lower variability in buprenorphine Tₘₐₓ (as defined by the coefficient of variation or C.V.) than after the sublingual dosage form of buprenorphine, each given according to its intended route and method of administration.

In some preferred embodiments, the oral dosage form of the invention provides at least about 5%, or 10%, or 15%, or 20%, or 30% or 40%, or 50% or 60%, or 70%, or 80%, or 100% lower variability in the buprenorphine AUC₀₋₂₄, or AUC₀₋₃₆, or AUC₀₋₄₈, or AUC₀₋₇₂ (as defined by the coefficient of variation or C.V.) than after the sublingual dosage form of buprenorphine, each given according to its intended route and method of administration.

In some preferred embodiments, the oral dosage form of the invention provides a ratio of mean AUC₀₋₄₈ of norbuprenorphine to buprenorphine after the first dose which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% greater than said ratio after the same amount of sublingual dosage form of buprenorphine, each given according to its intended route and method of administration.

In some preferred embodiments, the oral dosage form of the invention provides a time to 75% mean Cₘₐₓ of buprenorphine after the first dose which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% or 400%, or 500%, or 600%, or 700%, or 1000% longer than said time to mean Cₘₐₓ after the same amount of sublingual dosage form of buprenorphine, each given according to its intended route and method of administration.

In some preferred embodiments, the oral dosage form of the invention provides a mean Tₘₐₓ of buprenorphine after the first dose which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% or 400%, or 500%, or 600%, or 700%, or 1000% longer than said Tₘₐₓ after the same amount of sublingual dosage form of buprenorphine, each given according to its intended route and method of administration.

In some preferred embodiments, the oral dosage form of the invention provides a mean Cₘₐₓ of buprenorphine which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% less than said Cₘₐₓ after the same amount of sublingual dosage form of buprenorphine, each given according to its intended route and method of administration.

In some preferred embodiments, the oral dosage form of the invention provides a mean Cₘₐₓ ratio of norbuprenorphine to buprenorphine which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% greater than said Cₘₐₓ ratio after the same amount of sublingual dosage form of buprenorphine, each given according to its intended route and method of administration.

In some preferred embodiments, the oral dosage form of the invention provides a time to 75% mean Cₘₐₓ of buprenorphine which is about 100% to about 2000% of the time to 75% mean Cₘₐₓ after the same amount of a commercially available immediate release sublingual formulation of buprenorphine listed in FDA's Orange Book, each given according to its intended route and method of administration.

In some preferred embodiments, a controlled release dosage form of the invention provides a time to mean Cₘₐₓ of buprenorphine which is at least about 1.25, 1.5, 2, 3, 4, 5, 6, 7, 8, 9, 10 or 12 fold greater than the time to mean Cₘₐₓ after the same amount of an oral immediate release buprenorphine solution.

In some preferred embodiments, the oral dosage form of the invention provides a time to mean Cₘₐₓ of buprenorphine which is at least about 1.25, 1.5, 2, 3, 4, 5, 6, 7, 8, 9, 10 or 12 fold greater than the time to mean Cₘₐₓ after the same amount of a commercially available immediate release sublingual formulation of buprenorphine listed in FDA's Orange Book, each given according to its intended route and method of administration.

In some preferred embodiments, the oral dosage form of the invention provides a mean Cₘₐₓ of buprenorphine which is at least about 5%, 10%, 15%, 20%, 30%, 40%, 50%, 60%, 70%, 80%, 90%, 100%, 120%, 140%, 150%, 180%, 200%, 230%, 260%, or 300% lower than the mean Cₘₐₓ after the same amount of a commercially available immediate release sublingual formulation of buprenorphine listed in FDA's Orange Book, each given according to its intended route and method of administration.

In some preferred embodiments, the oral dosage form of the invention provides a ratio of mean AUC₀₋₁₄ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral dosage form of the invention provides a ratio of mean AUC₀₋₂₄ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral dosage form of the invention provides a ratio of mean AUC₀₋₃₆ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral dosage form of the invention provides a ratio of mean AUC₀₋₄₈ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral dosage form of the invention provides a ratio of mean AUC₀₋₆₀ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral immediate release and oral extended release dosage forms provide a ratio of mean AUC₀₋₂₄, or AUC₀₋₃₆, or AUC₀₋₄₈, or AUC₀₋₇₂ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the transdermal buprenorphine, each given according to its intended route and method of administration.

In some preferred embodiments, the oral immediate release and oral extended release dosage forms provide a ratio of mean AUC₀₋₁₂, or AUC₀₋₂₄, or AUC₀₋₄₈ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after intranasal buprenorphine, each given according to its intended route and method of administration.

In some preferred embodiments, the oral immediate release and oral extended release dosage forms provides a ratio of mean AUC₀₋₂₄, or AUC₀₋₃₆, or AUC₀₋₄₈, or AUC₀₋₇₂ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after a sublingual formulation of buprenorphine, each given according to its intended route and method of administration.

In some preferred embodiments, the oral extended release dosage form provides a ratio of mean AUC₀₋₂₄, or AUC₀₋₃₆, or AUC₀₋₄₈, or AUC₀₋₇₂ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% less than the said ratio after the same amount of an oral immediate release formulation of buprenorphine.

In some preferred embodiments, the oral extended release dosage form provides a mean extent of absorption (as measured by AUC₀₋₂₄, or AUC₀₋₃₆, or AUC₀₋₄₈, or AUC₀₋₇₂) of buprenorphine after the first dose which is at least about 5%, 10%, 15%, 20%, 30%, or 40% greater than the said ratio after the same amount of an oral immediate release formulation of buprenorphine.

In some preferred embodiments, the ratio of the mean ratio of the extent of absorption (as measured by AUC₀₋₂₄, or AUC₀₋₃₆, or AUC₀₋₄₈, or AUC₀₋₇₂) of norbuprenorphine to buprenorphine for oral extended release buprenorphine after the first dose is at least about 5%, 10%, 15%, 20%, 30%, 40%, 50%, 60%, 70%, 80%, 90%, 100%, 120%, 140%, 150%, 180%, 200%, 230%, 260%, or 300% less than the said ratio after the first dose of the same amount of an oral immediate release formulation of buprenorphine.

In some preferred embodiments, the oral dosage form of the invention provides a mean drowsiness score in buprenorphine and opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than the mean drowsiness score after the same amount of a commercially available immediate release sublingual formulation of buprenorphine listed in FDA's Orange Book, each given according to its intended route and method of administration.

In some preferred embodiments, the oral dosage form of the invention provides a mean nausea score which is at least about 5%, 10%, 15%, 20%, 30%, 40%, 50%, 60%, 70%, 80%, 90%, 100%, 120%, 140%, 150%, 180%, 200%, 230%, 260%, or 300% lower than the mean nausea score after the same amount of a commercially available immediate release sublingual formulation of buprenorphine listed in FDA's Orange Book, each given to buprenorphine and opioid naïve subjects according to its intended route of administration.

In some preferred embodiments, the oral dosage form of the invention provides a mean driving simulation impairment score in buprenorphine and opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than the mean driving simulation impairment score after the same amount of a commercially available immediate release sublingual formulation of buprenorphine listed in FDA's Orange Book, each given to buprenorphine and opioid naïve subjects according to its intended route of administration.

In some preferred embodiments, the oral dosage form of the invention provides a mean number needed to harm (NNH) due to moderate to severe sedation or drowsiness in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean number needed to harm (NNH) due to moderate or severe nausea in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean number needed to harm (NNH) due to dizziness in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean number needed to harm (NNH) due to dry mouth in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean “drug effects” score in opioid abusers or recreational opioid users which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean “drug liking” score in opioid abusers or recreational opioid users which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean “take again” score in opioid abusers or recreational opioid users which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean “coasting” score in opioid abusers or recreational opioid users which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean “critical tracking task” impairment score in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean “stop signal task” impairment score in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a mean “Tower of London” (TOL) impairment score in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book, each given to according to its intended route of administration.

In some preferred embodiments, the oral dosage form of the invention provides a relative mean Cₘₐₓ whose 90% confidence interval is outside the 80.00% to 125.00, under single-dose fasted test conditions in healthy subjects, when compared with immediate release sublingual formulation of buprenorphine listed in FDA's Orange Book.

In some preferred embodiments, the oral dosage form of the invention provides a relative mean Cₘₐₓ whose 90% confidence interval is outside the 80.00% to 125.00, under single-dose fasted test conditions in healthy subjects, when compared with immediate release sublingual formulation of buprenorphine listed in FDA's Orange Book, each given to according to its intended route of administration.

In some preferred embodiments, the oral dosage form of the invention provides a relative mean Cₘₐₓ whose 90% confidence interval is outside the 80.00% to 125.00, under single-dose fasted test conditions in healthy subjects, when compared with other available buprenorphine dosage forms.

In some preferred embodiments, the oral dosage form of the invention provides a relative mean Cₘₐₓ whose 90% confidence interval is outside the 80.00% to 125.00, under single-dose fasted test conditions in healthy subjects, when compared with intranasal dosage forms of buprenorphine dosage forms.

In some preferred embodiments, the oral dosage form of the invention provides a relative mean Cₘₐₓ whose 90% confidence interval is outside the 80.00% to 125.00, under single-dose fasted test conditions in healthy subjects, when compared with transdermal dosage forms of buprenorphine dosage forms.

In some preferred embodiments, the oral dosage form of the invention provides a relative mean Cₘₐₓ whose 90% confidence interval is outside the 80.00% to 125.00, under single-dose fasted test conditions in healthy subjects, when compared with buccal dosage forms of buprenorphine dosage forms.

In some preferred embodiments, the oral dosage form of the invention provides a relative mean Cₘₐₓ whose 90% confidence interval is outside the 80.00% to 125.00, under single-dose fasted test conditions in healthy subjects, when compared with immediate release oral buprenorphine.

In some preferred embodiments, the oral dosage form of the invention provides a relative mean Cₘₐₓ whose 90% confidence interval is outside the 80.00% to 125.00, under single-dose fasted test conditions in healthy subjects, when compared with immediate release oral buprenorphine solution.

In some preferred embodiments, the oral dosage form of the invention provides a relative mean AUC_{0-τ} whose 90% confidence interval is outside the 80.00% to 125.00, under single-dose fasted test conditions in healthy subjects, when compared with immediate release sublingual formulation of buprenorphine listed in FDA's Orange Book.

In some preferred embodiments, the oral dosage form of the invention provides a relative mean AUC_{0-τ} whose 90% confidence interval is outside the 80.00% to 125.00, under single-dose fasted test conditions in healthy subjects, when compared with immediate release sublingual formulation of buprenorphine listed in FDA's Orange Book, each given to according to its intended route of administration.

In some preferred embodiments, the oral dosage form of the invention provides a relative mean AUC_{0-τ} whose 90% confidence interval is outside the 80.00% to 125.00, under single-dose fasted test conditions in healthy subjects, when compared with other available buprenorphine dosage forms.

In some preferred embodiments, the oral dosage form of the invention provides a relative mean AUC_{0-τ} whose 90% confidence interval is outside the 80.00% to 125.00, under single-dose fasted test conditions in healthy subjects, when compared with intranasal dosage forms of buprenorphine dosage forms.

In some preferred embodiments, the oral dosage form of the invention provides a relative mean AUC_{0-τ} whose 90% confidence interval is outside the 80.00% to 125.00, under single-dose fasted test conditions in healthy subjects, when compared with transdermal dosage forms of buprenorphine dosage forms.

In some preferred embodiments, the oral dosage form of the invention provides a relative mean AUC_{0-τ} whose 90% confidence interval is outside the 80.00% to 125.00, under single-dose fasted test conditions in healthy subjects, when compared with buccal dosage forms of buprenorphine dosage forms.

In some preferred embodiments, the oral dosage form of the invention provides a relative mean AUC_{0-τ} whose 90% confidence interval is outside the 80.00% to 125.00, under single-dose fasted test conditions in healthy subjects, when compared with immediate release oral buprenorphine.

In some preferred embodiments, the oral dosage form of the invention provides a relative mean AUC_{0-τ} whose 90% confidence interval is outside the 80.00% to 125.00, under single-dose fasted test conditions in healthy subjects, when compared with immediate release oral buprenorphine solution.

In some preferred embodiments, the oral dosage form of the invention provides a relative mean AUC_{0-inf} whose 90% confidence interval is outside the 80.00% to 125.00, under single-dose fasted test conditions in healthy subjects, when compared with immediate release sublingual formulation of buprenorphine listed in FDA's Orange Book.

In some preferred embodiments, the oral dosage form of the invention provides a relative mean AUC_{0-inf} whose 90% confidence interval is outside the 80.00% to 125.00, under single-dose fasted test conditions in healthy subjects, when compared with immediate release sublingual formulation of buprenorphine listed in FDA's Orange Book, each given to according to its intended route of administration.

In some preferred embodiments, the oral dosage form of the invention provides a relative mean AUC_{0-inf} whose 90% confidence interval is outside the 80.00% to 125.00, under single-dose fasted test conditions in healthy subjects, when compared with other available buprenorphine dosage forms.

In some preferred embodiments, the oral dosage form of the invention provides a relative mean AUC_{0-inf} whose 90% confidence interval is outside the 80.00% to 125.00, under single-dose fasted test conditions in healthy subjects, when compared with intranasal dosage forms of buprenorphine dosage forms.

In some preferred embodiments, the oral dosage form of the invention provides a relative mean AUC_{0-inf} whose 90% confidence interval is outside the 80.00% to 125.00, under single-dose fasted test conditions in healthy subjects, when compared with transdermal dosage forms of buprenorphine dosage forms.

In some preferred embodiments, the oral dosage form of the invention provides a relative mean AUC_{0-inf} whose 90% confidence interval is outside the 80.00% to 125.00, under single-dose fasted test conditions in healthy subjects, when compared with buccal dosage forms of buprenorphine dosage forms.

In some preferred embodiments, the oral dosage form of the invention provides a relative mean AUC_{0-inf} whose 90% confidence interval is outside the 80.00% to 125.00, under single-dose fasted test conditions in healthy subjects, when compared with immediate release oral buprenorphine.

In some preferred embodiments, the oral dosage form of the invention provides a relative mean AUC_{0-inf} whose 90% confidence interval is outside the 80.00% to 125.00, under single-dose fasted test conditions in healthy subjects, when compared with immediate release oral buprenorphine solution.

In some preferred embodiments, the dosage from maintains a plasma buprenorphine and norbuprenorphine concentration within 50% of Cₘₐₓ for about 1 to about 9 hours, or about 2 to about 9 hours, or about 3 to about 9 hours, or about 4 to about 9 hours, or about 5 to about 9 hours, or about 6 to about 9 hours, or about 1 to about 8 hours, or about 2 to about 8 hours, or about 3 to about 8 hours, or about 4 to about 8 hours, or about 5 to about 8 hours, or about 6 to about 8 hours, or about 2 to about 7 hours, or about 2 to about 6 hours, or about 2 to about 5 hours, or about 2 to about 4 hours, or about 1 to about 4 hours, or about 1 to about 3 hours, or about 2 to about 6 hours, or about 3 to about 5 hours.

In some preferred embodiments, the dosage from maintains a plasma buprenorphine and norbuprenorphine concentration within 50% of Cₘₐₓ for about 1 to about 9 hours, or about 2 to about 9 hours, or about 3 to about 9 hours, or about 4 to about 9 hours, or about 5 to about 9 hours, or about 6 to about 9 hours, or about 1 to about 8 hours, or about 2 to about 8 hours, or about 3 to about 8 hours, or about 4 to about 8 hours, or about 5 to about 8 hours, or about 6 to about 8 hours, or about 2 to about 7 hours, or about 2 to about 6 hours, or about 2 to about 5 hours, or about 2 to about 4 hours, or about 1 to about 4 hours, or about 1 to about 3 hours, or about 2 to about 6 hours, or about 3 to about 5 hours during a 12 hour dosing interval.

In some preferred embodiments, the dosage from maintains a plasma buprenorphine and norbuprenorphine concentration within 50% of Cₘₐₓ for about 1 to about 20 hours, or about 1 to about 18 hours or about 1 to about 16 hours, or about 1 to about 14 hours or about 1 to about 10 hours, or about 1 to about 8 hours or about 1 to about 6 hours, or about 1 to about 5 hours or about 2 to about 20 hours, or about 4 to about 20 hours or about 4 to about 18 hours, or about 5 to about 18 hours or about 6 to about 18 hours, or about 7 to about 18 hours or about 8 to about 18 hours, or about 10 to about 18 hours or about 12 to about 18 hours, or about 14 to about 18 hours or about 4 to about 16 hours, or about 4 to about 12 hours or about 4 to about 10 hours, or about 4 to about 8 hours or about 5 to about 15 hours, or about 5 to about 10 hours or about 6 to about 18 hours, or about 6 to about 12 hours or about 6 to about 10 hours, or about 8 to about 18 hours or about 8 to about 16 hours, or about 10 to about 18 hours.

In some preferred embodiments, the dosage from maintains a plasma buprenorphine and norbuprenorphine concentration within 50% of Cₘₐₓ for about 1 to about 20 hours, or about 1 to about 18 hours or about 1 to about 16 hours, or about 1 to about 14 hours or about 1 to about 10 hours, or about 1 to about 8 hours or about 1 to about 6 hours, or about 1 to about 5 hours or about 2 to about 20 hours, or about 4 to about 20 hours or about 4 to about 18 hours, or about 5 to about 18 hours or about 6 to about 18 hours, or about 7 to about 18 hours or about 8 to about 18 hours, or about 10 to about 18 hours or about 12 to about 18 hours, or about 14 to about 18 hours or about 4 to about 16 hours, or about 4 to about 12 hours or about 4 to about 10 hours, or about 4 to about 8 hours or about 5 to about 15 hours, or about 5 to about 10 hours or about 6 to about 18 hours, or about 6 to about 12 hours or about 6 to about 10 hours, or about 8 to about 18 hours or about 8 to about 16 hours, or about 10 to about 18 hours, during a 24 hour dosing interval.

In some preferred embodiments, the oral pharmaceutical composition of buprenorphine or a pharmaceutically dosage from provides a buprenorphine Tₘₐₓ greater than about 0.25 hours, or greater than about 0.5 hours, or greater than about 0.75 hours, or greater than about 1 hour, or greater than about 1.5 hours, or greater than about 2 hours, or greater than about 2.5 hours, or greater than about 3 hours, or greater than about 3.5 hours, or greater than about 4 hours, or greater than about 4.5 hours, or greater than about 5 hours, or greater than about 6 hours, or greater than about 8 hours, or greater than about 10 hours, or greater than about 12 hours, or greater than about 14 hours, or greater than about 16 hours, or greater than about 17 hours, or greater than about 18 hours, or greater than about 20 hours, or greater than about 22 hours, or greater than about 24 hours.

In some preferred embodiments, the oral pharmaceutical composition of buprenorphine or a pharmaceutically dosage from provides a buprenorphine Tₘₐₓ of about 0.25 to about 8 hours, about 0.5 to about 30 hours, or about 0.5 to about 26 hours, or about 0.5 to about 22 hours, or about 0.5 to about 20 hours, or about 0.5 to about 18 hours, or about 0.5 to about 16 hours, or about 0.5 to about 14 hours, or about 0.5 to about 12 hours, or about 0.5 to about 10 hours, or about 0.5 to about 9 hours, or about 0.5 to about 8 hours, or about 0.5 to about 7 hours, or about 0.5 to about 6 hours, or about 0.5 to about 5 hours, or about 0.5 to about 4 hours, or about 0.5 to about 3 hours, or about 0.5 to about 2 hours, or about 0.5 to about 1 hour, or about 1 to about 30 hours, or about 2 to about 30 hours, or about 3 to about 30 hours, or about 4 to about 30 hours, or about 5 to about 30 hours, or about 6 to about 30 hours, or about 1 to about 24 hours, or about 2 to about 24 hours, or about 3 to about 24 hours, or about 4 to about 24 hours, or about 5 to about 24 hours, or about 6 to about 24 hours, or about 8 to about 24 hours, or about 10 to about 24 hours, or about 12 to about 24 hours, or about 14 to about 24 hours, or about 1.5 to about 16 hours, or about 2 to about 16 hours, or about 2.5 to about 16 hours, or about 3 to about 16 hours, or about 3.5 to about 16 hours, or about 4 to about 16 hours, or about 5 to about 16 hours, or about 6 to about 16 hours, or about 7 to about 16 hours, or about 8 to about 16 hours, or about 10 to about 16 hours, or about 12 to about 16 hours, or about 2 to about 10 hours, or about 2.5 to about 10 hours, or about 3 to about 10 hours, or about 3.5 to about 10 hours, or about 4 to about 10 hours, or about 4.5 to about 10 hours, or about 5 to about 10 hours, or about 6 to about 10 hours, or about 7 to about 10 hours, or about 7.5 to about 10 hours.

In some preferred embodiments, the oral pharmaceutical composition of buprenorphine or a pharmaceutically dosage from provides a norbuprenorphine Tₘₐₓ greater than about 0.25 hours, or greater than about 0.5 hours, or greater than about 0.75 hours, or greater than about 1 hour, or greater than about 1.5 hours, or greater than about 2 hours, or greater than about 2.5 hours, or greater than about 3 hours, or greater than about 3.5 hours, or greater than about 4 hours, or greater than about 4.5 hours, or greater than about 5 hours, or greater than about 6 hours, or greater than about 8 hours, or greater than about 10 hours, or greater than about 12 hours, or greater than about 14 hours, or greater than about 16 hours, or greater than about 17 hours, or greater than about 18 hours, or greater than about 20 hours, or greater than about 22 hours, or greater than about 24 hours.

In some preferred embodiments, the oral pharmaceutical composition of buprenorphine or a pharmaceutically dosage from provides a norbuprenorphine Tₘₐₓ of about 0.25 to about 8 hours, about 0.5 to about 30 hours, or about 0.5 to about 26 hours, or about 0.5 to about 22 hours, or about 0.5 to about 20 hours, or about 0.5 to about 18 hours, or about 0.5 to about 16 hours, or about 0.5 to about 14 hours, or about 0.5 to about 12 hours, or about 0.5 to about 10 hours, or about 0.5 to about 9 hours, or about 0.5 to about 8 hours, or about 0.5 to about 7 hours, or about 0.5 to about 6 hours, or about 0.5 to about 5 hours, or about 0.5 to about 4 hours, or about 0.5 to about 3 hours, or about 0.5 to about 2 hours, or about 0.5 to about 1 hour, or about 1 to about 30 hours, or about 2 to about 30 hours, or about 3 to about 30 hours, or about 4 to about 30 hours, or about 5 to about 30 hours, or about 6 to about 30 hours, or about 1 to about 24 hours, or about 2 to about 24 hours, or about 3 to about 24 hours, or about 4 to about 24 hours, or about 5 to about 24 hours, or about 6 to about 24 hours, or about 8 to about 24 hours, or about 10 to about 24 hours, or about 12 to about 24 hours, or about 14 to about 24 hours, or about 1.5 to about 16 hours, or about 2 to about 16 hours, or about 2.5 to about 16 hours, or about 3 to about 16 hours, or about 3.5 to about 16 hours, or about 4 to about 16 hours, or about 5 to about 16 hours, or about 6 to about 16 hours, or about 7 to about 16 hours, or about 8 to about 16 hours, or about 10 to about 16 hours, or about 12 to about 16 hours, or about 2 to about 10 hours, or about 2.5 to about 10 hours, or about 3 to about 10 hours, or about 3.5 to about 10 hours, or about 4 to about 10 hours, or about 4.5 to about 10 hours, or about 5 to about 10 hours, or about 6 to about 10 hours, or about 7 to about 10 hours, or about 7.5 to about 10 hours.

In some preferred embodiments, the oral dosage form of the invention provides a mean in vivo extent of absorption of buprenorphine from 0 to 4 hours which is at least 20% of the mean in vivo extent of absorption from to 0 to 12 hours, wherein the mean in vivo extent of absorption is the area under the plasma or serum buprenorphine concentration time curve from the time of drug administration to the specified time point.

In some preferred embodiments, the oral dosage form of the invention provides a mean in vivo extent of absorption of buprenorphine from 0 to 8 hours which is at least 20% of the mean in vivo extent of absorption from to 0 to 24 hours, wherein the mean in vivo extent of absorption is the area under the plasma or serum buprenorphine concentration time curve from the time of drug administration to the specified time point.

In some preferred embodiments, the oral dosage form of the invention provides a mean in vivo extent of absorption of buprenorphine and norbuprenorphine over the dosing interval (e.g., from 0 to 12 hours or from 0 to 24 hours) which is at least 40% of the mean in vivo extent of absorption from to 0 to infinity, wherein the mean in vivo extent of absorption is the area under the plasma or serum buprenorphine and norbuprenorphine concentration time curves (AUC) from the time of drug administration to the specified time point and where AUC infinity is the sum of AUC from time “0” to time “t” (the last quantifiable time point which has been sampled) plus the extrapolated AUC from the last quantifiable sampling time point to infinity.

In some preferred embodiments, the oral dosage form of the invention provides an in-vitro release rate by weight of buprenorphine of less than 5% at one hour when measured by the USP Basket Method at 100 rpm in 700 ml of Simulated Gastric Fluid (SGF) at 37° C. In other embodiments, said release rate from 2% to about 50% at one hour, or from 5% to about 40% at one hour, or from 5% to about 40% at one hour, or from 5% to about 45% at one hour, or from 10% to about 40% at one hour, or from 20% to about 40% at one hour, or from 1% to about 40% at one hour, or from 1% to about 60% at one hour, or from 1% to about 80% at one hour, or from 1% to about 90% at one hour, or from 1% to about 100% at one hour, or greater than about 1% at one hour, or greater than about 5% at one hour, or greater than about 10% at one hour, or greater than about 20% at one hour, or greater than about 30% at one hour, or greater than about 40% at one hour, or greater than about 50% at one hour, or greater than about 60% at one hour, or greater than about 70% at one hour, or greater than about 80% at one hour, or greater than about 90% at one hour.

In some preferred embodiments, the oral dosage form of the invention provides an in-vitro release of between 0% to about 50% by weight of the buprenorphine or a pharmaceutically acceptable salt of buprenorphine from the dosage form at one hour when measured by the USP Basket or Paddle Method at 100 rpm in 700 ml of Simulated Gastric Fluid (SGF) at 37° C.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of more than about 40% at 10 minutes, more than about 60% at 20 minutes and more than about 80% at 30 minutes

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 80% at 0.5 hours, and greater than about 40% at 1 hour.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 90% at 0.5 hours, and greater than about 60% at 1 hour.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 5% to about 90% at 10 minutes, and greater than about 60% at 20 minutes.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 5% to about 100% at 10 minutes, and greater than about 60% at 30 minutes.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 100% at 0.5 hours, and greater than about 70% at 1 hour.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 90% at 1 hour, and greater than about 40% at 2 hours.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 90% at 1 hour, and greater than about 70% at 2 hours.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 50% at 1 hour, and greater than about 30% at 2 hours.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 30% at 1 hour, and greater than about 25% at 2 hours.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 100% at 0.5 hours, and greater than about 60% at 1 hour.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 100% at 1 hour, and greater than about 60% at 2 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing a mean in vitro controlled release rate of buprenorphine of 0.15 mg per hour to 35.0 mg per hour. In some preferred embodiments, when the oral dosage form is extended release and intended to provide a therapeutic effect of about 6, 8, 12 or 24 hours, said release rate is about 0.20 mg per hour to about 8.33 mg per hour, more preferably 0.42 mg per hour to 4.2 mg per hour, and most preferably 0.2 mg per hour to 2.5 mg per hour. In other preferred embodiments, the oral dosage form provides a therapeutic effect for about 6, 8, 12 or 24 hours and provide said release rate of about 0.20 mg per hour to about 8.33 mg per hour, more preferably 0.42 mg per hour to 4.2 mg per hour, and most preferably 0.2 mg per hour to 2.5 mg per hour.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% to about 40% at 1 hour, from about 5% to about 60% at 2 hours, from about 10% to about 75% at 4 hours, from about 20% to about 75% at 6 hours, from about 30% to about 80% at 9 hours, and greater than about 70% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 1% and about 45% at 1 hour, between about 5% and about 70% at 2 hours, between about 10% and about 90% at 4 hours, between about 20% and about 90% at 8 hours, greater than about 60% at 12 hours, greater than about 80% at 18 hours, and greater than about 85% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 5% and about 60% at 1 hour, between about 12.5% and about 80% at 2 hours, between about 25% and about 95% at 4 hours, between about 45% and about 100% at 8 hours, greater than about 55% at 12 hours, greater than about 65% at 18 hours, and greater than about 70% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 40% at 1 hour, between about 0% and about 70% at 2 hours, between about 5% and about 95% at 4 hours, between about 12.5% and about 100% at 8 hours, between about 20% and about 100% at 12 hours, between about 35% and about 100% at 16 hours, between about 55% and about 100% at 24 hours, and greater than about 75% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 60% at 1 hour, between about 0% and about 75% at 2 hours, between about 5% and about 95% at 4 hours, between about 12.5% and about 100% at 8 hours, between about 15% and about 100% at 12 hours, between about 25% to about 100% at 16 hours, between about 30% and about 100% hours at 24 hours and greater than 60% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release from the dosage form at one hour when measured by the USP Basket or Paddle Methods at 100 rpm in 700 ml of Simulated Gastric Fluid (SGF) at 37° C. of between 0% to about 50% by weight of buprenorphine. In other preferred embodiments, said release rate is between 0% to about 1%, or 0% to about 3%, or 0% to about 5%, or 0% to about 10%, or 0% to about 15%, or 0% to about 20%, 0% to about 30%, or 0% to about 40%, or 0% to about 60%, or 0% to about 70%, or 0% to about 80%, or 0% to about 90%, 0% to about 100%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release from the dosage form at one hour when measured by the USP Basket or Paddle Methods at 100 rpm in 900 mL aqueous buffer at a pH of between 1.6 and 7.2 at 37° C. of between 0% and about 60% at 1 hour, between about 0% and about 80% at 2 hours, between about 3% and about 95% at 4 hours and between about 10% and about 100% at 8 hours. In other preferred embodiments, said release rate is between 0% and about 10% at 1 hour, between about 0% and about 20% at 2 hours, between about 2% and about 80% at 4 hours and between about 5% and about 100% at 8 hours; or between 0% and about 20% at 1 hour, between about 0% and about 40% at 2 hours, between about 0% and about 80% at 4 hours and between about 2% and about 100% at 8 hours; or between 0% and about 40% at 1 hour, between about 0% and about 60% at 2 hours, between about 5% and about 85% at 4 hours and between about 5% and about 90% at 8 hours and greater than 20% at 12 hours; or between 0% and about 50% at 1 hour, between about 0% and about 50% at 2 hours, between about 10% and about 90% at 4 hours and between about 15% and about 90% at 8 hours and greater than 30% at 12 hours; or between 0% and about 70% at 1 hour, between about 0% and about 70% at 2 hours, between about 10% and about 75% at 4 hours and between about 15% and about 90% at 8 hours and greater than 30% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release from the dosage form at one hour when measured by the USP Basket or Paddle Methods at 100 rpm in 900 mL aqueous buffer at a pH of between 1.6 and 7.2 at 37° C. of between 10% and about 65% at 1 hour, between about 20% and about 75% at 2 hours, between about 30% and about 95% at 4 hours and between about 40% and about 100% at 8 hours. In other preferred embodiments, said release rate is between 2% and about 70% at 1 hour, between about 5% and about 80% at 2 hours, between about 10% and about 90% at 4 hours and between about 20% and about 100% at 8 hours; or between 5% and about 60% at 1 hour, between about 10% and about 75% at 2 hours, between about 15% and about 85% at 4 hours and between about 30% and about 100% at 8 hours; or between 20% and about 70% at 1 hour, between about 20% and about 75% at 2 hours, between about 20% and about 90% at 4 hours and between about 40% and about 100% at 8 hours; or between 30% and about 80% at 1 hour, between about 40% and about 85% at 2 hours, between about 40% and about 90% at 4 hours and between about 60% and about 100% at 8 hours; or between 1% and about 20% at 1 hour, between about 5% and about 20% at 2 hours, between about 10% and about 40% at 4 hours and between about 20% and about 40% at 8 hours and greater than 40% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours. In other preferred embodiments, said release rate is between 0% to about 30% at 1 hour, from about 5% to about 45% at 2 hours, from about 10% to about 60% at 4 hours, from about 15% to about 70% at 6 hours, from about 25% to about 80% at 9 hours, and greater than about 50% at 12 hours; or between 0% to about 20% at 1 hour, from about 2% to about 35% at 2 hours, from about 5% to about 50% at 4 hours, from about 10% to about 60% at 6 hours, from about 15% to about 70% at 9 hours, and greater than about 40% at 12 hours; or between 0% to about 10% at 1 hour, from about 1% to about 30% at 2 hours, from about 5% to about 40% at 4 hours, from about 10% to about 60% at 6 hours, from about 15% to about 70% at 9 hours, and greater than about 40% at 12 hours; or between 0% to about 5% at 1 hour, from about 0% to about 10% at 2 hours, from about 2% to about 20% at 4 hours, from about 5% to about 30% at 6 hours, from about 10% to about 40% at 9 hours, and greater than about 30% at 12 hours; or between 0% to about 50% at 1 hour, from about 15% to about 70% at 2 hours, from about 20% to about 75% at 4 hours, from about 30% to about 80% at 6 hours, from about 30% to about 90% at 9 hours, and greater than about 70% at 12 hours; or between 0% to about 60% at 1 hour, from about 15% to about 80% at 2 hours, from about 25% to about 85% at 4 hours, from about 35% to about 90% at 6 hours, from about 40% to about 90% at 9 hours, and greater than about 80% at 12 hours; or between 0% to about 70% at 1 hour, from about 20% to about 80% at 2 hours, from about 25% to about 80% at 4 hours, from about 35% to about 80% at 6 hours, from about 40% to about 80% at 9 hours, and greater than about 60% at 12 hours; or between 0% to about 75% at 1 hour, from about 30% to about 80% at 2 hours, from about 35% to about 90% at 4 hours, from about 50% to about 90% at 6 hours, from about 55% to about 95% at 9 hours, and greater than about 70% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 5% and about 50% at 1 hour, between about 10% and about 75% at 2 hours, between about 20% and about 95% at 4 hours, between about 40% and about 100% at 8 hours, greater than about 50% at 12 hours, greater than about 70% at 18 hours, and greater than about 80% at 24 hours. In other preferred embodiments, said release rate is between 2% and about 50% at 1 hour, between about 5% and about 75% at 2 hours, between about 15% and about 75% at 4 hours, between about 30% and about 90% at 8 hours, greater than about 40% at 12 hours, greater than about 60% at 18 hours, and greater than about 70% at 24 hours; or between 1% and about 40% at 1 hour, between about 2% and about 60% at 2 hours, between about 10% and about 65% at 4 hours, between about 20% and about 80% at 8 hours, greater than about 30% at 12 hours, greater than about 40% at 18 hours, and greater than about 60% at 24 hours; or between 5% and about 60% at 1 hour, between about 15% and about 80% at 2 hours, between about 25% and about 95% at 4 hours, between about 45% and about 100% at 8 hours, greater than about 60% at 12 hours, greater than about 80% at 18 hours, and greater than about 90% at 24 hours; or between 10% and about 65% at 1 hour, between about 20% and about 85% at 2 hours, between about 30% and about 100% at 4 hours, between about 60% and about 100% at 8 hours, greater than about 70% at 12 hours, greater than about 90% at 18 hours, and greater than about 95% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours. In other preferred embodiments, said release rate is between 0% to about 20% at 1 hour, from about 5% to about 50% at 4 hours, from about 10% to about 60% at 8 hours, from about 15% to about 70% at 12 hours, from about 25% to about 90% at 18 hours, and greater than about 55% at 24 hours; or between 0% to about 10% at 1 hour, from about 5% to about 40% at 4 hours, from about 8% to about 50% at 8 hours, from about 10% to about 60% at 12 hours, from about 22% to about 80% at 18 hours, and greater than about 45% at 24 hours; or between 0% to about 35% at 1 hour, from about 15% to about 70% at 4 hours, from about 25% to about 75% at 8 hours, from about 30% to about 85% at 12 hours, from about 40% to about 100% at 18 hours, and greater than about 75% at 24 hours; or between 0% to about 40% at 1 hour, from about 20% to about 70% at 4 hours, from about 30% to about 80% at 8 hours, from about 35% to about 90% at 12 hours, from about 45% to about 100% at 18 hours, and greater than about 80% at 24 hours; or between 0% to about 45% at 1 hour, from about 25% to about 75% at 4 hours, from about 35% to about 85% at 8 hours, from about 40% to about 90% at 12 hours, from about 50% to about 100% at 18 hours, and greater than about 90% at 24 hours; or between 0% to about 50% at 1 hour, from about 30% to about 80% at 4 hours, from about 40% to about 90% at 8 hours, from about 45% to about 95% at 12 hours, from about 60% to about 100% at 18 hours, and greater than about 95% at 24 hours; or between 0% to about 60% at 1 hour, from about 40% to about 80% at 4 hours, from about 45% to about 90% at 8 hours, from about 50% to about 100% at 12 hours, from about 70% to about 100% at 18 hours, and greater than about 80% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 50% at 1 hour, between about 0% and about 75% at 2 hours, between about 3% and about 95% at 4 hours, between about 10% and about 100% at 8 hours, between about 25% and about 100% at 12 hours, between about 30% and about 100% at 16 hours, between about 50% and about 100% at 24 hours, and greater than about 80% at 36 hours. In other preferred embodiments, said release rate is between 0% and about 40% at 1 hour, between about 0% and about 65% at 2 hours, between about 2% and about 85% at 4 hours, between about 8% and about 90% at 8 hours, between about 20% and about 95% at 12 hours, between about 25% and about 95% at 16 hours, between about 40% and about 90% at 24 hours, and greater than about 70% at 36 hours; or between 0% and about 30% at 1 hour, between about 0% and about 50% at 2 hours, between about 1% and about 75% at 4 hours, between about 5% and about 80% at 8 hours, between about 10% and about 85% at 12 hours, between about 15% and about 90% at 16 hours, between about 30% and about 80% at 24 hours, and greater than about 70% at 36 hours; or between 0% and about 60% at 1 hour, between about 0% and about 80% at 2 hours, between about 5% and about 100% at 4 hours, between about 15% and about 100% at 8 hours, between about 35% and about 100% at 12 hours, between about 40% and about 100% at 16 hours, between about 60% and about 100% at 24 hours, and greater than about 85% at 36 hours; or between 0% and about 65% at 1 hour, between about 0% and about 85% at 2 hours, between about 10% and about 100% at 4 hours, between about 20% and about 100% at 8 hours, between about 40% and about 100% at 12 hours, between about 50% and about 100% at 16 hours, between about 70% and about 100% at 24 hours, and greater than about 90% at 36 hours; or between 0% and about 70% at 1 hour, between about 0% and about 90% at 2 hours, between about 20% and about 100% at 4 hours, between about 30% and about 100% at 8 hours, between about 50% and about 100% at 12 hours, between about 60% and about 100% at 16 hours, between about 80% and about 100% at 24 hours, and greater than about 95% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 20% and about 50% at 1 hour, between about 40% and about 75% at 2 hours, between about 60% and about 95% at 4 hours, between about 80% and about 100% at 8 hours and between about 90% and about 100% at 12 hours. In other preferred embodiments, said release rate is between 15% and about 45% at 1 hour, between about 35% and about 70% at 2 hours, between about 55% and about 90% at 4 hours, between about 75% and about 90% at 8 hours and between about 80% and about 95% at 12 hours; or between 10% and about 40% at 1 hour, between about 30% and about 65% at 2 hours, between about 50% and about 85% at 4 hours, between about 70% and about 85% at 8 hours and between about 75% and about 90% at 12 hours; or between 5% and about 35% at 1 hour, between about 25% and about 60% at 2 hours, between about 45% and about 80% at 4 hours, between about 65% and about 80% at 8 hours and between about 70% and about 85% at 12 hours; or between 25% and about 55% at 1 hour, between about 45% and about 80% at 2 hours, between about 65% and about 95% at 4 hours, between about 85% and about 100% at 8 hours and between about 95% and about 100% at 12 hours; or between 30% and about 60% at 1 hour, between about 50% and about 80% at 2 hours, between about 70% and about 95% at 4 hours, between about 90% and about 100% at 8 hours and between about 95% and about 100% at 12 hours; or between 35% and about 60% at 1 hour, between about 50% and about 80% at 2 hours, between about 80% and about 95% at 4 hours, between about 90% and about 100% at 8 hours and between about 95% and about 100% at 12 hours; or between 20% and about 40% at 1 hour, between about 40% and about 65% at 2 hours, between about 60% and about 85% at 4 hours, between about 70% and about 90% at 8 hours and between about 80% and about 100% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 50% at 1 hour, between about 0% and about 75% at 2 hours, between about 10% and about 95% at 4 hours, between about 35% and about 100% at 8 hours, between about 55% and about 100% at 12 hours, between about 70% to about 100% at 16 hours, and greater than about 90% at 24 hours. In other preferred embodiments, said release rate is between 0% and about 40% at 1 hour, between about 0% and about 65% at 2 hours, between about 8% and about 85% at 4 hours, between about 30% and about 90% at 8 hours, between about 45% and about 100% at 12 hours, between about 60% to about 100% at 16 hours, and greater than about 80% at 24 hours; or between 0% and about 30% at 1 hour, between about 0% and about 55% at 2 hours, between about 5% and about 75% at 4 hours, between about 20% and about 80% at 8 hours, between about 35% and about 100% at 12 hours, between about 50% to about 100% at 16 hours, and greater than about 70% at 24 hours; or between 0% and about 20% at 1 hour, between about 0% and about 45% at 2 hours, between about 5% and about 65% at 4 hours, between about 10% and about 70% at 8 hours, between about 25% and about 80% at 12 hours, between about 40% to about 100% at 16 hours, and greater than about 60% at 24 hours; or between 0% and about 60% at 1 hour, between about 0% and about 80% at 2 hours, between about 15% and about 95% at 4 hours, between about 40% and about 100% at 8 hours, between about 60% and about 100% at 12 hours, between about 75% to about 100% at 16 hours, and greater than about 90% at 24 hours; or between 0% and about 65% at 1 hour, between about 0% and about 85% at 2 hours, between about 20% and about 90% at 4 hours, between about 45% and about 100% at 8 hours, between about 65% and about 100% at 12 hours, between about 80% to about 100% at 16 hours, and greater than about 90% at 24 hours; or between 0% and about 40% at 1 hour, between about 0% and about 50% at 2 hours, between about 10% and about 80% at 4 hours, between about 25% and about 70% at 8 hours, between about 40% and about 80% at 12 hours, between about 60% to about 100% at 16 hours, and greater than about 90% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 30% at 1 hour, between about 0% and about 45% at 2 hours, between about 3% and about 55% at 4 hours, between about 10% and about 65% at 8 hours, between about 20% and about 75% at 12 hours, between about 30% to about 88% at 16 hours, between about 50% and about 100% hours at 24 hours and greater than 80% at 36 hours. In other preferred embodiments, said release rate is between 0% and about 25% at 1 hour, between about 0% and about 40% at 2 hours, between about 2% and about 50% at 4 hours, between about 8% and about 60% at 8 hours, between about 10% and about 70% at 12 hours, between about 25% to about 80% at 16 hours, between about 45% and about 100% hours at 24 hours and greater than 75% at 36 hours; or between 0% and about 20% at 1 hour, between about 0% and about 35% at 2 hours, between about 1% and about 45% at 4 hours, between about 5% and about 55% at 8 hours, between about 8% and about 65% at 12 hours, between about 20% to about 75% at 16 hours, between about 40% and about 100% hours at 24 hours and greater than 70% at 36 hours; or between 0% and about 15% at 1 hour, between about 0% and about 30% at 2 hours, between about 0% and about 40% at 4 hours, between about 5% and about 50% at 8 hours, between about 8% and about 60% at 12 hours, between about 15% to about 70% at 16 hours, between about 35% and about 100% hours at 24 hours and greater than 60% at 36 hours; or between 0% and about 10% at 1 hour, between about 0% and about 25% at 2 hours, between about 0% and about 35% at 4 hours, between about 5% and about 45% at 8 hours, between about 10% and about 50% at 12 hours, between about 10% to about 60% at 16 hours, between about 30% and about 90% hours at 24 hours and greater than 70% at 36 hours; or between 0% and about 35% at 1 hour, between about 0% and about 50% at 2 hours, between about 5% and about 60% at 4 hours, between about 15% and about 70% at 8 hours, between about 25% and about 80% at 12 hours, between about 35% to about 90% at 16 hours, between about 55% and about 100% hours at 24 hours and greater than 85% at 36 hours; or between 0% and about 40% at 1 hour, between about 0% and about 55% at 2 hours, between about 10% and about 65% at 4 hours, between about 20% and about 75% at 8 hours, between about 30% and about 85% at 12 hours, between about 40% to about 100% at 16 hours, between about 55% and about 100% hours at 24 hours and greater than 90% at 36 hours; or between 0% and about 45% at 1 hour, between about 0% and about 60% at 2 hours, between about 15% and about 70% at 4 hours, between about 25% and about 80% at 8 hours, between about 35% and about 90% at 12 hours, between about 45% to about 100% at 16 hours, between about 60% and about 100% hours at 24 hours and greater than 60% at 36 hours; or between 0% and about 50% at 1 hour, between about 5% and about 65% at 2 hours, between about 20% and about 75% at 4 hours, between about 30% and about 85% at 8 hours, between about 40% and about 95% at 12 hours, between about 50% to about 100% at 16 hours, between about 70% and about 100% hours at 24 hours and greater than 70% at 36 hours; or between 0% and about 30% at 1 hour, between about 5% and about 40% at 2 hours, between about 10% and about 60% at 4 hours, between about 20% and about 70% at 8 hours, between about 30% and about 100% at 12 hours, between about 40% to about 100% at 16 hours, between about 60% and about 100% hours at 24 hours and greater than 90% at 36 hours; or between 0% and about 30% at 1 hour, between about 0% and about 30% at 2 hours, between about 0% and about 30% at 4 hours, between about 5% and about 70% at 8 hours, between about 10% and about 80% at 12 hours, between about 20% to about 100% at 16 hours, between about 40% and about 100% hours at 24 hours and greater than 50% at 36 hours; or between 0% and about 20% at 1 hour, between about 0% and about 20% at 2 hours, between about 0% and about 20% at 4 hours, between about 0% and about 20% at 8 hours, between about 5% and about 40% at 12 hours, between about 10% to about 80% at 16 hours, between about 40% and about 100% hours at 24 hours and greater than 60% at 36 hours; or between 0% and about 10% at 1 hour, between about 0% and about 20% at 2 hours, between about 0% and about 40% at 4 hours, between about 5% and about 60% at 8 hours, between about 10% and about 80% at 12 hours, between about 20% to about 100% at 16 hours, between about 40% and about 100% hours at 24 hours and greater than 50% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 50% at 1 hour, between about 0% and about 75% at 2 hours, between about 3% and about 95% at 4 hours, between about 10% and about 100% at 8 hours, between about 20% and about 100% at 12 hours, between about 30% to about 100% at 16 hours, between about 50% and about 100% hours at 24 hours and greater than 80% at 36 hours. In other preferred embodiments, said release rate is between 0% and about 45% at 1 hour, between about 0% and about 70% at 2 hours, between about 3% and about 90% at 4 hours, between about 8% and about 100% at 8 hours, between about 15% and about 100% at 12 hours, between about 25% to about 100% at 16 hours, between about 45% and about 100% hours at 24 hours and greater than 80% at 36 hours; or between 0% and about 40% at 1 hour, between about 0% and about 65% at 2 hours, between about 0% and about 80% at 4 hours, between about 5% and about 80% at 8 hours, between about 10% and about 90% at 12 hours, between about 20% to about 100% at 16 hours, between about 40% and about 100% hours at 24 hours and greater than 70% at 36 hours; or between 0% and about 35% at 1 hour, between about 0% and about 60% at 2 hours, between about 0% and about 70% at 4 hours, between about 3% and about 70% at 8 hours, between about 5% and about 80% at 12 hours, between about 15% to about 100% at 16 hours, between about 30% and about 100% hours at 24 hours and greater than 40% at 36 hours; or between 0% and about 60% at 1 hour, between about 0% and about 80% at 2 hours, between about 5% and about 100% at 4 hours, between about 15% and about 100% at 8 hours, between about 30% and about 100% at 12 hours, between about 40% to about 100% at 16 hours, between about 60% and about 100% hours at 24 hours and greater than 70% at 36 hours; or between 0% and about 50% at 1 hour, between about 0% and about 75% at 2 hours, between about 5% and about 95% at 4 hours, between about 25% and about 80% at 8 hours, between about 30% and about 100% at 12 hours, between about 40% to about 100% at 16 hours, between about 60% and about 100% hours at 24 hours and greater than 60% at 36 hours; or between 0% and about 60% at 1 hour, between about 0% and about 85% at 2 hours, between about 5% and about 100% at 4 hours, between about 10% and about 100% at 8 hours, between about 20% and about 100% at 12 hours, between about 30% to about 100% at 16 hours, between about 50% and about 100% hours at 24 hours and greater than 80% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 15% and about 25% at 1 hour, between about 25% and about 35% at 2 hours, between about 30% and about 45% at 4 hours, between about 40% and about 60% at 8 hours, between about 55% and about 70% at 12 hours and between about 60% to about 75% at 16 hours. In other preferred embodiments, said release rate is between 10% and about 20% at 1 hour, between about 20% and about 30% at 2 hours, between about 25% and about 40% at 4 hours, between about 30% and about 50% at 8 hours, between about 50% and about 65% at 12 hours and between about 55% to about 65% at 16 hours; or between 5% and about 15% at 1 hour, between about 15% and about 25% at 2 hours, between about 20% and about 35% at 4 hours, between about 25% and about 45% at 8 hours, between about 45% and about 60% at 12 hours and between about 50% to about 60% at 16 hours; or between 15% and about 30% at 1 hour, between about 20% and about 40% at 2 hours, between about 20% and about 50% at 4 hours, between about 30% and about 70% at 8 hours, between about 60% and about 80% at 12 hours and between about 70% to about 90% at 16 hours; or between 0% and about 50% at 1 hour, between about 5% and about 50% at 2 hours, between about 5% and about 70% at 4 hours, between about 10% and about 80% at 8 hours, between about 20% and about 100% at 12 hours and between about 40% to about 100% at 16 hours; or between 15% and about 40% at 1 hour, between about 15% and about 45% at 2 hours, between about 20% and about 60% at 4 hours, between about 20% and about 80% at 8 hours, between about 30% and about 90% at 12 hours and between about 40% to about 100% at 16 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said in-vitro release rate being substantially independent of pH in that a difference, at any given time, between an amount of said buprenorphine released at one pH and an amount released at any other pH, when measured in-vitro using the USP Basket or Paddle Methods of USP Drug Release test of U.S. Pharmacopeia (2003) at 100 rpm in 900 ml aqueous buffer, is no greater than 30%. In other preferred embodiments, the difference, at any given time, between an amount of buprenorphine released at one pH and an amount released at any other pH using the aforementioned methods is no greater than 50%, or no greater than 40%, or no greater than 35%, or no greater than 25%, or no greater than 20%, or no greater than 15%, or no greater than 10%, or no greater than 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing in-vitro release rates by weight of between 0% to about 50% by weight of buprenorphine from the dosage form at one hour when measured by the USP Basket or Paddle Methods at 100 rpm in 700 ml of Simulated Gastric Fluid (SGF) at 37° C. In other preferred embodiments, said release rate at one hour is between 0% to about 10% by weight, or 0% to about 20% by weight, or is between 0% to about 30% by weight, or 0% to about 40% by weight, or between 0% to about 60% by weight, or 0% to about 70% by weight, or 0% to about 80% by weight, or 0% to about 90% by weight, or 10% to about 50% by weight, or 10% to about 60% by weight, or 10% to about 70% by weight, or 10% to about 90% by weight, or 10% to about 100% by weight, or 30% to about 100% by weight, or 50% to about 100% by weight.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage forms providing in-vitro release rates by weight of buprenorphine, when measured by the USP Basket or Paddle Methods at 100 rpm in 900 mL aqueous buffer at a pH of between 1.6 and 7.2 at 37° C. of between 0% to about 80% at 0.5 hours, and greater than about 40% at 1 hour. In other preferred embodiments, said release rate is between 0% to about 40% at 0.5 hours, and greater than about 60% at 1 hour; or between 0% to about 20% at 0.5 hours, and greater than about 40% at 1 hour; or between 0% to about 20% at 0.5 hours, and greater than about 20% at 1 hour; or between 0% to about 90% at 0.5 hours, and greater than about 60% at 1 hour; or between 0% to about 100% at 0.5 hours, and greater than about 60% at 1 hour; or between 0% to about 90% at 1 hour, and greater than about 40% at 2 hours; or between 0% to about 100% at 1 hour, and greater than about 60% at 2 hours; or between 0% to about 60% at 1 hour, and greater than about 40% at 2 hours; or between 0% to about 40% at 1 hour, and greater than about 30% at 2 hours; or between 0% to about 50% at 1 hour, and greater than about 40% at 2 hours; or between 0% to about 30% at 1 hour, and greater than about 20% at 2 hours; or between 0% and about 50% at 1 hour, between about 0% and about 80% at 2 hours, between about 5% and about 100% at 4 hours and between about 10% and about 100% at 8 hours; or between 10% and about 60% at 1 hour, between about 15% and about 75% at 2 hours, between about 20% and about 95% at 4 hours and between about 30% and about 100% at 8 hours.

In certain embodiments, the dosage forms of the invention contains one or more substances in sufficient quantity to render said dosage form controlled release, such that: (i) in-vitro release rate of buprenorphine by weight of any commercially available sublingual formulations of buprenorphine at the time of this invention (e.g., as listed in the FDA's Orange Book, the EMEA website, Martindale: The Complete Drug Reference, 35th Edition, Pharmaceutical Press) compared with in-vitro release rate by weight of buprenorphine from the dosage form of the invention is at least about 10%, or at least about 15%, or at least about 20%, or at least about 30%, or at least about 40%, or at least about 50%, or at least about 60%, or at least about 70%, or at least about 80%, or at least about 90% faster, or at least 100% faster, or at least 200% faster, or at least 300% faster, or at least 400% faster, or at least 500% faster, or at least 600% faster, or at least 700% faster, or at least 800% faster, or at least 1000% faster, at 0.25 hour, or at 0.5 hours, or at 1 hour, or at 2 hours, or at 1 to 2 hours, when measured by the USP Basket or Paddle Methods at 100 rpm in 900 mL aqueous buffer at a pH of between 1.6 and 7.2 at 37° C.; and/or (ii) in-vivo release rate of buprenorphine by weight of any commercially available sublingual formulations of buprenorphine at the time of this invention (e.g., as listed in the FDA's Orange Book, the EMEA website, Martindale: The Complete Drug Reference, 35th Edition, Pharmaceutical Press) compared with the in-vivo release rate of buprenorphine from the dosage form of the invention is at least about 10%, or at least about 15%, or at least about 20%, or at least about 30%, or at least about 40%, or at least about 50%, or at least about 60%, or at least about 70%, or at least about 80%, or at least about 90% faster, or at least 100% faster, or at least 200% faster, or at least 300% faster, or at least 400% faster, or at least 500% faster, or at least 600% faster, or at least 700% faster, or at least 800% faster, or at least 1000% faster, said in-vivo release rate quantified by the Cₘₐₓ of buprenorphine after single dose administration to human subjects, each dosage form administered according to its approved route of administration.

In some preferred embodiments, the oral dosage from is a controlled release material suitable for extended release oral administration to a human patient of the dosage form comprises a matrix. In some preferred embodiments, the said matrix is a plurality of multiparticulate matrices. In some preferred embodiments, the multiparticulates are compressed into a tablet. In some preferred embodiments, the multiparticulates are disposed in a pharmaceutically acceptable capsule.

In some preferred embodiments, the in vivo pharmacokinetic parameters of the specifications and claims are derived or determined from first administration. In other preferred embodiments, the in vivo pharmacokinetic parameters are derived or determined from steady state administration.

In some preferred embodiments, the in vivo pharmacokinetic parameters of the specifications and claims are derived or determined under fed conditions. In other preferred embodiments, the in vivo pharmacokinetic parameters are derived or determined under fasted conditions.

In some preferred embodiments, the in vivo pharmacokinetic parameters of the specifications and claims are derived or determined from an individual subject. In other preferred embodiments, the in vivo pharmacokinetic parameters are derived or determined from a population of subjects.

In some preferred embodiments, the in vivo pharmacokinetic parameters of the specifications and claims are derived or determined in subjects having a Body Mass Index (BMI) between 18 and 26 kg/m², inclusive (BMI=[weight in kg/height in m²]×10,000). In some preferred embodiments, the in vivo pharmacokinetic parameters of the specifications and claims are derived or determined in subjects having a Body Mass Index (BMI)≧38 kg/m².

Also disclosed are methods for the treatment of addiction disorders in a human patient suffering comprising administering a therapeutically effective amount of oral buprenorphine or a pharmaceutically acceptable salt of buprenorphine or a mixture thereof. Preferably, the addiction disorder is an opioid addiction disorder or a poly-substance abuse disorder.

Also disclosed are methods for the treatment of medical conditions amenable to treatment with buprenorphine or opioid agonists in a human patient suffering comprising administering a therapeutically effective amount of oral buprenorphine or a pharmaceutically acceptable salt of buprenorphine or a mixture thereof.

Also disclosed are methods for preventing and treating pain in a human patient suffering comprising administering a therapeutically effective amount of oral buprenorphine or a pharmaceutically acceptable salt of buprenorphine or a mixture thereof.

All pain states are contemplated by this invention, regardless of etiology, mechanisms, duration, prior treatment response and anatomic location, including acute pain, inflammatory pain, chronic pain, cancer pain, visceral pain and neuropathic pain.

Also disclosed are methods of providing relief in a human patient suffering from neuropathic and chronic pain comprising a therapeutically effective amount of oral buprenorphine or a pharmaceutically acceptable salt buprenorphine or a mixture thereof. In some preferred embodiments, the dosage form of the invention is intended for the treatment of neuropathic pain, peripheral neuropathic pain, central neuropathic pain, chronic pain, osteoarthritis, back pain, cancer pain, fibromyalgia, and chronic inflammatory pain.

Also disclosed are methods of providing relief in a human patient suffering from acute pain comprising a therapeutically effective amount of oral buprenorphine or a pharmaceutically acceptable salt buprenorphine or a mixture thereof.

Also disclosed are methods of providing relief in a human patient suffering from an addiction disorder comprising a therapeutically effective amount of oral buprenorphine or a pharmaceutically acceptable salt buprenorphine or a mixture thereof.

All kinds of kits of the present invention are contemplated. In some preferred embodiments, also provided are kits for use in treating or preventing the pain with the oral administration of buprenorphine or a pharmaceutically acceptable salt of buprenorphine, or a mixture thereof for a subject in need of such treatment, comprising: (i) a dosage form of the invention; (ii) a container for the dosage form; and optionally, any of (iii) to (vi): (iii) a container for individual units of the dosage form (e.g., individual tablets or capsules in blisters); (iv) educational instructions in any media about various medical conditions, their etiology, pathophysiology, consequences and treatment, including information on the potential for abuse and diversion and methods for prevention of same and information on the proper use and disposal of the medication; (v) containers or bags for the safe disposal of any used or remaining unused dosage form, preferably child proof and flushable; (vi) tamper evident and child proof packaging for the kit and its contents.

The amount of buprenorphine in the oral dosage form will vary depending on variety of physiologic, pharmacologic, pharmacokinetic, pharmaceutical and physicochemical factors, including: (i) the choice of buprenorphine as the base, pharmaceutically acceptable salt or mixtures thereof; (ii) the nature of the oral dosage form (e.g, immediate release or extended release); (iii) the anatomical location of the pain relieving target; (iv) the intensity and intractability of the pain; (v) the contribution of different mechanism to the initiation, propagation, summation and maintenance of the pain; (vi) the absorption, metabolism, distribution and excretion of orally administered buprenorphine in healthy subjects and in patients with various diseases and disorders, including renal and hepatic impairment; (vii) the presence of comorbid pathology; (viii) the patient's risk of iatrogenic side effects; (ix) the tolerability of the dose, including the patient's propensity for buprenorphine associated CNS and gastrointestinal side effects; (x) use of concurrent analgesics; (xi) the efficiency of the dosage form.

The invention is also directed to methods of preparing the dosage forms disclosed herein.

In certain preferred embodiments, the buprenorphine in the dosage form is combined with one or more other drugs for the treatment of the same medical condition as the buprenorphine or for the treatment of a different medical condition. All modes of co-administration are contemplated, including via an oral, subcutaneous, direct intravenous, slow intravenous infusion, continuous intravenous infusion, intravenous or epidural patient controlled analgesia (PCA and PCEA), intramuscular, intrathecal, epidural, intracisternal, intramuscular, intraperitoneal, transdermal, topical, transmucosal, buccal, sublingual, inhalation, intranasal, epidural, intra-atricular, intranasal, rectal or ocular routes.

The term “first administration” means administration of a dose of the present invention at the initiation of therapy to an individual patient or a patient population.

The term “steady state” means that the amount of the drug reaching the system is approximately the same as the amount of the drug leaving the system. Thus, at “steady-state”, the patient's body eliminates the drug at approximately the same rate that the drug becomes available to the patient's system through absorption into the blood stream.

As used herein the terms: (i) “AUC₀₋ₜ” or “AUC_{0-τ}” means area under the plasma drug concentration-time curve from time zero to the intended dosing frequency of the dosage form (e.g., 8 hours, 12 hours or 24 hours) or to the last quantifiable sampling time point; (ii) “AUC_{0-inf}” means area under the plasma drug concentration-time curve from time zero to infinity; (iii) “AUC₀₋₆” means area under the plasma drug concentration-time curve from time zero to 6 hours after dosing; (iv) “AUC₀₋₈” means area under the plasma drug concentration-time curve from time zero to 8 hours after dosing; (v) “AUC₀₋₁₂” means area under the plasma drug concentration-time curve from time zero to 12 hours after dosing; (vi) “AUC₀₋₂₄” means area under the plasma drug concentration-time curve from time zero to 24 hours after dosing; (vii) “Cₘₐₓ” means the maximum observed plasma drug concentration; (viii) “C₆” means the plasma drug concentration at 6 hours after dosing; (ix) “C₈” means the plasma drug concentration at 8 hours after dosing; (x) “C₁₂” means the plasma drug concentration at 12 hours after dosing; (xi) “C₂₄” means the plasma drug concentration at 24 hours after dosing; (xii) “tₘₐₓ” or “Tₘₐₓ” means the time of the observed maximum drug concentration (also known as time to achieve Cₘₐₓ); (xiii) “Cₘᵢₙ” means the minimum observed drug concentration following the maximum plasma concentration or the concentration at the end of the intended dosing interval; (xiv) “half value duration” or “HVD” means the duration over the dosing interval during which plasma concentration of drug are greater than or equal to one-half of Cₘₐₓ, obtained by calculating the time interval beginning when the interpolated concentration first equals or exceeds one-half of Cₘₐₓ and ending at the first time point for which the interpolated concentration falls below one-half of Cₘₐₓ; (xv) “W₅₀” means the duration of the dosing interval over which the plasma concentrations are equal to or greater than 50% of the peak concentration; (xvi) “steady state” is a state of equilibrium wherein the amount of the drug reaching the system is approximately the same as the amount of the drug leaving the system or put another way, the patient's body eliminates the drug at approximately the same rate that the drug becomes available to the patient's system through absorption into the blood stream, said “time to steady state” measured by calculating the Cₘᵢₙ after each sequential dosing of drug administered at the intended dosing frequency until two consecutive Cₘᵢₙ's are not statistically different at a 10% significance level (p=0.10); (xvii) “percent fluctuation” means the variation in plasma concentrations of the drug computed as: (a) (Cₘₐₓ−Cₘᵢₙ)/Cₘᵢₙ×100 (for an individual patient) and (mean Cₘₐₓ−mean Cₘᵢₙ)/mean Cₘᵢₙ×100 (for a population); or (b) (Cₘₐₓ−Cₘᵢₙ)/Cₐᵥ×100 (for an individual patient) and (mean Cₘₐₓ−mean Cₘᵢₙ)/mean Cₐᵥ×100 (for a population); (xviii) “accumulation index” or “AI” means the ratio of the plasma concentration of the drug at the end of the intended dosing interval (i.e., 8 hours for a Q8H dosage form, 12 hours for a Q12H dosage form, and 24 hours for a Q24H dosage form) after administration, determined at steady-state (Cₛₛₘᵢₙ) to the plasma concentration of the drug at the end of the intended dosing interval determined at first administration (i.e., after the first dose).

Pharmacokinetic parameters of the invention are be computed from single dose (i.e., first administration) and steady state pharmacokinetic studies conducted in an individual subject or in a population of subjects in the fasted or fed states. The AI and percent of steady state computations requires both single dose (i.e., first administration) and steady state pharmacokinetic assessment.

In certain preferred embodiments of the present invention, an effective amount of buprenorphine in immediate release form is included in the controlled release unit dose buprenorphine formulation to be administered. The immediate release form of the buprenorphine is preferably included in an amount which is effective to shorten the time to Cₘₐₓ of the buprenorphine in the blood (e.g., plasma). In such embodiments, an effective amount of the buprenorphine in immediate release form may be coated onto the substrates of the present invention. For example, where the extended release buprenorphine from the formulation is due to a controlled release coating, the immediate release layer would be overcoated on top of the controlled release coating. On the other hand, the immediate release layer maybe coated onto the surface of substrates wherein the buprenorphine is incorporated in a controlled release matrix. Where a plurality of the sustained release substrates comprising an effective unit dose of the buprenorphine (e.g., multiparticulate systems including pellets, spheres, beads and the like) are incorporated into a hard gelatin capsule, the immediate release portion of the buprenorphine dose may be incorporated into the gelatin capsule via inclusion of the sufficient amount of immediate release buprenorphine as a powder or granulate within the capsule. Alternatively, the gelatin capsule itself may be coated with an immediate release layer of the buprenorphine. One skilled in the art would recognize still other alternative manners of incorporating the immediate release buprenorphine into the unit dose. Such alternatives are deemed to be encompassed by the appended claims. By including such an effective amount of immediate release buprenorphine in the unit dose, the experience of relatively higher levels of pain in patients may be significantly reduced.

For purposes of the invention, the term “a patient” in reference to pharmacokinetic parameters means that the discussion (or claim) is directed to the pharmacokinetic parameters of an individual patient or subject.

The term “population of patients” or “patient population” means that the discussion (or claim) is directed to the mean pharmacokinetic parameters of at least two patients or subjects.

In certain preferred embodiments, any one or all of the above in-vivo parameters are achieved after a first administration (often referred to as “single dose administration”) of the dosage form to a human patient or a population of human patients.

In certain alternative embodiments, any one or all of the above in-vivo parameters are achieved after steady state administration of the dosage form to a human patient or a population of human patients.

In certain preferred embodiments, the amount of buprenorphine in the dosage form is about 0.001 mg to 1500 mg. In other more preferred embodiments, the amount of buprenorphine in the dosage form is about 0.1 mg to 1000 mg. In most preferred embodiments, the amount of buprenorphine in the dosage form is about 0.5 mg to about 500 mg or about 1 mg to about 200 mg, or 2 mg to about 100 mg or 1 mg to about 60 mg.

The term “USP Paddle or Basket Method” is the Paddle and Basket Method described, e.g., in specified in the United States Pharmacopeia, USP-28 NF-23 (2005), published by the United States Pharmacopeial Convention, Inc, herein incorporated by reference.

The term “pH-dependent” for purposes of the present invention is defined as having characteristics (e.g., dissolution) which vary according to environmental pH.

The term “pH-independent” for purposes of the present invention is defined as having characteristics (e.g., dissolution) which are substantially unaffected by pH.

The term “bioavailability” is defined for purposes of the present invention as the extent to which the drug (e.g., buprenorphine) is absorbed from the unit dosage forms.

As used herein with respect to the buprenorphine dosage form of the invention, the term “oral”, “oral dosage form”, “oral pharmaceutical dosage form”, “oral administration”, and “oral route” and the like all refer to any method of administration through the mouth for rapid deposit into the stomach or alimentary canal. The oral dosage form of the invention is usually ingested intact, although it may be ingested un-intact or tampered (e.g., crushed) and usually with the aid of water or a beverage to hasten passage through the mouth. Specifically excluded from this invention and the forgoing definition of oral dosage forms are buprenorphine dosage forms and pharmaceutical compositions which are administered by the lingual, sublingual, oro-mucosal, transmucosal and buccal routes. Lingual, sublingual, oro-mucosal, transmucosal and buccal routes are intended to provide absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach). Such formulations and their method of administration are well known in the art and include lozenges, transmucosal films, buccal products, mucoretentive products, orally disintegrating tablets, fast dissolving tablets, fast dispersing tablets, fast disintegrating dosage forms, provided they are administered for absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach).

All oral pharmaceutical dosage forms of the invention are contemplated, including oral suspensions, tablets, capsules, effervescent tablets, effervescent powders, powders, solutions, powders for reconstitution, oral gastroretentive tablets and capsules, administered as immediate release, modified release, enteric coated, sustained release, controlled release, pulsatile release and extended release dosage form.

As used herein, “controlled release” is interchangeable with “extended release”, “sustained release”, “modified release”, “delayed release” and the like.

Controlled release dosage forms of the present invention release of buprenorphine from the oral dosage form at slower rate than immediate release formulations. In some preferred embodiments, controlled release dosage forms of release buprenorphine at such a rate that blood (e.g., plasma) concentrations (levels) or therapeutic effects are maintained within the therapeutic range (above the minimum effective therapeutic concentration) but below toxic levels for intended duration (e.g., over a period of 1 to 24 hours, preferably over a period of time indicative of a Q3, Q4, Q6, Q8, Q12 or Q24H administration). Notwithstanding the foregoing, in some preferred embodiments, the controlled release formulations of the present invention provide therapeutic effects for a duration that is longer or substantially longer than the duration of meaningful or detectable plasma concentrations of buprenorphine.

The term “immediate release buprenorphine” for purposes of the present invention, is buprenorphine for oral administration in a dosage form which is formulated to release the active drug from the dosage form immediately (i.e., without an attempt to delay or prolong the release of the active drug from the dosage form as is the case for extended release dosage forms). In the absence of a commercially available oral immediate release buprenorphine product, an available parenteral formulation of buprenorphine or a salt thereof may be used orally or a solution of buprenorphine or a salt thereof may be prepared for the purpose of in vivo testing requiring immediate release buprenorphine.

In the absence of a commercially available immediate release sublingual formulation of buprenorphine listed in FDA's Orange Book, alternative sublingual formulations may be substituted (for example, commercially available sublingual formulations approved by the EMEA or available in the European Union, or sublingual formulations listed in Martindale: The Complete Drug Reference, 35th Edition (or more recent), Pharmaceutical Press).

For purposes of the invention, the oral controlled release formulations disclosed herein and the oral immediate release control formulations are dose proportional. In such formulations, the pharmacokinetic parameters (e.g., AUC and Cₘₐₓ) generally increase linearly from one dosage strength to another. Therefore the pharmacokinetic parameters of a particular dose can be inferred from the parameters of a different dose of the same formulation.

In certain situations involving pharmacokinetic evaluations, it may not be possible to provide the same amount of drug by different routes of administration due to the lack of commercially available dosage strengths or because such administration would require testing outside the approved method of administration (e.g., simultaneous sublingual administration o five tablets). Under such circumstances, the term “after the same amount of an oral immediate release formulation of buprenorphine” may be waived and different amounts of drug may be evaluated, provided the data are dose normalized using pharmacokinetic approaches well known in the art.

The term “agonist” means a ligand that binds to a receptor and alters the receptor state resulting in a biological response. Conventional agonists increase receptor activity, whereas inverse agonists reduce it (See Neubig et al, IUPHAR Committee on Receptor Nomenclature and Classification, Pharmacol Rev, 2003; Howlett et al., Mol Pharmacol, 1988).

The term “opioid agonist” means a molecule that causes a specific physiologic, pathophysiologic or pharmacologic effect after binding to an opioid receptor.

An “antagonist” is a drug or ligand that reduces the action of another drug or ligand, generally an agonist. Many antagonists act at the same receptor macromolecule as the agonist. (See Neubig et al, IUPHAR Committee on Receptor Nomenclature and Classification, Pharmacol Rev, 2003; Howlett et al., Mol Pharmacol, 1988).

The term “receptor” means a molecule within a cell, on a cell surface, on a membrane, in tissue, in fluid or otherwise found in humans that serve as a recognition or binding site to cause specific physiologic, pathophysiologic or pharmacologic effects. The term “receptor” also means a cellular macromolecule, or an assembly of macromolecules, that is concerned directly and specifically in chemical signaling between and within cells. Combination of a hormone, neurotransmitter, drug, ligand, or intracellular messenger with its receptor(s) initiates a change in cell function (Neubig et al, IUPHAR Committee on Receptor Nomenclature and Classification, Pharmacol Rev, 2003).

The term “opioid receptor” includes mu (μ) delta (δ) and kappa (κ) opioid receptors, their subtypes and splice variants such as mu₁, mu₂, delta₁, delta₂, kappa₁, kappa₂ and kappa₃, etc.

Opioid antagonists are known or readily determined by individuals who practice the art. Preferably, the opioid antagonists useful for the present invention may be selected from the group consisting of naltrexone, methylnaltrexone, nalbuphine, naloxone, nalmefene, cyclazocine, cyclorphan, oxilorphan nalorphine, nalorphine dinicotinate, nalmefene, nadide and levallorphan.

In certain preferred embodiments of the present invention, the invention allows for the use of lower doses of buprenorphine by virtue of the inclusion or co-administration of an additional drug for the prevention or treatment of pain. By using lower amounts of either or both drugs, the side effects associated with treatment in humans are reduced.

The term “buprenorphine” means buprenorphine base, as well as their pharmaceutically acceptable salts, prodrugs, esters, analogs, derivatives, solvates, complexes, polymorphs, and hydrates, as racemates or an individual diastereoisomers or enantiomeric isomers thereof or mixtures thereof. In some preferred embodiments, the dosage form comprises buprenorphine base or their pharmaceutically acceptable salts, or mixtures thereof. In some even more preferred embodiments, the dosage form comprises buprenorphine base or buprenorphine HCl, or mixtures thereof.

The phrase “comprising a therapeutically effective amount of buprenorphine” means “comprising a therapeutically effective amount of buprenorphine or a pharmaceutically acceptable salt of buprenorphine, or prodrugs, esters, analogs, derivatives, solvates, complexes, polymorphs and hydrates thereof, as racemates or an individual diastereoisomers or enantiomeric isomers thereof or mixtures thereof.

When the dosage form includes a pharmaceutically acceptable salt, any salt may be use. Preferably, the salt is the hydrochloride salt of buprenorphine.

The singular forms “a,” “an” and “the” include plural referents unless the context clearly dictates otherwise. Thus, for example, reference to “a polymer” includes a single polymer as well as a mixture of two or more different polymers, reference to “a permeation enhancer” includes a single permeation enhancer as well as two or more different permeation enhancer in combination, and the like.

Some of the drugs disclosed herein may contain one or more asymmetric centers and may thus give rise to enantiomers, diastereomers, and other stereoisomeric forms. The present invention is also meant to encompass all such possible forms as well as their racemic and resolved forms and mixtures thereof. When the compounds described herein contain olefinic double bonds or other centers of geometric asymmetry, and unless specified otherwise, it is intended to include both E and Z geometric isomers. All tautomers are intended to be encompassed by the present invention as well.

As used herein, the term “stereoisomers” is a general term for all isomers of individual molecules that differ only in the orientation of their atoms is space. It includes enantiomers and isomers of compounds with more than one chiral center that are not mirror images of one another (diastereomers).

The term “chiral center” refers to a carbon atom to which four different groups are attached.

The term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule.

The “NNH” or “number needed to harm” is a measure that indicates how many patients would require a specific treatment to cause harm in one patient. As used herein, the “NNH or “number needed to harm” is a measure that includes: (i) how many opioid naive healthy subjects would require treatment to cause moderate or severe sedation (or drowsiness) in one subject, where moderate to severe sedation or drowsiness is defined as a VAS score of ≧50 mm on a 100 mm scale bounded on the left by “no sedation or drowsiness” and on the right by “extreme sedation or drowsiness”; (ii) how many opioid naive healthy subjects would require treatment to cause moderate or severe nausea in one subject, where moderate to severe nausea is defined as a VAS score of ≧50 mm on a 100 mm scale bounded on the left by “no nausea” and on the right by “extreme nausea”; (iii) how many opioid naive healthy subjects would require treatment to cause dizziness in one subject, where dizziness is defined as unsteadiness, imbalance, lightheadedness, spinning sensation or sensation that one is falling; (iv) how many opioid naive healthy subjects would require treatment to cause a sensation of dry mouth in one subject, where dry mouth is defined as abnormal dryness of the mouth associated with decreased secretion of saliva.

The “drug effects” questionnaire assesses the extent to which subjects currently felt a drug effect, on a scale of 1 to 5 (1=“I feel no effect from it at all”; 2=“I think I feel a mild effect, but I'm not sure”; 3=“I feel an effect, but it is not real strong”; 4=“I feel a strong effect”; 5=“I feel a very strong effect”). This questionnaire can be used to examine the overall drug effects, preferably in drug abusers and recreational drug users.

The “drug liking” questionnaire assesses the extent to which subjects currently like the effects of the drug on a 100-mm VAS, bounded on the left by “0=dislike a lot”, bounded on the right by “100=like a lot”. This questionnaire can be used to examine the overall drug liking of, preferably in drug abusers and recreational drug users.

The “take again” questionnaire assesses whether subjects would take the drug again if given the opportunity. The patient is asked “If given an opportunity, would you take this drug again? (circle one: YES or NO). This questionnaire can be used to examine the overall desirability of the drug experience, preferably in drug abusers and recreational drug users.

On the “coasting” questionnaire the patient is asked to put a mark on a horizontal line that best describes their response to the question: “Do you feel like you are coasting or spaced out? The horizontal line is a visual analog scale (VAS) bounded on the left by “not at all” and on the right by “extremely”. This questionnaire can be used to examine the degree to which subjects feel like they are coasting or spaced out, preferably in drug abusers and recreational drug users.

Three performance tasks may be employed for measuring skills related to driving.

The “critical tracking task” measures the patient's ability to control a displayed error signal in a first-order compensatory tracking task. The error is displayed as a horizontal deviation of a cursor from the midpoint on a horizontal, linear scale. Compensatory joystick movements correct the error by returning the cursor to the midpoint. The frequency at which the patient loses the control is the critical frequency. The critical tracking task measures the psychomotor control during a closed loop operation. It is a laboratory analog to on-the-road tracking performance.

The “stop signal task” measures motor impulsivity, which is defined as the inability to inhibit an activated or pre-cued response leading to errors of commission. The task requires patients to make quick key responses to visual go signals, i.e. the letters ABCD presented one at a time in the middle of the screen, and to inhibit any response when a visual stop signal, i.e. “*” in one of the four corners of the screen, is presented at predefined delays. The main dependent variable is the stop reaction time on stop signal trials that represents the estimated mean time required to inhibit a response.

The Tower of London (TOL) is a decision-making task that measures executive function and planning. The task consists of computer generated images of begin- and end-arrangements of three colored balls on three sticks. The subject's task is to determine as quickly as possible, whether the end-arrangement can be accomplished by “moving” the balls in two to five steps from the beginning arrangement by pushing the corresponding number coded button. The total number of correct decisions is the main performance measure.

In some embodiments, the dosage form of the invention, one or more or all of the specifications and claims applicable to the prevention and treatment of pain or addiction disorders is also applicable to the prevention or treatment of any other disease or disorder that responds to opioid agonists or to buprenorphine.

The prevention and treatment of all diseases and disorders is contemplated by the use of this invention, including without limitation, (i) pain; (ii) addiction disorders; (iii) opioid substitution and opioid maintenance therapy; (iv) restless leg syndrome; (v) cough; (vi) urinary incontinence; (vii) fibromyalgia; (viii) pain associated with sickle cell disease, including vaso-occlusive crisis; (ix) peripheral and central neuropathic pain; (x) cancer pain; (xi) breakthrough pain; (xii) visceral pain; (xiii) dyspnea and respiratory distress; (xiv) infectious, immunologic, cardiovascular, pulmonary, gastrointestinal, hepatic, biliary, nutritional, metabolic, endocrine, hematologic, oncologic, musculoskeletal, rheumatic, neurologic, psychiatric, genitourinary, gynecologic, obstetric, pediatric, otolaryngogologic, ophthalmic, dermatologic, dental, oral, and genetic disorders, diseases and maladies and signs and symptoms thereof; (xv) depression, schizophrenia, influenza, common colds, anxiety, panic attacks, agoraphobia, ADHD, insomnia, sleep disorders, nasal congestion, headaches, migraine, urinary incontinence, constipation, allergies, cough, pneumonia, COPD, asthma, fluid retention, acid reflux, peptic ulcers, hypertension, cardiac arrhythmias, hypercholesterolemia, CHF, fever, diarrhea, back pain, myofascial pain, osteoarthritis, neuropathic pain, cancer pain, acute pain, diabetes, muscle spasms, and rheumatoid arthritis, and signs and symptoms thereof; and (xvi) disorders, diseases and maladies, and signs and symptoms thereof referred to in Harrison's Principles of Internal Medicine, 16th Edition, 2004, Kasper D L, Braunwald W, Fauci A, Hauser S, Longo D, and Jameson J L (eds)], which is hereby incorporated in its entirety by reference; said disorders, diseases and maladies, and signs and symptoms thereof comprising buprenorphine responsive medical conditions.

In some preferred embodiments, the oral pharmaceutical dosage forms of buprenorphine are used to treat pain, cough, dyspnea, opioid addiction disorders, restless leg syndrome, fibromyalgia, acute herpes zoster, visceral pain, breakthrough pain, opioid dependence and urinary incontinence.

As used herein, the term “pain” includes: (i) peripheral neuropathic pain, e.g., acute and chronic inflammatory demeyelinating polyradiculopathy, alcoholic polyneuropathy, chemotherapy-induced polyneuropathy, complex regional pain syndrome (CRPS) Type I and Type II, entrapment neuropathies (e.g., carpal tunnel syndrome), HIV sensory neuropathy, iatrogenic neuralgias (e.g., postthoracotomy pain, postmastectomy pain), idiopathic sensory neuropathy, painful diabetic neuropathy, phantom limb pain, postherpetic neuralgia, trigeminal neuralgia, radiculopathy (e.g., cervical thoracic, lumbosacral), sciatica, acute herpes zoster pain, temporomandibular joint disorder pain and postradiation plexopathy; and (ii) central neuropathic pain, e.g., compressive myelopathy from spinal stenosis, HIV myelopathy, multiple sclerosis pain, Parkinson's disease pain, postischemic myelopathy, post postradiation myelopathy, poststroke pain, posttraumatic spinal cord injury and syringomyelia; and (iii) cancer associated neuropathic pain, e.g., chemotherapy induced polyneuropathy, neuropathy secondary to tumor infiltration or nerve compression, phantom breast pain, postmastectomy pain, postradiation plexopathy and myelopathy; (iv) chronic pain, e.g., back pain, rheumatoid arthritis, osteoarthritis, inflammatory pain, non-inflammatory pain, myofascial pain, fibromyalgia, cancer pain, visceral pain, somatic pain, pelvic pain, musculoskeletal pain, post-traumatic pain, bone pain and idiopathic pain; (v) acute pain, e.g, acute postsurgical pain (including laparoscopic, laparatomy, gynecologic, urologic, cardiothoracic, arthroscopic, gastrointestinal, neurologic, orthopedic, oncologic, maxillofacial, ophthalmic, otolaryngologic, soft tissue, plastic, cosmetic, vascular and podiatric surgery, including abdominal surgery, abdominoplasty, adenoidectomy, amputation, angioplasty, appendectomy, arthrodesis, arthroplasty, arthroscopy, bilateral cingulotomy, biopsy, brain surgery, breast biopsy, cauterization, cesarean section, cholecystectomy, circumcision, commissurotomy, cordotomy, corneal transplantation, cricothoracotomy, discectomy, diverticulectomy, episiotomy, endarterectomy, endoscopic thoracic sympathectomy, foreskin restoration, fistulotomy, frenectomy, frontalis lift, fundectomy, gastrectomy, grafting, heart transplantation, hemicorporectomy, hemorrhoidectomy, hepatectomy, hernia repair, hypnosurgery, hysterectomy, kidney transplantation, laminectomy, laparoscopy, laparotomy, laryngectomy, lithotripsy, lobotomy, lumpectomy, lung transplantation, mammectomy, mammoplasty, mastectomy, mastoidectomy, mentoplasty, myotomy, mryingotomy, nephrectomy, nissen fundoplication, oophorectomy, orchidectomy, parathyroidectomy, penectomy, phalloplasty, pneumotomy, pneumonectomy, prostatectomy, psychosurgery, radiosurgery, ritidoplasty, rotationplasty, sigmoidostomy, sphincterotomy, splenectomy, stapedectomy, thoracotomy, thrombectomy, thymectomy, thyroidectomy, tonsillectomy, tracheotomy, tracheostomy, tubal ligation, ulnar collateral ligament reconstruction, ureterosigmoidostomy, vaginectomy, vasectomy, vulvectomy; renal colic; incisional pain; inflammatory incisional pain; nociceptive incisional pain; acute neuropathic incisional pain following surgery), renal colic, trauma, acute back pain, burn pain, burn dressing change pain, migraine pain, tension headache pain, acute musculoskeletal pain, acute exacerbation or flare of chronic back pain, acute exacerbation or flare of osteoarthritis, acute exacerbation or flare of chronic pain, breakthrough chronic non-cancer pain, breakthrough cancer pain, acute exacerbation or flare of fibromylagia, acute exacerbation or flare of rheumatoid arthritis, acute exacerbation or flare of myofacsial pain, acute exacerbation or flare of chronic idiopathic pain, acute exacerbation or flare of neuropathic pain, procedure related pain (e.g., arthroscopy, laparoscopy, endoscopy, intubation, bone marrow biopsy, soft tissue biopsy, catheterization), and other self-limiting pain states.

As used herein, the term “acute pain” refers to self-limiting pain that subsides over time and usually lasting less that about 30 days and more preferably lasting less than about 21 days. Acute pain does not include chronic conditions such as chronic neuropathy, chronic neuropathic pain and chronic cancer and non-cancer pain.

As used herein, “neuropathic pain” is pain initiated or caused by a primary lesion or dysfunction of the nervous system and includes (i) peripheral neuropathic pain and (ii) central neuropathic pain.

As used herein, the term “chronic pain” includes all non-neuropathic pain usually lasting more than 30 days, including inflammatory pain, non-inflammatory pain, muscle pain, joint pain, fascia pain, visceral pain, bone pain and idiopathic pain.

The term “analgesic effectiveness” is defined for purposes of the present invention as a satisfactory prevention, reduction in or elimination of pain, along with a tolerable level of side effects, as determined by the human patient.

According to the American Academy of Pain Medicine, the American Pain Society and the American Society of Addiction Medicine “addiction” and “addiction disorder” is a primary, chronic, neurobiologic disease, with genetic, psychosocial, and environmental factors influencing its development and manifestations. It is characterized by behaviors that include one or more of the following: impaired control over medication use, compulsive use, continued use despite harm, and craving (Sloan and Babul, Expert Opinion on Drug Delivery 2006; 3:489-97). The pharmaceutical composition of the present invention is in some embodiments intended to treat addiction disorder, particularly opioid addiction disorder and poly-substance abuse involving opioids. In some embodiments, the dosage form of the invention is intended to reduce or eliminate the craving or desire for opioids and the antisocial, medically harmful and potentially criminal behavior of the patient with the addiction disorder. The use of sublingual buprenorphine for the treatment of addiction disorder has been well established in the literature.

The term “therapeutic effectiveness” is defined for purposes of the present invention as a satisfactory prevention, reduction in or elimination of signs and symptoms of the medical disorder, disease or syndrome (e.g., pain, addiction disorder), along with a tolerable level of side effects, as determined by the human patient.

As used herein, the “Orange Book” as it is commonly known is the database of Approved Drug Products with Therapeutic Equivalence Evaluations maintained by or on behalf of the US Food and Drug Administration, (http://www.fda.gov/cder/ob/default.htm, accessed Feb. 15, 2008), the content of which is hereby incorporated by reference.

“Drug”, “drug substance”, “substance”, “therapeutic agent”, “pharmacological agent”, “pharmaceutical agent”, “active agent” and “agent” are used interchangeably and are intended to have their broadest interpretation as to any therapeutically active substance which is delivered to a living organism to produce a desired, usually beneficial effect. In general, this includes therapeutic agents in all of the major therapeutic areas.

The term “subject” for purposes of treatment is used interchangeably with “patient”, “male”, “female”, and includes any human subject.

“Pharmaceutically or therapeutically acceptable excipient or carrier” or “excipient” refers to a substance which does not interfere with the effectiveness or the biological activity of the active ingredients and which is not toxic to the subject. In some embodiments of the present invention, pharmaceutically or therapeutically acceptable excipients or carriers may play a role in imparting or optimizing the rate and extent of absorption or buprenorphine or additional drugs in the pharmaceutical composition. In some embodiments of the present invention, pharmaceutically or therapeutically acceptable excipients or carriers may play a role in stabilizing the buprenorphine or additional drugs in the pharmaceutical composition. Excipients are widely known in the art (see, for example, FDA EAFUS database (http://vm.cfsan.fda.gov/˜dms/eafus.html); FDA Food Additives Status List (http://www.cfsan.fda.gov/˜dms/opa-appa.html); FDAGRAS list and database; FDA Color Additive Status List (http://www.cfsan.fda.gov/˜dms/opa-appc.html); FDA Inactive Ingredients Database (http://www.accessdata.fda.gov/scripts/cder/iig/index.cfm); Rowe, Sheskey and Owen, Handbook of Pharmaceutical Excipients, APhA Publications; 5th edition (2006); Goodman & Gilman's The Pharmacological Basis of Therapeutics (Brunton, Lazo and Parker, eds, 11th ed., McGraw Hill (2005); Remington: The Science and Practice of Pharmacy, 21st ed, Lippincott Williams & Wilkins (2005); Martindale: The Complete Drug Reference, 35th Edition, Pharmaceutical Press (2007); United States Pharmacopeia-National Formulary (USP-NF), (USP 30-NF 25, 2007), the International Programme on Chemical Safety (http://www.inchem.org/) and Health Canada's List of Acceptable Non-medicinal Ingredients (http://www.hc-sc.gc.ca/dhp-mps/prodnatur/legislation/docs/nmi-imn_list1_e.html), all hereby incorporated by reference in their entirety).

In certain preferred embodiments of the present invention, an effective amount of buprenorphine in immediate release form is included in the controlled release unit dose buprenorphine formulation to be administered. The immediate release form of the buprenorphine is preferably included in an amount which is effective to shorten the time to Cₘₐₓ of the buprenorphine in the blood (e.g., plasma). One skilled in the art would recognize various means of incorporating the immediate release buprenorphine into the unit dose. By including such an effective amount of immediate release buprenorphine in the unit dose, patients may experience superior relief of pain and neuropathy symptoms.

In certain preferred embodiments of the present invention, the dosage form may include, in addition to buprenorphine or a pharmaceutically acceptable salt thereof, abuse deterrent or abuse resistant substances, process or technologies known in the art, including one or more aversive agents. All kinds of aversive agents are contemplated, including, without limitation, antagonists of abusable drugs, laxatives, cutaneous vasodilators, headache producing agents, emetics, emetogenic compound, nausea producing compounds, bittering agents, drugs that cause burning on irritation when in contact with tissue or mucous membranes (e.g., naso-mucosal irritants, oro-mucosal irritants, respiratory irritants), tissue irritants, gastrointestinal irritants, drugs that precipitate withdrawal effects, tissue dyes, lakes and colorants, beverage dyes, lakes and colorants, non-tissue staining beverage dyes, lakes and colorants (i.e., that do not stain or discolor the skin upon ingestion), fecal discolorants, urine discolorants, malodorous agents, opioid antagonists, benzodiazepine antagonists (e.g., flumazenil), cannabinoid antagonists and pharmacologic antagonists to co-abused drugs not contained in the dosage form. Such aversive agents may be in the dosage form in a releasable, partially releasable or a non-releasable form (i.e., sequestered), the latter being released on tampering the dosage form (e.g., mechanical, thermal, chemical, solvent tampering, ingestion in ways not recommended, and the like). Further, in some embodiments, such aversive agents may be in the dosage form in an amount that does not produce an aversive effect or aversion in any, many or substantially all patients when taken in accordance with the prescribing information or the manufacturer's instructions (for example, in small quantities), but which produce an aversive effect when taken in excess (e.g., higher dose or more frequently).

In some embodiments, one or more aversive agents may be added to the formulation in an amount of less than about 80% by weight, preferably less than about 60% by weight, more preferably less than about 40% by weight of the dosage form, even more preferably less than about 20% by weight of the dosage form, and most preferably less than about 10 by weight of the dosage form (e.g., 0.000000000000001% to 1%, or 0.000000001% to 3%, or 0.0001% to 10%, or 0.001% to 5%, or 1% to 10%, or 0.001% to 2%, or 1% or 10%, or 2% to 7%) depending on the particular aversive agent used.

In some embodiments, the aversive agent in the dosage form may be about 0.00000000001 mg to about 2000 mg, or about 0.0000001 mg to about 1500 mg, or about 0.000001 mg to about 1000 mg, or about 0.0001 mg to about 1000 mg, or about 0.001 mg to about 1000 mg, or about 0.01 mg to about 1000 mg, or about 0.1 mg to about 1500 mg, or 1 mg to about 800 mg, or about 1 mg to about 500 mg, or about 1 mg to about 300 mg, or about 1 mg to about 150 mg, or about 5 mg to about 400 mg, or about 5 mg to about 200 mg, or about 0.00000000001 mg to about 200 mg, or about 0.00000000001 mg to about 100 mg, or about 0.00000000001 mg to about 50 mg, or about 0.0000001 mg to about 200 mg, or about 0.0000001 mg to about 100 mg, or about 0.00001 mg to about 400 mg, or about 0.0001 mg to about 300 mg.

As described above, the present invention can include one or more aversive agents, selected from the group including, without limitation antagonists of abusable drugs, laxatives, cutaneous vasodilators headache producing agents, emetics, emetogenic compound, nausea producing compounds, bittering agents, drugs that cause burning on irritation when in contact with tissue or mucous membranes (e.g., naso-mucosal irritants, oro-mucosal irritants, respiratory irritants), tissue irritants, gastrointestinal irritants, drugs that precipitate withdrawal effects, tissue dyes, lakes and colorants, beverage dyes, lakes and colorants, non-tissue staining beverage dyes, lakes and colorants, fecal discolorants, urine discolorants, malodorous agents, opioid antagonists, benzodiazepine antagonists, cannabinoid antagonists, and pharmacologic antagonists to co-abused drugs not contained in the dosage form. Preferably, the aversive agent is a pharmaceutically acceptable agent that produces an aversive effect only when the dosage form of the invention containing the aversive agent is abused, for example, when taken in excess of medically approved doses, taken in excess of approved doses in the manufacturer's prescribing information, taken after tampering of the dosage form (e.g., mechanical, thermal, chemical, solvent tampering), ingestion in ways not medically recommended, administration by routes not approved for the dosage form (e.g., intranasal, inhalation, intravenous) or in a manner inconsistent with the manufacturer's prescribing information.

In some embodiments, the amount of aversive agent in the dosage form of the present invention can be a fixed ratio in relation to the amount of abusable drug in the dosage form. By appropriately selecting the quantity of the aversive agent in the dosage form, aversive effects can be avoided under conditions of proper medical use (e.g., manufacturers prescribing directions). However, under some conditions of abuse, for example excessive intake of the dosage form of the invention, the quantity of aversive agent consumed will exceed the “no effect” or “minimum effect” threshold, thereby producing one or more aversive effects, for example, e.g., nausea, emesis, diarrhea, laxation, cutaneous vasodilation, headache, bitter taste, naso-mucosal irritation, oro-mucosal irritation, precipitation of abstinence from the abusable drug of the dosage form, precipitation of abstinence from a co-abused drug which is not part of the dosage form, reduction of the pleasurable, mood altering, rewarding, reinforcing, stimulant, depressant or other psychic and physiologic effects of the abusable drug or a co-abused drug, etc.).

In some embodiments, the “no effect” or “minimum effect” threshold amount of aversive agent can be exceeded when the dosage form of the invention is taken in excess of the manufacturer's recommendation by a factor of about 1.5, or about 2, or about 2.5, or about 3, or about 4, or about 5, or about 6, or about 7, or about 8, or about 10, or more than 10. In some embodiments, the production of an aversive effect can reduce or stop further abuse of the dosage form, thereby reducing the harm or toxicity of the drug in the subject who is tampering, misusing or abusing the dosage form, e.g., addicts, drug abusers and recreational drug users.

Various bittering agents can be employed including, for example and without limitation, T2R or TAS2R receptor agonists, phenylthiourea (phenylthiocarbamide), natural, artificial and synthetic flavor oils and flavoring aromatics and/or oils, oleoresins and extracts derived from plants, leaves, flowers, fruits, and so forth, and combinations thereof. Nonlimiting representative flavor oils include spearmint oil, peppermint oil, eucalyptus oil, oil of nutmeg, allspice, mace, oil of bitter almonds, menthol and the like. Also useful bittering agents are artificial, natural and synthetic fruit flavors such as citrus oils including lemon, orange, lime, grapefruit, and fruit essences and so forth. Additional bittering agents include sucrose derivatives (e.g., sucrose octaacetate), chlorosucrose derivatives, quinine and quinine salts, quinidine and quinidine salts and the like. The preferred bittering agent for use in the present invention is denatonium, denatonium benzoate and denatonium saccharide. A dosage form including a bittering agent preferably discourages improper usage of the tampered dosage form by imparting a disagreeable taste to the tampered dosage form.

In some embodiments, the aversive agent in the dosage form may be denatonium, denatonium saccharide or denatonium benzoate, in a quantity expressed as mg of denatonium, of about 0.00000001 mg to about 100 mg, or about 0.000001 mg to about 100 mg, or about 0.0001 mg to about 100 mg, or about 0.0001 mg to about 20 mg, or about 0.0001 mg to about 10 mg, or about 0.0001 mg to about 5 mg, or about 0.0001 mg to about 2 mg, or about 0.0001 mg to about 1 mg, about 0.0001 mg to about 50 mg, or about 0.00000001 mg to about 50 mg, or about 0.00000001 mg to about 20 mg, or about 0.01 mg to about 20 mg, or about 0.01 mg to about 10 mg, or about 0.01 mg to about 5 mg, or about 0.01 mg to about 2 mg, or about 0.01 mg to about 1 mg, or about 0.01 mg to about 1 mg, or about 0.01 mg to about 0.5 mg, or about 0.1 mg to about 20 mg, or about 0.1 mg to about 10 mg, or about 0.1 mg to about 7 mg, or about 0.1 mg to about 5 mg, or about 0.1 mg to about 3 mg, or about 0.1 mg to about 2 mg.

In some embodiments, the aversive agent in the dosage form may be quinine or a pharmaceutically acceptable salt of quinine, in a quantity expressed as mg of quinine, of about 0.00001 mg to about 300 mg, or about 0.00001 mg to about 200 mg, or about 0.00001 mg to about 100 mg, or about 0.00001 mg to about 75 mg, or about 0.00001 mg to about 50 mg, or about 0.00001 mg to about 25 mg, or about 0.00001 mg to about 20 mg, or about 0.00001 mg to about 10 mg, or about 0.00001 mg to about 5 mg, or about 0.00001 mg to about 2.5 mg, or about 0.00001 mg to about 1 mg, or about 0.001 mg to about 300 mg, or about 0.001 mg to about 200 mg, or about 0.001 mg to about 100 mg, or about 0.001 mg to about 75 mg, or about 0.001 mg to about 50 mg, or about 0.001 mg to about 25 mg, or about 0.001 mg to about 20 mg, or about 0.001 mg to about 10 mg, or about 0.001 mg to about 5 mg, or about 0.001 mg to about 2.5 mg, or about 0.001 mg to about 1 mg, or about 1 mg to about 300 mg, or about 1 mg to about 200 mg, or about 1 mg to about 100 mg, or about 1 mg to about 75 mg, or about 1 mg to about 50 mg, or about 1 mg to about 25 mg, or about 1 mg to about 20 mg, or about 1 mg to about 10 mg, or about 1 mg to about 5 mg, or about 1 mg to about 2.5 mg.

Various emetic agents can be employed including, for example and without limitation, zinc and pharmaceutically acceptable salts thereof (e.g., zinc oxide, zinc gluconate, zinc acetate, zinc sulfate, zinc carbonate), dopamine agonists, apomorphine, ipecac, ipecacuanha, emetine, emetine (methylcephaeline), cephaeline, psychotrine, O-methylpsychotrine, ammonium chloride, potassium chloride, magnesium sulfate, ferrous gluconate, ferrous sulfate, aloin, algarot or antimonious oxychloride, antimony trichloride, folate, folic acid, niacin (niacin) and nicotinamide.

In some embodiments, the aversive agent in the dosage form may be zinc in the form of elemental zinc or a pharmaceutically acceptable salt of zinc, in a quantity expressed as mg of elemental zinc, of about 1 mg to about 400 mg, or about 1 mg to about 300 mg, or about 1 mg to about 200 mg, or about 1 mg to about 150 mg, or about 1 mg to about 100 mg, or about 1 mg to about 90 mg, or about 1 mg to about 80 mg, or about 1 mg to about 70 mg, or about 1 mg to about 60 mg, or about 1 mg to about 50 mg, or about 1 mg to about 45 mg, or about 1 mg to about 40 mg, or about 1 mg to about 40 mg, or about 1 mg to about 35 mg, or about 1 mg to about 30 mg, or about 1 mg to about 25 mg, or about 1 mg to about 20 mg, or about 1 mg to about 10 mg, or about 1 mg to about 5 mg, or about 5 mg to about 400 mg, or about 5 mg to about 300 mg, or about 5 mg to about 200 mg, or about 5 mg to about 150 mg, or about 5 mg to about 100 mg, or about 10 mg to about 150 mg, or about 10 mg to about 100 mg, or about 5 mg to about 80 mg, or about 5 mg to about 60 mg, or about 5 mg to about 50 mg, or about 5 mg to about 45 mg, or about 5 mg to about 40 mg, or about 5 mg to about 40 mg, or about 5 mg to about 35 mg, or about 5 mg to about 30 mg, or about 5 mg to about 25 mg, or about 5 mg to about 20 mg, or about 5 mg to about 10 mg, or about 10 mg to about 90 mg, or about 10 mg to about 80 mg, or about 10 mg to about 60 mg, or about 10 mg to about 50 mg, or about 10 mg to about 45 mg, or about 10 mg to about 40 mg, or about 10 mg to about 40 mg, or about 10 mg to about 35 mg, or about 10 mg to about 30 mg, or about 10 mg to about 25 mg, or about 10 mg to about 20 mg, or about 20 mg to about 100 mg, or about 20 mg to about 90 mg, or about 20 mg to about 80 mg, or about 20 mg to about 60 mg, or about 20 mg to about 50 mg, or about 20 mg to about 45 mg, or about 20 mg to about 40 mg, or about 20 mg to about 35 mg, or about 20 mg to about 30 mg, or about 15 mg to about 50 mg, or about 15 mg to about 40 mg, or about 15 mg to about 35 mg, or a quantity sufficient to be produce an aversive effect vasodilation when abused but not under conditions of medically appropriate use.

Various irritants can be employed including, for example and without limitation transient receptor potential vanilloid 1 (TRPV1 or VR1) agonists (including resiniferanoids, capsaicinoids, phorboid vanilloids, and terpenoid 1,4-unsaturated dialdehydes, capsaicin, capsaicin analogs and derivatives, resiniferatoxin, olvanil, piperine, zingerone, anandamide, 12- and 15-(S)-hydroperoxy-eicosatetraenoic acids, 5 and 15-(S)-hydroxyeicosatetraenoic acids, phorbol 12-phenylacetate 13-acetate 20-homovanillate, 2 phorbol 12,13-didecanoate 20-homovanillate, leukotriene B(4), tinyatoxin, heptanoylisobutylamide, N-(3-acyloxy-2-benzylpropyl)-N′-dihydroxytetrahydrobenzazepine, tetrahydroisoquinoline thiourea analogs, heptanoyl guaiacylamide, other isobutylamides or guaiacylamides, dihydrocapsaicin, homovanillyl octylester and nonanoyl vanillylamide), acids such as acids with one or more carboxyl moieties (e.g., formic acid, acetic acid, propionic acidy, butyric acid, valeric acid, caproic acid, caprillic acid, capric acid, oxalic acid, malonic acid, succicnic acid, glutaric acid, adipic acid, maleic acid, fumaric acid, and citric acid), sodium lauryl sulfate, poloxamer, sorbitan monoesters, glyceryl monooleates, niacin, mustard, allyl isothiocyaanate and p-hydroxybenzyl isothiocyanate, acetylsalicylic acid.

Various cutaneous vasodilators can be employed including, for example and without limitation, niacin acid, nicotinuric acid, beta-hydroxybutyrate and nicotinic receptor (e.g., HM74A or GPR109A) agonists.

In some embodiments, the aversive agent in the dosage form may be niacin, in a quantity of about 1 mg to about 400 mg, or about 1 mg to about 300 mg, or about 1 mg to about 200 mg, or about 1 mg to about 150 mg, or about 1 mg to about 100 mg, or about 1 mg to about 90 mg, or about 1 mg to about 80 mg, or about 1 mg to about 70 mg, or about 1 mg to about 60 mg, or about 1 mg to about 50 mg, or about 1 mg to about 45 mg, or about 1 mg to about 40 mg, or about 1 mg to about 40 mg, or about 1 mg to about 35 mg, or about 1 mg to about 30 mg, or about 1 mg to about 25 mg, or about 1 mg to about 20 mg, or about 1 mg to about 10 mg, or about 1 mg to about 5 mg, or about 5 mg to about 400 mg, or about 5 mg to about 300 mg, or about 5 mg to about 200 mg, or about 5 mg to about 150 mg, or about 5 mg to about 100 mg, or about 10 mg to about 150 mg, or about 10 mg to about 100 mg, or about 5 mg to about 80 mg, or about 5 mg to about 60 mg, or about 5 mg to about 50 mg, or about 5 mg to about 45 mg, or about 5 mg to about 40 mg, or about 5 mg to about 40 mg, or about 5 mg to about 35 mg, or about 5 mg to about 30 mg, or about 5 mg to about 25 mg, or about 5 mg to about 20 mg, or about 5 mg to about 10 mg, or about 10 mg to about 90 mg, or about 10 mg to about 80 mg, or about 10 mg to about 60 mg, or about 10 mg to about 50 mg, or about 10 mg to about 45 mg, or about 10 mg to about 40 mg, or about 10 mg to about 40 mg, or about 10 mg to about 35 mg, or about 10 mg to about 30 mg, or about 10 mg to about 25 mg, or about 10 mg to about 20 mg, or about 20 mg to about 100 mg, or about 20 mg to about 90 mg, or about 20 mg to about 80 mg, or about 20 mg to about 60 mg, or about 20 mg to about 50 mg, or about 20 mg to about 45 mg, or about 20 mg to about 40 mg, or about 20 mg to about 35 mg, or about 20 mg to about 30 mg, or about 15 mg to about 50 mg, or about 15 mg to about 40 mg, or about 15 mg to about 35 mg, or a quantity sufficient to be produce an aversive effect when abused but not under conditions of medically appropriate use.

Various tissue dyes, lakes and colorants, beverage dyes, lakes and colorants, non-tissue staining beverage dyes, lakes and colorants, fecal discolorants, urine discolorants can be employed as aversive agents including, for example and without limitation, Curcumin, Riboflavin, Tartrazine, Quinoline yellow, Sunset yellow FCF, Carmine, Carmoisine, Amaranth, Ponceau 4R, Erythrosine, Allura red AC, Patent blue V, Indigo carmine, Brilliant blue FCF, Chlorophylls, Copper complexes of chlorophylls and chlorophyllins, Green S, Caramel, Brilliant black BN, Vegetable carbon, Carotenoids, Alpha-, beta-, gamma-carotene, Capsanthin, Capsorubin, Lycopene, Beta-apo-8′ carotenal, Ethyl ester of beta-apo-8′ carotenoic acid, Xanthophylls, Lutein, Canthaxanthin, Beetroot red, Anthocyanins, Cyanidin, Delphidin, Malvidin, Pelargonidin, Peonidin, Petunidin, Calcium carbonate, Titanium dioxide, Iron oxides and hydroxides, Aluminum, Brilliant blue FCF, Indigotine, Alphazurine FG, Indanthrene blue, Fast green FCF, Alizarin cyanine green F, Quinizarine green SS, Pyranine concentrated, Orange II, Dibromofluorescein, Diiodofluorescein, Erythrosine yellowish Na, Erythrosine, Ponceau SX, Lithol rubin B, Lithol rubin B Ca, Toney red, Tetrabromofluorescein, Eosine, Tetrachlorotetrabromofluorescein, Phloxine B, Helindone pink CN, Brilliant lake red R, Acid fuchsine, Lake bordeaux B, Flaming red, Alba red, Allura red AC, Allura Red AC, Alizurol purple SS, Tartrazine, Sunset yellow, FCF, Fluorescein, Naphthol yellow S, Uranine, Quinoline yellow WS, Quinoline yellow SS, Brilliant blue FCF, Indigotine, Alphazurine FG, Alizurol purple SS, Sunset yellow FCF, Alumina, Aluminum powder, Annatto extract, Beta-carotene, Bismuth oxychloride, Bronze powder, Calcium carbonate, Canthaxanthin, Caramel, Chromium-cobalt-aluminum oxide, Chromium hydroxide green, Chromium oxide green, Cochineal extract, carmine, Copper powder, Dihydroxyacetone, Ferric ammonium citrate, Ferric ammonium ferrocyanide, Ferric ferrocyanide, Guanine, Iron oxides synthetic, Logwood extract, Mica, Potassium sodium copper chlorophyllin, Pyrogallol, Pyrophyllite, Talc, Titanium dioxide, Zinc oxide, FD&C blue #1, FD&C blue #2, D&C blue #4, D&C blue #9, FD&C green #3, D&C green #5, D&C green #6, D&C green #8, D&C orange #4, D&C orange #5, D&C orange #10, D&C orange #11, FD&C red #3, FD&C red #4, D&C red #6, D&C red #7, D&C red #17, D&C red #21, D&C red #22, D&C red #27, D&C red #28, D&C red #30, D&C red #31, D&C red #33, D&C red #34, D&C red #36, D&C red #39, FD&C red #40, FD&C red #40 lake, D&C violet #2, FD&C yellow #5, FD&C yellow #6, D&C yellow #7, Ext. D&C yellow #7, D&C yellow #8, D&C yellow #10, D&C yellow #11, FD&C lakes, D&C lakes, Ext. D&C lakes, FD&C blue #1 lake, FD&C blue #2 lake, D&C blue #4 lake, FD&C green #3 lake, D&C green #5 lake, D&C green #6 lake, D&C orange #4 lake, D&C orange #5 lake, D&C orange #10 lake, D&C orange #11 lake, FD&C red #4 lake, D&C red #6 lake, D&C red #7 lake, D&C red #17 lake, D&C red #21 lake, D&C red #22 lake, D&C red #27 lake, D&C red #28 lake, D&C red #30 lake, D&C red #31 lake, D&C red #33 lake, D&C red #34 lake, D&C red #36 lake, D&C violet #2 lake, FD&C yellow #5 lake, FD&C yellow #6 lake, D&C yellow #7 lake, Ext. D&C yellow #7 lake, D&C yellow #8 lake, D&C yellow #10 lake, Turmeric, Lactoflavin, Cochineal, carminic acid, Indigotine, Magnesium chlorophyll, Brilliant green BS, Black PN, Carbo medicinalis vegetabilis, Paprika oleoresin, Paprika oleoresin, Betanin, Beta-carotene, indigo carmine, iron oxides, sunset yellow FCF, titanium dioxide, E100, E101, E102, E104, E110, E120, E122, E123, E124, E127, E129, E131, E132, E133, E140, E141, E142, E150, E151, E153, E160, E161, E162, E163, E170, E171, E172, E173 and phenazopyridine.

As used herein, “dyes”, “lakes”, “colorants” and “discolorants” are used interchangeably and refer to one or more pharmaceutically acceptable dyes, lakes or colorants which may be: (i) tissue staining; (ii) non-tissue staining; (iii) beverage staining; (iv) urine discolorant; and/or (v) fecal discolorant.

Various laxatives can be employed as aversive agents including, for example and without limitation, Bis(p-hydroxyphenyl)pyridyl-2-methane, Bisacodyl, bisoxatin, anthraquinone, anthraquinone analogs and derivatives (e.g., buckthorn, casanthranol, cascara, hydroxyanthracene, glucofrangulin), dantron, danthron, docusate (e.g., docusate sodium, docusate calcium, docusate potassium), gastrointestinal chloride channel activators (e.g., chloride channel subtype 2 activators), lubiprostone, magenesium salts (e.g., magnesium citrate, magnesium hydroxide, magnesium oxide), mannitol, oxyphenisatine, polyethylene glycol, poly(ethylene oxide) [PEO-1500], sodium phosphate, phenolphthalein, senna, senna constituents and derivatives (e.g., sennoside A, sennoside B) and sodium picosulfate.

In some embodiments, the aversive agent in the dosage form may be a laxative in the amount of about 0.001 mg to about 300 mg, or about 0.001 mg to about 200 mg, or about 0.001 mg to about 100 mg, or about 0.001 mg to about 75 mg, or about 0.001 mg to about 50 mg, or about 0.001 mg to about 25 mg, or about 0.001 mg to about 20 mg, or about 0.001 mg to about 10 mg, or about 0.001 mg to about 5 mg, or about 0.001 mg to about 2.5 mg, or about 0.001 mg to about 1 mg, or about 1 mg to about 300 mg, or about 1 mg to about 200 mg, or about 1 mg to about 100 mg, or about 1 mg to about 75 mg, or about 1 mg to about 50 mg, or about 1 mg to about 25 mg, or about 1 mg to about 20 mg, or about 1 mg to about 10 mg, or about 1 mg to about 5 mg, or about 1 mg to about 2.5 mg.

In some embodiments, the aversive agent in the dosage form may be an opioid antagonist. Opioid antagonists are well known in the art and include naltrexone, methylnaltrexone, naloxone, nalmefene, cyclazocine, cyclorphan, oxilorphan nalorphine and levallorphan or pharmaceutically acceptable salt thereof or mixture thereof. In a preferred embodiment, said antagonist is naltrexone or naloxone. In a most preferred embodiment, said antagonist is naloxone. In some embodiments, the aversive agent in the dosage form may be an opioid antagonist in the amount of about 0.00001 mg to about 800 mg, or about 0.001 mg to about 400 mg, or about 0.01 mg to about 200 mg, or about 0.2 mg to about 100 mg, or about 0.2 mg to about 50 mg.

Aversive agents may include compounds found on the FDA EAFUS database (http://vm.cfsan.fda.gov/˜dms/eafus.html); FDA Food Additives Status List (http://www.cfsan.fda.gov/˜dms/opa-appa.html); FDAGRAS list and database; FDA Color Additive Status List (http://www.cfsan.fda.gov/˜dms/opa-appc.html); FDA Inactive Ingredients Database (http://www.accessdata.fda.gov/scripts/cder/iig/index.cfm); Rowe, Sheskey and Owen, Handbook of Pharmaceutical Excipients, APhA Publications; 5th edition (2006); Goodman & Gilman's The Pharmacological Basis of Therapeutics (Brunton, Lazo and Parker, eds, 11th ed., McGraw Hill (2005); Remington: The Science and Practice of Pharmacy, 21st ed, Lippincott Williams & Wilkins (2005); Martindale: The Complete Drug Reference, 35th Edition, Pharmaceutical Press (2007); United States Pharmacopeia-National Formulary (USP-NF), (USP 30-NF 25, 2007), the International Programme on Chemical Safety (http://www.inchem.org/) and Health Canada's List of Acceptable Non-medicinal Ingredients (http://www.hc-sc.gc.ca/dhp-mps/prodnatur/legislation/docs/nmi-imn_list1_e.html), all hereby incorporated by reference in their entirety.

It should be noted that the above mentioned aversive agents may, in some embodiments be used in the dosage form of the invention for purposes other than as aversive agents, or for both aversive and non-aversive purposes. Such non-aversive uses can include, without limitation, pharmaceutical purposes and pharmacologic purposes. For example, in some embodiments, the laxative agent may be used to counteract the constipating effects of the abusable dosage form of the invention. In some embodiments, zinc and pharmaceutically acceptable salts of zinc and niacin may be used for pharmaceutical purposes (e.g., pharmaceutical optimization, drug release and drug stability).

In one embodiment of the invention, the dosage form includes both an immediate release and extended release component.

In one embodiment of the invention, the dosage form includes a capsule within a capsule, each capsule containing a different drug or the same drug intended for treating the same or a different malady. In some preferred embodiments, the outer capsule may be an enteric coated capsule or a capsule containing an immediate release formulation to provide rapid plasma concentrations or a rapid onset of effect or a loading dose and the inner capsule contains an extended release formulation. In some preferred embodiments, up to 3 capsules within a capsule are contemplated as part of the invention. In one embodiment of the invention, the dosage form involves one or more tablets within a capsule, wherein the buprenorphine is either in the tablet and/or in one of the capsules.

In one embodiment of the invention, the formulation is ingested orally as a tablet or capsule, preferably as a capsule. In another embodiment of the invention, the formulation is administered bucally. In yet another embodiment of the invention, the formulation is administered sublingually.

“Therapeutically effective amount” or “therapeutically-effective” refers to the amount of an active agent sufficient to induce a desired biological result. That result may be alleviation of the signs, symptoms, or causes of a disease, or any other desired alteration of a biological system.

“Therapeutically effective amount of buprenorphine” refers to the amount of oral buprenorphine sufficient to prevent, to cure, or at least partially arrest a medical disorder, disease, sign or symptom for which the buprenorphine has been prescribed to a subject.

The term “effective amount” means the quantity of a compound according to the invention necessary to prevent, to cure, or at least partially arrest a medical disorder, disease, sign or symptom for which the buprenorphine has been prescribed to a subject.

The term “pharmaceutically acceptable salt” as used herein refers to a salt which is toxicologically safe for human and animal administration. Nonlimiting examples of salts include hydrochlorides, hydrobromides, hydroiodides, sulfates, bisulfates, nitrates, citrates, tartrates, bitartrates, phosphates, malates, maleates, napsylates, fumarates, succinates, acetates, terephlhalates, pamoates and pectinates.

It is contemplated that the present invention may be used alone or in combination with other drugs to provide additive, complementary, or synergistic therapeutic effects or for the treatment of entirely different medical conditions.

Other pharmaceutically active ingredients from various therapeutic classes may also be used in combination with the present invention. In some embodiment, co-administered may be used to provide additive, complementary, superadditive or synergistic therapeutic effects. In some embodiment, co-administered may be used to provide a different therapeutic effects from the present invention or to treat the side effects of the present invention. They include, but are not limited to decongestants, analgesics, analgesic adjuvants, antihistamines, expectorants, antitussives, diuretics, anti-inflammatory agents, antipyretics, antirheumatics, antioxidants, laxatives, proton pump inhibitors, motility modifying agents, vasodilators, inotropes, beta blockers, beta adrenergic agonists, drugs to treat asthma and COPD, antiinfectives, antihypertensives, antianginal agents, anticoagulants, lipid and cholesterol lowering drugs, anti-diabetic drugs, hormones, smooth muscle relaxants, skeletal muscle relaxants, bronchodilators, vitamins, trace minerals, amino acids, biological peptides, and drugs to treat disorders, diseases and maladies, and signs and symptoms thereof referred to in Harrison's Principles of Internal Medicine, 16th Edition, 2004, Kasper DL, Braunwald W, Fauci A, Hauser S, Longo D, and Jameson J L (eds)], which is hereby incorporated in its entirety by reference The drug being used in combination therapy with the present invention can be administered by any route, including parenterally, orally, topically, transdermally, sublingually, and the like.

The terms “medical condition”, “malady”, “disease”, “disorder” and “pathological states” are used interchangeably and are intended to have their broadest interpretation to refer to any physiologic, pathologic or pathophysiologic state in a human or other mammal that can be prevented, treated, managed or altered to produce a desired, usually beneficial effect.

In some embodiments, the oral buprenorphine is intended to prevent or treat pain. A co-administered drug (in the same or different dosage form, by any route of administration) may be used to provide additive, complementary, superadditive or synergistic therapeutic analgesic effects, including other NSAIDs, NO-NSAIDs, COX-2 selective inhibitors, acetaminophen, tramadol, local anesthetics, antidepressants, beta adrenergic agonists, alpha-2 agonists, selective prostanoid receptor antagonists, cannabinoid agonists, other opioid receptor agonists, NMDA receptor antagonists, gabapentin, pregabalin, gabapentinoids, neuronal nicotinic receptor agonists, calcium channel antagonists, sodium channel blockers, superoxide dismutase mimetics, p38 MAP kinase inhibitors, TRPV1 agonists, dextromethorphan, dextrorphan, ketamine, glycine receptor antagonists, antiepileptics, and any other drugs that can be shown by a person proficient in the art to prevent or treat pain.

In other embodiments, particularly preferred combinations include buprenorphine with acetaminophen.

In other embodiments, particularly preferred combinations include buprenorphine with an NSAID. Nonsteroidal anti-inflammatory drugs typically have analgesic, anti-inflammatory, and antipyretic properties. Their mode of action appears to involve inhibition of cyclooxygenases (COX-1 and COX-2), leukotriene biosynthesis, and antibradykinin activity. NSAIDs may be non-selective (inhibit COX-1 and COX-2 isozymes) or COX-2 selective (preferentially inhibit the COX-2 isozymes). Non-limiting examples of NSAIDs or COX-2 selective inhibitor include ibuprofen, tiaprofenic acid, diclofenac, piroxicam, loxoprofen, fenoprofen, indoprofen, oxaprozin, tenoxicam, lornoxicam, acetylsalicylic acid, mefenamic acid, naproxen, flurbiprofen, flubufen, ketoprofen, indoprofen, carprofen, pramoprofen, muroprofen, trioxaprofen, aminoprofen, tiaprofenic acid, fluprofen, niflumic acid, tolfenamic acid, diflunisal, etodolac, fenbufen, indomethacin, isoxicam, sudoxicam, pirprofen, sulindac, tolmetin, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflunisal, flufenisal, meloxicam and nabumetone, celecoxib, valdecoxib, etoricoxib, rofecoxib, and lumiracoxib, and as well as their pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In other embodiments, particularly preferred combinations include buprenorphine with NMDA antagonists.

The expression “N-methyl-D-aspartate receptor” shall be understood to include all of the binding site subcategories associated with the NMDA receptor, e.g., the glycine-binding site, the phenylcyclidine (PCP)-binding site, etc., as well as the NMDA channel. Thus, the invention herein contemplates the use of nontoxic substances that block an NMDA receptor binding site. NMDA receptor antagonists are substances known to those skilled in the art that block the N-methyl-D-aspartate receptor that block a major intracellular consequence of NMDA receptor activation, see U.S. Pat. Nos. 5,321,012; 5,654,281 and 5,869,498, all of which are hereby incorporated by reference in their entireties, and particularly for the purpose of describing NMDA receptor antagonists and their uses. The NMDA receptor antagonist may be a mixture. Non-limiting examples of preferred NMDA receptor antagonists include dextromethorphan ((+)-3-hydroxy-N-methylmorphinan), its metabolite dextrorphan ((+)-3-hydroxy-N-methylmorphinan), amantadine (1-amino adamantine), memantine (3,5 dimethylaminoadamantone), amitriptyline, D,L-2-amino-5-phosphono valeric acid, ketamine, methadone, racemorphan, levorphanol, and as well as their pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In other embodiments, particularly preferred combinations include buprenorphine with antiepileptics.

Non-limiting examples of anti-epileptic compounds include gabapentin, pregabalin, carbamazepine, oxcarbazepine, lamotrigine, phenyloin, fosphenyloin, valproate, valproic acid, tiagabine, topiramate, divalproex, harkoseride, and levetiracetam, in unsalified form or as pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In some preferred embodiment of the present invention, anti-epileptic compounds having pain alleviating properties include those that have the following Formula I

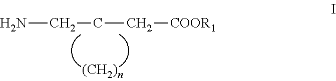

wherein R₁ is hydrogen or a lower alkyl, n is an integer of from 4 to 6; and the cyclic ring is optionally substituted, and the pharmaceutically acceptable salts thereof. The term lower alkyl includes straight or branched chain alkyl groups of up to eight carbon atoms. An especially preferred embodiment utilizes a compound of Formula I where R₁ is hydrogen and n is 5, which compound is 1-(aminomethyl)cyclohexane acetic acid, known generically as gabapentin.

Other preferred compounds of Formula I above include, but are not limited to: ethyl b 1-aminomethyl-1-cyclohexaneacetate; 1-amino methyl-1-cycloheptane-acetic acid; 1-aminomethyl-1-cyclopentane-acetic acid; methyl-1-aminomethyl-1-cyclohexane-acetate; n-butyl 1-aminomethyl-1-cyclohexaneacetate; methyl 1-aminomethyl-1-cycloheptaneacetate; n-butyl 1-aminomethyl-1-cycloheptane-acetate toluene sulfonate; 1-aminomethyl-1-cyclopentaneacetate benzene-sulfonate; and n-butyl 1-aminomethyl-1-cyclopentane-acetate.

Other preferred compounds of Formula I above, wherein the cyclic ring is substituted for example with alkyl such as methyl or ethyl, include, but are not limited to (1-aminomethyl-3-methylcyclohexyl)acetic acid, (1-aminomethyl-3-methylcyclopentyl)acetic acid, and (1-aminomethyl-3,4-dimethylcyclopentyl)acetic acid.

In another preferred embodiment of the present invention, anti-epileptic compounds having pain alleviating properties include those that are included in Formula II:

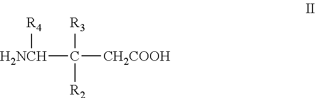

wherein R₂ is a straight or branched alkyl of from 1 to 6 carbon atoms, phenyl, or cycloalkyl having from 3 to 6 carbon atoms, R₃ is hydrogen or methyl, and R₄ is hydrogen, methyl, or carboxyl, or an individual diastereomeric or enantiomeric isomer thereof or a pharmaceutically acceptable salt thereof.

The most preferred compound of Formula II is where R₃ and R₄ are both hydrogen and R₂ is —(CH₂)ₘ-iC₄H₉ as an (R), (S) or (R,S) isomer, wherein m is 0 to 2. A more preferred embodiment of the invention utilizes 3-aminomethyl-5-methyl-hexanoic acid, and especially (S)-3-(aminomethyl)-5-methylhexanoic acid, now known generically as pregabalin. Another preferred compound is 3-(1-aminoethyl)-5-methylhexanoic acid.

In other embodiments, particularly preferred combinations include buprenorphine with antidepressants.

Antidepressants are well known in the art. Non-limiting examples of antidepressants include drugs from the following classes: tricyclic antidepressants, tetaracyclic antidepressants, SRI's, SSRI's, SNRI's and NSRI's. Non-limiting examples of specific antidepressants include amitriptyline, bupropion, citalopram, protriptyline, nortriptyline, desipramine, doxepin, imipramine, clomipramine, fluoxetine, paroxetine, sertraline, venlafaxine, duloxetine, trazodone, nefazodone, maprotiline and mirtazpine in unsalified form or as pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In other embodiments, particularly preferred combinations include buprenorphine with calcium channel blockers.

Calcium channel blockers are well or can be readily determined by established methods. Included calcium channel blockers are those disclosed or referenced in U.S. Pat. Nos. 7,064,128, 6,989,448, 6,951,862, 6,951,860, 6,949,554, 6,946,475, 6,852,742, 6,818,200, 6,617,322, 6,541,479, 6,495,715, 6,492,375, 6,469,038, 6,423,689, 6,387,897, 6,334,997, 6,310,059, 6,294,533, 6,267,945, 6,251,919, 6,251,918, 6,221,335, 6,191,151, 6,166,052, 6,124,280, 6,117,841, 6,106,856, 6,090,631, 6,011,035, 5,981,539, 5,932,573, 5,886,012, 5,767,129, 5,698,549, 5,623,051, 5,492,904, 5,484,789, 5,457,132, 5,360,809, 5,354,765, 5,350,771, 5,314,903, 5,160,734, 5,132,119, 5,070,088, 4,963,671, 4,880,824, 4,833,162, 4,808,718 and 4,791,117 and in United States patent application No. 20060084660, 20060063775, 20050227999, 20050197351, 20050191245, 20050165065, 20050159455, 20050153953, 20050014748, 20040259866, 20040253300, 20040209872, 20040204404, 20040192703, 20040147529, 20040028734, 20040006110, 20030199523, 20030060632, 20030060419, 20030045530, 20020160995, 20020115655, 20020094995, 20010029258 and 20010023249, all of which are hereby incorporated by reference in their entirety.

In other embodiments, particularly preferred combinations include buprenorphine with sodium channel modulators.

Sodium channel modulators are well or can be readily determined by established methods. Included sodium channel modulators are those disclosed or referenced in U.S. Pat. Nos. 7,078,515, 7,067,629, 7,063,953, 7,041,704, 7,022,714, 6,994,993, 6,872,741, 6,828,311, 6,770,484, 6,756,400, 6,646,012, 6,559,154, 6,479,498, 6,479,259, 6,420,354, 6,335,172, 6,221,887, 6,184,349, 6,172,085, 6,030,810, 5,776,859, and 5,437,982 and in United States patent application No. 20060223117, 20060205738, 20060183897, 20060183785, 20060142581, 20060142306, 20060063780, 20060052395, 20060052394, 20060040954, 20060020006, 20060008541, 20050260576, 20050234072, 20050233957, 20050228182, 20050228033, 20050227974, 20050227270, 20050203190, 20050186627, 20050165072, 20050130966, 20050113390, 20050113389, 20050113388, 20050112633, 20050095225, 20050090547, 20050089559, 20050080092, 20050080091, 20050059676, 20050037442, 20050020564, 20040265788, 20040248240, 20040248207, 20040229884, 20040220170, 20040213842, 20040204460, 20040204425, 20040204424, 20040198749, 20040198748, 20040198747, 20040198746, 20040198745 and 20040198744 all of which are hereby incorporated by reference in their entirety.

In other embodiments, particularly preferred combinations include buprenorphine with cannabinoid agonists.

The term “cannabinoid agonist” means a substance that binds to one or more cannabinoid receptor to exert an agonist or partial agonist effect.

A number of assays are available to determine whether a drug is a cannabinoid agonist, using in vivo and in vitro bioassay systems (Howlett et al., Mol Pharmacol, 1988; International Union of Pharmacology [IUPHAR], http://www.iuphar.org/index.html; Subcommittees on Cannabinoid Receptors The International Committee of Pharmacology Committee on Receptor Nomenclature and Classification [NC-IUPHAR], http://www.iuphar.org/nciuphar.html)

Cannabinoid agonists are known or readily determined by individuals who practice the art. Preferably, the cannabinoid agonist useful for the present invention may be selected from the group consisting of THC, THC analogs and derivatives, cannabidiol, 9-THC propyl analog, cannabidiol, cannabidiol propyl analog, cannabinol, cannabichromene, cannabichromene propyl analog, cannabigerol, cannabinoid terpenoids, cannabinoid flavonoids, endocannabinoids, anandamide and 2-arachidonoylglycerol, THC-like ABC tricyclic cannabinoid analogues, exemplified by HU210 and desacetyllevonantradol; synthetic AC bicyclic and ACD tricyclic cannabinoid analogues, exemplified by CP55940, and CP55244 and aminoalkylindole compounds, exemplified by WIN55212-2 (Little et al., Pharmacol. Biochem. Behav, 1989; Howlett et al., Neuropharmacology, 1990; Johnson et al, In: Cannabinoids as Therapeutic Agents (Mechoulam, R., ed.), CRC Press, 1986; Howlett et al., Mol Pharmacol, 1988; D'Ambra et al., J Med Chem, 1992; Pacheco et al., J Pharmacol Exp Ther, 1991; Compton et al, J Pharmacol Exp Ther, 1992; Howlett et al, Pharmacol Rev, 2002).

Preferably, the cannabinoid agonist may be selected from compounds disclosed or referenced in U.S. Pat. Nos. 7,071,213, 7,067,539, 7,057,051, 7,037,910, 6,995,187, 6,977,266, 6,949,582, 6,943,266, 6,930,122, 6,916,838, 6,914,072, 6,903,137, 6,864,291, 6,864,285, 6,790,365, 6,653,304, 6,642,258, 6,563,009, 6,525,087, 6,509,367, 6,475,478, 6,448,288, 6,403,126, 6,344,474, 6,328,992, 6,284,788, 6,113,940, 6,100,259, 5,990,170, 5,948,777, 5,939,429, 5,925,768, 5,872,148, 5,817,766, 5,747,524, 5,605,906, 5,596,106, 5,532,237, 5,440,052, 4,816,415 and in U.S. patent Application No. 20060172019, 20060160850, 20060160776, 20060155126, 20060154958, 20060154956, 20060154955, 20060128738, 20060128673, 20060122230, 20060115816, 20060100228, 20060100208, 20060094774, 20060094123, 20060089378, 20060089356, 20060084659, 20060079556, 20060052411, 20060030563, 20060009528, 20050282861, 20050267161, 20050250769, 20050239828, 20050239133, 20050228034, 20050192278, 20050187253, 20050171110, 20050165118, 20050143381, 20050137197, 20050137173, 20050096379, 20050095674, 20050080087, 20050065216, 20050065189, 20050054659, 20050026986, 20050020544, 20050014786, 20050009903, 20050009870, 20040259887, 20040248956, 20040248881 and 20040242593.

The term “cannabinoid receptor” means a molecule that causes a specific physiologic, pathophysiologic or pharmacologic effect after binding to CB₁, CB₂, non-CB₁/CB₂ cannabinoid sites, TRPV₁ receptors, as well as other G protein-coupled receptors (GPCRs) that form part of the endocannabinoid system (Wiley and Martin, Chemistry Physics of Lipids, 2002; Begg et al., Pharmacol Ther, 2005; Howlett et al., Neuropharmacol, 2004; Pertwee, AAPS Journal, 2005; International Union of Pharmacology (IUPHAR) Receptor Database; Howlett et al., Mol Pharmacol, 1988; International Union of Pharmacology [IUPHAR], http://www.iuphar.org/index.html; Subcommittees on Cannabinoid Receptors The International Committee of Pharmacology Committee on Receptor Nomenclature and Classification [NC-IUPHAR], http://www.iuphar.org/nciuphar.html).

Notwithstanding the above definitions, for the purposes of the present invention, drugs that enhance the effect of cannabinoid agonists by inhibiting their metabolism or reuptake (for example, anandamide amidase inhibitors) are also considered to be cannabinoid agonists.

In other embodiments, particularly preferred combinations include buprenorphine with other opioids.

Opioid agonists include alfentanil, allylprodine, alphaprodine, anileridine, apomorphine, apocodeine, benzylmorphine, bezitramide, buprenorphine, butorphanol, carfentanil, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, nociceptin/orphanin FQ (N/OFQ), normorphine, norpipanone, ohmefentanyl, opium, oxycodone, oxymorphone, papavereturn, pentazocine, phenadoxone, phenomorphan, phenazocine, phenoperidine, pholcodine, piminodine, piritramide, propheptazine, promedol, profadol, properidine, propiram, propoxyphene, racemorphan, remifentanil, sufentanil, tapentadol, tramadol, tilidine, methylnaltrexone, naloxone methiodide, naloxonazine, nalmexone, nalbuphine, nalorphine dinicotinate, naltrindole (NTI), naltrindole isothiocyanate, (NTII), naltriben (NTB), nor-binaltorphimine (nor-BNI), beta-funaltrexamine (b-FNA), BNTX, cyprodime, ICI-174,864, LY117413, MR2266, etorphine, DAMGO, CTOP, diprenorphine, naloxone benzoylhydrazone, bremazocine, ethylketocyclazocine, U50,488, U69, 593, spiradoline, DPDPE, [D-Ala2,Glu4] deltorphin, DSLET, Met-enkephalin, Leu-enkephalin, (3-endorphin, dynorphin A, dynorphin B, a-neoendorphin, or an opioid having the same pentacyclic nucleus as nalmefene, naltrexone, buprenorphine, levorphanol, meptazinol, pentazocine, dezocine, or their pharmaceutically acceptable salts, prodrugs, esters, analogs, derivatives, solvates, complexes, polymorphs, hydrates and metabolites, as racemates or an individual

In other embodiments, particularly preferred combinations include buprenorphine with muscle relaxants, including cyclobezaprine and drugs selected from the class of benzodiazepine agonists (e.g, diazepam, lorazepam, tamazepam, nitrazepam, flurazepam, etc).

### DRAWINGS

The included drawings are illustrative but not limiting of the methods and composition of the present invention. Other suitable modifications and adaptations of the variety of conditions and parameters normally encountered and obvious to those skilled in the art are within the spirit and scope of the invention.

FIG. 1: Time-effect curves depicting antinociceptive effect of orally administered buprenorphine in the tail flick test. The percent maximum possible effect (% MPE) is plotted versus time.

FIG. 2: Dose-response curves depicting the antinociceptive effect of buprenorphine administered orally in the tail flick test. The percent area under (analgesic effect) curve (AUC) from the time-effect curves is plotted versus log [dose].

FIG. 3: Time-effect curves depicting antinociceptive effect of orally administered buprenorphine in the hotplate test. The percent maximum possible effect (% MPE) is plotted versus time.

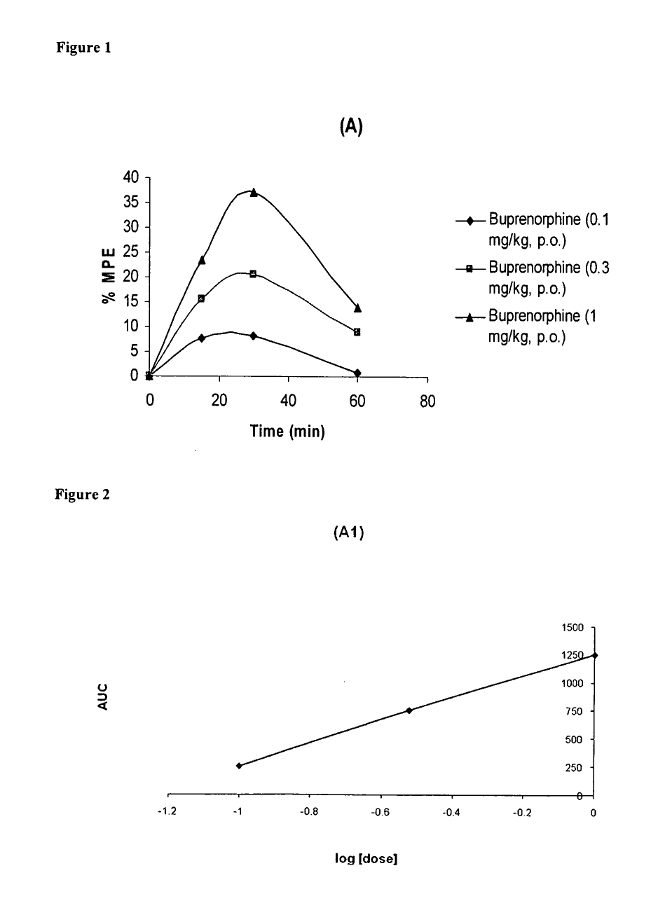

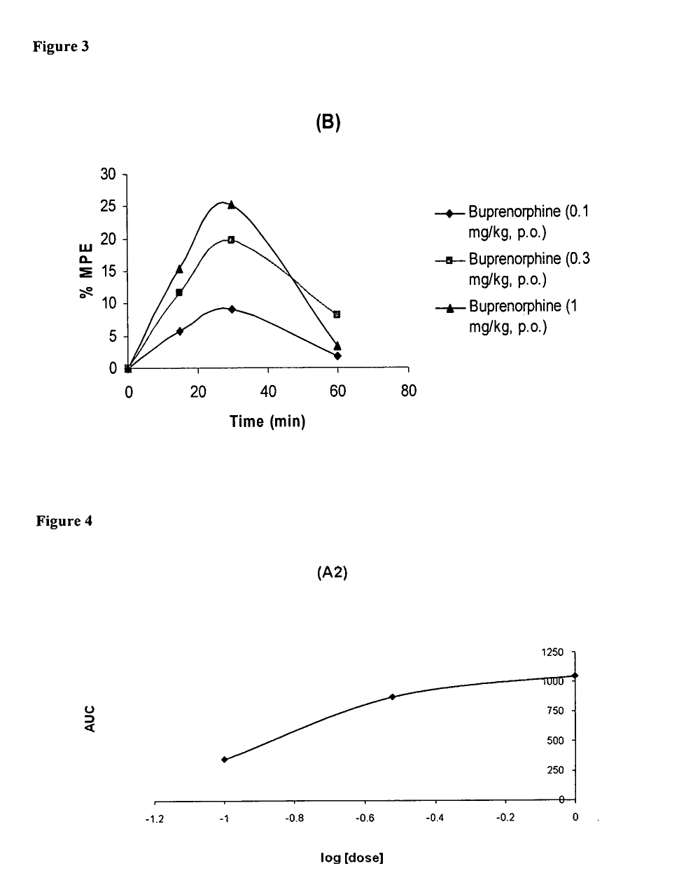

FIG. 2: Dose-response curves depicting the antinociceptive effect of buprenorphine administered orally in the hot plate test. The percent area under (analgesic effect) curve (AUC) from the time-effect curves is plotted versus log [dose].

### METHODS OF CARRYING OUT THE INVENTION

### Dosage Forms

Pharmaceutical composition and methods of the present invention contain buprenorphine base or pharmaceutically acceptable salts in racemic or enantiomeric form, or mixtures thereof intended for oral administration.

All oral pharmaceutical dosage forms of the invention are contemplated, including oral suspensions, tablets, capsules, lozenges, effervescent tablets, effervescent powders, powders, solutions, powders for reconstitution, gastroretentive tablets and capsules, orally disintegrating tablets, oral fast dissolving tablets, oral fast dispersing tablets, oral fast disintegrating dosage forms, each administered as immediate release, modified release, enteric coated, sustained release, controlled release, pulsatile release or extended release dosage form.

The formulation may optionally comprise excipients. Non-limiting examples of these auxiliary materials (or pharmaceutically acceptable excipients) are (i) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like; (ii) Disintegrants such as starches, pregelatinized corn starch, pregelatinized starch, celluloses, cross-linked carboxymethylcellulose, crospovidone, cross-linked polyvinylpyrrolidone, a calcium or a sodium alginate complex, clays, alginates, gums, or sodium starch glycolate, and any disintegration agents used in tablet preparations; (iii) Filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like; (iv) Stabilizers such as any antioxidation agents, buffers, or acids, and the like; (v) Lubricants such as magnesium stearate, calcium hydroxide, talc, colloidal silicon dioxide, sodium stearyl fumarate, hydrogenated vegetable oil, stearic acid, glyceryl behenate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like; (vi) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like; (vii) Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like; (viii) Anti-adherents or glidants such as talc, corn starch, DL-leucine, sodium lauryl sulfate, and magnesium, calcium, or sodium stearates, and the like; (ix) Pharmaceutically compatible carriers such as acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like; and (x) exipients referred to herein.

### Oral Immediate Release Dosage Forms

Pharmaceutical composition and methods of the present invention contain buprenorphine base or pharmaceutically acceptable salts in racemic or enantiomeric form, or mixtures thereof in and they are intended for oral administration.

All oral immediate release pharmaceutical dosage forms of the invention are contemplated. The preparation of oral immediate release dosage forms has been described in the art—see Remington: the science of Pharmacy Practice, 21^{st} Edition, 2006, Lippincott, Williams & Wilkins, Baltimore, Md.; Pharmaceutical Preformulation and Formulation: A Practical Guide from Candidate Drug Selection to Commercial Dosage Form. Gibson, M (ed). CRC Press, 2001; Niazi, S. Handbook of Pharmaceutical Manufacturing Formulations: Uncompressed Solid Products (Volume 2 of 6), CRC Press, 2004; Niazi, S. Handbook of Pharmaceutical Manufacturing Formulations: Compressed Solid Products (Volume 1 of 6), CRC Press, 2004; Mollet, H, Grubenmann A, Payne H. Formulation Technology: Emulsions, Suspensions, Solid Forms, Wiley-VCH, 2001; Niazi S and Niazi SK (all of which are hereby incorporated by reference). A majority of oral dosage forms commercially available world-wide are formulated as immediate release products.

### Controlled-Release Dosage Forms

All oral extended release pharmaceutical dosage forms of the invention are contemplated. The preparation of oral extended release pharmaceutical dosage forms has been described in the art—see—see for example, (i) Remington: the science of Pharmacy Practice, 21^{st} Edition, 2006, Lippincott, Williams & Wilkins, Baltimore, Md.; and (ii) Pharmaceutical Preformulation and Formulation: A Practical Guide from Candidate Drug Selection to Commercial Dosage Form. Gibson, M (ed). CRC Press, 2001; and (iii) Niazi, S. Handbook of Pharmaceutical Manufacturing Formulations: Compressed Solid Products (Volume 1 of 6), CRC Press, 2004; and (iv) Mollet, H, Grubenmann A, Payne H. Formulation Technology: Emulsions, Suspensions, Solid Forms, Wiley-VCH, 2001; (v) Donald Wise, Handbook of Pharmaceutical Controlled Release Technology, CRC; 1st edition (Aug. 15, 2000); (vi) Cherng-ju Kim, Controlled Release Dosage Form Design, Informa Healthcare (Oct. 25, 1999); (vii) Xiaoling Li, Design of Controlled Release Drug Delivery Systems, McGraw-Hill Professional; 1 edition (Nov. 3, 2005); (viii) Jean-Maurice Vergnaud, Controlled Drug Release Of Oral Dosage Forms, CRC (Jul. 31, 1993); (ix) L. T. Fan and S. K. Singh. Controlled Release: A Quantitative Treatment, Springer-Verlag (July 1989); (x) Tapash K. Ghosh and Bhaskara R. Jasti (eds). Theory and Practice of Contemporary Pharmaceutics, CRC; 2Rev Ed edition (Nov. 23, 2004); (xi) Xiaoling Li and Bhaskara R. Jasti (eds). Design of Controlled release Drug Delivery Systems; (xii) Anya M. Hillery, Andrew W. Lloyd and James Swarbrick (eds). Drug Delivery and Targeting: For Pharmacists and Pharmaceutical Scientists, CRC (Sep. 27, 2001); (xiii) Ram I. Mahato. Pharmaceutical Dosage Forms and Drug Delivery, CRC; 1 edition (Jun. 7, 2007); (xiv) Vasant V. Ranade and Mannfred A. Hollinger. Drug Delivery Systems, Second Edition, CRC; 2 edition (Aug. 26, 2003); (xv) Ashok Katdare and Mahesh Chaubal (eds). Excipient Development for Pharmaceutical, Biotechnology, and Drug Delivery Systems, Informa Healthcare; 1 edition (Jul. 28, 2006); and (xvi) Binghe Wang, Teruna J. Siahaan and Richard A. Soltero. Drug Delivery: Principles and Applications, Wiley-Interscience (Mar. 28, 2005), all of which are hereby incorporated in their entirety by reference.

The preparation of oral extended release pharmaceutical dosage forms has also been described in the art. Nonlimiting examples are provided in U.S. Pat. Nos. 7,427,414; 7,422,758; 7,413,750; 7,413,749; 7,387,793; 7,316,821; 7,229,642; 7,198,803; 7,189,414; 7,125,567; 7,074,430; 7,070,806; 7,052,706; 6,979,463; 6,936,275; 6,932,981; 6,905,709; 6,902,742; 6,793,936; 6,733,783; 6,730,325; 6,726,931; 6,716,449; 6,709,677; 6,699,508; 6,699,506; 6,692,769; 6,692,766; 6,682,759; 6,667,060; 6,645,527; 6,599,529; 6,579,536; 6,517,868; 6,440,458; 6,387,404; 6,344,215; 6,342,250; 6,326,027; 6,319,520; 6,306,438; 6,274,599; 6,254,887; 6,245,356; 6,245,351; 6,228,398; 6,221,399; 6,210,714; 6,162,463; 6,159,501; 6,156,342; 6,153,623; 6,143,353; 6,143,322; 6,132,772; 6,103,261; 6,074,674; 6,048,548; 6,039,980; 6,034,085; 6,030,642; 6,024,982; 5,952,005; 5,885,616; 5,869,100; 5,858,408; 5,795,882; 5,773,025; 5,681,585; 5,674,533; 5,656,295; 5,656,291; 5,639,476; 5,614,218; 5,591,452; 5,589,190; 5,582,837; 5,580,578; 5,549,912; 5,520,931; 5,512,293; 5,508,042; 5,500,227; 5,484,607; 5,472,712; 5,451,409; 5,399,358; 5,378,474; 5,334,392; 5,330,766; 5,314,697; 5,281,415; 5,262,164; 5,242,910; 5,229,135; 5,202,128; 5,198,220; 5,196,202; 5,186,930; 5,173,299; 5,128,144; 5,114,718; 5,084,267; 5,077,051; 5,047,248; and 5,043,165, and in U.S. Patent application No. 20090060994; 20090017127; 20090017126; 20080318910; 20080312309; 20080305160; 20080299196; 20080299189; 20080274181; 20080274177; 20080268057; 20080234352; 20080226713; 20080221174; 20080206335; 20080138411; 20080124399; 20080124398; 20080118556; 20080113025; 20080102121; 20080095853; 20080075785; 20080075781; 20080070972; 20080069888; 20080069873; 20080063711; 20080057123; 20080026052; 20080020039; 20080003281; 20070298101; 20070275065; 20070275062; 20070264325; 20070219175; 20070207214; 20070196500; 20070196396; 20070185218; 20070166375; 20070160663; 20070149590; 20070148153; 20070128276; 20070122481; 20070077297; 20070071819; 20070048377; 20070003617; 20060269603; 20060269601; 20060251721; 20060240105; 20060233880; 20060228413; 20060210631; 20060210630; 20060204578; 20060193912; 20060193911; 20060165808; 20060165807; 20060165792; 20060165791; 20060147527; 20060128806; 20060110463; 20060099262; 20060099261; 20060073204; 20060068009; 20060057203; 20060024366; 20050244498; 20050106247; 20040213844; 20040197405; 20040132826; 20040122104; 20040076665; 20040052851; 20030170304; 20030129237 and 20020054907, all of which are hereby incorporated in their entirety by reference.

The controlled-release dosage form may optionally include a controlled release material which is incorporated into a matrix along with the buprenorphine, or which is applied as a sustained release coating over a substrate comprising the drug (the term “substrate” encompassing beads, pellets, spheroids, tablets, tablet cores, etc). The controlled release material may be hydrophobic or hydrophilic as desired. The oral dosage form according to the invention may be provided as, for example, granules, spheroids, pellets or other multiparticulate formulations. An amount of the multiparticulates which is effective to provide the desired dose of buprenorphine over time may be placed in a capsule or may be incorporated in any other suitable oral solid form, e.g., compressed into a tablet. On the other hand, the oral dosage form according to the present invention may be prepared as a tablet core coated with a controlled-release coating, or as a tablet comprising a matrix of drug and controlled release material, and optionally other pharmaceutically desirable ingredients (e.g., diluents, binders, colorants, lubricants, etc.). The controlled release dosage form of the present invention may also be prepared as a bead formulation or an osmotic dosage formulation.

In certain preferred embodiments of the present invention, the controlled-release formulation is achieved via a matrix (e.g. a matrix tablet) which includes a controlled-release material as set forth below. A dosage form including a controlled-release matrix provides in-vitro dissolution rates of buprenorphine within the preferred ranges and that releases the buprenorphine in a pH-dependent or pH-independent manner. The materials suitable for inclusion in a controlled-release matrix will depend on the method used to form the matrix. The oral dosage form may contain between 1% and 99% (by weight) of at least one hydrophilic or hydrophobic controlled release material.

A non-limiting list of suitable controlled-release materials which may be included in a controlled-release matrix according to the invention include hydrophilic and/or hydrophobic materials, such as gums, cellulose ethers, acrylic resins, protein derived materials, waxes, shellac, and oils such as hydrogenated castor oil, hydrogenated vegetable oil hydrogenated Type I or Type II vegetable oils, polyoxyethylene stearates and distearates, glycerol monostearate, and non-polymeric, non-water soluble liquids carbohydrate-based substances or poorly water soluble, high melting point (mp=40 to 100° C.) waxes and mixtures thereof.

Hydrogenated vegetable oils of the present invention may include hydrogenated cottonseed oil (e.g., Akofine®; Lubritab®; Sterotex® NF), hydrogenated palm oil (Dynasan® P60; Softisan® 154), hydrogenated soybean oil (Hydrocote®; Lipovol HS-K®; Sterotex® HM) and hydrogenated palm kernel oil (e.g., Hydrokote® 112).

Polyoxyethylene stearates and distearates of the present invention include Polyoxyl 2, 4, 6, 8, 12, 20, 30, 40, 50, 100 and 150 stearates (e.g., Hodag® DGS; PEG-2 stearate; Acconon® 200-MS; Hodag® 20-S; PEG-4 stearate; Cerasynt® 616; Kessco® PEG 300 Monostearate; Acconon® 400-MS; Cerasynt® 660; Cithrol® 4MS; Hodag® 60-S; Kessco® PEG 600 Monostearate; Cerasynt® 840; Hodag 100-S; Myrj® 51; PEG-30 stearate; polyoxyethylene (30) stearate; Crodet® S40; E431; Emerest® 2672; Atlas G-2153; Crodet® S50) and polyoxyl 4, 8, 12, 32 and 150 distearates (e.g, Lipo-PEG® 100-S; Myrj® 59; Hodag® 600-S; Ritox® 59; Hodag® 22-S; PEG-4 distearate; Hodag® 42-S; Kessco® PEG 400 DS; Hodag® 62-S; Kessco® PEG 600 Distearate; Hodag® 154-S; Kessco® PEG 1540 Distearate; Lipo-PEG® 6000-DS; Protamate® 6000-DS).

In one embodiment of the present invention, the buprenorphine is combined with beeswax, hydroxypropyl methyl cellulose (e.g, HPMC K15M), silicon dioxide (alone or in combination with Al₂O₃; e.g, Aerosil®, Aerosil® 200, Aerosil® COK84).

In one embodiment of the present invention, the buprenorphine is combined with hydrogenated cottonseed oil (e.g., Sterotex® NF), hydroxypropyl methyl cellulose (e.g, HPMC K15M), coconut oil and silicon dioxide (alone or in combination with Al₂O₃; e.g, Aerosil®, Aerosil® 200, Aerosil® COK84).

In another embodiment of the present invention, the buprenorphine is combined with glycerol monostearate (e.g., Cithrol® GMS), hydroxypropyl methyl cellulose (e.g, HPMC K100M) and silicon dioxide (alone or in combination with Al₂O₃; e.g, Aerosil®, Aerosil® 200, Aerosil® COK84).

In yet another embodiment of the present invention, the buprenorphine is combined with hydrogenated palm kernel oil (e.g., Hydrokote® 112), hydroxypropyl methyl cellulose (e.g, HPMC K15M) and silicon dioxide (alone or in combination with Al₂O₃; e.g, Aerosil®, Aerosil® 200, Aerosil® COK84).

In one embodiment of the present invention, release rate modifiers, including hydroxypropyl methyl cellulose (e.g, HPMC K15M) may incorporated. Release rate modifiers can also have additional useful properties that optimize the formulation.

A variety of agents may be incorporated into the invention as thixotropes (e.g., fumed silicon dioxides, Aerosil®, Aerosil® COK84, Aerosil® 200, etc.). Thixotropes enhance the pharmaceutical formulations of the invention by increasing the viscosity of solutions complementing the action of HPMCs.

Any pharmaceutically acceptable hydrophobic or hydrophilic controlled-release material which is capable of imparting controlled-release of the buprenorphine may be used in accordance with the present invention. Preferred controlled-release polymers include alkylcelluloses such as ethylcellulose, acrylic and methacrylic acid polymers and copolymers, and cellulose ethers, especially hydroxyalkylcelluloses (e.g., hydroxypropylmethylcellulose) and carboxyalkylcelluloses. Preferred acrylic and methacrylic acid polymers and copolymers include methyl methacrylate, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polymethacrylate, polyacrylamide, poly(methacrylic acid anhydride), and glycidyl methacrylate copolymers. Certain preferred embodiments utilize mixtures of any of the foregoing controlled-release materials in the matrices of the invention.

The matrix also may include a binder. In such embodiments, the binder preferably contributes to the controlled-release of the buprenorphine from the controlled-release matrix.

Preferred hydrophobic binder materials are water-insoluble with more or less pronounced hydrophilic and/or hydrophobic trends. Preferred hydrophobic binder materials which may be used in accordance with the present invention include digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, natural and synthetic waxes and polyalkylene glycols. Preferably, the hydrophobic binder materials useful in the invention have a melting point from about 30 to about 200° C., preferably from about 45 to about 90° C. When the hydrophobic material is a hydrocarbon, the hydrocarbon preferably has a melting point of between 25 and 90° C. Of the long chain (C₈-C₅₀) hydrocarbon materials, fatty (aliphatic) alcohols are preferred. The oral dosage form may contain up to 98% (by weight) of at least one digestible, long chain hydrocarbon.

The oral dosage form contains up to 98% (by weight) of at least one polyalkylene glycol. The hydrophobic binder material may comprise natural or synthetic waxes, fatty alcohols (such as lauryl, myristyl, stearyl, cetyl or preferably cetostearyl alcohol), fatty acids, including but not limited to fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material which is normally solid at room temperature and has a melting point of from about 30 to about 100° C.

In certain preferred embodiments, a combination of two or more hydrophobic binder materials are included in the matrix formulations. If an additional hydrophobic binder material is included, it is preferably selected from natural and synthetic waxes, fatty acids, fatty alcohols, and mixtures of the same. Examples include beeswax, carnauba wax, stearic acid and stearyl alcohol. This list is not meant to be exclusive.

One particular suitable controlled-release matrix comprises at least one water soluble hydroxyalkyl cellulose, at least one C₁₂-C₃₆, preferably C₁₄-C₂₂, aliphatic alcohol and, optionally, at least one polyalkylene glycol. The hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the present oral dosage form will be determined, inter alia, by the precise rate of the buprenorphine release required. The aliphatic alcohol may be, for example, lauryl alcohol, myristyl alcohol or stearyl alcohol. In particularly preferred embodiments of the present oral dosage form, however, the at least one aliphatic alcohol is cetyl alcohol or cetostearyl alcohol. The amount of aliphatic alcohol in the present oral dosage form will be determined, as above, by the precise rate of the buprenorphine release required. It will also depend on whether at least one polyalkylene glycol is present in or absent from the oral dosage form. In the absence of at least one polyalkylene glycol, the oral dosage form preferably contains between 20% and 50% (by wt) of the aliphatic alcohol. When a polyalkylene glycol is present in the oral dosage form, then the combined weight of the aliphatic alcohol and the polyalkylene glycol preferably constitutes between 20% and 50% (by wt) of the total dosage.

In one preferred embodiment, the ratio of, e.g., the at least one hydroxyalkyl cellulose or acrylic resin to the at least one aliphatic alcohol/polyalkylene glycol determines, to a considerable extent, the release rate of the buprenorphine from the formulation. A ratio of the hydroxyalkyl cellulose to the aliphatic alcohol/polyalkylene glycol of between 1:2 and 1:4 is preferred, with a ratio of between 1:3 and 1:4 being particularly preferred.

The polyalkylene glycol maybe, for example, polypropylene glycol or, which is preferred, polyethylene glycol. The number average molecular weight of the at least one polyalkylene glycol is preferred between 1,000 and 15,000 especially between 1,500 and 12,000.

Another suitable controlled-release matrix comprises an alkylcellulose (especially ethylcellulose), a C₁₂ to C₃₆ aliphatic alcohol and, optionally, a polyalkylene glycol.

Another method of producing the dosage form of the invention involves liquid fill compositions, including hydrogenated Type I or Type II vegetable oils (e.g., Hydrokote™ 112), polyoxyethylene stearates and distearates, glycerol monostearate (e.g., Cithrol™ GMS), non-polymeric, non-water soluble liquids carbohydrate-based substances, poorly water soluble, high melting point (mp=40 to 100° C.) waxes.

Hydrogenated vegetable oils may include hydrogenated cottonseed oil (e.g., Akofine™; Lubritab™; Sterotex™ NF), hydrogenated palm oil (Dynasan™ P60; Softisan™ 154), hydrogenated soybean oil (Hydrocote™; Lipovol HS-K™; Sterotex™ HM) and hydrogenated palm kernel oil (e.g., Hydrokote™ 112).

Polyoxyethylene stearates and distearates may include Polyoxyl 2, 4, 6, 8, 12, 20, 30, 40, 50, 100 and 150 stearates (e.g., Hodag™ DGS; PEG-2 stearate; Acconon™ 200-MS; Hodag™ 20-S; PEG-4 stearate; Cerasynt™ 616; Kessco™ PEG 300 Monostearate; Acconon™ 400-MS; Cerasynt™ 660; Cithrol™ 4MS; Hodag™ 60-S; Kessco™ PEG 600 Monostearate; Cerasynt™ 840; Hodag 100-S; Myrj™ 51; PEG-30 stearate; polyoxyethylene (30) stearate; Crodet™ S40; E431; Emerest™ 2672; Atlas G-2153; Crodet™ S50) and polyoxyl 4, 8, 12, 32 and 150 distearates (e.g, Lipo-PEG™ 100-S; Myrj™ 59; Hodag™ 600-S; Ritox™ 59; Hodag™ 22-S; PEG-4 distearate; Hodag™ 42-S; Kessco™ PEG 400 DS; Hodag™ 62-S; Kessco™ PEG 600 Distearate; Hodag™ 154-S; Kessco™ PEG 1540 Distearate; Lipo-PEG™ 6000-DS; Protamate™ 6000-DS).

In one embodiment of the present invention, release rate modifiers, including hydroxypropyl methyl cellulose (e.g, HPMC K15M) may be incorporated. Release rate modifiers can also have additional useful properties that optimize the formulation.

A variety of agents may be incorporated into the invention as thixotropes (e.g., fumed silicon dioxides, Aerosil™, Aerosil™ COK84, Aerosil™ 200, etc.). Thixotropes enhance the pharmaceutical formulations of the invention by increasing the viscosity of solutions during attempted extraction, complementing the action of HPMCs. They may also provide a tamper resistance by helping to retain the structure of dosage units that have been heated to temperatures greater than the melting point of the base excipient (Aerosils are unaffected by heat).

In addition to the above ingredients, a controlled-release matrix may also contain suitable quantities of other materials, e.g., diluents, lubricants, binders, granulating aids, colorants, flavorants and glidants that are conventional in the pharmaceutical art.

In order to facilitate the preparation of a solid, controlled-release oral dosage form according to the invention there is provided, in a further aspect of the present invention, a process for the preparation of a solid, controlled-release oral dosage form according to the present invention comprising incorporating the buprenorphine or a salt thereof in a controlled-release matrix. Incorporation in the matrix may be effected, for example, by (a) forming granules comprising at least one hydrophobic and/or hydrophilic material as set forth above (e.g., a water soluble hydroxyalkyl cellulose) together with the buprenorphine; (b) mixing the at least one hydrophobic and/or hydrophilic material-containing granules with at least one C₁₂-C₃₆ aliphatic alcohol, and (c) optionally, compressing and shaping the granules.

The granules may be formed by any of the procedures well-known to those skilled in the art of pharmaceutical formulation. For example, in one preferred method, the granules may be formed by wet granulating hydroxyalkyl cellulose/buprenorphine with water. In a particular preferred embodiment of this process, the amount of water added during the wet granulation step is preferably between 1.5 and 5 times, especially between 1.75 and 3.5 times, the dry weight of the buprenorphine.

In certain embodiments, the dosage form comprises a plurality of matrices described above.

The matrices of the present invention may also be prepared via a melt pellitization technique. In such circumstance, the buprenorphine in finely divided form is combined with a binder (also in particulate form) and other optional inert ingredients, and thereafter the mixture is pelletized, e.g., by mechanically working the mixture in a high shear mixer to form the pellets (granules, spheres). Thereafter, the pellets (granules, spheres) may be sieved in order to obtain pellets of the requisite size. The binder material is preferably in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like.

Controlled-release matrices can also be prepared by, e.g., melt-granulation or melt-extrusion techniques. Generally, melt-granulation techniques involve melting a normally solid hydrophobic binder material, e.g. a wax, and incorporating a powdered drug therein. To obtain a controlled release dosage form, it may be necessary to incorporate a hydrophobic controlled release material, e.g. ethylcellulose or a water-insoluble acrylic polymer, into the molten wax hydrophobic binder material.

The hydrophobic binder material may comprise one or more water-insoluble wax-like thermoplastic substances possibly mixed with one or more wax-like thermoplastic substances being less hydrophobic than said one or more water-insoluble wax-like substances. In order to achieve controlled release, the individual wax-like substances in the formulation should be substantially non-degradable and insoluble in gastrointestinal fluids during the initial release phases. Useful water-insoluble wax-like binder substances may be those with a water-solubility that is lower than about 1:5,000 (w/w).

In addition to the above ingredients, a controlled release matrix may also contain suitable quantities of other materials, e.g., diluents, lubricants, binders, granulating aids, colorants, flavorants and glidants that are conventional in the pharmaceutical art in amounts up to about 50% by weight of the particulate if desired. The quantities of these additional materials will be sufficient to provide the desired effect to the desired formulation.

The preparation of a suitable melt-extruded matrix according to the present invention may, for example, include the steps of blending the buprenorphine, together with a controlled release material and preferably a binder material to obtain a homogeneous mixture. The homogeneous mixture is then heated to a temperature sufficient to at least soften the mixture sufficiently to extrude the same. The resulting homogeneous mixture is then extruded, e.g., using a twin-screw extruder, to form strands. The extrudate is preferably cooled and cut into multiparticulates by any means known in the art. The strands are cooled and cut into multiparticulates. The multiparticulates are then divided into unit doses. The extrudate preferably has a diameter of from about 0.1 to about 5 mm and provides controlled release of the therapeutically active agent for a time period of from about 6 to at least about 24 hours.

An optional process for preparing the melt extrusioned formulations of the present invention includes directly metering into an extruder a hydrophobic controlled release material, a therapeutically active agent, and an optional binder material; heating the homogenous mixture; extruding the homogenous mixture to thereby form strands; cooling the strands containing the homogeneous mixture; cutting the strands into particles having a size from about 0.1 mm to about 12 mm; and dividing said particles into unit doses. In this aspect of the invention, a relatively continuous manufacturing procedure is realized.

Plasticizers, such as those described herein, may be included in melt-extruded matrices. The plasticizer is preferably included as from about 0.1 to about 30% by weight of the matrix. Other pharmaceutical excipients, e.g., talc, mono or poly saccharides, colorants, flavorants, lubricants and the like may be included in the controlled release matrices of the present invention as desired. The amounts included will depend upon the desired characteristic to be achieved.

The diameter of the extruder aperture or exit port can be adjusted to vary the thickness of the extruded strands. Furthermore, the exit part of the extruder need not be round; it can be oblong, rectangular, etc. The exiting strands can be reduced to particles using a hot wire cutter, guillotine, etc.

A melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, preferably within a range of similar size and/or shape and containing one or more active agents and one or more excipients, preferably including a hydrophobic controlled release material as described herein. Preferably the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agent without the need of a spheronization step.

In one preferred embodiment, oral dosage forms are prepared that include an effective amount of melt-extruded multiparticulates within a capsule. For example, a plurality of the melt-extruded multiparticulates may be placed in a gelatin capsule in an amount sufficient to provide an effective controlled release dose when ingested and contacted by gastric fluid.

In another preferred embodiment, a suitable amount of the multiparticulate extrudate is compressed into an oral tablet using conventional tableting equipment using standard techniques. Techniques and compositions for making tablets (compressed and molded), capsules (hard and soft gelatin) and pills are also described in Remington's Pharmaceutical Sciences, 21^{st} ed., 2005 incorporated by reference herein.

In yet another preferred embodiment, the extrudate can be shaped into tablets as set forth in U.S. Pat. No. 4,957,681, hereby incorporated by reference.

Optionally, the controlled-release matrix multiparticulate systems or tablets can be coated, or the gelatin capsule can be further coated, with a controlled release coating such as the controlled release coatings described above. Such coatings preferably include a sufficient amount of hydrophobic and/or hydrophilic controlled-release material to obtain a weight gain level from about 2 to about 25 percent, although the overcoat may be greater depending upon, e.g., the physical properties of the drug and the desired release rate, among other things.

The dosage forms of the present invention may further include combinations of melt-extruded multiparticulates containing one or more drugs. Furthermore, the dosage forms can also include an amount of an immediate release therapeutically active agent for prompt therapeutic effect. The immediate release therapeutically active agent may be incorporated, e.g., as separate pellets within a gelatin capsule, or may be coated on the surface of, e.g., melt extruded multiparticulates. The unit dosage forms of the present invention may also contain a combination of, e.g., controlled release beads and matrix multiparticulates to achieve a desired effect.

The controlled-release formulations of the present invention preferably slowly release the therapeutically active agent, e.g., when ingested and exposed to gastric fluids, and then to intestinal fluids. The controlled-release profile of the melt-extruded formulations of the invention can be altered, for example, by varying the amount of controlled-release material, by varying the amount of plasticizer relative to other matrix constituents, hydrophobic material, by the inclusion of additional ingredients or excipients, by altering the method of manufacture, etc.

In other embodiments of the invention, melt-extruded formulations are prepared without the inclusion of the therapeutically active agent, which is added thereafter to the extrudate. Such formulations typically will have the therapeutically active agent blended together with the extruded matrix material, and then the mixture would be tableted in order to provide a slow release formulation. Such formulations may be advantageous, for example, when the therapeutically active agent included in the formulation is sensitive to temperatures needed for softening the hydrophobic material and/or the retardant material.

Typical melt-extrusion production systems suitable for use in accordance with the present invention include a suitable extruder drive motor having variable speed and constant torque control, start-stop controls, and ammeter. In addition, the production system will include a temperature control console which includes temperature sensors, cooling means and temperature indicators throughout the length of the extruder. In addition, the production system will include an extruder such as twin-screw extruder which consists of two counter-rotating intermeshing screws enclosed within a cylinder or barrel having an aperture or die at the exit thereof. The feed materials enter through a feed hopper and are moved through the barrel by the screws and are forced through the die into strands which are thereafter conveyed such as by a continuous movable belt to allow for cooling and being directed to a pelletizer or other suitable device to render the extruded ropes into the multiparticulate system. The pelletizer can consist of rollers, fixed knife, rotating cutter and the like. Suitable instruments and systems will be apparent to those of ordinary skill in the art.

A further aspect of the invention is related to the preparation of melt-extruded multiparticulates as set forth above in a manner which controls the amount of air included in the extruded product. By controlling the amount of air included in the extrudate, the release rate of the therapeutically active agent from the, e.g., multiparticulate extrudate, can be altered significantly. In certain embodiments, the pH dependency of the extruded product can be altered as well.

Thus, in a further aspect of the invention, the melt-extruded product is prepared in a manner which substantially excludes air during the extrusion phase of the process. This may be accomplished, for example, by using a Leistritz extruder having a vacuum attachment. In certain preferred embodiments the extruded multiparticulates prepared according to the invention using the Leistritz extruder under vacuum provides a melt-extruded product having different physical characteristics. In particular, the extrudate is substantially non-porous when magnified, e.g., using a scanning electron microscope which provides an SEM (scanning electron micrograph). Such substantially non-porous formulations provide a faster release of the therapeutically active agent, relative to the same formulation prepared without vacuum. SEMs of the multiparticulates prepared using an extruder under vacuum appear very smooth, and the multiparticulates tend to be more robust than those multiparticulates prepared without vacuum. In certain formulations, the use of extrusion under vacuum provides an extruded multiparticulate product which is more pH-dependent than its counterpart formulation prepared without vacuum. Alternatively, the melt-extruded product is prepared using a Werner-Pfleiderer twin screw extruder.

In certain embodiments, a spheronizing agent is added to a granulate or multiparticulates of the present invention and then spheronized to produce controlled release spheroids. The spheroids are then optionally overcoated with a controlled release coating by methods such as those described herein.

Spheronizing agents which may be used to prepare the multiparticulate formulations of the present invention include any art-known spheronizing agent. Cellulose derivatives are preferred, and microcrystalline cellulose is especially preferred. A suitable microcrystalline cellulose is, for example, the material sold as Avicel™ PH 101. The spheronizing agent is preferably included as about 1 to about 99% of the multiparticulate by weight.

In addition to the active ingredient and spheronizing agent, the spheroids may also contain a binder. Suitable binders, such as low viscosity, water soluble polymers, will be well known to those skilled in the pharmaceutical art. However, water soluble hydroxy lower alkylcellulose, such as hydroxypropylcellulose, are preferred.

In addition to the buprenorphine and spheronizing agent, the multiparticulate formulations of the present invention may include a controlled release material such as those described hereinabove. Preferred controlled-release materials for inclusion in the multiparticulate formulations include acrylic and methacrylic acid polymers or copolymers, and ethylcellulose. When present in the formulation, the controlled-release material will be included in amounts of from about 1 to about 80% of the multiparticulate, by weight. The controlled-release material is preferably included in the multiparticulate formulation in an amount effective to provide controlled release of the buprenorphine from the multiparticulate.

Pharmaceutical processing aids such as binders, diluents, and the like may be included in the multiparticulate formulations. Amounts of these agents included in the formulations will vary with the desired effect to be exhibited by the formulation.

Specific examples of pharmaceutically acceptable carriers and excipients that may be used to formulate oral dosage forms of the present invention are described in the Handbook of Pharmaceutical Excipients, APhA Publications; 5 edition (Jan. 5, 2006) incorporated by reference herein.

The multiparticulates may be overcoated with a controlled-release coating including a controlled-release material such as those described hereinabove. The controlled-release coating is applied to a weight gain of from about 5 to about 30%. The amount of the controlled-release coating to be applied will vary according to a variety of factors, e.g., the composition of the multiparticulate and the chemical and/or physical properties of the drug.

Matrix multiparticulates may also be prepared by granulating the spheronizing agent together with the buprenorphine, e.g. by wet granulation. The granulate is then spheronized to produce the matrix multiparticulates. The matrix multiparticulates are then optionally overcoated with the controlled release coating by methods such as those described hereinabove.

Another method for preparing matrix multiparticulates, for example, by (a) forming granules comprising at least one water soluble hydroxyalkyl cellulose and the buprenorphine or the buprenorphine salt; (b) mixing the hydroxyalkyl cellulose containing granules with at least one C₁₂-C₃₆ aliphatic alcohol; and (c) optionally, compressing and shaping the granules. Preferably, the granules are formed by wet granulating the hydroxyalkyl cellulose/buprenorphine with water. In a particularly preferred embodiment of this process, the amount of water added during the wet granulation step is preferably between 1.5 and 5 times, especially between 1.75 and 3.5 times, the dry weight of the buprenorphine.

In yet other alternative embodiments, a spheronizing agent, together with the active ingredient can be spheronized to form spheroids. Microcrystalline cellulose is preferred. A suitable microcrystalline cellulose is, for example, the material sold as Avicel™ PH 101. In such embodiments, in addition to the active ingredient and spheronizing agent, the spheroids may also contain a binder. Suitable binders, such as low viscosity, water soluble polymers, will be well known to those skilled in the pharmaceutical art. However, water soluble hydroxy lower alkyl cellulose, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate co-polymer, or ethyl cellulose. In such embodiments, the sustained-release coating will generally include a water insoluble material such as (a) a wax, either alone or in admixture with a fatty alcohol; or (b) shellac or zein.

Spheroids of the present invention comprise a matrix formulation as described above or bead formulation as described hereinafter having a diameter of between 0.1 mm and 2.5 mm, especially between 0.5 mm and 2 mm.

The spheroids are preferably film coated with a controlled release material that permits release of the buprenorphine (or salt) at a controlled rate in an aqueous medium. The film coat is chosen so as to achieve, in combination with the other stated properties, the in-vitro release rate outlined above (e.g., at least about 12.5% released after 1 hour). The controlled-release coating formulations of the present invention preferably produce a strong, continuous film that is smooth and elegant, capable of supporting pigments and other coating additives, non-toxic, inert, and tack-free.

### Preparation of Coated Bead Formulations

In certain preferred embodiments of the present invention the oral solid controlled release dosage form of the present invention comprises a plurality of coated substrates, e.g., inert pharmaceutical beads such as nu pariel 18/20 beads. An aqueous dispersion of hydrophobic material is used to coat the beads to provide for the controlled release of the buprenorphine. In certain preferred embodiments a plurality of the resultant stabilized solid controlled-release beads may be placed in a gelatin capsule in an amount sufficient to provide an effective controlled-release dose when ingested and contacted by an environmental fluid, e.g., gastric fluid or dissolution media.

The stabilized controlled-release bead formulations of the present invention slowly release the buprenorphine, e.g., when ingested and exposed to gastric fluids, and then to intestinal fluids. The controlled-release profile of the formulations of the invention can be altered, for example, by varying the amount of overcoating with the aqueous dispersion of hydrophobic controlled release material, altering the manner in which the plasticizer is added to the aqueous dispersion of hydrophobic controlled release material, by varying the amount of plasticizer relative to hydrophobic controlled release material, by the inclusion of additional ingredients or excipients, by altering the method of manufacture, etc. The dissolution profile of the ultimate product may also be modified, for example, by increasing or decreasing the thickness of the controlled release coating.

Substrates coated with a therapeutically active agent are prepared, e.g. by dissolving the therapeutically active agent in water and then spraying the solution onto a substrate, for example, nu pariel 18/20 beads, using a Wuster insert. Optionally, additional ingredients are also added prior to coating the beads in order to assist the binding of the buprenorphine to the beads, and/or to color the solution, etc. For example, a product which includes hydroxypropyl methylcellulose, etc. with or without colorant (e.g., Opadry™) may be added to the solution and the solution mixed (e.g., for about 1 hour) prior to application of the same onto the substrate. The resultant coated substrate may then be optionally overcoated with a barrier agent, to separate the therapeutically active agent from the hydrophobic controlled-release coating.

An example of a suitable barrier agent is one which comprises hydroxypropyl methylcellulose. However, any film-former known in the art may be used. It is preferred that the barrier agent does not affect the dissolution rate of the final product.

The substrates may then be overcoated with an aqueous dispersion of the hydrophobic controlled release material as described herein. The aqueous dispersion of hydrophobic controlled release material preferably further includes an effective amount of plasticizer, e.g. tri-ethyl citrate. Pre-formulated aqueous dispersions of ethylcellulose, such as Aquacoat™ or Surelease™, may be used. If Surelease™ is used, it is not necessary to separately add a plasticizer. Alternatively, pre-formulated aqueous dispersions of acrylic polymers such as Eudragit™ can be used.

The coating solutions of the present invention preferably contain, in addition to the film-former, plasticizer, and solvent system (i.e., water), a colorant to provide elegance and product distinction. Color may be added to the solution of the therapeutically active agent instead, or in addition to the aqueous dispersion of hydrophobic material. For example, color can be added to Aquacoat™ via the use of alcohol or propylene glycol based color dispersions, milled aluminum lakes and opacifiers such as titanium dioxide by adding color with shear to water soluble polymer solution and then using low shear to the plasticized Aquacoat™. Alternatively, any suitable method of providing color to dioxide and color pigments, such as iron oxide pigments. The incorporation of pigments, may, however, increase the retard effect of the coating.

The plasticized aqueous dispersion of hydrophobic controlled release material may be applied onto the substrate comprising the therapeutically active agent by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic material to obtain a predetermined controlled-release of said therapeutically active agent when said coated substrate is exposed to aqueous solutions, e.g. gastric fluid, is preferably applied, taking into account the physical characteristics of the therapeutically active agent, the manner of incorporation of the plasticizer, etc. After coating with the hydrophobic controlled release material, a further overcoat of a film-former, such as Opadry™, is optionally applied to the beads. This overcoat is provided, if at all, in order to substantially reduce agglomeration of the beads.

Another method of producing controlled release bead formulations suitable for about 24-hour administration is via powder layering. The powder-layered beads are prepared by spraying an aqueous binder solution onto inert beads to provide a tacky surface, and subsequently spraying a powder that is a homogenous mixture of the buprenorphine and hydrous lactose impalpable onto the tacky beads. The beads are then dried and coated with a hydrophobic material such as those described hereinabove to obtain the desired release of drug when the final formulation is exposed to environmental fluids. An appropriate amount of the controlled release beads are then, e.g. encapsulated to provide a final dosage form which provides effective plasma concentrations for the intended duration of effect or dosing frequency.

### Controlled Release Osmotic Dosage

Controlled release dosage forms according to the present invention may also be prepared as osmotic dosage formulations. The osmotic dosage forms preferably include a bilayer core comprising a drug layer and a delivery or push layer, wherein the bilayer core is surrounded by a semipermeable wall and optionally having at least one passageway disposed therein. In certain embodiments, the bilayer core comprises a drug layer with the buprenorphine or a salt thereof and a displacement or push layer. In certain preferred embodiments the drug layer may also comprise at least one polymer hydrogel. The polymer hydrogel may have an average molecular weight of between about 500 and about 6,000,000. Examples of polymer hydrogels include but are not limited to a maltodextrin polymer comprising the formula (C₆H₁₂O₅)ₙ. H2O, wherein n is 3 to 7,500, and the maltodextrin polymer comprises a 500 to 1,250,000 number-average molecular weight; a poly(alkylene oxide) represented by, e.g., a poly(ethylene oxide) and a poly(propylene oxide) having a 50,000 to 750,000 weight-average molecular weight, and more specifically represented by a poly(ethylene oxide) of at least one of 100,000, 200,000, 300,000 or 400,000 weight-average molecular weights; an alkali carboxyalkylcellulose, wherein the alkali is sodium or potassium, the alkyl is methyl, ethyl, propyl, or butyl of 10,000 to 175,000 weight-average molecular weight; and a copolymer of ethylene-acrylic acid, including methacrylic and ethacrylic acid of 10,000 to 500,000 number-average molecular weight.

In certain preferred embodiments of the present invention, the delivery or push layer comprises an osmopolymer. Examples of an osmopolymer include but are not limited to a member selected from the group consisting of a polyalkylene oxide and a carboxyalkylcellulose. The polyalkylene oxide possesses a 1,000,000 to 10,000,000 weight-average molecular weight. The polyalkylene oxide may be a member selected from the group consisting of polymethylene oxide, polyethylene oxide, polypropylene oxide, polyethylene oxide having a 1,000,000 average molecular weight, polyethylene oxide comprising a 5,000,000 average molecular weight, polyethylene oxide comprising a 7,000,000 average molecular weight, cross-linked polymethylene oxide possessing a 1,000,000 average molecular weight, and polypropylene oxide of 1,200,000 average molecular weight. Typical osmopolymer carboxyalkylcellulose comprises a member selected from the group consisting of alkali carboxyalkylcellulose, sodium carboxymethylcellulose, potassium carboxymethylcellulose, sodium carboxyethylcellulose, lithium carboxymethylcellulose, sodium carboxyethylcellulose, carboxyalkylhydroxyalkylcellulose, carboxymethylhydroxyethyl cellulose, carboxyethylhydroxyethylcellulose and carboxymethylhydroxypropylcellulose. The osmopolymers used for the displacement layer exhibit an osmotic pressure gradient across the semipermeable wall. The osmopolymers imbibe fluid into dosage form, thereby swelling and expanding as an osmotic hydrogel (also known as osmogel), whereby they push the buprenorphine or pharmaceutically acceptable salt thereof from the osmotic dosage form.

The push layer may also include one or more osmotically effective compounds also known as osmagents and as osmotically effective solutes. They imbibe an environmental fluid, for example, from the gastrointestinal tract, into dosage form and contribute to the delivery kinetics of the displacement layer. Examples of osmotically active compounds comprise a member selected from the group consisting of osmotic salts and osmotic carbohydrates. Examples of specific osmagents include but are not limited to sodium chloride, potassium chloride, magnesium sulfate, lithium phosphate, lithium chloride, sodium phosphate, potassium sulfate, sodium sulfate, potassium phosphate, glucose, fructose and maltose.

The push layer may optionally include a hydroxypropylalkylcellulose represented by a member selected from the group consisting of hydroxypropylmethylcellulose, hydroxypropylethylcellulose, hydroxypropyl isopropylcellulose, hydroxypropylbutylcellulose, and hydroxypropyl pentylcellulose.

The push layer optionally may comprise a nontoxic colorant or dye. Examples of colorants or dyes include but are not limited to Food and Drug Administration Colorant (FD&C), such as FD&C No. 1 blue dye, FD&C No. 4 red dye, red ferric oxide, yellow ferric oxide, titanium dioxide, carbon black, and indigo.

The push layer may also optionally comprise an antioxidant to inhibit the oxidation of ingredients. Some examples of antioxidants include but are not limited to a member selected from the group consisting of ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, and propylgallate.

In certain alternative embodiments, the dosage form comprises an homogenous core comprising the buprenorphine or a pharmaceutically acceptable salt thereof, a pharmaceutically acceptable polymer (e.g., polyethylene oxide), optionally a disintegrant (e.g., polyvinylpyrrolidone), optionally an absorption enhancer (e.g., a fatty acid, a surfactant, a chelating agent, a bile salt, etc.). The homogenous core is surrounded by a semipermeable wall having a passageway (as defined above) for the release of the buprenorphine or pharmaceutically acceptable salt thereof.

In certain embodiments, the semipermeable wall comprises a member selected from the group consisting of a cellulose ester polymer, a cellulose ether polymer and a cellulose ester-ether polymer. Representative wall polymers comprise a member selected from the group consisting of cellulose acylate, cellulose diacylate, cellulose triacylate, cellulose acetate, cellulose diacetate, cellulose triacetate, mono-, di- and tricellulose alkenylates, and mono-, di- and tricellulose alkinylates. The poly(cellulose) used for the present invention comprises a number-average molecular weight of 20,000 to 7,500,000.

Additional semipermeable polymers for the purpose of this invention comprise acetaldehyde dimethycellulose acetate, cellulose acetate ethylcarbamate, cellulose acetate methylcarbamate, cellulose diacetate, propylcarbamate, cellulose acetate diethylaminoacetate; semipermeable polyamide; semipermeable polyurethane; semipermeable sulfonated polystyrene; semipermeable cross-linked polymer formed by the coprecipitation of a polyanion and a polycation as disclosed in U.S. Pat. Nos. 3,173,876; 3,276,586; 3,541,005; 3,541,006 and 3,546,876; semipermeable polymers as disclosed by Loeb and Sourirajan in U.S. Pat. No. 3,133,132; semipermeable crosslinked polystyrenes; semipermeable cross-linked poly(sodium styrene sulfonate); semipermeable crosslinked poly(vinylbenzyltrimethyl ammonium chloride); and semipermeable polymers possessing a fluid permeability of 2.5×10⁻⁸ to 2.5×10⁻² (cm²/hr·atm) expressed per atmosphere of hydrostatic or osmotic pressure difference across the semipermeable wall. Other polymers useful in the present invention are known in the art in U.S. Pat. Nos. 3,845,770; 3,916,899 and 4,160,020; and in Handbook of Common Polymers, Scott, J. R. and W. J. Roff, 1971, CRC Press, Cleveland, Ohio.

In certain embodiments, preferably the semipermeable wall is nontoxic, inert, and it maintains its physical and chemical integrity during the dispensing life of the drug. In certain embodiments, the dosage form comprises a binder as described above.

In certain embodiments, the dosage form comprises a lubricant, which may be used during the manufacture of the dosage form to prevent sticking to die wall or punch faces. Examples of lubricants include but are not limited to magnesium stearate, sodium stearate, stearic acid, calcium stearate, magnesium oleate, oleic acid, potassium oleate, caprylic acid, sodium stearyl fumarate, and magnesium palmitate.

### Coatings

The dosage forms of the present invention may optionally be coated with one or more coatings suitable for the regulation of release or for the protection of the formulation. In one embodiment, coatings are provided to permit either pH-dependent or pH-independent release, e.g., when exposed to gastrointestinal fluid. When a pH-independent coating is desired, the coating is designed to achieve optimal release regardless of pH-changes in the environmental fluid, e.g., the GI tract. Other preferred embodiments include a pH-dependent coating that releases the buprenorphine in desired areas of the gastrointestinal (GI) tract, e.g., the stomach or small intestine, such that an absorption profile is provided which is capable of providing at least about twelve hour and preferably up to twenty-four hour analgesia to a patient. It is also possible to formulate compositions which release a portion of the dose in one desired area of the GI tract, e.g., the stomach, and release the remainder of the dose in another area of the GI tract, e.g., the small intestine.

Formulations according to the invention that utilize pH-dependent coatings may also impart a repeat-action effect whereby unprotected drug is coated over an enteric coat and is released in the stomach, while the remainder, being protected by the enteric coating, is released further down the gastrointestinal tract. Coatings which are pH-dependent may be used in accordance with the present invention include a controlled release material such as, e.g., shellac, cellulose acetate phthalate (CAP), polyvinyl acetate phthalate (PVAP), hydroxypropyl methylcellulose phthalate, and methacrylic acid ester copolymers, zein, and the like.

In another preferred embodiment, the present invention is related to a stabilized solid controlled dosage form comprising the buprenorphine coated with a hydrophobic controlled release material selected from (i) an alkylcellulose; (ii) an acrylic polymer; or (iii) mixtures thereof. The coating may be applied in the form of an organic or aqueous solution or dispersion.

In certain preferred embodiments, the controlled release coating is derived from an aqueous dispersion of the hydrophobic controlled release material. The coated substrate containing the buprenorphine (e.g., a tablet core or inert pharmaceutical beads or spheroids) is then cured until an endpoint is reached at which the substrate provides a stable dissolution. The curing endpoint may be determined by comparing the dissolution profile (curve) of the dosage form immediately after curing to the dissolution profile (curve) of the dosage form after exposure to accelerated storage conditions of, e.g., at least one month at a temperature of 40° C. and a relative humidity of 75%.

In preferred embodiments, the controlled release coatings include a plasticizer such as those described herein.

In certain embodiments, it is necessary to overcoat the substrate comprising the buprenorphine with a sufficient amount of the aqueous dispersion of e.g., alkylcellulose or acrylic polymer, to obtain a weight gain level from about 2 to about 50%, e.g., about 2 to about 25% in order to obtain a controlled-release formulation. The overcoat may be lesser or greater depending upon the physical properties of the therapeutically active agent and the desired release rate, the inclusion of plasticizer in the aqueous dispersion and the manner of incorporation of the same, for example.

### Alkylcellulose Polymers

Cellulosic materials and polymers, including alkylcelluloses are controlled release materials well suited for coating the substrates, e.g., beads, tablets, etc. according to the invention. Simply by way of example, one preferred alkylcellulosic polymer is ethylcellulose, although the artisan will appreciate that other cellulose and/or alkylcellulose polymers may be readily employed, singly or on any combination, as all or part of a hydrophobic coating according to the invention.

One commercially-available aqueous dispersion of ethylcellulose is Aquacoat™. Aquacoat™ is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat™ with a suitable plasticizer prior to use.

Another aqueous dispersion of ethylcellulose is commercially available as Surelease™. This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly onto substrates.

### Acrylic Polymers

In other preferred embodiments of the present invention, the controlled release material comprising the controlled-release coating is a pharmaceutically acceptable acrylic polymer, including but not limited to acrylic acid and methacrylic acid copolymers, methyl methacrylate copolymers, ethoxyethyl methacrylates, cynaoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit™. There are several different types of Eudragit™. For example, Eudragit™ E is an example of a methacrylic acid copolymer which swells and dissolves in acidic media. Eudragit™ L is a methacrylic acid copolymer which does not swell at about pH<5.7 and is soluble at about pH>6. Eudragit™ S does not swell at about pH<6.5 and is soluble at about pH>7. Eudragit™ RL and Eudragit™ RS are water swellable, and the amount of water absorbed by these polymers is pH-dependent, however, dosage forms coated with Eudragit™ RL and RS are pH-independent.

In certain preferred embodiments, the acrylic coating comprises a mixture of two acrylic resin lacquers commercially available as Eudragit™ RL30D and Eudragit™ RS30D, respectively. Eudragit™ RL30D and Eudragit™ RS30D are copolymers of acrylic and methacrylic esters with a low content of quaternary ammonium groups, the molar ratio of ammonium groups to the remaining neutral (meth)acrylic esters being 1:20 in Eudragit™ RL30D and 1:40 in Eudragit™ RS30D. The mean molecular weight is about 150,000. The code designations RL (high permeability) and RS (low permeability) refer to the permeability properties of these agents. Eudragit™ RL/RS mixtures are insoluble in water and in digestive fluids. However, coatings formed from the same are swellable and permeable in aqueous solutions and digestive fluids.

The Eudragit™ RL/RS dispersions of the present invention may be mixed together in any desired ratio in order to ultimately obtain a controlled-release formulation having a desirable dissolution profile. Desirable controlled-release formulations may be obtained, for instance, from a retardant coating derived from 100% Eudragit™ RL, 50% Eudragit™ RL and 50% Eudragit™ RS, and 10% Eudragit™ RL:Eudragit™ 90% RS. Of course, one skilled in the art will recognize that other acrylic polymers may also be used, such as, for example, Eudragit™ L.

### Plasticizers

In embodiments of the present invention where the coating comprises an aqueous dispersion of a hydrophobic controlled release material, the inclusion of an effective amount of a plasticizer in the aqueous dispersion of hydrophobic material will further improve the physical properties of the controlled-release coating. For example, because ethylcellulose has a relatively high glass transition temperature and does not form flexible films under normal coating conditions, it is preferable to incorporate a plasticizer into an ethylcellulose coating containing controlled-release coating before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tibutyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit™ RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

In certain embodiments, the addition of a small amount of talc to the controlled release coating reduces the tendency of the aqueous dispersion to stick during processing, and acts as a polishing agent.

The release of the therapeutically active agent from the controlled-release formulation of the present invention can be further influenced, i.e., adjusted to a desired rate, by the addition of one or more release-modifying agents, or by providing one or more passageways through the coating. The ratio of hydrophobic controlled release material to water soluble material is determined by, among other factors, the release rate required and the solubility characteristics of the materials selected.

The release-modifying agents which function as pore-formers may be organic or inorganic, and include materials that can be dissolved, extracted or leached from the coating in the environment of use. The pore-formers may comprise one or more hydrophilic materials such as hydroxypropylmethylcellulose.

The controlled-release coatings of the present invention can also include erosion-promoting agents such as starch and gums.

The controlled-release coatings of the present invention can also include materials useful for making microporous lamina in the environment of use, such as polycarbonates comprised of linear polyesters of carbonic acid in which carbonate groups reoccur in the polymer chain.

The release-modifying agent may also comprise a semi-permeable polymer. In certain preferred embodiments, the release-modifying agent is selected from hydroxypropylmethylcellulose, lactose, metal stearates, and mixtures of any of the foregoing.

The controlled-release coatings of the present invention may also include an exit means comprising at least one passageway, orifice, or the like. The passageway may be formed by such methods as those disclosed in U.S. Pat. Nos. 3,845,770; 3,916,889; 4,063,064; and 4,088,864, all of which are hereby incorporated by reference. The passageway can have any shape such as round, triangular, square, elliptical, irregular, etc.

These and other embodiments of the present invention will readily occur to those of ordinary skill in the art in view of the disclosure herein.

A wide variety of materials can be used for preparing the dosage form according to this invention. This invention therefore contemplates the use of materials other than those specifically disclosed herein, including those which may hereafter become known to the art to be capable of performing the necessary functions.

### DETAILED DESCRIPTION OF THE PREFERRED EMBODIMENTS

The following examples illustrate various aspects of the present invention. They are not to be construed to limit the claims in any manner whatsoever. A wide variety of methods known in the art for the preparation of oral immediate release and oral controlled release dosage forms may be incorporated into the invention. Other suitable dosage forms may also be prepared by modification of the examples herein and by use of material other than those specifically disclosed herein, including those which may hereafter become known to the art to be capable of performing the necessary functions. Other suitable modifications and adaptations of the variety of conditions and parameters normally encountered and obvious to those skilled in the art are within the spirit and scope of the invention.

The percent loading of the buprenorphine onto the dosage form may be varied depending on the physiochemical and pharmaceutical properties of immediate release and controlled release material, excipients, the selected salt of buprenorphine and the desired release profile and duration of actions.

The ingredients used for the preparation of the buprenorphine dosage form agent may be modified depending on the selection, dose and desired duration of effect. In some embodiments, a change in the dose or amount buprenorphine will not require a significant change in amount of other ingredients. In other embodiments, a proportional change in the amount of other ingredients is required to maintain the desired properties. In yet other embodiments, a change in the dose or amount buprenorphine necessitates a change in the nature and/or amount of ingredients to provide the required characteristics of the buprenorphine (e.g., duration of effect, rate and extent of absorption, therapeutic concentrations and effect, etc.).

### EXAMPLES

### Example 1

Tablet Composition of Extended Release Buprenorphine HCl

| Ingredients | Qty./Unit | |
|---|---|---|
| 1. Buprenorphine HCl | 20 | mg |
| 2. HPMC 2208, USP | 150 | mg |
| 3. Carnauba wax | 30 | mg |
| 4. HPMC 2910, USP | 15 | mg |
| 5. Magnesium Stearate | 2 | mg |
| 6. Stearic acid | 8 | mg |
| 7. Talc | 3 | mg |

Place the ingredients 1, 2 and 3 in the granulator and mix for 15 minutes. Dissolve ingredient 4 in water (mix in hot water, then cool down) and spray into the fluidized mixture. Dry to approximately 5% moisture. Sequentially add ingredient 5, 6 and 7, with mixing steps between each addition. Compress using capsule shaped tooling.

### Example 2

Tablet Composition of Extended Release Buprenorphine HCl

| Ingredients | Qty./Unit | |
|---|---|---|
| 1. Buprenorphine HCl | 100 | mg |
| 2. HPMC 2208, USP | 250 | mg |
| 3. Carnauba wax | 50 | mg |
| 4. HPMC 2910, USP | 25 | mg |
| 5. Magnesium Stearate | 4 | mg |
| 6. Stearic acid | 14 | mg |
| 7. Talc | 5 | mg |

Place the ingredients 1, 2 and 3 in the granulator and mix for 15 minutes. Dissolve ingredient 4 in water (mix in hot water, then cool down) and spray into the fluidized mixture. Dry to approximately 5% moisture. Sequentially add ingredient 5, 6 and 7, with mixing steps between each addition. Compress using capsule shaped tooling.

### Example 3

Tablet Composition of Extended Release Buprenorphine HCl

| Ingredients | Qty./Unit | |
|---|---|---|
| 1. Buprenorphine HCl | 500 | mg |
| 2. HPMC 2208, USP | 250 | mg |
| 3. Carnauba wax | 50 | mg |
| 4. HPMC 2910, USP | 25 | mg |
| 5. Magnesium Stearate | 4 | mg |
| 6. Stearic acid | 14 | mg |
| 7. Talc | 5 | mg |

Place the ingredients 1, 2 and 3 in the granulator and mix for 15 minutes. Dissolve ingredient 4 in water (mix in hot water, then cool down) and spray into the fluidized mixture. Dry to approximately 5% moisture. Sequentially add ingredient 5, 6 and 7, with mixing steps between each addition. Compress using capsule shaped tooling.

### Example 4

Tablet Composition of Extended Release Buprenorphine Base

| Ingredients | Amt/Unit (mg) |
|---|---|
| Buprenorphine Base | 20 |
| Spray Dried Lactose | 60 |
| Povidone | 5 |
| Eudragit RS30D (solids) | 10 |
| Triacetin | 2 |
| Stearyl Alcohol | 25 |
| Talc | 2.5 |
| Magnesium Stearate | 1.25 |
| Opadry Pink Y-S-14518A | 4.0 |

1. Granulation: Spray the Eudragit/Triacetin dispersion onto the Buprenorphine, Spray Dried Lactose and Povidone using a fluid bed granulator. 2. Milling: Discharge the granulation and pass through a mill. 3. Waxing: Melt the stearyl alcohol and add to the milled granulation using a mixer. Allow to cool. 4. Milling: Pass the cooled granulation through a mill. 5. Lubrication: Lubricate the granulation with talc and magnesium stearate using a mixer. 6. Compression: Compress the granulation into tablets using a tablet press. 7. Film coating: Apply an aqueous film coat to the tablets.

### Example 5

Tablet Composition of Extended Release Buprenorphine HCl

| Ingredients | Amt/Unit (mg) |
|---|---|
| Buprenorphine HCl | 30 |
| Spray Dried Lactose | 60 |
| Povidone | 5 |
| Eudragit RS30D (solids) | 10 |
| Triacetin | 2 |
| Stearyl Alcohol | 25 |
| Talc | 2.5 |
| Magnesium Stearate | 1.25 |
| Opadry Pink Y-S-14518A | 4.0 |

1. Granulation: Spray the Eudragit/Triacetin dispersion onto the Buprenorphine, Spray Dried Lactose and Povidone using a fluid bed granulator. 2. Milling: Discharge the granulation and pass through a mill. 3. Waxing: Melt the stearyl alcohol and add to the milled granulation using a mixer. Allow to cool. 4. Milling: Pass the cooled granulation through a mill. 5. Lubrication: Lubricate the granulation with talc and magnesium stearate using a mixer. 6. Compression: Compress the granulation into tablets using a tablet press. 7. Film coating: Apply an aqueous film coat to the tablets.

### Example 6

Capsule Composition of Extended Release Buprenorphine Base

| Ingredients | Amt/Unit (mg) |
|---|---|
| Buprenorphine Base | 20 |
| Eudragit RSPO | 76 |
| Eudragit RLPO | 4 |
| Stearyl Alcohol | 25 |

1. Blend milled Stearyl Alcohol, Eudragit RLPO, Buprenorphine, and Eudragit RSPO using a Hobart Mixer. 2. Extrude the granulation using a Powder Feeder, Melt Extruder (equipped with the 6×1 mm die head), Conveyor, Lasermike, and Pelletizer. Powder feed rate-40 g/min; vacuum-about 980 mBar; Conveyor, such that diameter of extrudate is 1 mm, Pelletizer, such that pellets are cut to 1 mm in length. Screen pellets using #16 mesh and #20 mesh screens. Collect material that passes through the #16 mesh screen and is retained on the #20 mesh screen. 4. Fill capsules with the pellets.

### Example 7

Capsule Composition of Extended Release Buprenorphine Base

| Ingredients | Amt/Unit (mg) |
|---|---|
| Buprenorphine Base | 200 |
| Eudragit RSPO | 150 |
| Eudragit RLPO | 10 |
| Stearyl Alcohol | 40 |

1. Blend milled Stearyl Alcohol, Eudragit RLPO, Buprenorphine, and Eudragit RSPO using a Hobart Mixer. 2. Extrude the granulation using a Powder Feeder, Melt Extruder (equipped with the 6×1 mm die head), Conveyor, Lasermike, and Pelletizer. Powder feed rate-40 g/min; vacuum-about 980 mBar; Conveyor, such that diameter of extrudate is 1 mm, Pelletizer, such that pellets are cut to 1 mm in length. Screen pellets using #16 mesh and #20 mesh screens. Collect material that passes through the #16 mesh screen and is retained on the #20 mesh screen. 4. Fill capsules with the pellets.

### Example 8

Capsule Composition of Extended Release Buprenorphine Base

| Ingredients | Amt/Unit (mg) |
|---|---|
| Buprenorphine Base | 15 |
| Eudragit RSPO | 77 |
| Ethocel | 4.5 |
| Stearic acid | 27 |

Blend milled Stearic acid, Ethocel, Buprenorphine Base, and Eudragit RSPO using a V-blender. 2. Extrude the mixture using a Powder Feeder, Melt Extruder (equipped with the 6×1 mm die head), Conveyor, Lasermike, and Pelletizer. Powder feed rate, 1.2 kg/hr; vacuum, about 980 mBar; Conveyor, such that diameter of extrudate is 1 mm; Pelletizer, such that pellets are cut to 1 mm in length. 3. Screen pellets using #16 mesh and #20 mesh screens. Collect material that passes through the #16 mesh screen and is retained on the #20 mesh screen. Fill pellets in capsules.

### Example 9

Capsule Composition of Extended Release Buprenorphine HCl

| Steps | Ingredients | Amt/unit (mg) |
|---|---|---|
| 1 | Buprenorphine HCl | 12 |
| | Non-pareil beads (30/35 mesh) | 45 |
| | Opadry Clear | 2.5 |
| 2 | Eudragit RS3-D (dry) | 7.2 |
| | Eudragit RL30D (dry) | 0.4 |
| | Triethyl citrate | 1.5 |
| | Cabosil | 0.4 |
| 3 | Opadry Clear (HPMC) | 1.9 |
| | Cabosil | 0.28 |

1. Dissolve Buprenorphine HCl and Opadry (HPMC) in water. Spray the drug solution onto nonpareil beads in a fluid bed coater with Wurster insert. 2. Disperse Eudragit RS, Eudragit RL, triethyl citrate, and Cabosil in water. Spray the dispersion onto the beads in the fluid bed coater. 3. Dissolve Opadry in water. Spray the solution onto the beads in the fluid bed coater. 4. Cure the beads at 60.degree. C. for 24 hours.

### Example 10

Tablet Composition of Extended Release Buprenorphine HCl

| Ingredient | Amt/unit (mg) |
|---|---|
| Buprenorphine HCl | 75 |
| Anhydrous Dicalcium | 60 |
| Phosphate (Powdered) | 80 |
| Microcrystalline Cellulose | 80 |
| Glyceryl Behenate | 40 |
| Magnesium Stearate | 4 |
| Opadry Red | 17 |
| Purified Water | 900* |

*Remains in product as residual moisture only.

1. Pass the Stearyl Alcohol flakes through an oscillating mill. 2. Mix the Buprenorphine HCl, milled Stearyl Alcohol, Anhydrous Dicalcium Phosphate, Microcrystalline Cellulose, and Glyceryl Behenate in a twin shell blender. 3. Continuously feed the blended material into a twin screw extruder and collect the resultant heated material on a conveyor. 4. Allow the extrudate to cool on the conveyor. 5. Mill the cooled extrudate using an oscillating mill. 6. Blend the milled extrudate and Magnesium Stearate. 7. Compress the resultant granulation using a tablet press, preferably into a caplet. 8. Prepare a film coating solution by dispersing the Opadry in Purified Water and applying it to the tablet.

### Example 11

Capsule Composition of Extended Release Buprenorphine HCl

| Ingredient | Amt/unit (mg) |
|---|---|
| Buprenorphine HCl | 20 |
| Eudragit RSPO | 76.5 |
| Ethylcellulose | 4.5 |
| Stearyl Alcohol | 27 |

1. Pass Stearyl Alcohol flakes through an impact mill. 2. Mix the Buprenorphine HCl, Eudragit, Ethylcellulose and milled Stearyl Alcohol in a twin shell blender. 3. Continuously feed the blended material into a twin screw extruder and collect the resultant strands on a conveyor. 4. Allow the strands to cool on the conveyor. 5. Cut the cooled strands into pellets using a Pelletizer. 6. Screen the pellets and collect desired sieve portion. 7. Fill the extruded pellets into capsules.

Example 12 to 23 may be prepared as follows: (i) Dispense the specified hydrophobic controlled release material (e.g., hydrogenated Type I vegetable oil, hydrogenated Type II vegetable oil, polyoxyethylene stearates, polyoxyethylene distearates, glycerol monostearate, poorly water soluble, or high melting point waxes) into a mixer; (ii) Heat until fully melted; (iii) dispense the hydroxypropyl methyl cellulose (HPMC) into the mixer; (iv) Mix until dispersed; (v) Dispense the Aerosil into the same vessel; (vi) Mix until dispersed; (vii) Dispense the buprenorphine into the same vessel; (viii) Stir thoroughly with a high shear mixer; (ix) Transfer the mix into a liquid filling machine; (x) Fill into hard gelatin (or HPMC) capsule; (xi) Optionally, transfer the capsules to a banding machine and band the capsules.

### Example 12

Capsule Composition of Extended Release Buprenorphine HCl

| Ingredients | Quantity (mg)/Dose |
|---|---|
| Sterotex ® NF | 200 |
| Fractionated coconut oil | 70 |
| Methocel ® K 15M | 81 |
| Aerosil ® COK 84 | 4 |
| Buprenorphine HCl | 50 |

### Example 13

Capsule Composition of Extended Release Buprenorphine HCl

| Ingredients | Quantity (mg)/Dose |
|---|---|
| Beeswax | 200 |
| HPMC, K15M | 80 |
| Aerosil COK 84 | 8 |
| Buprenorphine HCl | 40 |

### Example 14

Capsule Composition of Extended Release Buprenorphine Base

| Ingredients | Quantity (mg)/Dose |
|---|---|
| Sterotex NF | 150 |
| HPMC, K15M | 75 |
| Coconut oil | 75 |
| Aerosil COK 84 | 5 |
| Buprenorphine | 100 |

### Example 15

Capsule Composition of Extended Release Buprenorphine Base

| Ingredients | Quantity (mg)/Dose |
|---|---|
| Cithrol GMS | 275 |
| HPMC, K100M | 40 |
| Aerosil COK 84 | 10 |
| Buprenorphine | 75 |

### Example 16

Capsule Composition of Extended Release Buprenorphine HCl

| Ingredients | Quantity (mg)/Dose |
|---|---|
| Hydrokote 112 | 250 |
| HPMC, K15M | 60 |
| Aerosil COK 84 | 10 |
| Buprenorphine HCl | 150 |

### Example 17

Capsule Composition of Extended Release Buprenorphine Base

| Ingredients | Quantity (mg)/Dose |
|---|---|
| Beeswax | 200 |
| HPMC, Pharmacoat 606 | 62.5 |
| Aerosil COK 84 | 7.5 |
| Buprenorphine Base | 200 |

### Example 18

Capsule Composition of Extended Release Buprenorphine Base

| Ingredients | Quantity (mg)/Dose |
|---|---|
| Gelucire 50/02 | 190 |
| Methocel K 100M | 35 |
| Aerosil COK 84 | 10 |
| Buprenorphine Base | 250 |

### Example 19

Capsule Composition of Extended Release Buprenorphine Base

| Ingredients | Quantity (mg)/Dose |
|---|---|
| Cetyl alcohol | 280 |
| Methocel K 100M | 50 |
| Aerosil COK 84 | 10 |
| Buprenorphine Base | 100 |

### Example 20

Capsule Composition of Extended Release Buprenorphine

| Ingredients | Quantity (mg)/Dose |
|---|---|
| Sterotex NF | 320 |
| Methocel K 15M | 60 |
| Aerosil COK 84 | 10 |
| Buprenorphine HCl | 300 |

### Example 21

Capsule Composition of Extended Release Buprenorphine HCl

| Ingredients | Quantity (mg)/Dose |
|---|---|
| Cithrol GMS | 320 |
| Methocel K 100M | 55 |
| Aerosil COK 84 | 15 |
| Buprenorphine HCl | 150 |

### Example 22

Capsule Composition of Extended Release Buprenorphine HCl

| Ingredients | Quantity (mg)/Dose |
|---|---|
| Sterotex ® NF | 100 |
| Fractionated coconut oil | 70 |
| Beeswax | 100 |
| Methocel ® K 15M | 81 |
| Aerosil ® COK 84 | 4 |
| Buprenorphine HCl | 200 |

### Example 23

Capsule Composition of Extended Release Buprenorphine HCl

| Ingredients | Quantity (mg)/Dose |
|---|---|
| Glyceryl behenate | 135 |
| Fractionated coconut oil | 50 |
| Methocel ® K 15M | 60 |
| Aerosil ® COK 84 | 3 |
| Buprenorphine HCl | 50 |

### Example 24

Tablet Composition of Extended Release Buprenorphine HCl

| Ingredients | Quantity (mg)/Dose |
|---|---|
| Lactose (spray dried) | 230 |
| Eudragit ™ RS PM | 60 |
| Purified water | q.s.* |
| Stearyl Alcohol | 90 |
| Talc | 8 |
| Magnesium Stearate | 4 |
| Buprenorphine HCl | 300 |

*Remains in product as residual moisture only.

In Example 24, the required quantities of buprenorphine HCl, spray-dried lactose, and Eudragit™ RS PM are transferred into an appropriate-size mixer, and mixed for approximately 5 minutes. While the powders are mixing, the mixture is granulated with enough water to produce a moist granular mass. The granules are then dried in a fluid bed dryer at 60° C., and then passed through an 8-mesh screen. Thereafter, the granules are re-dried and pushed through a 12-mesh screen. The required quantity of stearyl alcohol is melted at approximately 60 to 70° C., and while the granules are mixing, the melted stearyl alcohol is added. The warm granules are returned to the mixer. The coated granules are removed from the mixer and allowed to cool. The granules are then passed through a 12-mesh screen. The granulate is then lubricated by mixing the required quantity of talc and magnesium stearate in a suitable blender. Tablets are compressed on a suitable tableting machine.

In some embodiments, oral immediate release compressed tablets of buprenorphine can be formulated using conventional wet granulation procedures and equipment.

### Example 25

Immediate Release Tablet Composition of Buprenorphine HCl

| Ingredients | Qty./Unit | |
|---|---|---|
| 1. Buprenorphine HCl | 100 | mg |
| 2. Polyvinylpyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | mg |

### Example 26

Immediate Release Tablet Composition of Buprenorphine HCl

| Ingredients | Qty./Unit | |
|---|---|---|
| 1. Buprenorphine HCl | 50 | mg |
| 2. Polyvinylpyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | mg |

### Example 27

Immediate Release Tablet Composition of Buprenorphine HCl

| Ingredients | Qty/Unit | |
|---|---|---|
| 1. Buprenorphine HCl | 75 | mg |
| 2. Polyvinylpyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | mg |

In Example 25 to 27, blend 1, 2 and 3 together; pass through a 40-mesh screen. Add 4 slowly and knead well. Screen wet mass through a 4-mesh screen. Dry the granulation at 50° C. overnight. Screen the dried granulation through a 20-mesh screen. Bolt 5, 6 and 7 through a 60-mesh screen prior to mixing by tumbling with granulation. Compress using a concave punch.

In some embodiments, oral immediate release compressed tablets of buprenorphine can be formulated using conventional dry granulation procedures and equipment.

### Example 28

Tablet Composition of Buprenorphine HCl

| Ingredients | Qty./Unit |
|---|---|
| 1 Buprenorphine HCl | 20 mg |
| 2 Lactose (granular, 12-mesh) | 25 mg |
| 3 Starch | 20 mg |
| 4 Talc | 20 mg |
| 5 Magnesium Stearate | 0.3 mg |

In Example 28, mix ingredients 1 to 5 thoroughly. Compress into slugs. Grind and screen to 14- to 16-mesh granules. Recompress into tablets using a concave punch.

In some embodiments, oral sustained release compressed tablets of buprenorphine can be formulated using conventional fluid-bed granulation procedures and equipment.

### Example 29

Tablet Composition of Buprenorphine HCl

| Ingredients | Qty./Unit | |
|---|---|---|
| 1. Buprenorphine HCl | 50 | mg |
| 2. HPMC 2208, USP | 150 | mg |
| 3. Carnauba wax | 30 | mg |
| 4. HPMC 2910, USP | 15 | mg |
| 5. Magnesium Stearate | 2 | mg |
| 6. Stearic acid | 8 | mg |
| 7. Talc | 3 | mg |

In Example 29, place the ingredients 1, 2 and 3 in the granulator and mix for 15 minutes. Dissolve ingredient 4 in water (mix in how water, then cool down) and spay into the fluidized mixture. Dry to approximately 5% moisture. Sequentially add ingredient 5, 6 and 7, with mixing steps between each addition. Compress using capsule shaped tooling.

### Antinociceptive Effects of Oral Buprenorphine

### Radiant Heat Tail Flick Test

Background

The tail flick test was first described by D'Amour and Smith (1941), and remains essentially unchanged in application. (See generally D'Amour, F. E. and Smith, D. L., “A method for determining loss of pain sensation”, J. Pharmacol. Exp. Therap., 72:74-79 (1941); Dewey, D. L. and Harris, L. S., The Tail-flick test. In: S. Ehrenpreis and A. Neidle (Eds.), Methods in Narcotic Research, Marcel Dekker, Inc., New York, 1975, pp. 101-109; and Dubner, R. and Ren, K., “Assessing transient and persistent pain in animals.” In: P. D. Wall and R. Melzack (Eds.), Textbook of Pain, Churchill Livingstone, London, 1999, pp. 359-369). Quite simply, the tail of a rat or mouse is exposed to radiant heat, and the latency to withdraw is determined. The basal heat intensity is set so that naïve rats withdraw their tails within 2 to 3 sec. A cut-off latency of 10 sec (i.e., 3 to 4 times basal control value) is commonly employed to prevent tissue damage. An alternative to using radiant heat is to dip the tail into a water bath maintained at a fixed temperature, usually in the moderately noxious range of about 52° C. or 55° C. One advantage of a water bath is that the temperature is kept constant.

The tail-flick test is considered to be very robust in that weak analgesic agents are not detected by this test. In contrast, it is considered highly selective. There is a high degree of correlation between drugs that are identified as antinociceptive in the tail-flick test and clinically active analgesic agents. Importantly, agents that are sedating and may produce a positive response in the writhing test or hot plate test do not show antinociceptive activity in the tail-flick test. It is even possible to perform the tail-flick test in lightly anesthetized animals.

### Example 30

Aim: The aim of this study was to evaluate the analgesic effect of the oral administration of buprenorphine in rats using the tail flick test of nociception. Dose-response relationships of orally administered buprenorphine were established in order to calculate the ED₅₀ value. The primary measurements were made using the tail flick test designed to detect effects of the treatment(s) on nociception in rats.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: Three groups were employed in the present study with each group comprising of 8 animals. Each rat was administered oral buprenorphine 0.1 mg/kg, 0.3 mg/kg, or 1.0 mg/kg, following which the nociceptive threshold assessment was made. Tail flick test was performed 0, 15, 30, and 60 minutes after administration of the buprenorphine. Area under the curve was calculated using a graph between % MPE and time. Area under the curve was employed as a quantitative measure of the analgesic effect of the test drugs.

Measurement of Effect: Nociceptive threshold was measured with the tail flick test (D'Amour and Smith, 1941). The tail flick latency was considered as the time between exposure of the tail to radiant heat and tail withdrawal. Electrically heated nichrome wire was used as a source of radiant heat in the analgesiometer. The intensity of radiant heat was regulated in order to obtain a pretreatment latency between 2 and 4 sec in the animals. A cut-off latency time was fixed at 10 sec. Tail flick latency is expressed as a percentage of the maximum possible effect (MPE):

$$MPE ( \% ) = \frac{( Post treatment latency - pre treatment latency )}{( Cut \text{-} off time - pre treatment latency )} \times 100$$

Data Analysis: The anti-nociceptive effect at a particular dose level was assessed in terms of area under the curve calculated from a graph having time (relative to dosing time point) on the x-axis and percent maximum possible effect (% MPE) on the y-axis. Then a dose response curve was obtained from a graph having log dose(s) levels tested on the x-axis and their respective area under the curve on the y-axis. Then the dose response curve thus obtained was used to further calculate the ED₅₀ value of the test drug for the respective behavioral assessment method(s).

Results: Oral administration of buprenorphine caused a significant increase in the tail flick latency in rats in comparison to the control readings. This effect of buprenorphine was observed to be dependent of the dose. Thus, buprenorphine demonstrated a marked dose dependent analgesic effect in rats as also assessed in terms of its ED₅₀. The ED₅₀ values of orally administered buprenorphine as assessed using the tail flick test was 0.24 mg/kg. The peak time of the analgesic effect of oral buprenorphine was found to be about 30 minutes post-treatment.

### Hot Plate Test

Background

Heat is often used as a noxious stimulus in models of pain. Typically, the latency of the rat's response to the thermal stimulus is recorded as the dependent variable. To determine whether a test compound has analgesic properties, the latency responses of treated and control rats are evaluated. A significant increase in the latency to respond to the thermal stimulus after a treatment relative to a control treatment is interpreted as an antinociceptive or analgesic response. The latency of response will vary depending on the type of heat stimulus used. In the hot-plate assay, the rat is placed on a heated surface and the latency for the rat to respond to the stimulus, by licking its paw or jumping, is recorded.

### Example 31

Aim: The aim of this study was to evaluate the analgesic effect of the oral administration of buprenorphine in rats using the hot plate test of nociception. Dose-response relationships of orally administered buprenorphine were established in order to calculate the ED₅₀ value. The primary measurements were made using the hot plate test designed to detect effects of the treatment(s) on nociception in rats.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: Three groups were employed in the present study with each group comprising of 8 animals. Each rat was administered oral buprenorphine 0.1 mg/kg, 0.3 mg/kg, or 1.0 mg/kg, following which the nociceptive threshold assessment was made. The hot plate test was performed 0, 15, 30, and 60 minutes after administration of the buprenorphine. Area under the curve was calculated using a graph between % MPE and time. Area under the curve was employed as a quantitative measure of the analgesic effect of the test drugs.

Measurement of Effect: Nociceptive threshold was measured with the Eddy's hot plate test. The reaction time was considered as the time between placing of the rat on the Eddy's hot plate heated to a temperature of 52.5° C. and the reaction of the animal in the form of a jump or fore paw licking reflex. A cut-off latency time was fixed at 10 sec. Reaction time is expressed as a percentage of the maximum possible effect (MPE):

$$MPE ( \% ) = \frac{( Post treatment latency - pre treatment latency )}{( Cut \text{-} off time - pre treatment latency )} \times 100$$

Data Analysis: The anti-nociceptive effect at a particular dose level was assessed in terms of area under the curve calculated from a graph having time (relative to dosing time point) on the x-axis and percent maximum possible effect (% MPE) on the y-axis. Then a dose response curve was obtained from a graph having log dose(s) levels tested on the x-axis and their respective area under the curve on the y-axis. Then the dose response curve thus obtained was used to further calculate the ED₅₀ value of the test drug for the respective behavioral assessment method(s).

Results: Oral administration of buprenorphine caused a significant increase in the hot plate latency in rats in comparison to the control readings. This effect of buprenorphine was observed to be dependent of the dose. Thus, buprenorphine demonstrated a marked dose dependent analgesic effect in rats as also assessed in terms of its ED₅₀. The ED₅₀ values of orally administered buprenorphine as assessed using the hot plate test was 0.16 mg/kg. The peak time of the analgesic effect of oral buprenorphine was found to be about 30 minutes post-treatment.

### Pharmacokinetics of Oral Buprenorphine

The rate and extent of absorption (bioavailability) of oral, extended release buprenorphine may evaluated as shown in the prophetic pharmacokinetic study example below comparing sublingual buprenorphine and oral extended release buprenorphine. The pharmacokinetics of a drug can vary considerably from study to study due to both study and patient related variables, most notably, the intra- and inter-patient variability is drug liberation, absorption, distribution, metabolism, and excretion (LADME). This is an issue of particular concern for buprenorphine pharmacokinetics, particularly when given by the sublingual route of administration. Consequently, the results below should be considered illustrative but not limiting of the expected pharmacokinetics of oral, extended release buprenorphine and oral immediate release buprenorphine.

### Pharmacokinetics of Oral Extended Release Buprenorphine

| | |
|---|---|
| Design | Single-dose, fasted, randomized cross-over (2 week |
| | washout) evaluation of oral extended release |
| | buprenorphine and sublingual buprenorphine under |
| | naltrexone protection. |
| Subjects | 8 healthy volunteers |
| Study Test Drugs | Oral Buprenorphine HCl Extended Release 2 x 10 |
| | mg (20 mg of buprenorphine HCl) or Sublingual |
| | Buprenorphine HCl 1 x 8 mg (8 mg of |
| | buprenorphine base) |
| Other Study Drugs | Naltrexone HCl 25 mg 12 hours prior to, 2 hours |
| | prior to and 10 hours after dosing with study |
| | test drugs. |
| Pharmacokinetic | Blood samples are collected for the drug under study |
| Sampling | at pre-dose and at 0.25, 0.5, 0.75, 1, 1.5, 2, 2.5, 3, |
| | 4, 5, 6, 7, 8, 10, 12, 14, 16, 20, 24, 28, 36, and 48 |
| | hours post-dose. |
| Analytical Method | LCMS/MS |
| Data Analysis | Plasma concentrations dose normalized to 8 mg |
| | buprenorphine base. |

| Pharmacokinetic | Buprenorphine HCl | Buprenorphine HCl |
|---|---|---|
| Parameter | (Extended Release | (Sublingual |
| (Buprenorphine) | 2 x 10 mg) | 1 x 8 mg) |
| Cmax (pg/mL) | 653 | 2819 |
| Tmax (hours) | 3.71 | 1.23 |
| AUC₀₋₄₈ (pg · hr/mL) | 10129 | 19736 |
| AUC_{0-inf} (pg · hr/mL) | 18472 | 28429 |
| Pharmacokinetic | | |
| Parameter | Buprenorphine HCl | Sublingual |
| (Norbuprenorphine) | Extended Release | Buprenorphine HCl |
| Cmax (pg/mL) | 598 | 801 |
| Tmax (hours) | 4.55 | 5.17 |
| AUC₀₋₄₈ (pg · hr/mL) | 18235 | 16483 |
| AUC_{0-inf} (pg · hr/mL) | 39394 | 69411 |

These and other embodiments of the present invention will readily occur to those of ordinary skill in the art in view of the disclosure herein.

The included examples are illustrative but not limiting of the methods and composition of the present invention. Other suitable modifications and adaptations of the variety of conditions and parameters normally encountered and obvious to those skilled in the art are within the spirit and scope of the invention.

A wide variety of materials can be used for preparing the dosage form according to this invention. This invention therefore contemplates the use of materials other than those specifically disclosed herein, including those which may hereafter become known to the art to be capable of performing the necessary functions.

In all instances wherein prophetic examples are provided, these compositions are intended to be exemplary and it should be understood that the specific procedures, constituents, amounts thereof and the like can be varied in order to obtain a composition possessing desired properties.

Having now fully described the invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

1-167. (canceled)

168. A pharmaceutical composition for orally administering buprenorphine to a mammal having a buprenorphine responsive medical condition, the composition
comprising a therapeutically effective amount of buprenorphine and
being in a dosage form formulated such that no substantial absorption of buprenorphine occurs in the oral cavity of the mammal when the dosage form is orally administered to the mammal.

169. The composition of claim 168, further comprising a controlled release material in an amount sufficient to render the dosage form an extended release dosage form.

170. The composition of claim 168, wherein the composition further comprises a release rate modifier.

171. The composition of claim 170, wherein the release rate modifier is selected from the group consisting of alkyl celluloses, methacrylic acid polymers and copolymers, and cellulose ethers.

172. The composition of claim 170, wherein the release rate modifier is a hydroxyalkyl methylcellulose.

173. The composition of claim 170, wherein the release rate modifier is a hydroxyproyl methylcellulose.

174. The composition of claim 171, wherein the release rate modifier is an alkyl cellulose and the composition further comprises an aliphatic alcohol selected from the group consisting of C₁₂ to C₃₆ aliphatic alcohols.

175. The composition of claim 174, wherein the aliphatic alcohol is selected from the group consisting of C₁₄ to C₂₂ aliphatic alcohols.

176. The composition of claim 174, wherein the composition further comprises a polyalkylene glycol.

177. The composition of claim 170, further comprising a thixotrope.

178. The composition of claim 177, wherein the thixotrope is a fumed silicon dioxide.

179. The composition of claim 168, further comprising a hydrophobic binder.

180. The composition of claim 179, wherein the binder is selected from the group consisting of fatty acids, fatty alcohols, glyceryl esters of fatty acids, mineral oils, vegetable oils, waxes, and polyalkylene glycols, and combinations of these.

181. The composition of claim 179, wherein the binder has a melting point in the range from about 25 to 200 degrees Celsius.

182. The composition of claim 179, wherein the binder is a polyalkylene glycol.

183. The composition of claim 168, further comprising an oil.

184. The composition of claim 183, wherein the oil is selected from the group consisting of hydrogenated Type I vegetable oils, hydrogenated Type II vegetable oils, polyoxyethylene stearates, polyoxyethylene distearates, glycerol monostearate, poorly water soluble waxes having a melting point in the range from 50 to 100 degrees Celsius, and combinations of these.

185. The composition of claim 183, wherein the oil is selected from the group consisting of hydrogenated cottonseed oil, hydrogenated palm oil, hydrogenated soybean oil, and hydrogenated palm kernel oil.

186. The composition of claim 183, further comprising a thixotrope.

187. The composition of claim 186, wherein the thixotrope is selected from the group consisting of silicon dioxides and aluminas.

188. The composition of claim 168, wherein the dosage form comprises multiple granules that include buprenorphine.

189. The composition of claim 188, wherein the dosage form is a tablet formed by compressing the granules.

190. The composition of claim 188, wherein the dosage form is a capsule containing the granules.

191. The composition of claim 168, wherein the dosage form comprises a substrate coated with buprenorphine.

192. The composition of claim 191, wherein the substrate further comprises a hydrophobic controlled-release coating overcoating the buprenorphine.

193. The composition of claim 191, wherein the dosage form comprises a plurality of the substrate, the substrate being in the form of a bead.

194. The composition of claim 168, wherein the dosage form is an osmotic dosage form comprising
(a) a bilayer core that comprises (i) a buprenorphine layer and (ii) a displacement layer comprising an osmopolymer; and
(b) a semipermeable wall surrounding the bilayer core, the wall having a passageway disposed therein through which buprenorphine is released upon swelling of the displacement layer induced by imbibement of an environmental fluid by the displacement layer.

195. The composition of claim 168, wherein the dosage form includes an outside coating that substantially prevents direct contact of buprenorphine with the oral cavity during oral administration of the dosage form.

196. The composition of claim 195, wherein the outside coating is an enteric coat.

197. A method of treating a mammal having a buprenorphine responsive medical condition, the method comprising orally administering to the mammal a therapeutically effective amount of buprenorphine in a dosage form that substantially prevents absorption of buprenorphine in the oral cavity of the mammal.

198. The method of claim 196, wherein the condition is selected from the group consisting of pain, opioid addiction disorders, polysubstance abuse, opioid dependence, cough, dyspnea, restless leg syndrome, fibromyalgia, urinary incontinence, and acute herpes zoster.
